(19) United States
(12) Patent Application Publication
(10) Pub. No.: US 2023/0340112 A1
(43) Pub. Date: Oct. 26, 2023

(54) ANTI-SIRPA ANTIBODIES AND METHODS OF USE

(71) Applicant: Apexigen, Inc., San Carlos, CA (US)

(72) Inventors: Minu K. SRIVASTAVA, San Carlos, CA (US); Swati JALGAONKAR, San Carlos, CA (US); Christine TAN, San Carlos, CA (US); George HUANG, San Carlos, CA (US); Rena BAHJAT, San Carlos, CA (US); Xiaodong YANG, San Carlos, CA (US); Erin L. FlLBERT, San Carlos, CA (US); Ryan ALVARADO, San Carlos, CA (US); Sushma KRISHNAN, San Carlos, CA (US)

(21) Appl. No.: 17/798,738

(22) PCT Filed: Feb. 26, 2021
(86) PCT No.: PCT/US2021/020097

§ 371 Date: Aug. 10, 2022

Related U.S. Application Data
(60) Provisional application No. 62/983,081, filed on Feb. 28, 2020.
(60) Provisional application No. 63/042,742, filed on Jun. 23, 2020.
(60) Provisional application No. 63/069,570, filed on Aug. 24, 2020.
(60) Provisional application No. 63/108,547, filed on Nov. 2, 2020.

(51) Int. Cl. C07K 16/28; A61P 37/02

(52) U.S. Cl. CPC C07K 16/2803; A61P 37/02; C07K 2317/565; C07K 2317/24; C07K 2317/92

(57) ABSTRACT

Provided are anti-signal regulatory protein α (SIRPα) antibodies and related compositions, which may be used in any of a variety of therapeutic or diagnostic methods, including the treatment or diagnosis of oncological diseases and others.

### CROSS-REFERENCE TO RELATED APPLICATIONS

This application claims the benefit under 35 U.S.C. § 119(e) to U.S. Provisional Application No. 62/983,081, filed Feb. 28, 2020; U.S. Provisional Application No. 63/042,742, filed Jun. 23, 2020; U.S. Provisional Application No. 63/069,570, filed Aug. 24, 2020; and U.S. Provisional Application No. 63/108,547, filed Nov. 2, 2020; each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is APEX_025_04 WO_ST25.txt. The text file is about 291 KB, created on Feb. 25, 2021, and is being submitted electronically via EFS-Web.

### BACKGROUND

### Technical Field

The present disclosure relates to anti-signal regulatory protein α (SIRPα) antibodies and related compositions, which may be used in any of a variety of therapeutic or diagnostic methods, including the treatment or diagnosis of oncological diseases and others.

### Description of the Related Art

SIRPα belongs to the SIRP family of transmembrane receptors, which are primarily expressed within the myeloid cell lineage. SIRPα contains an intracellular, cytoplasmic immunoreceptor tyrosine-based inhibitory-phosphorylated motif (ITIM). Upon ligand cross-linking, tyrosine-phosphorylated ITIM sites recruit and activate SHP phosphatases to negatively regulate cellular functions of myeloid cells, such as phagocytosis and inflammatory cytokine release.

CD47 serves as the primary ligand for SIRPα, and CD47 is broadly expressed on many cell types including endothelial cells, leukocytes and erythrocytes and mediates a “don't-eat-me” signal to protect healthy cells from phagocyte-dependent clearance. Dysregulation of SIRPα and CD47 expression contributes to immune-associated diseases such as cancer. Tumors increase CD47 expression compared to healthy cells in order to evade the immune surveillance mechanisms such as phagocytosis by macrophages. CD47 expression in tumors is inversely correlated with patient overall survival and constitutes an adverse prognostic factor for several cancer types.

Studies in syngeneic mouse models using anti-SIRPα antibodies have shown single-agent anti-tumor activity and in combination with immune checkpoint inhibitors in multiple tumor settings including Renal carcinoma (RENCA), Colorectal (CT26 & MC38), Melanoma (B16), and Breast carcinoma (4T1). Moreover, several ongoing clinical trials to target CD47 have shown promising results in reducing tumor growth. However, agents targeting CD47 (anti-CD47 or SIRPα-Fc) present hematological toxicities and present a huge antigen sink preventing achievement of an optimum therapeutic window. CD47 regulates red blood cell homeostasis and blockade has resulted in anemia or thrombocytopenia in clinical studies. In addition to SIRPα, CD47 interacts with multiple receptors including another close member of the SIRP family, SIRP gamma (SIRPγ, CD172g) that is expressed in a restricted manner on T lymphocytes, as opposed to SIRPα expression which is restricted to myeloid cells. The SIRPγ-CD47 interaction mediates cell-cell adhesion and T cell migration, enhances T cell mediated proliferation, and co-stimulates T cell activation. CD47 is also a ligand for thrombospondin-1 (TSP1) and blocking this interaction with anti-CD47 antibodies may have additional undesirable consequences.

Thus, there remains a need in the art for therapeutic antibodies that effectively inhibit or otherwise antagonize SIRPα, including antibodies that do so without significantly interfering with SIRPγ-CD47 interactions and signaling.

### BRIEF SUMMARY

Embodiments of the present disclosure include an isolated antibody, or an antigen-binding fragment thereof, that binds to signal regulatory protein α (SIRPα), comprising:
a heavy chain variable (VH) region comprising VHCDR1, VHCDR2, and VHCDR3 regions set forth respectively in SEQ ID NOs: 1-3; and a light chain variable (VL) region comprising VLCDR1, VLCDR2, and VLCDR3 regions set forth respectively in SEQ ID NOs: 4-6;
a VH region comprising VHCDR1, a VHCDR2, and VHCDR3 regions set forth respectively in SEQ ID NOs: 7-9; and a VL region comprising VLCDR1, VLCDR2, and VLCDR3 regions set forth respectively in SEQ ID NOs: 10-12;
a VH region comprising VHCDR1, VHCDR2, and VHCDR3 regions set forth respectively in SEQ ID NOs: 13-15; and a VL region comprising VLCDR1, VLCDR2, and VLCDR3 regions set forth respectively in SEQ ID NOs: 16-18;
a VH region comprising VHCDR1, VHCDR2, and VHCDR3 regions set forth respectively in SEQ ID NOs: 19-21; and a VL region comprising VLCDR1, VLCDR2, and VLCDR3 regions set forth respectively in SEQ ID NOs: 22-24;
a VH region comprising VHCDR1, VHCDR2, and VHCDR3 regions set forth respectively in SEQ ID NOs: 25-27; and a VL region comprising VLCDR1, VLCDR2, and VLCDR3 regions set forth respectively in SEQ ID NOs: 28-30;
a VH region comprising VHCDR1, VHCDR2, and VHCDR3 regions set forth respectively in SEQ ID NOs: 31-33; and a VL region comprising VLCDR1, VLCDR2, and VLCDR3 regions set forth respectively in SEQ ID NOs: 34-36;
a VH region comprising VHCDR1, VHCDR2, and VHCDR3 regions set forth respectively in SEQ ID NOs: 37-39; and a VL region comprising VLCDR1, VLCDR2, and VLCDR3 regions set forth respectively in SEQ ID NOs: 40-42;
a VH region comprising VHCDR1, VHCDR2, and VHCDR3 regions set forth respectively in SEQ ID NOs: 43-45; and a VL region comprising VLCDR1, VLCDR2, and VLCDR3 regions set forth respectively in SEQ ID NOs: 225 and 47-48, 226 and 47-48, or 46-48;
a VH region comprising VHCDR1, VHCDR2, and VHCDR3 regions set forth respectively in SEQ ID NOs: 49-51; and a VL region comprising VLCDR1, VLCDR2, and VLCDR3 regions set forth respectively in SEQ ID NOs: 52-54;
a VH region comprising VHCDR1, VHCDR2, and VHCDR3 regions set forth respectively in SEQ ID NOs: 55-57; and a VL region comprising VLCDR1, VLCDR2, and VLCDR3 regions set forth respectively in SEQ ID NOs: 58-60;
a VH region comprising VHCDR1, VHCDR2, and VHCDR3 regions set forth respectively in SEQ ID NOs: 61-63; and a VL region comprising VLCDR1, VLCDR2, and VLCDR3 regions set forth respectively in SEQ ID NOs: 64-66;
a VH region comprising VHCDR1, VHCDR2, and VHCDR3 regions set forth respectively in SEQ ID NOs: 67-69; and a VL region comprising VLCDR1, VLCDR2, and VLCDR3 regions set forth respectively in SEQ ID NOs: 70-72;
a VH region comprising VHCDR1, VHCDR2, and VHCDR3 regions set forth respectively in SEQ ID NOs: 73-75; and a VL region comprising VLCDR1, VLCDR2, and VLCDR3 regions set forth respectively in SEQ ID NOs: 76-78;
a VH region comprising VHCDR1, VHCDR2, and VHCDR3 regions set forth respectively in SEQ ID NOs: 79-81; and a VL region comprising VLCDR1, VLCDR2, and VLCDR3 regions set forth respectively in SEQ ID NOs: 82-84;
a VH region comprising VHCDR1, VHCDR2, and VHCDR3 regions set forth respectively in SEQ ID NOs: 85-87; and a VL region comprising VLCDR1, VLCDR2, and VLCDR3 regions set forth respectively in SEQ ID NOs: 88-90;
a VH region comprising VHCDR1, VHCDR2, and VHCDR3 regions set forth respectively in SEQ ID NOs: 91-93; and a VL region comprising VLCDR1, VLCDR2, and VLCDR3 regions set forth respectively in SEQ ID NOs: 94-96; or a VH region comprising VHCDR1, VHCDR2, and VHCDR3 regions set forth respectively in SEQ ID NOs: 97-99; and a VL region comprising VLCDR1, VLCDR2, and VLCDR3 regions set forth respectively in SEQ ID NOs: 100-102;
a VH region comprising VHCDR1, VHCDR2, and VHCDR3 regions set forth respectively in SEQ ID NOs: 103-105; and a VL region comprising VLCDR1, VLCDR2, and VLCDR3 regions set forth respectively in SEQ ID NOs: 106-108;
a VH region comprising VHCDR1, VHCDR2, and VHCDR3 regions set forth respectively in SEQ ID NOs: 109-111; and a VL region comprising VLCDR1, VLCDR2, and VLCDR3 regions set forth respectively in SEQ ID NOs: 112-114;
a VH region comprising VHCDR1, VHCDR2, and VHCDR3 regions set forth respectively in SEQ ID NOs: 115-117; and a VL region comprising VLCDR1, VLCDR2, and VLCDR3 regions set forth respectively in SEQ ID NOs: 118-120;
a VH region comprising VHCDR1, VHCDR2, and VHCDR3 regions set forth respectively in SEQ ID NOs: 121-123; and a VL region comprising VLCDR1, VLCDR2, and VLCDR3 regions set forth respectively in SEQ ID NOs: 124-126;
a VH region comprising VHCDR1, VHCDR2, and VHCDR3 regions set forth respectively in SEQ ID NOs: 127-129; and a VL region comprising VLCDR1, VLCDR2, and VLCDR3 regions set forth respectively in SEQ ID NOs: 130-132;
a VH region comprising VHCDR1, VHCDR2, and VHCDR3 regions set forth respectively in SEQ ID NOs: 133-135; and a VL region comprising VLCDR1, VLCDR2, and VLCDR3 regions set forth respectively in SEQ ID NOs: 136-138;
a VH region comprising VHCDR1, VHCDR2, and VHCDR3 regions set forth respectively in SEQ ID NOs: 139-141; and a VL region comprising VLCDR1, VLCDR2, and VLCDR3 regions set forth respectively in SEQ ID NOs: 142-144;
a VH region comprising VHCDR1, VHCDR2, and VHCDR3 regions set forth respectively in SEQ ID NOs: 145-147; and a VL region comprising VLCDR1, VLCDR2, and VLCDR3 regions set forth respectively in SEQ ID NOs: 148-150;
a VH region comprising VHCDR1, VHCDR2, and VHCDR3 regions set forth respectively in SEQ ID NOs: 151-153; and a VL region comprising VLCDR1, VLCDR2, and VLCDR3 regions set forth respectively in SEQ ID NOs: 154-156;
a VH region comprising VHCDR1, VHCDR2, and VHCDR3 regions set forth respectively in SEQ ID NOs: 157-159; and a VL region comprising VLCDR1, VLCDR2, and VLCDR3 regions set forth respectively in SEQ ID NOs: 160-162;
a VH region comprising VHCDR1, VHCDR2, and VHCDR3 regions set forth respectively in SEQ ID NOs: 163-165; and a VL region comprising VLCDR1, VLCDR2, and VLCDR3 regions set forth respectively in SEQ ID NOs: 166-168, or or a variant of said antibody, or an antigen-binding fragment thereof, comprising heavy and light chain variable regions identical to the heavy and light chain variable regions of (i) and (ii) except for up to 1, 2, 3, 4, 5, 6, 7, or 8 total amino acid substitutions across said CDR regions.

In some embodiments, the VH region comprises an amino acid sequence having at least 90%, 95%, 96%, 97%, 98%, 99%, or 100% identity to a sequence selected from SEQ ID NOs: 169, 171, 173, 175, 177, 179, 181, 183, 185, 187, 189, 191, 193, 195, 197, 199, 201, 203, 205, 207, 209, 211, 213, 215, 217, 219, 221, and 223. In some embodiments, the VL region comprises an amino acid sequence having at least 90%, 95%, 96%, 97%, 98%, 99%, or 100% identity to a sequence selected from SEQ ID NOs: 168, 170, 172, 174, 176, 178, 180, 182, 184 or 227, 186, 188, 190, 192, 194, 196, 198, 200, 202, 204, 206, 208, 210, 212, 214, 216, 218, 220, 222, and 224.

In some embodiments, the isolated antibody, or antigen-binding fragment thereof, comprises:
the VH region set forth in SEQ ID NO: 169, and the VL region set forth in SEQ ID NO: 170;
the VH region set forth in SEQ ID NO: 171, and the VL region set forth in SEQ ID NO: 172;
the VH region set forth in SEQ ID NO: 173, and the VL region set forth in SEQ ID NO: 174;
the VH region set forth in SEQ ID NO: 175, and the VL region set forth in SEQ ID NO: 176;
the VH region set forth in SEQ ID NO: 177, and the VL region set forth in SEQ ID NO: 178;
the VH region set forth in SEQ ID NO: 179, and the VL region set forth in SEQ ID NO: 180;
the VH region set forth in SEQ ID NO: 181, and the VL region set forth in SEQ ID NO: 182;
the VH region set forth in SEQ ID NO: 183, and the VL region set forth in SEQ ID NO: 184 or 227;
the VH region set forth in SEQ ID NO: 185, and the VL region set forth in SEQ ID NO: 186;
the VH region set forth in SEQ ID NO: 187, and the VL region set forth in SEQ ID NO: 188;
the VH region set forth in SEQ ID NO: 189, and the VL region set forth in SEQ ID NO: 190;
the VH region set forth in SEQ ID NO: 191, and the VL region set forth in SEQ ID NO: 192;
the VH region set forth in SEQ ID NO: 193, and the VL region set forth in SEQ ID NO: 194;
the VH region set forth in SEQ ID NO: 195, and the VL region set forth in SEQ ID NO: 196;
the VH region set forth in SEQ ID NO: 197, and the VL region set forth in SEQ ID NO: 198;
the VH region set forth in SEQ ID NO: 199, and the VL region set forth in SEQ ID NO: 200;
the VH region set forth in SEQ ID NO: 201, and the VL region set forth in SEQ ID NO: 202;
the VH region set forth in SEQ ID NO: 203, and the VL region set forth in SEQ ID NO: 204;
the VH region set forth in SEQ ID NO: 205, and the VL region set forth in SEQ ID NO: 206;
the VH region set forth in SEQ ID NO: 207, and the VL region set forth in SEQ ID NO: 208;
the VH region set forth in SEQ ID NO: 209, and the VL region set forth in SEQ ID NO: 210;
the VH region set forth in SEQ ID NO: 211, and the VL region set forth in SEQ ID NO: 212;
the VH region set forth in SEQ ID NO: 213, and the VL region set forth in SEQ ID NO: 214;
the VH region set forth in SEQ ID NO: 215, and the VL region set forth in SEQ ID NO: 216;
the VH region set forth in SEQ ID NO: 217, and the VL region set forth in SEQ ID NO: 218;
the VH region set forth in SEQ ID NO: 219, and the VL region set forth in SEQ ID NO: 220;
the VH region set forth in SEQ ID NO: 221, and the VL region set forth in SEQ ID NO: 222; or

the VH region set forth in SEQ ID NO 223:, and the VL region set forth in SEQ ID NO: 224. Also included is an isolated antibody, or an antigen-binding fragment thereof, that binds to signal regulatory protein α (SIRPα), comprising a heavy chain variable (VH) region which comprises an amino acid sequence having at least 90%, 95%, 96%, 97%, 98%, 99%, or 100% identity to a sequence selected from SEQ ID NOs: 169, 171, 173, 175, 177, 179, 181, 183, 185, 187, 189, 191, 193, 195, 197, 199, 201, 203, 205, 207, 209, 211, 213, 215, 217, 219, 221, and 223, and, respectively, a light chain variable (VL) region which comprises an amino acid sequence having at least 90%, 95%, 96%, 97%, 98%, 99%, or 100% identity to a sequence selected from SEQ ID NO: 168, 170, 172, 174, 176, 178, 180, 182, 184 or 227, 186, 188, 190, 192, 194, 196, 198, 200, 202, 204, 206, 208, 210, 212, 214, 216, 218, 220, 222, and 224.

Some embodiments include an isolated antibody, or an antigen-binding fragment thereof, that binds to signal regulatory protein α (SIRPα) at an epitope selected from:
(a) an epitope that comprises, consists, or consists essentially of one or more residues selected from S68, L70, T71, R73, S79, K100, S102, and T114, as defined by the human SIRPα sequence of SEQ ID NO: 228, optionally wherein the epitope comprises, consists, or consists essentially of one or more residues selected from SDLTKRNNMDFS (SEQ ID NO: 232) and KGSPDDVEFKSGAGT (SEQ ID NO: 233); and
(b) an epitope that comprises, consists, or consists essentially of one or more residues selected from H319 and S332, as defined by the human SIRPα sequence of SEQ ID NO: 228, optionally wherein the epitope comprises, consists, or consists essentially of HDLKVSAHPKEQGS (SEQ ID NO: 234).

Some embodiments include an isolated antibody, or an antigen-binding fragment thereof, that binds to signal regulatory protein β (SIRPβL) at an epitope selected from:

(a) an epitope that comprises, consists, or consists essentially of one or more residues selected from SDLTKRNNMDFS (SEQ ID NO: 232) and KGSPDDVEFKSGAGT (SEQ ID NO: 233); and
(b) an epitope that comprises, consists, or consists essentially of HDLKVSAHPKEQGS (SEQ ID NO: 234).

Some embodiments include an isolated antibody, or an antigen-binding fragment thereof, that binds to signal regulatory protein βL (SIRβ) at an epitope selected from:
(a) an epitope that comprises, consists, or consists essentially of one or more residues selected from SDLTKRNNMDFS (SEQ ID NO: 232) and KGSPDDVEFKSGAGT (SEQ ID NO: 233); and
(b) an epitope that comprises, consists, or consists essentially of HDLKVSAHPKEQGS (SEQ ID NO: 234).

In some embodiments, the antibody, or antigen-binding fragment thereof, of (a) comprises a heavy chain variable (VH) region comprising VHCDR1, VHCDR2, and VHCDR3 regions set forth respectively in SEQ ID NOs: 43, 44, and 45; and a light chain variable (VL) region comprising VLCDR1, VLCDR2, and VLCDR3 regions set forth respectively in SEQ ID NOs: 226, 47, and 48; or the antibody, or antigen-binding fragment thereof, of (b) comprises VHCDR1, VHCDR2, and VHCDR3 regions set forth respectively in SEQ ID NOs: 49, 50, and 51; and VLCDR1, VLCDR2, and VLCDR3 regions set forth respectively in SEQ ID NOs: 52, 53, and 54, including variants of said antibody, or antigen-binding fragment thereof, comprising up to 1, 2, 3, 4, 5, 6, 7, or 8 total amino acid substitutions across said CDR regions.

In some embodiments, the antibody, or antigen-binding fragment thereof, of (a) comprises a VH region having an amino acid sequence with at least 90%, 95%, 96%, 97%, 98%, 99%, or 100% identity to SEQ ID NO: 183, and/or a VL region having an amino acid sequence with at least 90%, 95%, 96%, 97%, 98%, 99%, or 100% identity to SEQ ID NO: 227; or the antibody, or antigen-binding fragment thereof, of (b) comprises a VH region having an amino acid sequence with at least 90%, 95%, 96%, 97%, 98%, 99%, or 100% identity to SEQ ID NO: 185, and/or a VL region having an amino acid sequence with at least 90%, 95%, 96%, 97%, 98%, 99%, or 100% identity to SEQ ID NO: 186.

In some embodiments, the antibody, or antigen-binding fragment thereof, of (a) comprises the VH region set forth in SEQ ID NO: 183, and/or the VL region set forth in SEQ ID NO: 227; or the antibody, or antigen-binding fragment thereof, of (b) comprises the VH region set forth in SEQ ID NO: 185, and/or the VL region set forth in SEQ ID NO: 186.

Certain embodiments include an isolated antibody, or an antigen-binding fragment thereof, that binds to signal regulatory protein α (SIRPα), comprising a heavy chain variable (VH) region comprising VHCDR1, VHCDR2, and VHCDR3 regions selected from the underlined sequences in Table R1; and, respectively, a light chain variable (VL) region comprising VLCDR1, VLCDR2, and VLCDR3 regions selected from underlined sequences in Table R2. Some embodiments comprise a VH region which comprises an amino acid sequence selected from Table R1, and, respectively, a VL region which comprises an amino acid sequence selected from Table R2, optionally as defined in Table R3.

In some embodiments, an isolated antibody, or an antigen-binding fragment thereof, binds to human SIRPα, including soluble and cell-expressed human SIRPα, optionally the V1, V2, and/or V8 variants of human SIRPα. In some embodiments, an isolated antibody, or an antigen-binding fragment thereof, binds to at least one human SIRPα polypeptide or domain or epitope selected from Table S1.

In some embodiments, the antibody is humanized. In some embodiments, the antibody is selected from the group consisting of a single chain antibody, a scFv, a univalent antibody lacking a hinge region, a minibody, and a probody. In some embodiments, the antibody is a Fab or a Fab′ fragment. In some embodiments, the antibody is a F(ab′)2 fragment. In some embodiments, the antibody is a whole antibody.

In some embodiments, an isolated antibody, or an antigen-binding fragment thereof, comprises a human IgG constant domain. In some embodiments, the IgG constant domain comprises an IgG1 CH1 domain. In some embodiments, the IgG constant domain comprises an IgG1 Fc region or IgG4 Fc region, optionally a modified Fc region, optionally modified by one or more amino acid substitutions such as an S228P substitution.

In some embodiments, an isolated antibody, or antigen-binding fragment thereof, binds to human SIRPα, optionally at least one SIRPα polypeptide or domain or epitope from Table S1, with a K_{D} of 0.4 nM or lower. In some embodiments, an isolated antibody, or antigen-binding fragment thereof, comprises an IgG1 Fc region and binds to SIRPα, optionally at least one SIRPα polypeptide or domain or epitope from Table S1, with a K_{D} of about 0.16 to about 2.5 nM. In some embodiments, an isolated antibody, or antigen-binding fragment thereof, comprises an IgG4 Fc region having an S228P substitution and binds SIRPα, optionally at least one SIRPα polypeptide or domain or epitope from Table S1, with a K_{D} of about 0.09 to about 1.66 nM, or about 0.088 nM, about 0.2643 nM, about 0.3778 nM, about 0.672 nM, about 0.6864 nM, or about 1.66 nM, optionally as measured by flow cytometric analysis of binding to cell surface SIRPα expressed on dendritic cells.

In some embodiments, the isolated antibody, or antigen-binding fragment thereof:
(a) selectively binds to SIRPα with high affinity, and does not significantly bind to SIRPβ;
(b) binds to SIRPα variants including V1, V2, and/or V8 SIRPα variants;
(c) binds to SIRPα and SIRPβ, and does not substantially bind to SIRPβ;
(d) binds to SIRPα and SIRPγ, and does not substantially bind to SIRPβ;
(e) binds to SIRPα, SIRPβ, and SIRPβ;
(f) binds to SIRPα and SIRPβL;
(g) binds to SIRPα and does not substantially bind to SIRPβL;
(h) inhibits binding of SIRPα to its ligand CD47;
inhibits SIRPα-CD47-mediated signaling;
induces and/or increases macrophage-mediated phagocytosis of cancer cells, optionally by reducing SIRPα-mediated inhibition of phagocytosis;
(k) increases antibody-dependent cell phagocytosis (ADCP);
(l) does not significantly inhibit binding of SIRPα to its ligand CD47 and does not significantly inhibit SIRPα-CD47-mediated signaling;
(m) inhibits macrophage-medicated phagocytosis;
(n) cross-reactively binds to human SIRPα and cynomolgus monkey SIRPα;
(o) binds to myeloid cells and does not significantly bind to primary T cells; or
(p) induces and/or increases dendritic cell activation of cytotoxic T cells; or
(q) a combination of any one or more of (a)-(o).

In some embodiments, an isolated antibody, or antigen-binding fragment thereof, is a SIRPα antagonist. In some embodiments, an isolated antibody, or antigen-binding fragment thereof, is a SIRPα agonist. In some embodiments, an isolated antibody, or antigen-binding fragment thereof, is a bi-specific or multi-specific antibody.

Also included is an isolated polynucleotide encoding an isolated antibody, or antigen-binding fragment thereof, as described herein, an expression vector comprising the isolated polynucleotide, or an isolated host cell comprising the vector.

Certain embodiments include a composition comprising a physiologically acceptable carrier and a therapeutically effective amount of an isolated antibody, or antigen-binding fragment thereof, described herein.

Also included is a method for treating, inhibiting the progression of, ameliorating the symptoms of, a cancer in a patient in need thereof, comprising administering to the patient a composition described herein, optionally wherein the antibody, or antigen-binding fragment thereof, is a SIRPα antagonist, thereby inhibiting the progression of, ameliorating the symptoms of, or treating the cancer. In some embodiments, the cancer is associated with aberrant SIRPα and/or CD47 expression. In some embodiments, the cancer is associated with SIRPα-mediated and/or CD47-mediated immune suppression. In some embodiments, the immune suppression comprises inhibition of phagocytosis by innate immune cells, optionally macrophages and/or dendritic cells. In some embodiments, the composition increases an immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to a control or reference. In some embodiments, the immune response comprises macrophage or dendritic cell-mediated phagocytosis of cancer cells. In some embodiments, the immune response comprises antibody-dependent cell phagocytosis (ADCP) of cancer cells.

In some embodiments, the cancer is selected from one or more of lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g., hepatocellular carcinoma), and skin (e.g., melanoma or malignant melanoma).

Also included is a method of treating, reducing the severity of, or preventing an infectious disease in a patient in need thereof, comprising administering to the patient the composition described herein, optionally wherein the antibody, or antigen-binding fragment thereof, is a SIRPα antagonist, thereby treating, reducing the severity of, or preventing the infectious disease. In some embodiments, the infectious disease is selected from viral, bacterial, fungal optionally yeast, and protozoal infections.

Some embodiments include a method of treating an autoimmune or inflammatory disease in a subject in need thereof, comprising administering to the patient a composition described herein, optionally wherein the antibody, or antigen-binding fragment thereof, is a SIRPα agonist, thereby treating the autoimmune or inflammatory disease. In some embodiments, the autoimmune or inflammatory disease is associated with aberrant macrophage activation and phagocytosis.

Some embodiments include a method of improving transplantation in a patient in need thereof, comprising administering to the patient a composition described herein in combination with transplanted cells, optionally wherein the antibody, or antigen-binding fragment thereof, is a SIRPα agonist that reduces phagocytosis of the transplanted cells, thereby improving transplantation in the patient. In some embodiments, the transplanted cells comprises hematopoietic stem cells, progenitor stem cells, or a solid organ. Certain embodiments comprise administering the composition to the subject prior to administration of transplanted cells, concurrently with administration of transplanted cells, or shortly after administration of transplanted cells.

### BRIEF DESCRIPTION OF THE DRAWINGS

FIG. 1 shows a schematic for the proposed mechanism of action of anti-SIRPα antibodies in cancer immunotherapy.

FIG. 2 illustrates the rabbit immunization and antibody screening strategy described herein for the anti-SIRPα antibody generation campaign.

FIGS. 3A-3D show binding analysis of the first set of humanized anti-SIRPα antibodies derived from B-cell panning to recombinant human SIRPα (3A), SIRPβ (3B), SIRPγ (3C) and recombinant Cynomolgus SIRPα (3D) by ELISA.

FIGS. 4A-4D show analysis of binding to recombinant human SIRPα for the second set of hybridoma-derived humanized anti-SIRPα antibodies (4A), SIRPβ (4B), SIRPγ (4C) and recombinant Cynomolgus SIRPα (4D) by ELISA.

FIGS. 5A-5B show analysis for ligand blocking activity of humanized anti-SIRPα antibodies—first set (A) and second set (B)—by ELISA.

FIGS. 6A-6C show binding analysis of anti-SIRPα antibodies to SIRPα expressed on the surface of human cells (6A), SIRPβ (6B), and SIRPγ (6C) by flow cytometry (FACs).

FIGS. 7A-7B show a comparison of ligand blocking activity of humanized anti-SIRPα antibodies containing IgG1 backbone (7A) and IgG4 S228P backbone (7B) on the cell surface.

FIGS. 8A-8B show analysis of signaling activities by humanized anti-SIRPα antibodies. FIG. 8A shows the results of antagonist activity testing, and FIG. 8B shows the results of agonist activity testing.

FIGS. 9A-9C show binding analysis of humanized anti-SIRPα antibodies to recombinant human SIRPα variants—variant 1 (9A), variant 2 (9B) and variant 8 (9C)—by ELISA.

FIGS. 10A-10B show an assessment of single agent phagocytosis by humanized anti-SIRPα antibodies in the IgG4 S228P backbone, as indicated by percent (%) phagocytosis (10A), and phagocytosis index (10B).

FIGS. 11A-11B show an assessment of antibody-dependent cellular phagocytosis (ADCP) by humanized anti-SIRPα antibodies in the IgG4 S228P backbone, as indicated by percent (%) phagocytosis (11A), and phagocytosis index (11B).

FIGS. 12A-12C show a comparison of single agent phagocytosis by humanized anti-SIRPα antibodies in the IgG4 S228P backbone. In 12A, phagocytosis Index is reported as CFSE positive VTD positive cells (double positive cells) divided by total macrophage cells (i.e. CFSE positive+double positive cells) multiplied by hundred. Experiment was repeated with macrophages from 3 different donors and the average is shown. In 12B, percent phagocytosis is reported as CFSE positive VTD positive cells (double positive cells) divided by total tumor cells (i.e. VTD positive+double positive cells) multiplied by one hundred. Experiment was repeated with macrophages from 2 different donors and the average is shown. In 12C, reduction in total macrophage number is represented as % reduction by subtracting total macrophage % from 100 from the same experiment as in B. The concentration of 14H2B was determined to be 4.3 times the concentration of the other test articles.

FIG. 13 shows an assessment of single agent phagocytosis by humanized anti-SIRPα antibodies in the IgG1 and IgG4 S228P backbone. Representative graph from one experiment is shown. The concentration of 14H2B was determined to be 4.3 times the concentration of the other test articles.

FIGS. 14A-14B show an assessment of single agent phagocytosis by humanized anti-SIRPα antibodies in the IgG4 S228P backbone using a different colon cancer cell line, LS-174T. Data is represented as Phagocytosis Index, that is, as CFSE positive VTD positive cells (double positive cells) divided by total macrophage cells (i.e., CFSE positive+double positive cells) multiplied by one hundred.

FIGS. 15A-15B show an assessment of ADCP potentiation by humanized anti-SIRPα antibodies in the IgG4 S228P backbone using a different colon cancer cell line, LS-174T. Experiment was repeated with macrophages from 2 donors. Data is represented as Phagocytosis Index, that is, as CFSE positive VTD positive cells (double positive cells) divided by total macrophage cells (i.e., CFSE positive+double positive cells) multiplied by one hundred.

FIGS. 16A-16B show binding analysis of anti-SIRPα antibodies 14H2A-IgG4 S228P and 14H2B-IgG4 S228P to human cell surface SIRPα from two donors by flow cytometry (FACs).

FIGS. 17A-17B show a comparison of ligand blocking activity of humanized anti-SIRPα antibodies 14H2A-IgG4 S228P and 14H2B-IgG4 S228P on human cell surface from two donors by flow cytometry.

FIGS. 18A-18B show a comparison of single agent phagocytosis by humanized anti-SIRPα antibodies 14H2A and 14H2B in the IgG4 S228P backbone in two donors. Phagocytosis Index is reported as CFSE positive VTD positive cells (double positive cells) divided by total macrophage cells (i.e. CFSE positive+double positive cells) multiplied by hundred. Experiment was repeated with macrophages from 3 different donors and the average is shown.

FIGS. 19A-19C show that anti-SIRPα antibodies significantly enhanced the phagocytosis activity of cetuximab on A431 cells. Light colored lines indicate single agent titration of anti-SIRP antibodies and darker lines indicate combination with 1 μg/mL Cetuximab.

FIGS. 20A-20C show that anti-SIRPα antibodies significantly enhanced the phagocytosis activity of trastuzumab on OE-19 cells. Light colored lines indicate single agent titration of anti-SIRP antibodies and darker lines indicate combination with 1 μg/mL Trastuzumab.

FIG. 21 shows that anti-SIRPα antibodies significantly enhanced the phagocytosis activity of rituximab on Raji cells. Dark colored bars indicate combination of anti-SIRPα antibodies with Rituximab with both antibodies at 6.6 nM. Bars with slanted lines show single agent activity of anti-SIRPα antibodies. Rituximab only condition is shown light bar.

FIG. 22 shows binding analysis of humanized anti-SIRPα antibodies to recombinant human SIRP-βL by ELISA.

FIG. 23 shows the epitope sites between humanized anti-SIRPα antibody 14H2B and non-contiguous regions of human SIRPα (HFPRVTTVSDLTKRNNMDFSI (SEQ ID NO: 235); and KGSPDDVEFKSGAGTELSVRA (SEQ ID NO: 236)), which epitope sites include residues 68, 70, 71, 73, 79, 100, 102, and 114 of the extracellular domain of SIRPα, as defined by SEQ ID NO: 228.

FIGS. 24A-24J show the interaction between antibody 14H2B and human SIRPα. The SIRPα-his PDB structure 2WNG is colored in dark gray on the epitope site Amino acids colored in dark gray correspond to residues 68-79 (SDLTKRNNMDFS (SEQ ID NO: 232)) and residues 100-114 (KGSPDDVEFKSGAGT (SEQ ID NO: 233)) of human SIRPα, as defined by SEQ ID NO: 228. A, B, C, D, E: ribbon/Surface representation of front view (A); back view (B), side view 1 (C), side view 2 (D) and top view (E). F, G, H, I, J: ribbon representation of front view (F); back view (G), side view 1 (H), side view 2 (I) and top view (J).

FIG. 25 shows the epitope site between humanized anti-SIRPα antibody 89H and a region of human SIRPα (DGQPAVSKSHDLKVSAHPKEQGSNTA (SEQ ID NO: 237)), which epitope site includes residues 319 and 332 of the extracellular domain of SIRPα, as defined by SEQ ID NO: 228.

FIGS. 26A-26J show the interaction between antibody 89H and human SIRPα. The SIRPα-his PDB structure 2WNG is colored dark gray on the epitope site. Amino acids colored in dark gray correspond to residues 319-332 (HDLKVSAHPKEQGS (SEQ ID NO: 234)) of SIRPα, as defined by SEQ ID NO: 228, and the 2WNG PDB structure only allows coloring of residues 319-322. A, B, C, D, E: ribbon/Surface representation of front view (A); back view (B), side view 1 (C), side view 2 (D) and top view (E). F, G, H, I, J: ribbon representation of front view (F); back view (G), side view 1 (H), side view 2 (I) and top view (J).

FIG. 27 shows the effects of anti-SIRPα antibodies on T cell activation from three human PBMC donors, as measured by IFN-γ production.

FIG. 28 shows the effects of anti-SIRPα antibodies on T cell proliferation following CD3/CD28 stimuation from three donors, as measured by flow cytometry.

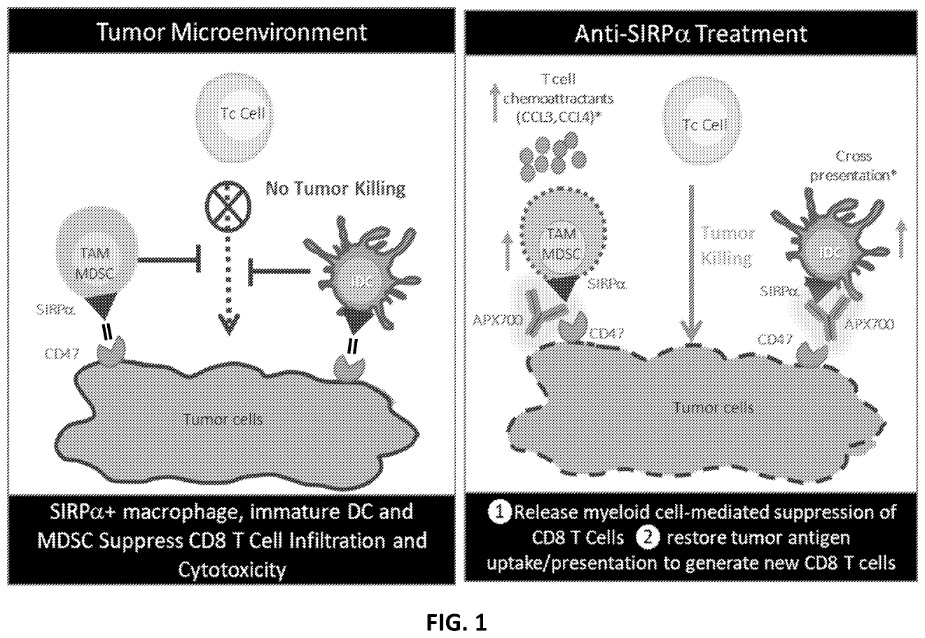

### DETAILED DESCRIPTION

The present disclosure relates to antibodies, and antigen-binding fragments thereof, which specifically bind to signal regulatory protein α (SIRPα), in particular antibodies having specific epitopic specificity and functional properties. Without being bound by any one theory, some embodiments relate to specific antibodies and fragments that bind to SIRPα, inhibit SIRPα binding to its ligand CD47, and reduce the SIRPα-mediated inhibition of tumor cell phagocytosis by innate immune cells. In certain embodiments, an anti-SIRPα antibody, or antigen-binding fragment thereof, is a SIRPα antagonist or inhibitor. In some instances, an antagonist of SIRPα enhances immune responses by increasing phagocytosis of tumor cells, for example, by directly inducing antibody-dependent cellular phagocytosis of tumor cells by macrophages or dendritic cells, and/or by inhibiting CD47-SIRPα signaling that otherwise negatively regulates such phagocytosis, among other possible mechanisms. In particular embodiments, an anti-SIRPα antibody, or antigen-binding fragment thereof, selectively binds to SIRPα and does not significantly bind to SIRPγ. SIRPα antagonist antibodies described herein are useful in the treatment and prevention of, for example, cancer, including SIRPα-and/or CD47-expressing cancers.

Some embodiments pertain to the use of anti-SIRPα antibodies, or antigen-binding fragments thereof, for the diagnosis, assessment, and treatment of diseases and disorders associated with SIRPα and/or CD47 activity or aberrant expression thereof. The subject antibodies are used in the treatment or prevention of cancer among other diseases.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y.(2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

As used in this specification and the appended claims, the singular forms “a,” “an” and “the” include plural references unless the content clearly dictates otherwise.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

An “antagonist” refers to an agent (e.g., antibody) that interferes with or otherwise reduces the physiological action of another agent or molecule. In some instances, the antagonist specifically binds to the other agent or molecule. Included are full and partial antagonists.

An “agonist” refers to an agent (e.g., antibody) that increases or enhances the physiological action of another agent or molecule. In some instances, the agonist specifically binds to the other agent or molecule. Included are full and partial agonists.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Each embodiment in this specification is to be applied mutatis mutandis to every other embodiment unless expressly stated otherwise.

The terms “modulating” and “altering” include “increasing,” “enhancing” or “stimulating,” as well as “decreasing”, “reducing”, or “inhibiting”, typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” or “inhibited” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

“Significantly” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

In certain embodiments, an antibody, or antigen-binding fragment thereof, is characterized by or comprises a heavy chain variable region (V_{H}) sequence that comprises complementary determining region V_{H}CDR1, V_{H}CDR2, and V_{H}CDR3 sequences, and a light chain variable region (V_{L}) sequence that comprises complementary determining region V_{L}CDR1, V_{L}CDR2, and V_{L}CDR3 sequences. Exemplary V_{H}CDR1, V_{H}CDR2, V_{H}CDR3, V_{L}CDR1, V_{L}CDR2, and V_{L}CDR3 sequences are provided in Table H1 below.

TABLE H1
Antibody CDR Sequences

| | | |
|---|---|---|
| | | SEQ ID |
| CDR | Sequence | NO: |

h700_3132_2

| | | |
|---|---|---|
| HCDR1 | GSDYIC | 1 |
| HCDR2 | CINVGIDNTYYASWATG | 2 |
| HCDR3 | GYSASAWGLDYFNL | 3 |
| LCDR1 | QASESIGNYLA | 4 |
| LCDR2 | GASTLES | 5 |
| LCDR3 | QCTYGTTSISTYT | 6 |

h700_4

| | | |
|---|---|---|
| HCDR1 | SYYFMC | 7 |
| HCDR2 | CIDAGYSGGSHYASWAKG | 8 |
| HCDR3 | AYEYGYGGYDL | 9 |
| LCDR1 | QASQSIDSNLA | 10 |
| LCDR2 | RASGLES | 11 |
| LCDR3 | QCTYGSNDSGNYANA | 12 |

h700_7

| | | |
|---|---|---|
| HCDR1 | SSDYMC | 13 |
| HCDR2 | CICTGHGDTAYATWAKG | 14 |
| HCDR3 | GWTSSADYYIYYLNL | 15 |
| LCDR1 | QASESIYNYLS | 16 |
| LCDR2 | GASNLES | 17 |
| LCDR3 | QQGYSGNNIDNI | 18 |

h700_8

| | | |
|---|---|---|
| HCDR1 | SSDYMC | 19 |
| HCDR2 | CINSGSDTTSYATWAKG | 20 |
| HCDR3 | GYGSAGANGYVIGMKYLNL | 21 |
| LCDR1 | QASQSIGSNLA | 22 |
| LCDR2 | TASTLES | 23 |
| LCDR3 | QCTYGSSSSGYP | 24 |

h700_9

| | | |
|---|---|---|
| HCDR1 | GSEYMS | 25 |
| HCDR2 | CIYFGISGSTYYANWAKG | 26 |
| HCDR3 | GYGDKYNLYYFNL | 27 |
| LCDR1 | QASEDIESYLA | 28 |
| LCDR2 | GASNLES | 29 |
| LCDR3 | QCTYGSSTTSNYGDA | 30 |

h700_42

| | | |
|---|---|---|
| HCDR1 | SYAMS | 31 |
| HCDR2 | IISASGANTWYATWAKG | 32 |
| HCDR3 | ISSGGWDYFNI | 33 |
| LCDR1 | QASQSISSYLS | 34 |
| LCDR2 | YSSTLAS | 35 |
| LCDR3 | QGYYYDRDSSSYGWA | 36 |

h700_44

| | | |
|---|---|---|
| HCDR1 | SYSMI | 37 |
| HCDR2 | ITYTGSSASYASWAKG | 38 |
| HCDR3 | ENSHDTFDP | 39 |
| LCDR1 | QSSQSVYTNYLS | 40 |
| LCDR2 | AASTLAS | 41 |
| LCDR3 | LGSYVSSGWYYA | 42 |

h700_14H2

| | | |
|---|---|---|
| HCDR1 | SYSMI | 43 |
| HCDR2 | IIYTGGSTDYASWAKG | 44 |
| HCDR3 | ENSHDTFDP | 45 |
| LCDR1 | QSSQSVYTNYXS, wherein X is | 225 |
| | any amino acid, optionally | |
| | L or Q | |
| LCDR2 | AASTLAS | 47 |
| LCDR3 | LGSYVSSGWYYA | 48 |

h700_14H2A

| | | |
|---|---|---|
| HCDR1 | SYSMI | 43 |
| HCDR2 | IIYTGGSTDYASWAKG | 44 |
| HCDR3 | ENSHDTFDP | 45 |
| LCDR1 | QSSQSVYTNYLS | 46 |
| LCDR2 | AASTLAS | 47 |
| LCDR3 | LGSYVSSGWYYA | 48 |

h700_14H2B

| | | |
|---|---|---|
| HCDR1 | SYSMI | 43 |
| HCDR2 | IIYTGGSTDYASWAKG | 44 |
| HCDR3 | ENSHDTFDP | 45 |
| LCDR1 | QSSQSVYTNYQS | 226 |
| LCDR2 | AASTLAS | 47 |
| LCDR3 | LGSYVSSGWYYA | 48 |

h700_89

| | | |
|---|---|---|
| HCDR1 | TYAMI | 49 |
| HCDR2 | IISPSGTTYYATWAKG | 50 |
| HCDR3 | AGSGGWDYFNI | 51 |
| LCDR1 | QASQSISSYLS | 52 |
| LCDR2 | YASNLAS | 53 |
| LCDR3 | QSYYYSNNYNWA | 54 |

h700_2830_2

| | | |
|---|---|---|
| HCDR1 | SYWMC | 55 |
| HCDR2 | CIYDGDGNTWYAYWVNG | 56 |
| HCDR3 | GGAGYLGYGYPFNL | 57 |
| LCDR1 | QASQSITSYLS | 58 |
| LCDR2 | RASTLAS | 59 |
| LCDR3 | QSYYFTSSNIYSYNA | 60 |

h700_12

| | | |
|---|---|---|
| HCDR1 | SYYYMC | 61 |
| HCDR2 | CIYAGDTGTTYYASWAKG | 62 |
| HCDR3 | GGYSYGYGGATYPTYFDL | 63 |
| LCDR1 | QASQSSSSYLA | 64 |
| LCDR2 | RASTLAS | 65 |
| LCDR3 | QSYYYSSSTSYDT | 66 |

h700_17

| | | |
|---|---|---|
| HCDR1 | SYAMS | 67 |
| HCDR2 | IIISSANTYYASWAKG | 68 |
| HCDR3 | GLAGFGYGGGAFKD | 69 |
| LCDR1 | QASQSISADYLA | 70 |
| LCDR2 | KASTLAS | 71 |
| LCDR3 | QYTGYDSVYIGA | 12 |

h700_56

| | | |
|---|---|---|
| HCDR1 | TYTMG | 73 |
| HCDR2 | ITYSGGNSYYASWAKG | 74 |
| HCDR3 | SSYDGYEYFNI | 75 |
| LCDR1 | QASQSISTYLA | 76 |
| LCDR2 | AASTLAS | 77 |
| LCDR3 | QQGYATTYVDNV | 78 |

h700_3

| | | |
|---|---|---|
| HCDR1 | SSYWGF | 79 |
| HCDR2 | SINGGTSGSTYYASWAKG | 80 |
| HCDR3 | DAGSSGYFFNL | 81 |
| LCDR1 | QASQSIGSNLA | 82 |
| LCDR2 | RASSLAS | 83 |
| LCDR3 | QNNYGITDNFGAA | 84 |

h700_48

| | | |
|---|---|---|
| HCDR1 | NMDTMC | 85 |
| HCDR2 | CIYIGDGVTFYASWVNG | 86 |
| HCDR3 | DAGDGGWYSFGL | 87 |
| LCDR1 | QASQSISGWLS | 88 |
| LCDR2 | SASTLAS | 3 |
| LCDR3 | QKNYGSSRGSSTNP | 90 |

h700_87

| | | |
|---|---|---|
| HCDR1 | TYTIS | 91 |
| HCDR2 | IIYTSGTTSYASWVKG | 92 |
| HCDR3 | DRSDGWYGTFNP | 93 |
| LCDR1 | QSSDNIGTYLA | 94 |
| LCDR2 | QASTLAS | 95 |
| LCDR3 | QQGYAVGHVDNT | 96 |

h700_2A1

| | | |
|---|---|---|
| HCDR1 | SYAMG | 97 |
| HCDR2 | IISPGGITYYATWAKG | 98 |
| HCDR3 | GWDYFNI | 99 |
| LCDR1 | QASQSISSYLA | 100 |
| LCDR2 | DASKLAS | 101 |
| LCDR3 | QSAYYGSSSTVNN | 102 |

h700_2A12

| | | |
|---|---|---|
| HCDR1 | NYYMC | 103 |
| HCDR2 | CIYSGSGGSTYHASWARG | 104 |
| HCDR3 | AYGVSVSGAYGHYFNL | 105 |
| LCDR1 | QASQSISTYLA | 106 |
| LCDR2 | GASNLES | 107 |
| LCDR3 | QSAYYGSSSTVNT | 108 |

h700_4E10

| | | |
|---|---|---|
| HCDR1 | NYEMI | 109 |
| HCDR2 | IIYVSRSTHYASWAKG | 110 |
| HCDR3 | YLASGGFNI | 111 |
| LCDR1 | QASHNIYSNLA | 112 |
| LCDR2 | DASKLAS | 113 |
| LCDR3 | QSYYGNSYP | 114 |

h700_1F10

| | | |
|---|---|---|
| HCDR1 | SYGVS | 115 |
| HCDR2 | YIHPDAGRIDYVNWVNG | 116 |
| HCDR3 | GNYDDYGDYFYFDL | 117 |
| LCDR1 | QASDFIYGNLA | 118 |
| LCDR2 | DASDLAS | 119 |
| LCDR3 | QSAYYSTSADMRNA | 120 |

h700_2H10

| | | |
|---|---|---|
| HCDR1 | SYAMG | 121 |
| HCDR2 | IIYIGDSAAYANWAKG | 122 |
| HCDR3 | SSYDGYEYFNI | 123 |
| LCDR1 | QASQSISSYLA | 124 |
| LCDR2 | AASTLAS | 125 |
| LCDR3 | QQGYATTYVDNP | 126 |

h700_AP28_S6G12.6

| | | |
|---|---|---|
| HCDR1 | RTYYLC | 127 |
| HCDR2 | CIYVSDNGKTYYASWARG | 128 |
| HCDR3 | DYSDDSVFLGL | 129 |
| LCDR1 | QASQNILYFLA | 130 |
| LCDR2 | DASNLAS | 131 |
| LCDR3 | QSVWYSSGAANL | 132 |

h700_AP28_S43F1.3

| | | |
|---|---|---|
| HCDR1 | SGYWIC | 133 |
| HCDR2 | CISIHTGKAHYASRAKG | 134 |
| HCDR3 | QGSDYADYNL | 135 |
| LCDR1 | QSSQSLSNAHVA | 136 |
| LCDR2 | FASRLAS | 137 |
| LCDR3 | QGEFDCSHGDCDA | 138 |

h700_AP30_50A8.15

| | | |
|---|---|---|
| HCDR1 | SSYWIC | 139 |
| HCDR2 | CISASSIPTTYYASWAKG | 140 |
| HCDR3 | YHTTNWAVDF | 141 |
| LCDR1 | QSSPSVASNYLS | 142 |
| LCDR2 | AASTLAS | 143 |
| LCDR3 | AGGYTGNIDTFV | 144 |

h700_AP32_N8D7.1

| | | |
|---|---|---|
| HCDR1 | SYVMN | 145 |
| HCDR2 | IIYTGGSASYANWAKG | 146 |
| HCDR3 | SPYDGTYYMDI | 147 |
| LCDR1 | QASQSIGAYFA | 148 |
| LCDR2 | SASTLAS | 149 |
| LCDR3 | QSYYATSTNT | 150 |

h700_AP32_S7F12.6

| | | |
|---|---|---|
| HCDR1 | NYDVT | 151 |
| HCDR2 | VITDDGHTYYAGWVNG | 152 |
| HCDR3 | DWRYFHI | 153 |
| LCDR1 | QASENIYSSLA | 154 |
| LCDR2 | DASDLAS | 155 |
| LCDR3 | QSYYGGTNVGYS | 156 |

h700_AP28_S35H11.8

| | | |
|---|---|---|
| HCDR1 | RGYYIC | 157 |
| HCDR2 | CIGVSSENVYYASWAKG | 158 |
| HCDR3 | GGSPGYSL | 159 |
| LCDR1 | QASQNTGGWLA | 160 |
| LCDR2 | EASKLAS | 161 |
| LCDR3 | QYTYGGSGGYG | 162 |

h700_AP28_S26G3.7

| | | |
|---|---|---|
| HCDR1 | NSYWMR | 163 |
| HCDR2 | YINTGSGGTYYASWAKG | 164 |
| HCDR3 | NGNL | 165 |
| LCDR1 | QSSQSVYDNNALA | 166 |
| LCDR2 | TASTLAS | 167 |
| LCDR3 | AGDFSGYIYD | 168 |

Thus, in certain embodiments, an antibody or antigen-binding fragment thereof, binds to SIRPα and comprises:
a heavy chain variable region (V_{H}) sequence that comprises complementary determining region V_{H}CDR1, V_{H}CDR2, and V_{H}CDR3 sequences selected from Table H1; and
a light chain variable region (V_{L}) sequence that comprises complementary determining region V_{L}CDR1, V_{L}CDR2, and V_{L}CDR3 sequences selected from Table H1, including variants thereof which specifically bind to at least one SIRPα polypeptide or domain or epitope (selected, for example, from Table S1).

In certain embodiments, the CDR sequences are as follows:
a heavy chain variable (VH) region comprising VHCDR1, VHCDR2, and VHCDR3 regions set forth respectively in SEQ ID NOs: 1-3; and a light chain variable (VL) region comprising VLCDR1, VLCDR2, and VLCDR3 regions set forth respectively in SEQ ID NOs: 4-6;
a VH region comprising VHCDR1, a VHCDR2, and VHCDR3 regions set forth respectively in SEQ ID NOs: 7-9; and a VL region comprising VLCDR1, VLCDR2, and VLCDR3 regions set forth respectively in SEQ ID NOs: 10-12;
a VH region comprising VHCDR1, VHCDR2, and VHCDR3 regions set forth respectively in SEQ ID NOs: 13-15; and a VL region comprising VLCDR1, VLCDR2, and VLCDR3 regions set forth respectively in SEQ ID NOs: 16-18;
a VH region comprising VHCDR1, VHCDR2, and VHCDR3 regions set forth respectively in SEQ ID NOs: 19-21; and a VL region comprising VLCDR1, VLCDR2, and VLCDR3 regions set forth respectively in SEQ ID NOs: 22-24;
a VH region comprising VHCDR1, VHCDR2, and VHCDR3 regions set forth respectively in SEQ ID NOs: 25-27; and a VL region comprising VLCDR1, VLCDR2, and VLCDR3 regions set forth respectively in SEQ ID NOs: 28-30;
a VH region comprising VHCDR1, VHCDR2, and VHCDR3 regions set forth respectively in SEQ ID NOs: 31-33; and a VL region comprising VLCDR1, VLCDR2, and VLCDR3 regions set forth respectively in SEQ ID NOs: 34-36;
a VH region comprising VHCDR1, VHCDR2, and VHCDR3 regions set forth respectively in SEQ ID NOs: 37-39; and a VL region comprising VLCDR1, VLCDR2, and VLCDR3 regions set forth respectively in SEQ ID NOs: 40-42;
a VH region comprising VHCDR1, VHCDR2, and VHCDR3 regions set forth respectively in SEQ ID NOs: 43-45; and a VL region comprising VLCDR1, VLCDR2, and VLCDR3 regions set forth respectively in SEQ ID NOs: 225 and 47-48, 226 and 47-48, or 46-48;
a VH region comprising VHCDR1, VHCDR2, and VHCDR3 regions set forth respectively in SEQ ID NOs: 49-51; and a VL region comprising VLCDR1, VLCDR2, and VLCDR3 regions set forth respectively in SEQ ID NOs: 52-54;
a VH region comprising VHCDR1, VHCDR2, and VHCDR3 regions set forth respectively in SEQ ID NOs: 55-57; and a VL region comprising VLCDR1, VLCDR2, and VLCDR3 regions set forth respectively in SEQ ID NOs: 58-60;
a VH region comprising VHCDR1, VHCDR2, and VHCDR3 regions set forth respectively in SEQ ID NOs: 61-63; and a VL region comprising VLCDR1, VLCDR2, and VLCDR3 regions set forth respectively in SEQ ID NOs: 64-66;
a VH region comprising VHCDR1, VHCDR2, and VHCDR3 regions set forth respectively in SEQ ID NOs: 67-69; and a VL region comprising VLCDR1, VLCDR2, and VLCDR3 regions set forth respectively in SEQ ID NOs: 70-72;
a VH region comprising VHCDR1, VHCDR2, and VHCDR3 regions set forth respectively in SEQ ID NOs: 73-75; and a VL region comprising VLCDR1, VLCDR2, and VLCDR3 regions set forth respectively in SEQ ID NOs: 76-78;
a VH region comprising VHCDR1, VHCDR2, and VHCDR3 regions set forth respectively in SEQ ID NOs: 79-81; and a VL region comprising VLCDR1, VLCDR2, and VLCDR3 regions set forth respectively in SEQ ID NOs: 82-84;
a VH region comprising VHCDR1, VHCDR2, and VHCDR3 regions set forth respectively in SEQ ID NOs: 85-87; and a VL region comprising VLCDR1, VLCDR2, and VLCDR3 regions set forth respectively in SEQ ID NOs: 88-90;
a VH region comprising VHCDR1, VHCDR2, and VHCDR3 regions set forth respectively in SEQ ID NOs: 91-93; and a VL region comprising VLCDR1, VLCDR2, and VLCDR3 regions set forth respectively in SEQ ID NOs: 94-96;
a VH region comprising VHCDR1, VHCDR2, and VHCDR3 regions set forth respectively in SEQ ID NOs: 97-99; and a VL region comprising VLCDR1, VLCDR2, and VLCDR3 regions set forth respectively in SEQ ID NOs: 100-102;
a VH region comprising VHCDR1, VHCDR2, and VHCDR3 regions set forth respectively in SEQ ID NOs: 103-105; and a VL region comprising VLCDR1, VLCDR2, and VLCDR3 regions set forth respectively in SEQ ID NOs: 106-108;
a VH region comprising VHCDR1, VHCDR2, and VHCDR3 regions set forth respectively in SEQ ID NOs: 109-111; and a VL region comprising VLCDR1, VLCDR2, and VLCDR3 regions set forth respectively in SEQ ID NOs: 112-114;
a VH region comprising VHCDR1, VHCDR2, and VHCDR3 regions set forth respectively in SEQ ID NOs: 115-117; and a VL region comprising VLCDR1, VLCDR2, and VLCDR3 regions set forth respectively in SEQ ID NOs: 118-120;
a VH region comprising VHCDR1, VHCDR2, and VHCDR3 regions set forth respectively in SEQ ID NOs: 121-123; and a VL region comprising VLCDR1, VLCDR2, and VLCDR3 regions set forth respectively in SEQ ID NOs: 124-126;
a VH region comprising VHCDR1, VHCDR2, and VHCDR3 regions set forth respectively in SEQ ID NOs: 127-129; and a VL region comprising VLCDR1, VLCDR2, and VLCDR3 regions set forth respectively in SEQ ID NOs: 130-132;
a VH region comprising VHCDR1, VHCDR2, and VHCDR3 regions set forth respectively in SEQ ID NOs: 133-135; and a VL region comprising VLCDR1, VLCDR2, and VLCDR3 regions set forth respectively in SEQ ID NOs: 136-138;
a VH region comprising VHCDR1, VHCDR2, and VHCDR3 regions set forth respectively in SEQ ID NOs: 139-141; and a VL region comprising VLCDR1, VLCDR2, and VLCDR3 regions set forth respectively in SEQ ID NOs: 142-144;
a VH region comprising VHCDR1, VHCDR2, and VHCDR3 regions set forth respectively in SEQ ID NOs: 145-147; and a VL region comprising VLCDR1, VLCDR2, and VLCDR3 regions set forth respectively in SEQ ID NOs: 148-150;
a VH region comprising VHCDR1, VHCDR2, and VHCDR3 regions set forth respectively in SEQ ID NOs: 151-153; and a VL region comprising VLCDR1, VLCDR2, and VLCDR3 regions set forth respectively in SEQ ID NOs: 154-156;
a VH region comprising VHCDR1, VHCDR2, and VHCDR3 regions set forth respectively in SEQ ID NOs: 157-159; and a VL region comprising VLCDR1, VLCDR2, and VLCDR3 regions set forth respectively in SEQ ID NOs: 160-162; or
a VH region comprising VHCDR1, VHCDR2, and VHCDR3 regions set forth respectively in SEQ ID NOs: 163-165; and a VL region comprising VLCDR1, VLCDR2, and VLCDR3 regions set forth respectively in SEQ ID NOs: 166-168.

Also included are variants thereof, including affinity matured variants, which bind to SIRPα, for example, variants having 1, 2, 3, 4, 5, 6, 7, or 8 total alterations across the CDR regions, for example, one or more the V_{H}CDR1, V_{H}CDR2, V_{H}CDR3, V_{L}CDR1, V_{L}CDR2, and/or V_{L}CDR3 sequences described herein. Exemplary “alterations” include amino acid substitutions, additions, and deletions.

In certain embodiments, an antibody, or antigen-binding fragment thereof, is characterized by or comprises a heavy chain variable region (V_{H}) sequence, and a light chain variable region (V_{L}) sequence. Exemplary humanized V_{H} and V_{L} sequences are provided in Table H2 below, exemplary rabbit V_{H} sequences are provided in Table R1 below (V_{H}CDR1, V_{H}CDR2, and V_{H}CDR3 regions are underlined), and exemplary rabbit V_{L} sequences are provided in Table R2 below (V_{L}CDR1, V_{L}CDR2, and V_{L}CDR3 regions are underlined).

TABLE H2
Heavy and Light Chain Sequences

| | | SEQ ID |
|---|---|---|
| Name | Sequence (CDRs underlined) | NO: |
| h700_3132_2H | EVQLVESGGGLVQPGGSLRLSCAASGFSFSGSDYICWVRQAPG | 169 |
| | KGLEWVACINVGIDNTYYASWATGRFTISRDNSKNTLYLQMNS | |
| | LRAEDTAVYYCARGYSASAWGLDYFNLWGQGTLVTVSS | |
| h700_3132_2K | DIQMTQSPSSLSASVGDRVTITCQASESIGNYLAWYQQKPGKA | 170 |
| | PKLLIYGASTLESGVPSRFSGSGSGTDFTLTISSLQPEDFATY | |
| | YCQCTYGTTSISTYTFGGGTKVEIK | |
| h700_4H | EVQLVESGGGLVQPGGSLRLSCAASGFSFSSYYFMCWVRQAPG | 171 |
| | KGLEWVACIDAGYSGGSHYASWAKGRFTISRDNSKNTLYLQMN | |
| | SLRAEDTAVYYCARAYEYGYGGYDLWGQGTLVTVSS | |
| h700_4K | DIQMTQSPSSLSASVGDRVTITCQASQSIDSNLAWYQQKPGKA | 172 |
| | PKLLIYRASGLESGVPSRFSGSGSGTDFTLTISSLQPEDFATY | |
| | YCQCTYGSNDSGNYANAFGGGTKVEIK | |
| h700_7H | EVQLVESGGGLVQPGGSLRLSCAASGFSFSSSDYMCWVRQAPG | 173 |
| | KGLEWVACICTGHGDTAYATWAKGRFTISRDNSKNTLYLQMNS | |
| | LRAEDTAVYYCARGWTSSADYYTYYLNLWGQGTLVTVSS | |
| h700_7K | AIQMTQSPSSLSASVGDRVTITCQASESIYNYLSWYQQKPGKA | 174 |
| | PKLLIYGASNLESGVPSRFSGSGSGTDFTLTISSLQPEDFATY | |
| | YCQQGYSGNNIDNIFGGGTKVEIK | |
| h700_8H | EVQLVESGGGLVQPGGSLRLSCAASGFSFSSSDYMCWVRQAPG | 175 |
| | KGLEWVGCINSGSDTTSYATWAKGRFTISRDNAKNTLYLQMNS | |
| | LRAEDTAVYYCARGYGSAGANGYVIGMKYLNLWGQGTLVTVSS | |
| h700_8K | DIQMTQSPSSLSASVGDRVTITCQASQSIGSNLAWYQQKPGKA | 176 |
| | PKLLIYTASTLESGVPSRFSGSGSGTDFTLTISSLQPEDFATY | |
| | YCQCTYGSSSSGYPFGGGTKVEIK | |
| h700_9H | EVQLVESGGGLVQPGGSLRLSCAASGFSFSGSEYMSWVRQAPG | 177 |
| | KGLEWVGCIYFGISGSTYYANWAKGRFTISRDNSKNTLYLQMN | |
| | SLRAEDTAVYYCARGYGDKYNLYYFNLWGQGTLVTVSS | |
| h700_9K | DIQMTQSPSSLSASVGDRVTITCQASEDIESYLAWYQQKPGKA | 178 |
| | PKLLIYGASNLESGVPSRFSGSGSGTDFTLTISSLQPEDFATY | |
| | YCQCTYGSSTTSNYGDAFGGGTKVEIK | |
| h700_42H | EVQLVESGGGLVQPGGSLRLSCAASGVDLSSYAMSWVRQAPGK | 179 |
| | GLEWVGIISASGANTWYATWAKGRFTISRDNSKNTLYLQMNSL | |
| | RAEDTAVYYCAGISSGGWDYFNIWGQGTLVTVSS | |
| h700_42K | DIQMTQSPSSLSASVGDRVTITCQASQSISSYLSWYQQKPGKA | 180 |
| | PKLLIYYSSTLASGVPSRFSGSGSGTDFTLTISSLQPEDFATY | |
| | YCQGYYYDRDSSSYGWAFGGGTKVEIK | |
| h700_44H1 | EVQLVESGGGLVQPGGSLRLSCAASGFSLSSYSMIWVRQAPGK | 181 |
| | GLEWVGIIYTGSSASYASWAKGRFTISRDNSKNTLYLQMNSLR | |
| | AEDTAVYYCARENSHDTFDPWGQGTLVTVSS | |
| h700_44K2 | DIQMTQSPSSLSASVGDRVTITCQSSQSVYTNYLSWYQQKPGK | 182 |
| | APKLLIYAASTLASGVPSRFSGSGSGTDFTLTISSLQPEDFAT | |
| | YYCLGSYVSSGWYYAFGGGTKVEIK | |
| h700_14H2_H | EVQLVESGGGLVQPGGSLRLSCAASGIDLSSYSMIWVRQAPGK | 183 |
| | GLEWIGIIYTGGSTDYASWAKGRFTISRDNSKNTLYLQMNSLR | |
| | AEDTAVYYCARENSHDTFDPWGQGTLVTVSS | |
| h700_14H2A_K | DIQMTQSPSSLSASVGDRVTITCQSSQSVYTNYLSWYQQKPGK | 184 |
| | APKLLIYAASTLASGVPSRFSGSGSGTDFTLTISSLQPEDFAT | |
| | YYCLGSYVSSGWYYAFGGGTKVEIK | |
| h700_14H2B_K | DIQMTQSPSSLSASVGDRVTITCQSSQSVYTNYQSWYQQKPGK | 227 |
| | APKLLIYAASTLASGVPSRFSGSGSGTDFTLTISSLQPEDFAT | |
| | YYCLGSYVSSGWYYAFGGGTKVEIK | |
| h700_89H | EVQLVESGGGLVQPGGSLRLSCAASGFSLSTYAMIWVRQAPGK | 185 |
| | GLEWVGIISPSGTTYYATWAKGRFTISRDNSKNTLYLQMNSLR | |
| | AEDTAVYYCARAGSGGWDYFNIWGQGTLVTVSS | |
| h700_89K | DIQMTQSPSSLSASVGDRVTITCQASQSISSYLSWYQQKPGKA | 186 |
| | PKLLIYYASNLASGVPSRFSGSGSGTDFTLTISSLQPEDFATY | |
| | YCQSYYYSNNYNWAFGGGTKVEIK | |
| h700_2830_2H | QVQLVESGGGLVQPGGSLRLSCSASGFTLSSYWMCWVRQAPGK | 187 |
| | GLELIACIYDGDGNTWYAYWVNGRFTISRDNSKNTLYLQMNSL | |
| | RAEDTAVYYCARGGAGYLGYGYPFNLWGQGTLVTVSS | |
| h700_2830_2K | DIQMTQSPSSLSASVGDRVTITCQASQSITSYLSWYQQKPGKA | 188 |
| | PKLLIYRASTLASGVPSRFSGSGSGTDFTLTISSLQPEDFATY | |
| | YCQSYYFTSSNIYSYNAFGGGTKVEIK | |
| h700_12H | EVQLVESGGGLVQPGGSLRLSCAASGFDLSSYYYMCWVRQAPG | 189 |
| | KGLGLIACIYAGDTGTTYYASWAKGRFTISRDNSKNTLYLQMN | |
| | SLRAEDTAVYYCARGGYSYGYGGATYPTYFDLWGQGTLVTVSS | |
| h700_12K | DIQMTQSPSTLSASVGDRVTITCQASQSSSSYLAWYQQKPGKA | 190 |
| | PKLLIYRASTLASGVPSRFSGSGSGTEFTLTISSLQPDDFATY | |
| | YCQSYYYSSSTSYDTFGGGTKVEIK | |
| h700_17H | EVQLVESGGGLVQPGGSLRLSCAASGIDLSSYAMSWVRQAPGK | 191 |
| | GLEWVGIIISSANTYYASWAKGRFTISRDNSKNTLYLQMNSLR | |
| | AEDTAVYYCARGLAGFGYGGGAFKDWGQGTLVTVSS | |
| h700_17K | DIQMTQSPSSVSASVGDRVTITCQASQSISADYLAWYQQKPGK | 192 |
| | APKLLIYKASTLASGVPSRFSGSGSGTDFTLTISSLQPEDFAT | |
| | YYCQYTGYDSVYIGAFGGGTKVEIK | |
| h700_56H | EVQLVESGGGLVQPGGSLRLSCAASGFSLSTYTMGWVRQAPGK | 193 |
| | GLEWVGIIYSGGNSYYASWAKGRFTISRDNSKNTLYLQMNSLR | |
| | AEDTAVYYCARSSYDGYEYFNIWGQGTLVTVSS | |
| h700_56K | DIQMTQSPSSLSASVGDRVTITCQASQSISTYLAWYQQKPGKA | 194 |
| | PKQLIYAASTLASGVPSRFSGSGSGTDFTLTISSLQPEDFATY | |
| | YCQQGYATTYVDNVFGGGTKVEIK | |
| h700_3H | EVQLVESGGGLVQPGGSLRLSCAASGFSFSSSYWGFWVRQAPG | 195 |
| | KGLEWVASINGGTSGSTYYASWAKGRFTISRDNSKNTLYLQMN | |
| | SLRAEDTAVYYCARDAGSSGYFFNLWGQGTLVTVSS | |
| h700_3K | DIQMTQSPSSVSASVGDRVTITCQASQSIGSNLAWYQQKPGKA | 196 |
| | PKLLIYRASSLASGVPSRFSGSGSGTDFTLTISSLQPEDFATY | |
| | YCQNNYGITDNFGAAFGGGTKVEIK | |
| h700_48H | EVQLVESGGGLVQPGGSLRLSCAASGFIFSNMDTMCWVRQAPG | 197 |
| | KGLEWVGCIYIGDGVTFYASWVNGRFTISRDNSKNTLYLQMNS | |
| | LRAEDTAVYYCARDAGDGGWYSFGLWGQGTLVTVSS | |
| h700_48K | DIQMTQSPSTLSASVGDRVTITCQASQSISGWLSWYQQKPGKA | 198 |
| | PKRLIYSASTLASGVPSRFSGSGSGTEFTLTISSLQPDDFATY | |
| | YCQKNYGSSRGSSTNPFGGGTKVEIK | |
| h700_87H | EVQLVESGGGLVQPGGSLRLSCAASGIDLSTYTISWVRQAPGK | 199 |
| | GLEWVGIIYTSGTTSYASWVKGRFTISRDNSKNTLYLQMNSLR | |
| | AEDTAVYYCARDRSDGWYGTFNPWGPGTLVTVSS | |
| h700_87K | DIQMTQSPSSLSASVGDRVTITCQSSDNIGTYLAWYQQKPGKA | 200 |
| | PKLLIYQASTLASGVPSRFSGSGSGTDFTLTISSLQPEDFATY | |
| | YCQQGYAVGHVDNTFGGGTKVEIK | |
| h700_2A1H | EVQLVESGGGLVQPGGSLRLSCAASGIDLSSYAMGWVRQAPGK | 201 |
| | GLEYVGIISPGGITYYATWAKGRFTISRDNSKNTLYLQMNSLR | |
| | AEDTAVYYCARGWDYFNIWGQGTLVTVSS | |
| h700_2A1K | AIQLTQSPSSLSASVGDRVTITCQASQSISSYLAWYQQKPGKA | 202 |
| | PKLLIYDASKLASGVPSRFSGSGSGTDFTLTISSLQPEDFATY | |
| | YCQSAYYGSSSTVNNFGGGTKVEIK | |
| h700_2A12H | EVQLVESGGGLVQPGGSLRLSCAASGSDISNYYMCWVRQAPGK | 203 |
| | GLEWVACIYSGSGGSTYHASWARGRFTISRDNSKNTLYLQMNS | |
| | LRAEDTAVYYCARAYGVSVSGAYGHYFNLWGQGTLVTVSS | |
| h700_2A12K | AIQLTQSPSSLSASVGDRVTITCQASQSISTYLAWYQQKPGKA | 204 |
| | PKLLIYGASNLESGVPSRFSGSGSGTDFTLTISSLQPEDFATY | |
| | YCQSAYYGSSSTVNTFGGGTKVEIK | |
| h700_4E10H | EVQLVESGGGLVQPGGSLRLSCAASGFSLSNYEMIWVRQAPGK | 205 |
| | GLEWVGIIYVSRSTHYASWAKGRFTISRDNSKNTLYLQMNSLR | |
| | AEDTAVYYCGRYLASGGFNIWGQGTLVTVSS | |
| h700_4E10K | DIQMTQSPSTLSASVGDRVTITCQASHNIYSNLAWYQQKPGKA | 206 |
| | PKLLIYDASKLASGVPSRFSGSGSGTEFTLTISSLQPDDFATY | |
| | YCQSYYGNSYPFGGGTKVEIK | |
| h700_1F10H | EVQLVESGGGLVQPGGSLRLSCAASGIDFSSYGVSWVRQAPGK | 207 |
| | GLEWVAYIHPDAGRIDYVNWVNGRFTISRDNAKNSLYLQMNSL | |
| | RAEDTAVYYCTIGNYDDYGDYFYFDLWGQGTLVTVSS | |
| h700_1F10K | DIQMTQSPSTLSASVGDRVTITCQASDFIYGNLAWYQQKPGKA | 208 |
| | PKLLIYDASDLASGVPSRFSGSGSGTEFTLTISSLQPDDFATY | |
| | YCQSAYYSTSADMRNAFGGGTKVEIK | |
| h700_2H10H | EVQLVESGGGLVQPGGSLRLSCAASGFSLSSYAMGWVRQAPGK | 209 |
| | GLEWVGIIYIGDSAAYANWAKGRFTISRDNSKNTLYLQMNSLR | |
| | AEDTAVYYCARSSYDGYEYFNIWGQGTLVTVSS | |
| h700_2H10K | DIQMTQSPSSVSASVGDRVTITCQASQSISSYLAWYQQKPGKA | 210 |
| | PKQLIYAASTLASGVPSRFSGSGSGTDFTLTISSLQPEDFATY | |
| | YCQQGYATTYVDNPFGGGTKVEIK | |
| h700_AP28_S6G12.6H | EVQLVESGGGLVQPGGSLRLSCTASGFSVSRTYYLCWVRQAPG | 211 |
| | KGLEWIACIYVSDNGKTYYASWARGRFTISRDNSKNTLYLQMN | |
| | SLRAEDTAVYFCARDYSDDSVFLGLWGPGTLVTVSS | |
| h700_AP28_S6G12.6K | DIQMTQSPSSLSASVGDRVTINCQASQNILYFLAWYQQKPGQR | 212 |
| | PKLLIYDASNLASGVPSRFSGSGSGTDFTLTISSLQPEDVATY | |
| | YCQSVWYSSGAANLFGGGTKVEIK | |
| h700_AP28_S43F1.3H | EVQLVESGGGLVQPGGSLRLSCTASGFSFSSGYWICWVRQAPG | 213 |
| | KGLEWIGCISIHTGKAHYASRAKGRFTISRDNSKNTLYLQMNS | |
| | LRAEDTAVYFCARQGSDYADYNLWGPGTLVTVSS | |
| h700_AP28_S43F1.3K | AIQLTQSPSSLSASVGDRVTINCQSSQSLSNAHVAWYQQKPGK | 214 |
| | PPKLLIYFASRLASGVPSRFSGSGSGTDFTLTISSLQPEDFAT | |
| | YYCQGEFDCSHGDCDAFGGGTKVEIK | |
| h700_AP30_50A8.15H | EVQLVESGGGLVQPGGSLRLSCAASGLDFSSSYWICWVRQAPG | 215 |
| | KGLEWIACISASSIPTTYYASWAKGRFTISRDNSKNTLYLQMN | |
| | SLRAEDTAVYYCWRYHTTNWAVDFWGPGTLVTVSS | |
| h700_AP30_50A8.15K | DIQMTQSPSSLSASVGDRVTITCQSSPSVASNYLSWYQQKPGK | 216 |
| | APKLLIYAASTLASGVPSRFSGSGSGTDFTLTISSLQPEDFAT | |
| | YYCAGGYTGNIDTFVFGGGTKVEIK | |
| h700_AP32_N8D7.1H | EVQLVESGGGLVQPGGSLRLSCTASGIDLSSYVMNWVRQAPGK | 217 |
| | GLEYIGIIYTGGSASYANWAKGRFTISRDNSKNTLYLQMNSLR | |
| | AEDTAVYFCARSPYDGTYYMDIWGPGTLVTVSS | |
| h700_AP32_N8D7.1K | DIQMTQSPSSLSASVGDRVTIKCQASQSIGAYFAWYQQKPGQP | 218 |
| | PKLLIHSASTLASGVPSRFSGSGSGTDFTLTISSLQPEDVATY | |
| | YCQSYYATSTNTFGGGTKVEIK | |
| h700_AP32_S7F12.6H | EVQLVESGGGLVQPGGSLRLSCTASGFSLNNYDVTWVRQAPGK | 219 |
| | GLEYIGVITDDGHTYYAGWVNGRFTISRDNSKNTLYLQMNSLR | |
| | AEDTATYFCARDWRYFHIWGPGTLVTVSS | |
| h700_AP32_S7F12.6K | DIQMTQSPSSLSASVGDRVTINCQASENIYSSLAWYQQKPGQP | 220 |
| | PKLLIYDASDLASGVPSRFSGSGSGTDFTLTISSLQPEDVATY | |
| | YCQSYYGGTNVGYSFGGGTKVEIK | |
| h700_AP28_S35H11.8H | EVQLLESGGGLVQPGGSLRLSCTASGFSFSRGYYICWVRQAPG | 221 |
| | KGLEWIACIGVSSENVYYASWAKGRFTISRDNSKNTLYLQMNS | |
| | LRAEDTAVYYCARGGSPGYSLWGPGTLVTVSS | |
| h700_AP28_S35H11.8K | DIQMTQSPSSLSASVGDRVTINCQASQNTGGWLAWYQQKPGQR | 222 |
| | PKLLIYEASKLASGVPSRFSGSGSGTDFTLTISSLQPEDVATY | |
| | YCQYTYGGSGGYGFGGGTKVEIK | |
| h700_AP28_S26G3.7H | EVQLVESGGGLVQPGGSLRLSCTASGFTLSNSYWMRWVRQAPG | 223 |
| | KGLEWIGYINTGSGGTYYASWAKGRFTISRDNSKNTLYLQMNS | |
| | LRAEDTAVYFCARNGNLWGPGTLVTVSS | |
| h700_AP28_S26G3.7K | AIQLTQSPSSLSASVGDRVTITCQSSQSVYDNNALAWYQQKPG | 224 |
| | KAPKLLIHTASTLASGVPSRFSGSGSGTDFTLTISSLQPEDFA | |
| | TYYCAGDFSGYIYDYGGGTKVEIK | |

TABLE R1
Exemplary Rabbit Antibody Heavy Chain Sequences
(HCDRs 1-3 are underlined)

| | | SEQ ID |
|---|---|---|
| Name | Sequence | NO: |
| rab700_AP2729_1H2 | QEQLEESGGDLVKPEGSLTLTCTASGFSFSSGYWICWVRQAPG | 238 |
| | KGLEWIGCISIANGKAYYASRAKGRFTISKTSSTTVTLQMTSL | |
| | TGADTATYFCARQGSDYADYNLWGPGTLVTVSS | |
| rab700_AP2729_77H1 | QSLEESGGRLVTPGTPLTLTCTVSGIDLSSYTMGWVRQAPGKG | 239 |
| | LEYIGIIYTGGSTYHTTWAKGRFTISKTSTAVDLKITSPTTED | |
| | TATYFCARSSYDGYEYFNIWGPGTLVTVSS | |
| rab700_AP2729_95H8 | QSLEESGGDLVKPGASLTLTCTASGSDISNYYMCWVRQAPGKG | 240 |
| | LEWIACIYAGSGGSTYYASWARGRFTISKTSSTTVTLQMTSLT | |
| | AADTATYFCARAYGVSVSGAYGHYFNLWGPGTLVTVSS | |
| rab700_AP2729_117H12 | QSLEESGGRLVTPGTPLTLTCTVSGFSLSTYAMGWVRQAPGKG | 241 |
| | LEYIGFIYTGDSTYYPSWAKGRFTISKTSTTVDLKITSPTTED | |
| | TATYFCARSSYSGYEYFNIWGPGTLVTVSS | |
| rab700_AP28_30_3H2 | QERVEESGGDLVQPEGSLTLTCTASGFSFSSSYWGFWVRQAPG | 242 |
| | KGLEWIACINGGTSGSTYYASWAKGRFTISKTSSTTVTLQMTS | |
| | LTAADTATYFCARDAGSSGYFFNLWGPGTLVTVSS | |
| rab700_AP28_30_48H1 | QSLEESGGDLVKPGASLTLTCTASGFIFSNMDTMCWVRQAPRK | 243 |
| | GLEWIGCIYIGDGVTFYASWVNGRFTISKTSSTTVTLQMTSLT | |
| | DADTATYFCARDAGDGGWYSFGLWGPGTLVTVSS | |
| rab700_AP28_30_79H2 | QSVEESGGRLVTPGGSLTLTCTVSGIDLNTYTISWVRQAPGKG | 244 |
| | LEWIGIIYTSGSTSYASWVNGRFTISKTSTTVDLKMTSLTAAD | |
| | TATYFCARDRSNGWYGIFNPWGPGTLVTVSS | |
| rab700_AP28_30_87H2 | QSVEESGGRLVTPGTPLTLTCTVSGIDLSTYTISWVRQAPGKG | 245 |
| | LEWIGIIYTSGTTSYASWVKGRFTISKTSTTVDLKMTSLTAAD | |
| | TATYFCARDRSDGWYGTFNPWGPGTLVTVSS | |
| rab700_AP28_30_90H2 | QSVEESGGRLVTPGTPLTLTCTVSGIDLSTYTISWVRQAPGKG | 246 |
| | LEWIGIIYTSGTTSYASWVKGRFTISKTSTTVDLKMTSLTAAD | |
| | TATYFCARDRSDGWYGTFNPWGPGTLVTVSS | |
| rab700_AP28_30_2H1 | QQQLEESGGGLVKPGGTLTLTCKASEFTLSSYWMCWVRQAPGK | 247 |
| | GLELIACIYDGDGNTWYAYWVNGRFTISRSASLNTVTLQMTSL | |
| | TAADTATYFCARGGAGYLGYGYPFNLWGPGTLVTVSS | |
| rab700_AP28_30_3H1 | QSLEESGGDLVKPGASLTLTCTVSGFSFSANIYMCWVRQAPGK | 248 |
| | GLEWVACIYAGSSGSTYYASWAKGRFTISKSSSTTVTLQMTSL | |
| | TAADTATYFCARGWTGIYGDSPYYFNLWGPGTLVTVSS | |
| rab700_AP28_30_11H2 | QSLEESGGDLVKPGASLTLTCTASGFSFSLYYYMCWVRQAPGK | 249 |
| | GLELIACIYTNSDSAYYASWAKGRFTISKTSSTTVTLQMASLT | |
| | AADTATYFCARDSYDDDGYFLIFSLWGPGTLVTVSS | |
| rab700_AP28_30_12H1 | QSLEESGGDLVKPGASLTLTCKASGFDLSSYYYMCWVRQAPGK | 250 |
| | GLGLIACIYAGDTGTTYYASWAKGRFTISKTSSTTVTLQMTSL | |
| | TAADTATYFCARGGYSYGYGGATYPTYFDLWGPGTLVTVSS | |
| rab700_AP28_30_13H2 | QSLEESGGDLVKPGASLTLTCTASGFSFSSSYWICWVRQAPGK | 251 |
| | GLELIACIKGGSSGSTYHASWAKGRFTISKTSSTTVTLQMTSL | |
| | TAADTATYFCARANDSTTYFNLWGPGTLVTVSS | |
| rab700_AP28_30_17H1 | QSVEESGGRLVTPGTPLTLTCTVSGIDLSSYAMSWVRQAPGKG | 252 |
| | LEWIGIIISSANTYYASWAKGRFTISKTSTTVSLQMNSPTTED | |
| | TATYFCARGLAGFGYGGGAFKDWGPGTLVTVSS | |
| rab700_AP28_30_56H1 | QSLEESGGRLVTPGTPLTLTCTVSGFSLSTYTMGWVRQAPGKG | 253 |
| | LEYIGIIYSGGNSYYASWAKGRFTISKTSTTVDLKITSPTTED | |
| | TATYFCARSSYDGYEYFNIWGPGTLVTVSS | |
| rab700_AP28_30_120H2 | QEQLVESGGGLVQPEGSLTLTCTVSGFSFSLYYYMCWVRQAPG | 254 |
| | KGLEWIGCIYTNNGVTWYASWAKGRFTISKTSSTTVTLQLNSL | |
| | TAADTATYFCARDNYDDSGYYFLFNLWGPGTLVTVSS | |
| rab700_AP30_2A1_H2 | QSLEESGGRLVTPGTPLTLTCTVSGIDLSSYAMGWVRQAPGKG | 255 |
| | LEYIGIISPGGITYYATWAKGRFTISKTSTTVDLRVTDLQPSD | |
| | TATYFCARGWDYFNIWGPGTLVTVSS | |
| rab700_AP30_2A12H2 | QSLEESGGDLVKPGASLTLTCTASRSDISNYYMCWVRQAPGKG | 256 |
| | LEWIACIYSGSGGSTYHASWARGRFTISNASSTTVTLEMTSLT | |
| | AADTATYFCARAYGVSVSGAYGHYFNLWGPGTLVTVSS | |
| rab700_AP30_1F10H2 | QEQLVESGGGLVTLGGSLKLSCKASGIDFSSYGVSWVRQAPGK | 257 |
| | GLEWIAYIHPDAGRIDYVNWVNGRFTISLDNAQNTVFLQMTSL | |
| | TAADTATYFCTIGNYDDYGDYFYFDLWGPGTLVTVSS | |
| rab700_AP30_1G9H1 | QSLEESGGDLVKPGASLTLTCKASGFSFSGSDYLCWVRQAPGK | 258 |
| | GLEWIACTTIGYNGGTYYASWVNGRFTIAITSTVDLKMSTLTA | |
| | ADTATYFCARGFADYNNYGDGGINYFNLWGPGTLVTVSS | |
| rab700_AP30_2H10H1 | QSLEESGGRLVTPGTPLTLTCTVSGFSLSSYAMGWVRQAPGKG | 259 |
| | LEYIGIIYIGDSAAYANWAKGRFTISKTSTTVDLEITSPTTED | |
| | TATYFCARSSYDGYEYFNIWGPGTLVTVSS | |
| rab700_AP30_4E10H2 | QSVEESGGRLVTPGTPLTLTCTASGFSLSNYEMIWVRQAPGKG | 260 |
| | LEWIGIIYVSRSTHYASWAKGRITISKTSTTVDLKITSPTTED | |
| | TATYFCGRYLASGGFNIWGPGTLVTVSS | |
| rab700_AP30_4G7_H1 | QSVEESGGRLVTPGTPLTLTCTASGFSLSSYAMSWVRQAPGKG | 261 |
| | LEWIGTISPGGVIYYASWAKGRFTISKTSTTVDLKMTSPTTED | |
| | TATYFCARGYGSSSGAYNIWGPGTLVTVSS | |
| rab700_AP30_5A12H1 | ESLEESGGDLVKPGASLTLTCTASGFSFSINYYVCWVRQAPGK | 262 |
| | GLEWIACIYAGSGDHTYYATWVNGRFTISKTSSTTVTLQMTSL | |
| | TAADTATYFCARDLTYDTGTFTFWGPGTLVTVSS | |
| rab700_AP30_5C2_H2 | QSVEESGGGLVTPGGTLTLTCTVSGFSLSTYAMGWVRQAPGKG | 263 |
| | LEWIGIIDSSGRTYYASWAKGRFTISKTSSTTVDLKIASPTTE | |
| | DTATYFCGRYNGDNGGYFNIWGPGTLVTVSS | |
| rab700_AP3132_25H2 | QSVEESGGRLVTPGTPLTLTCTVSGFSISSYAMIWVRQAPGKG | 264 |
| | LEWIGVISGLARTYYASWAKGRFTISKTSTTVDLEITSPTTED | |
| | TATYFCARGGSGGWDYFNIWGPGTLVTVSS | |
| rab700_AP3132_39H1 | QSVEESGGRLVTPGTPLTLTCTVSGFSLSSYAMIWVRQAPGEG | 265 |
| | LEWIGTISANSGSTWYASWAKGRFTISKTSTTVDLKITSPTTE | |
| | DTATYLCARAGTGWDYFNIWGPGTLVTVSS | |
| rab700_AP3132_106H2 | QSVEESGGRLVTPGTPLTLTCTVSGFSLSNYAMSWVRQAPGKG | 266 |
| | LEYIGIIVSASGSTYYASWAKGRFTISKTSTTVDLKITSPTTE | |
| | DTATYFCARADAGYSEYFNIWGPGTLVTVSS | |
| rab700_AP3132_40H2 | QSVEESGGRLVTPGTPLTLTCTVSGFSLSNYAMSWVRQAPGKG | 267 |
| | LEYIGIIVSASGSTYYASWAKGRFTISKTSTAVDLKITSPTTE | |
| | DTATYFCARADAGYSEYFNIWGPGTLVTVSS | |
| rab700_AP3132_68H1 | QSVEESGGRLVTPGTPLTLTCTVSGFSLSNYAMSWVRQAPGKG | 268 |
| | LEYIGIIVSASGSTYYASWAKGRFTISKTSTTVDLKITSPTTE | |
| | DTATYFCARADAGYSEYFNIWGPGTLVTVSS | |
| rab700_AP3132_64H2 | QSVEESGGRLVTPGTPLTLTCTVSGFSLSNYAMSWVRQAPGKG | 269 |
| | LEYIGIITASGGTYYASWAKGRFTISKTSTTVDLKITSPTTED | |
| | TATYFCARADGAYSEYFNIWGPGTLVTVSS | |
| rab700_AP3132_89H2 | QSVEESGGRLVTPGTPLTLTCTASGFSLSTYAMIWVRQAPGKG | 270 |
| | LEYIGIISPSGTTYYATWAKGRFTISKSSTTVGLKITSPTTED | |
| | TATYFCARAGSGGWDYFNIWGPGTLVTVSS | |
| rab700_AP3132_42H2 | QSVEESGGRLVKPDETLTLTCTVSGVDLSSYAMSWVRQAPGKG | 271 |
| | LEWIGIISASGANTWYATWAKGRFTLSKTSTTMDLKITSPTTE | |
| | DTATYFCAGISSGGWDYFNIWGPGTLVTVSS | |
| rab700_AP3132_88H2 | QSVEESGGRLVTPGTPLTLTCTVSGVDLSIYAMSWVRQAPGKG | 272 |
| | LEWIGIISASGANTWYASWAKGRFTISKASTTMDLKITSPTTE | |
| | DTATYFCAGISSGGWDYFNIWGPGTLVTVSS | |
| rab700_AP3132_101H1 | QSVEESGGRLVMPGTPLTVTCTVSGFSLSSYAMIWVRQAPGKG | 273 |
| | LEWIGIISSGGNTWYASWVKGRFSVSKTSTTVDLKITSPTSGD | |
| | TATYFCARASDIGVDVYNIWGPGTLVTVSS | |
| rab700_AP3132_37H2 | QSVEESGGRLVTPGTPLTLTCTVSGFSISSYAMIWVRQAPGEG | 274 |
| | LEYIGFISAGGTTTYASWAKGRFTISKTSTTVDLKITSPTTED | |
| | TATYFCARALSVGIDAFDPWGPGTLVTVSS# | |
| rab700_AP3132_81H2 | QSVEESGGRLVTPGTPLTLTCTVSGFSISSYAMIWVRQAPGEG | 275 |
| | LEYIGFISAGGTTSYASWAKGRFTISKTSTTVDLKITSPTTED | |
| | TATYFCARALSVGIDAFDPWGPGTLVTVSS# | |
| rab700_AP3132_63H1 | QSVEESGGRLVTPGTPLTLTCTASGFSLSNYAMIWVRQAPGQG | 276 |
| | LEWIGMISSGGNTYYASWAKGRFTISKTSTTVDLKMTSLTTED | |
| | TATYFCAKAFNLGIDAFDPWGPGTLVTVSS | |
| rab700_AP3132_18H1 | QSVEESGGRLVTPGTPLTLTCTVSGFSLSSYHINWVRQAPGKG | 277 |
| | LEWIGFIYGSGSVGYASWAKGRFTISEASTTVDLKITSPTTED | |
| | TATYFCAGGYGGQSGSGYDRLDLWGQGTLVTVSS | |
| rab700_AP3132_27H2 | QSVEESGGRLVTPGTPLTLTCTVSGFSLSSYHINWVRQAPGKG | 278 |
| | LEWIGFIYGSGSIGYANWAKGRFTISETSTTVDLKITSPTTED | |
| | TATYFCAGGYGGQSGSGYDRLDLWGQGTLVTVSS | |
| rab700_AP3132_16H1 | QSVEESGGRLVTPGTPLTLTCTASGFSLSNYHIEWVRQAPGKG | 279 |
| | LEWIGFIYGSGSVGYANWAKGRFTISKTSSTTVDLKMTSLTTE | |
| | DTATYFCAGGYGGQSGSGYDRLDLWGQGTLVTVSS | |
| rab700_AP3132_33H1 | QSVEESGGRLVTPGTPLTLTYTASGFSLTSHAMTWVRQAPGKG | 280 |
| | LVWIGIIYGGGSTAYASWATGRFTISRTSTTVDLRITSPTTED | |
| | TATYFCARLFDSGWGDRLDLWGQGTLVTVSS | |
| rab700_AP3132_35H2 | QSVEESGGRLVTPGTPLTLTCTASGFSLTSHAMTWVRQAPGKG | 281 |
| | LEWIGIIYGGGSTAYASWAKGRFTISRTSTTVDLGITSPTTED | |
| | TATYFCARLFDSGWGDRLDLWGQGTLVTVSS | |
| rab700_AP3132_62H2 | QSVEESGGRLVTPGTPLTLTCTASGFSLSSHAMTWVRQAPGKG | 282 |
| | LEWIGLIYGGGSTAYARWAKGRFTISKTSTTVDLRITSPTTED | |
| | TATYFCARLFDSGWGDRLDLWGQGTLVTVSS | |
| rab700_AP3132_57H1 | QSVEESGGRLVTPGTPLTLTCTASGFSLSSYGMAWVRQAPGKG | 283 |
| | LEYIGIIYIGGSTAYASWAKGRFTISKTSTTVDLKITSPTTED | |
| | TATYFCARYADNSYDYFNIWGPGTLVTVSS | |
| rab700_AP3132_78H2 | QSVEESGGRLVTPGTPLTLTCTASGFSLTSYGMSWVRQAPGKG | 284 |
| | LEYIGIIYIGGSASYASWAKGRFTISKTSTTVDLKITSPTTED | |
| | TATYFCARYADNSYDYFNIWGPGTLVTVSS | |
| rab700_AP3132_17H2 | QSLEESGGRLVTPGTPLTLTCTISGFSLSNYGMSWVRQAPGKG | 285 |
| | LEYIGIIYIGGSTWYASWAKGRFTISKSSTTVDLKITSPTTED | |
| | TATYFCARYGDNSYDHFNIWGPGTLVTVSS | |
| rab700_AP3132_44H1 | QSVEESGGRLVTPGTPLTLTCTVSGFSLSSYSMIWVRQAPGKG | 286 |
| | LEWIGIIYTGSSASYASWAKGRFTISKTSTTVDLKITSPTTED | |
| | TATYFCARENSHDTFDPWGPGTLVTVSS | |
| rab700_AP3132_23H2 | QSVEESGGRLVTPGTPLTLTCTVSGIDLSSYAMGWVRQAPGKG | 287 |
| | LEYIGIIYTGGSASYASWVKGRFTISKTSTTVDLQMTSLPTED | |
| | TATYFCARFSSDGYSYFNIWGPGTLVTVSS | |
| rab700_AP3132_65H1 | QSVEESGGRLVTPGTPLTLTCTVSGIDLSSYAMGWVRQAPGKG | 288 |
| | LEYIGIIYTGGSASYASWVKGRFTISKTSTTVDLKMTSLPTED | |
| | TATYFCARFSSDGYSYFNIWGPGTLVTVSS | |
| rab700_AP3132_51H1 | QSLEESGGRLVTPGTPLTLTCTVSGIDLSSYAMGWVRQAPGKG | 289 |
| | LEYIGIIYTGGSASYASWVKGRFTISKTSTTVDLKMTSLPTED | |
| | TATYFCARFSSDGYSYFNIWGPGTLVTVSS | |
| rab700_AP3132_58H1 | QSLEESGGRLVTPGTPLTLTCTVSGIDLSSYAMGWVRQAPGKG | 290 |
| | LEYIGIIYTGGSASYGSWVKGRFTISKTSTTVDLKMTSLPTED | |
| | TATYFCARFSSDGYSYFNIWGPGTLVTVSS | |
| rab700_AP3132_52H1 | QSLEESGGRLVTPGTPLTLTCTVSGIDLSSYAMGWVRQAPGKG | 291 |
| | LEYIGIIYTGGSASYASWVKGRFTISKTSTTVDLKMTSLPTED | |
| | TATYFCAGFSSDGYSYFNIWGPGTLVTVSS | |
| rab700_AP3132_102H2 | QSVEESGGRLVTPGTPLTLTCTVSGIDLSSYAMGWVRQAPGKG | 292 |
| | LEYIGIIYTGGSSSYASWVKGRFTISKTSTTVDLKMTSLPTED | |
| | TATYFCARFSSDGYSYFNIWGPGTLVTVSS | |
| rab700_AP3132_32H2 | QSVEESGGRLVTPGTPLTLTCTVSGIDLSSYAMGWVCQAPGKG | 293 |
| | LEYIGIIYTGGSASYASWVKGRFTISKTSTTVDLKMTSLPTED | |
| | TATYFCARFSSEGYSYFNIWGPGTLVTVSS | |
| rab700_AP3132_107H1 | QSVEESGGRLVTPGTPLTLTCTVSGFSLSSYAMGWVRQAPGKG | 294 |
| | LEWIGIMYAGGSTYYASWAKGRFTISKTSTTLDLKVNSLTTED | |
| | TATYFCARHISSGWDYFNIWGPGTLVTVSS | |
| rab700_AP3132_19H2 | RSVEESGGRLVTPDETLTITCTVSGIDLHSNSMTWVRQAPGKG | 295 |
| | LEWVGIIYVNDNTDYASWAKGRFTISKTSTTVDLKMTSLTTAD | |
| | TATYFCVRDLYPSTDYYNIWGPGTLVTVSS | |
| rab700_AP3132_2H1 | QSLEESGGDLVQPGASLTLTCTASGFSFSGSDYICWVRQAPGS | 296 |
| | GLEWIACINVGIDNTYYASWATGRFPISRTSSTTVTLQMTSLT | |
| | AADTATYFCARGYSASAWGLDYFNLWGPGTLVTVSS | |
| rab700_AP3132_8H1 | QSLEESGGDLVKPGASLTLTCTASGFSFSSSDYMCWVRQAPGK | 297 |
| | GLEWIGCINSGSDTTSYATWAKGRFTISRTSSFTVTLQMTSLT | |
| | AADTATYFCARGYGSAGANGYVIGMKYLNLWGPGTLVTVSS | |
| rab700_AP3132_7H1 | QSVEESGGDLVKPEGSLTLTCTASGFSFSSSDYMCWVRQAPGK | 298 |
| | GLEWIACICTGHGDTAYATWAKGRFTISRTSSTTVTLQMTSLT | |
| | AADTATYFCARGWTSSADYYIYYLNLWGPGTLVTVSS | |
| rab700_AP3132_9H1 | QSLEESGGGLVQPEGSLTLTCKVSGFSFSGSEYMSWVRQAPGK | 299 |
| | GLEWIGCIYFGISGSTYYANWAKGRFTISKTSSTTVTLQMTSL | |
| | TAADMATYFCARGYGDKYNLYYFNLWGPGTLVTVSS | |
| rab700_AP3132_4H1 | QEQLVESGGGLVQPEGSLTLTCTASGFSFSSYYFMCWVRQAPG | 300 |
| | KGLEWIACIDAGYSGGSHYASWAKGRLIISKTSSTTVTLQMTG | |
| | LTAADTATYFCARAYEYGYGGYDLWGPGTLVTVSS | |
| rab700_AP3132_5H2 | EVQLVESGGGLVQPGGSLRLSCAASGFSFSGSYYMCWVRQAPG | 301 |
| | KGLEWIVCIYIGSGRTWYASWAKGRFTISRDNAKNSLYLQMNS | |
| | LRDEDTAVYFCARGGWVNWGLWGPGTLVTVSS | |
| rab700_AP030_2F9. | QSLEESGGRLVTPGTFLTLTCTASGFTISNKHMSWVRQAPGKG | 302 |
| 6H1 | LEWIGIIDDGGKTWYANWATGRFTISKTSPTTVALMIISPTTE | |
| | DTATYFCARGGGNDGFDPWGPGTLVTVSS | |
| rab700_AP030_2F9. | QSLEESGGRLVTPGTFLTLTCTASGFTISNKHMSWVRQAPGKG | 303 |
| 8H1 | LEWIGIIDDGGKTWYANWATGRFTISKTSPTTVALMIISPTTE | |
| | DTATYFCARGGGNDGFDPWGPGTLVTVSS | |
| rab700_AP030_50A8. | QEQLEESGGDLVKPEGSLTLTCKASGLDFSSSYWICWVRQAPG | 304 |
| 8H1 | KGLEWIACISASSIPTTYYASWAKGRFTISRTSSTTVTLQMTS | |
| | LTAADTATYFCWRYHTTNWAVDFWGPGTLVTVSS | |
| rab700_AP030_50A8. | QEQLEESGGDLVKPEGSLTLTCKASGLDFSSSYWICWVRQAPG | 305 |
| 10H1 | KGLEWIACISASSIPTTYYASWAKGRFTISRTSSTTVTLQMTS | |
| | LTAADTATYFCWRYHTTNWAVDFWGPGTLVTVSS | |
| rab700_AP030_63A5. | QSVEESGGRLVTPGTPLTLTCTVSGFSLSSYAMGWVRQAPGKG | 306 |
| 4H1 | LEWIGTITTGGGTYYATWAKGQFTISKTSTTVYLKMTSPTTED | |
| | TATYFCGRRYRDYSDAFDIWGPGTLVTVSS | |
| rab700_AP030_63A5. | QSVEESGGRLVTPGTPLTLTCTVSGFSLSSYAMGWVRQAPGKG | 307 |
| 7H1 | LEWIGTITTGGGTYYATWAKGQFTISKTSTTVYLKMTSPTTED | |
| | TATYFCGRRYRDYSDAFDIWGPGTLVTVSS | |
| rab700_AP28_N2A3.2H | QEQLEESGGGLVYPADSLTLTCKADGFSFSRGYYICWVRQAPG | 308 |
| | KGLEWIACIGVSSENTYYPSWAKGRFTISKTSSTTVTLRMASL | |
| | TAADTATYFCARGGSPGYSLWGPGTLVTVSS | |
| rab700_AP28_S35B1. | QSLEESGGDLVKPEGSLTLTCTASGFSISSTYYICWVRQAPGR | 309 |
| 6H | GLEWIACIYVANNGKTYYASWARGRFTISKTSSTTVTLQMTSL | |
| | TAADTATYFCARDYSDDSTYLGLWGPGTLVTVSS | |
| rab700_AP28_S24F8. | QSLEESGGDLVKPGASLTLTCTVSGFTLTNSYWMRWVRQAPGK | 310 |
| 11H | GLEWIGYMHTGSGGTYYASWAKGRFTISRTSSTTVTLQMTGLT | |
| | AADTATYFCARNGSLWGPGTLVTVSS | |
| rab700_AP28_S26G3. | QSLEESGGDLVKPGASLTLTCTASGFTLSNSYWMRWVRQAPGK | 311 |
| 7H | GLEWIGYINTGSGGTYYASWAKGRFTISKTSSTTVTLQMTGLT | |
| | AADTATYFCARNGNLWGPGTLVTVSS | |
| rab700_AP28_S54E11. | QEQLEESGGGLVHPADSLTLTCTASGFSFSRGYYICWVRQAPG | 312 |
| 2H | KGLEWIACIGVSSENIYYASWAKGRFTISKTSSTTVTLKTARL | |
| | TAADTATYFCARGGSPGYSLWGPGTLVTVYS | |
| rab700_AP28_S10C3. | QEQLEESGGGLVYPADSLTLTCKASGFSFSRGYYICWVRQAPG | 313 |
| 2H | KGLEWIACIGVSSENTYYPSWAKGRFTISKTSSTTVTLRMASL | |
| | TAADTATYFCARGGSPGYSLWGPGTLVTVSS | |
| rab700_AP28_S11B1. | QSLEESGGDLVKPGASLTLTCTASGFTLSNSYWMRWVRQAPGK | 314 |
| 5H | GLEWIGYINTGSGGTYYASWAKGRFTISKTSSTTVTLQMTGLT | |
| | AADTATYFCARNGNLWGPGTLVTVSS | |
| rab700_AP28_S45A9. | QEQLEESGGGLVYPADSLTLTCKASGFSFSRGYYICWVRQAPG | 315 |
| 7H | KGLEWIACIGVSSENIYYPSWAKGRFTISKTSSTTVTLRMARL | |
| | TAADTATYFCARGGSPGYSLWGPGTLVTVSS | |
| rab700_AP28_S43F1. | QGQLVESGGGLVQPEGSLTLTCTASGFSFSSGYWICWVRQAPG | 316 |
| 3H | KGLEWIGCISIHTGKAHYASRAKGRFTISKTSSTTVTLQMTSL | |
| | TGADTANYFCARQGSDYADYNLWGPGTLVTVSS | |
| rab700_AP28_S6G12. | QSLEESGGDLVKPEGSLTLTCTASGFSVSRTYYLCWVRQAPGR | 317 |
| 6H | GLEWIACIYVSDNGKTYYASWARGRFTISKTSSTTVTLQMTGL | |
| | TAADTATYFCARDYSDDSVFLGLWGPGTLVTVSS | |
| rab700_AP28_S3D4.7H | QSLEESGGGLVQPEGSLTLTCKASGLSFSSMYYMCWVRQAPGK | 318 |
| | GLEWIGCSYIGSDGSAYYASWAKGRFSISRTSSTTVTLQMTSL | |
| | TAADTATYFCARGNSAGVCVDLWGPGTLVTVSS | |
| rab700_AP28_S35H11. | QEQLEESGGGLVQPAGSLTLTCTASGFSFSRGYYICWVRQAPG | 319 |
| 8H | KGLEWIACIGVSSENVYYASWAKGRFTISKTSSTTVTLQMARL | |
| | TAADTATYFCARGGSPGYSLWGPGTLVTVSS | |
| rab700_AP28_S40C11. | QSLEESGGDLVKPEGSLTLTCKASGFDLSRTYYLCWVRQAPGR | 320 |
| 1H | GLEWVACIYVSDVGKTYYASWARGRFTISKTSSTTVSLQMTGL | |
| | TAADTATYFCARDYSDDSVFLGLWGPGTLVTVSS | |
| rab700_AP28_S9F8.3H | QEQLEESGGGLVYPADSLTLTCKASGFSFSRGYYICWVRQAPG | 321 |
| | KGLEWIACIGVSSENIYYPSWAKGRFTISKTSSTTVTLRMASL | |
| | TAADTATYFCARGGSPGYSLWGPGTLVTVSS | |
| rab700_AP28_S39A3. | QEQLEESGGGLVHPADSLTLTCTASGFSFSRGYYICWVRQAPG | 322 |
| 1H | KGLEWIACIGVSSENIYYASWAKGRFTISKTSSTTVTLKMASL | |
| | TAADTATYFCARGGSPGYSLWGPGTLVTVSS | |
| rab700_AP28_S3G1.5H | QEQLEESGGGLVYPAGSLTLTCKASGFSFSRGYYICWVRQAPG | 323 |
| | KGLEWIACIGVSSENIYYPSWAKGRFTISKTSSTTVTLRMASL | |
| | TAADTATYFCARGGSPGYSLWGPGTLVTVSS | |
| rab700_P32_N8D7.1H | QSVEESGGRLVTPGTPLTLTCTVSGIDLSSYVMNWVRQAPGKG | 324 |
| | LEYIGIIYTGGSASYANWAKGRFTISKTSTTVDLTITSPTTAD | |
| | TATYFCARSPYDGTYYMDIWGPGTLVTVSS | |
| rab700_AP32_S7F12. | QSLEESGGRLVTPGTPLTLTCTVSGFSLNNYDVTWVRQTPGKG | 325 |
| 6H | LEYIGVITDDGHTYYAGWVNGRFTISKTSTTVDLKITSPTSED | |
| | TATYFCARDWRYFHIWGPGTLVTVSS | |

TABLE R2
Exemplary Rabbit Antibody Light Chain Sequences
(LCDRs 1-3 are underlined)

| | | SEQ ID |
|---|---|---|
| Name | Sequence | NO: |
| rab700_AP2729_77H1_ | QIVMTQTPASVSEPVGGTVTIKCQASQSISYYLAWYQQKPGQP | 326 |
| Kappa | PKLLIYDASKLASGVPSRFSGSGSGTEFTLTISDLECADAATY | |
| | YCQQGYGTTNVENPFGGGTEVVVK | |
| rab700_AP2729_95H8_ | DVVMTQTPASVSEPVGGTVTIKCQASQSINGWLSWYQQRPGQR | 327 |
| Kappa | PKLLIYGASYLASGVPSRFTGSGSGTEYTLTISDLECDDAATY | |
| | YCQSAYYGSSSTVNTFGGGTEVVVK | |
| rab700_AP2729_117H12_ | QIVMTQTPASVSEPVGGTVTIKCQASQSISSYLAWYQQKPGQP | 328 |
| Kappa | PNQLIYAASTLASGVPSRFKGSGSGTQFTLTISDLECADAATY | |
| | YCQQGYGTTNVDNPFGGGTEVVVK | |
| rab700_AP28_30_3H2_ | ADIVMTQTPSSVSAAVGGTVTIKCQASQSIGSNLAWYQQKPGQ | 329 |
| Kappa | RPKLLIYRASSLASGVPSRFKGSGSGTEYTLTISDLECDDAAT | |
| | YYCQNNYGITDNFGAAFGGGTEVVVK | |
| rab700_AP28_30_48H1_ | ADIVMTQTPASVEAAVGGTVTIKCQASQSISGWLSWYQQKPGQ | 330 |
| Kappa | PPKRLIYSASTLASGVPSRFKGSGSGTEYTLTISDLECADAAT | |
| | YYCQKNYGSSRGSSTNPFGGGTEVVVK | |
| rab700_AP28_30_79H2_ | ALVMTQTPASVSAALGDTVTIKCQASQSISTYLSWYQQKPGQP | 331 |
| Kappa | PKLLIYQASTVASGVPSRFSSSGSGTEFTLTISGVECADAATY | |
| | YCQQGYAVGHVDNTFGGGTEVVVK | |
| rab700_AP28_30_87H2_ | ALVMTQTPASVEVAVGDTVTINCQSSDNIGTYLAWYQQKPGQP | 332 |
| Kappa | PKLLIYQASTLASGVPSRFSGSGSGTEFTLTISGVECGDAATY | |
| | YCQQGYAVGHVDNTFGGGTEVVVK | |
| rab700_AP28_30_90H2_ | ALVMTQTPASVEVAVGDTVTINCQSSDNIGTYLAWYQQKPGQP | 333 |
| Kappa | PKLLIYQASTLASGVPSRFSGSGSGTEFTLTISGVECGDAATY | |
| | YCQQGYAVGHVDNTFGGGTEVVVK | |
| rab700_AP28_30_2H1_ | ADIVMTQTPASVEAAVGGTVTIKCQASQSITSYLSWYQQKPGQ | 334 |
| Kappa | PPKLLIYRASTLASGVSSRFKGSGSGTEFTLTISDLECADAAT | |
| | YACQSYYFTSSNIYSYNAFGGGTEVVVK | |
| rab700_AP28_30_3H1_ | DAVMTQTPASVEAAVGGTVTIKCQASQSISSYLAWYQQKPGQP | 335 |
| Kappa | PKLLIYRASTLESGVPSRFKGSGSGTEFALTISDLECADAATY | |
| | YCQCTYGTTAGSSFTFGGGTEVVVK | |
| rab700_AP28_30_11H2_ | QVLTQTPSSVSAAVGGTVTINCQASEDIERYLAWYQQKPGQRP | 336 |
| Kappa | KLLIYRASTLASGVSSRFKGSGSGTQFTLTISDLECDDAATYY | |
| | CQQSYSISNLDNGFGGGTEVVVK | |
| rab700_AP28_30_12H1_ | VDIVMTQTPASVEAAVGGTVTIKCQASQSSSSYLAWYQQKPGQ | 337 |
| Kappa | PPKLLIYRASTLASGVPSRFKGSGSGTQFTLTISDLECADAAT | |
| | YYCQSYYYSSSTSYDTFGGGTEVVVK | |
| rab700_AP28_30_13H2_ | DVVMTQTPASVSAAVGGTVTIKCQASQSMSSSYLAWYQQKPGQ | 338 |
| Kappa | PPKLLIYKASTLTSGVSSRFKGGGSGTEFTLTISDLECADAAT | |
| | YYCQGYVAVSGSSYPFGGGTEVVVK | |
| rab700_AP28_30_17H1_ | DVVMTQTPASVSEPVGGAVTIKCQASQSISADYLAWYQQKPGQ | 339 |
| Kappa | PPNLLIYKASTLASGVSSRFKGSGSGTEFTLTISDLECADAAT | |
| | YFCQYTGYDSVYIGAFGGGTEVVVK | |
| rab700_AP28_30_56H1_ | QIVMTQTPASVSEPVGGTVTIKCQASQSISTYLAWYQQKPGQP | 340 |
| Kappa | PKQLIYAASTLASGVPSRFKGSGSGTEFTLTISDLECADAATY | |
| | YCQQGYATTYVDNVFGGGTEVVVK | |
| rab700_AP28_30_120H2_ | AFELTQTPSSVSEPVGGTVTIKCQASENIYSSLAWYQQKPGQP | 341 |
| Kappa | PKLLIYQASTLASGVSSRFRGSGSGTQFTLTISDLECADAATY | |
| | YCQNYYSTDSGAFGGGTEVVVK | |
| rab700_AP30_2A1_H2_ | AFELTQTPSSVEAAVGGTVTINCQASQSISSYLAWYQQKPGQP | 342 |
| Kappa | PKLLIYDASKLASGVPSRFSGSGSGTEYTLTISDLECDDAATY | |
| | YCQSAYYGSSSTVNNFGGGTEVVVK | |
| rab700_AP30_2A12H2_ | AFELTQTPASVEAAVGGTVTIKCQASQSISTYLAWYQQKPGQP | 343 |
| Kappa | PKLLIYGASNLESGVPSRFTGSGSGTEFTLTISDLECDDAATY | |
| | YCQSAYYGSSSTVNTFGGGTEVVVK | |
| rab700_AP30_1F10H2_ | IEMTQTPFSVSAALGGTVTINCQASDFTYGNLAWYQQKPGQPP | 344 |
| Kappa | KLLIYDASDLASGVPSRFKGSGSGTEYTLTISGVQCADAATYY | |
| | CQSAYYSTSADMRNAFGGGTEVVVK | |
| rab700_AP30_1G9H1_ | ADVVMTQTPASVEAAVGGTVTINCQASQNIRSYLVWYQQKPGQ | 345 |
| Kappa | RPELLIYRASNLESGVPSRFSGSGSGTEFTLTISDLECADAAT | |
| | YYCQNYYNIANYGNAFGGGTEVVVK | |
| rab700_AP30_2H10H1_ | QIVMTQTPASVSDPVGGTVTIKCQASQSISSYLAWYQQKPGQP | 346 |
| Kappa | PKQLIYAASTLASGVPSRFKGSGSGTEFTLTISDLECADAATY | |
| | YCQQGYATTYVDNPFGGGTEVVVK | |
| rab700_AP30_4E10H2_ | DSVMTQTLASVSEPVGGTVTIKCQASHNIYSNLAWYQQRPGQR | 347 |
| Kappa | PKLLIYDASKLASGVPSRFKGSGSGTQFTLTISDLECDDAATY | |
| | YCQSYYGNSYPFGGGTEVVVK | |
| rab700_AP30_4G7_H1_ | DVVMTQTPASVSEPVGGTVTIKCQASQNIGSELAWYQQKPGQP | 348 |
| Kappa | PKLLIYVASYLASGVSSRFKGSGSGTDFTLTISDLECADAATY | |
| | YCQCTYYGGIPIGTFGGGTEVVVK | |
| rab700_AP30_5A12H1_ | ADVVMTQTPASVSDPVGGTVTIKCQASQNIGSNLAWYQQKPGQ | 349 |
| Kappa | RPKLLIYDASKLASGVPSRFSGSGYGTRFTLTISDLECADAAT | |
| | YYCQCTYYNGIDYVFGGGTEVVVK | |
| rab700_AP30_5C2_H2_ | ADVVMTQTPASVSEPVGGTVTIKCQASQSISSYLSWYQQKPGQ | 350 |
| Kappa | PPKLLMYKASTLASGVSSRFKGSESGTEFTLTISDLECADAAT | |
| | YYCQNYYIIGIDGGAFGGGTEVVVK | |
| rab700_AP3132_25H2_ | ADIVMTQTPASVEAAVGGTVTIKCQASQSIGSYLNWYQQKPGQ | 351 |
| Kappa | PPKLLIYYTSNLASGVSSRFSGSGSGTQFTLTISDLECADAAT | |
| | YYCQSYYYSSTNSSYSWPFGGGTEVVVK | |
| rab700_AP3132_39H1_ | ADIVMTQTPASVEAAVGGTVTIKCQASQTISSFLAWYQQKPGQ | 352 |
| Kappa | PPKLLIYYASTLASGVPSRFKGSRSETQFTLTISDLECADAAT | |
| | YYCQSYYDSSSYNYAWAFGGGTEVVVK | |
| rab700_AP3132_106H2_ | AIKMTQTPSSVSAAVGGTVTINCRASEDIESYLAWYQQKPGQR | 353 |
| Kappa | PKLQIYAASTLASGVPSRFRGSGSGTEYTLTISGVQCDDAATY | |
| | YCQSAYYSSSADNAFGGGTEVVVK | |
| rab700_AP3132_40H2_ | AIKMTQTPSSVSAAVGGTVTINCRASEDIESYLAWYQQKPGQR | 354 |
| Kappa | PKLLIYAASTLASGVPSRFKGSGSGTEYTLTISGVQCDDAATY | |
| | YCQSAYYSSSADNAFGGGTEVVVK | |
| rab700_AP3132_68H1_ | IKMTQTPSSVSAAVGGTVTINCQASEDIESYLAWYQQKPGQRP | 355 |
| Kappa | KLQTYAASTLASGVPSRFRGSGSGTEYTLTISGVQCDDAATYY | |
| | CQSAYYSSSADNAFGGGTEVVVK | |
| rab700_AP3132_64H2_ | IKMTQTPSSVSAAVGGTVTINCRASEDIESYLAWYQQKPGQRP | 356 |
| Kappa | KLLIYSASTLASGVPSRFKGSGSGTEYTLTISGVQCDDAATYY | |
| | CQSAYYSSSADNAFGGGTEVVVK | |
| rab700_AP3132_89H2_ | ADIVMTQTPASVSEPVGGTVTIKCQASQSISSYLSWYQQKSGQ | 357 |
| Kappa | PPKLLIYYASNLASGVPSRFSGSGSGTEFTLTISGVQCDDAAT | |
| | YYCQSYYYSNNYNWAFGGGTEVVVK | |
| rab700_AP3132_42H2_ | ADIVMTQTPASVSEPVGGTVTIKCQASQSISSYLSWYQQKPGQ | 358 |
| Kappa | RPKLLIYYSSTLASGVSSRFSGSGSGTEFTLTISDLECDDAAT | |
| | YYCQGYYYDRDSSSYGWAFGGGTEVVVK | |
| rab700_AP3132_88H2_ | ADIVMTQTPASVEAAVGGTVTIKCQASQSISSYLSWYQQKPGQ | 359 |
| Kappa | RPKLLIYYSSTLASGVSSRFSGSGSGTEFTLTISDLECDDAAT | |
| | YYCQGYYYNRDSSSYGWAFGGGTEVVVK | |
| rab700_AP3132_101H1_ | ADIVVTQTPASVEAAVGGTVTIKCQASQSISSYLSWYQQKPGQ | 360 |
| Kappa | PPKVLIYYASTLASGVPSWFKGSGSGTEFTLTISDLECADAAT | |
| | YYCQSYDWDSSPTYTFGGGTEVVVK | |
| rab700_AP3132_37H2_ | ADIVMTQTPASVEAAVGGTVTMKCQASQSISNYCSWYQQKPGQ | 361 |
| Kappa | PPKLLIYYASTLASGVPSRFKGSGSGTQFTLTISDLECDDAAT | |
| | YYCQSYDWNSSPTYAFGGGTEVVVK | |
| rab700_AP3132_81H2_ | ADIVMTQTPASVEAAVGGTVTMKCQASQSISNYCSWYQQKPGQ | 362 |
| Kappa | PPKLLIYYASTLASGVPSRFKGSGSGTQFTLTISDLECADAAT | |
| | YYCQSYDWNSSPTYAFGGGTEVVVK | |
| rab700_AP3132_63H1_ | ADIVMTQTPASVEAAVGGTVTIKCQASQTISSYLSWYQQKPGQ | 363 |
| Kappa | PPKLLIYYASTLASGVPSRFSGSGSGTQFTLTISDLECADAAT | |
| | YYCQSYDWNSSPSYVFGGGTEVVVK | |
| rab700_AP3132_18H1_ | AYDMTQTPASVSAAVGGTVTIKCQASQSIDSWLSWYQQKPGQP | 364 |
| Kappa | PKLLIYRASTLASGVPSRFKGSGSGTEYSLTISGVECADAATY | |
| | YCQQGYMTNNVDNAFGGGTEVVVK | |
| rab700_AP3132_27H2_ | AYDMTQTPASVSAAVGGTVTIKCQASQSIDSWLSWYQQKPGQP | 365 |
| Kappa | PKLLIYKASTLASGVPSRFKGSGSGTEYSLTISGVECAGAATY | |
| | YCQQGYMTNNVDNAFGGGTEVVVK | |
| rab700_AP3132_16H1_ | AYDMTQTPASVSEPVGGTVTINCQASESISSYLSWYQQKSGQP | 366 |
| Kappa | PKLLIYRASDLASGVPSRFKGSGSGTEFTLTISDLECADAATY | |
| | YCQQGYNTNLVDNAFGGGTEVVVK | |
| rab700_AP3132_33H1_ | QIVMTQTPASVSAAVGGTVTIKCQASQSIDSWLAWYQQKPGQH | 367 |
| Kappa | PRLLIYRASTLASGVSSRFKGSGSGTEFTLTISDLECADAATY | |
| | YCQQGYAISYVHNVFGGGTEVVVK | |
| rab700_AP3132_35H2_ | QIVMTQTPASVSAAVGGTVTIKCQARQSIDSWLAWYQQKPGQH | 368 |
| Kappa | PRLLIYRASTLASGVSSRFKGSGSGTEFTLTISDLECADAATY | |
| | YCQQGYGISYVHNVFGGGTEVVVK | |
| rab700_AP3132_62H2_ | QIVMTQTPASVSAAVGGTVTIKCQASQSIGSWLAWYQQKPGQH | 369 |
| Kappa | PKLLIYRASTLASGVSSRFKGSGSGTQFTLTISGVECADAATY | |
| | YCQQGYAISYVDNVFGGGTEVVVK | |
| rab700_AP3132_57H1_ | AIDMTQTPASVEAAVGGTVTVKCQASQSISTYLNWYQQKPGQP | 370 |
| Kappa | PKLLIYRASTLASGVSSRFKGSGSGTQFTLTISGVECADAATY | |
| | YCQQGYSNTNLDNSFGGGTEVVVK | |
| rab700_AP3132_78H2_ | AIDMTQTPASVEAAVGGTVTVKCQASQSISSYLNWYQQKPGQP | 371 |
| Kappa | PKLLIYRASTLASGVSSRFKGSGSGTQFTLTISGVECADAATY | |
| | YCQQGYSNTNLYNSFGGGTEVVVK | |
| rab700_AP3132_17H2_ | AIDMTQTPASVEAAVGGTVTISCQASQSISSYLNWYQQKPGQP | 372 |
| Kappa | PKLLIYRASTLASGVSSRFKGSGSGTQFTLTISGVECADAATY | |
| | YCQQGYSNTNVDNTFGGGTEVVVK | |
| rab700_AP3132_44H1_ | ADVVMTQTPSSVSAAVGGTVTINCQSSQSVYTNYLSWYQQKPG | 373 |
| Kappa | QPPKLLIYAASTLASGVPSRFKGSGSGTQFTLTISGVQCDDAA | |
| | AYYCLGSYVSSGWYYAFGGGTEVVVK | |
| rab700_AP3132_23H2_ | ALVMTQTPASVEAAVGGTVTINCQASQNIGSWLAWYQQKPGQP | 374 |
| Kappa | PKLLIYDASKLASGVPSRFKGSGSGTEFTLTISGVQCDDAATY | |
| | YCQQGYATSYIDNAFGGGTEVVVK | |
| rab700_AP3132_65H1_ | ALVMTQTPASVEAAVGGTVTINCQASQNIGSWLAWYQQKPGQP | 375 |
| Kappa | PKLLIYQASKLSSGVPSRFSGSGSGTEFTLTISGVQCDDAATY | |
| | YCQQGYASSYIDNAFGGGTEVVVK | |
| rab700_AP3132_51H1_ | ALVMTQTPASVEAAVGGTVTINCQASQNIGSWLAWYQQKPGQP | 376 |
| Kappa | PKLLIYQASKLSSGVPSRFSGSGSGTEFTLTISGVQCDDAATY | |
| | YCQQGYATSYIDNAFGGGTEVVVK | |
| rab700_AP3132_58H1_ | ALVMTQTPASVEAAVGGTVTINCQASQNIGSWLAWYQQKPGQP | 377 |
| Kappa | PKLLIYQASKLSSGVPSRFSGSGSGTEFTLTISGVQCDDAATY | |
| | YCQQGYATSYIDNAFGGGTEVVVK | |
| rab700_AP3132_52H1_ | ALVMTQTPASVEAAVGGTVTINCQASQNIGSWLAWYQQKPGQP | 378 |
| Kappa | PKLLIYQASKLSSGVPSRFSGSGSGTEFTLTISGVQCDDAATY | |
| | YCQQGYATSYIDNAFGGGTEVVVK | |
| rab700_AP3132_102H2_ | ALVMTQTPASVEAAVGGTVTINCQASQNIGSWLAWYQQKPGQP | 379 |
| Kappa | PKLLIYQASKLSSGVPSRFSGSGSGTEFTLTISGVQCDDAATY | |
| | YCQQGYATSYIDNAFGGGTEVVVK | |
| rab700_AP3132_32H2_ | ALVMTQTPASVEAAVGGTVTINCQASQNIGSWLAWYQQKPGQP | 380 |
| Kappa | PKLLIYQASKLSSGVPSRFSGSGSGTEFTLTISGVQCDDAATY | |
| | YCQQGYATSYIDNAFGGGNEVVVK | |
| rab700_AP3132_107H1_ | QIVMTQTPASVSAAVGGTVTINCQASQSINSWLSWYQQKPGQR | 381 |
| Kappa | PKLLIYSASTLASGVSSRFRGSGSGTEFTLTISDLECDDAATY | |
| | YCQQCYGVSFVDNTFGGGTEVVVK | |
| rab700_AP3132_19H2_ | ALVMTQTPSPVSAAVGGTVTISCQASQSISNWLAWYQQKAGQR | 382 |
| Kappa | PKLLIYYTSNLASGVPSRFSGSGSGTQFTLTISDVQCADAATY | |
| | NCAGYKSYSNDDNGFGGGTEVVVK | |
| rab700_AP3132_2H1_ | DVVMTQTPASVEATVGGTVTIKCQASESIGNYLAWYQQKPGQP | 383 |
| Kappa | PKLLIYGASTLESGVPSRFSGSGSGTEFTLTISDLECADAATY | |
| | YCQCTYGTTSISTYTFGGGTEVVVK | |
| rab700_AP3132_8H1_ | DVVMTQTPASVEAAVGGTVTINCQASQSIGSNLAWYEQKPGQP | 384 |
| Kappa | PKLLIYTASTLESGVPSRFKGSGSGTEFTLTISDLECADAATY | |
| | YCQCTYGSSSSGYPFGGGTEVVVK | |
| rab700_AP3132_7H1_ | AYDMTQTPSSVSEPVGGTVTINCQASESTYNYLSWYQQKPGQP | 385 |
| Kappa | PKLLIYGASNLESGVPSRFKGSGSGTEYTLTISDLECADAATY | |
| | FCQQGYSGNNIDNIFGGGTEVVVK | |
| rab700_AP3132_9H1_ | DVVMTQTPASVEAAVGGTVTIKCQASEDIESYLAWYQQKPGQR | 386 |
| Kappa | PKLLIYGASNLESGVSSRFKGSGSGTEYTLTISDLECADAATY | |
| | YCQCTYGSSTTSNYGDAFGGGTEVVVK | |
| rab700_AP3132_4H1_ | ADIVMTQTPASVEAAVGGTVTIKCQASQSIDSNLAWYQQKPGQ | 387 |
| Kappa | PPKLLIYRASGLESGVPSRFRGSGSGTEFTLTISDLECADAAT | |
| | YYCQCTYGSNDSGNYANAFGGGTEVVVK | |
| rab700_AP3132_5H2_ | AFELTQTPSSVEAAVGGTVTIKCQASQSISNYLAWYQQKPGQP | 388 |
| Kappa | PKLLIYGASTLASGVSSRFKGSGSGTEFTLTISDLECADAATY | |
| | ACQTTYANTIGWAFGGGTEVVVK | |
| rab700_AP030_2F9.6H1_ | ADIVMTQTPGSVEAAVGGTVAIKCQASQYINGYLSWYQQKPGQ | 389 |
| Kappa | PPKLLIYRASTLASGVSSRFKGSGSGTEYTLTISDLECADAAT | |
| | FYCQSYYFNGGTLINPFGGGTEVVVK | |
| rab700_AP030_2F9.8H1_ | ADIVMTQTPGSVEAAVGGTVAIKCQASQYINGYLSWYQQKPGQ | 390 |
| Kappa | PPKLLIYRASTLASGVSSRFKGSGSGTEYTLTISDLECADAAT | |
| | FYCQSYYFNGGTLINPFGGGTEVVVK | |
| rab700_AP030_50A8.8H1_ | AAVLTQIPSSVSAGVGGTVTISCQSSPSVASNYLSWYQQKPGQ | 391 |
| Kappa | RPKLLIYAASTLASGVPSRFKGSGSGTQFTLTISDVQCADAAT | |
| | YYCAGGYTGNIDTFVFGGGTEVVVK | |
| rab700_AP030_50A8.10H1_ | AAVLTQTPSSVSAGVGGTVTISCQSSPSVASNYLSWYQQKPGQ | 392 |
| Kappa | RPKLLIYAASTLASGVPSRFKGSGSGTQFTLTISDVQCADAAT | |
| | YYCAGGYTGNIDTFVFGGGTEVVVK | |
| rab700_AP030_63A5.4H1_ | DVVMTQTPASVEAAVGGTVTIKCQASQSINSWFSWYQQKPGQR | 393 |
| Kappa | PKLLIYAAANLASGVPSRFKGSRSGTEYTLTISDLECGDAATY | |
| | YCQCNAGSDIDSNGDAFGGGTEVVVK | |
| rab700_AP030_63A5.7H1_ | DVVMTQTPASVEAAVGGTVTIKCQASQSINSWFSWYQQKPGQR | 394 |
| Kappa | PKLLIYAAANLASGVPSRFKGSRSGTEYTLTISDLECGDAATY | |
| | YCQCNAGSDIDSNGDAFGGGTEVVVK | |
| rab700_AP28_N2A3.2_ | DIVMTQTPASVEAAVGGTVTINCQASQNTGGWLAWYQQRPGQR | 395 |
| Kappa | PKLLIYEASKLASGVPSRFKGGGAGTEFTLNISDLECADAATY | |
| | YCQYTYGGSGGYGFGGGTEVVVK | |
| rab700_AP28_S35B1.6_ | AFELTQAPSSVEAAVGGTVTINCQASQSILYFLAWYQQKPGQR | 396 |
| Kappa | PKLLIYDASDLASGVPSRFSGSGSGTEFTLTISDLECDDAAIY | |
| | YCQSVWYSSGAANIFGGGTEVVVK | |
| rab700_AP28_S24F8.11_ | AQVLTQTSSPVSAAVGGTVTINCKTSQSVYDNNALAWYQQKPG | 397 |
| Kappa | QPPKLLIHTASTLASGVPSRFRGSGSGTQFTLTISDLECDDAA | |
| | TYYCGGDFNGYIYDYGGGTEVVVK | |
| rab700_AP28_S26G3.7_ | AQVLTQTASPVSAAVGGTITIKCQSSQSVYDNNALAWYQQKPG | 398 |
| Kappa | QPPKLLIHTASTLASGVPSRFKGSGSGTEFTLTISDLECDDAA | |
| | TYYCAGDFSGYIYDYGGGTEVVVK | |
| rab700_AP28_S54E11.2_ | DIVMTQTPASVEAAVGGTVTINCQASQNTGGWLAWYQKKPGQR | 399 |
| Kappa | PKLLIYEASKLTSGVPSRFKGGGSGTEFTLNISDLECADAAAY | |
| | YCQYTYGGSGGYGFGGGTEVVVK | |
| rab700_AP28_S10C3.2_ | DIVMTQTPASVEAAVGGTVTINCQASQNTGGWLAWYQQKPGQR | 400 |
| Kappa | PKLLIYEASKLASGVPSRFKGGGAGTEFTLNISDLECADAATY | |
| | YCQYTYGGSGGYGFGGGTEVVVK | |
| rab700_AP28_S11B1.5_ | AQVLTQTASPVSAAVGGTITIKCQSSQSVYDNNALAWYQQKPG | 401 |
| Kappa | QPPKLLIHTASTLASGVPSRFKGSGSGTEFTLTISDLECDDAA | |
| | TYYCAGDFSGYIYDYGGGTEVVVK | |
| rab700_AP28_S45A9.7_ | DIVMTQTPASVEAAVGGTVTINCQASQNTGGWLAWYQQKPGQP | 402 |
| Kappa | PKLLIYEASKLASGVPSRFKGSGAGTEFTLNISDLECADAAAY | |
| | YCQYTYGGSGGYGFGGGTEVVVK | |
| rab700_AP28_S43F1.3_ | AQVLTQTASPVSAAVGGTVTINCQSSQSLSNAHVAWYQQKPGQ | 403 |
| Kappa | PPKLLIYFASRLASGVSSRFTGSGSGTQFTLTISDLECDDAAT | |
| | YYCQGEFDCSHGDCDAFGGGTEVVVR | |
| rab700_AP28_S6G12.6_ | AFELTQAPSSVEAAVGGTVTINCQASQNILYFLAWYQQKPGQR | 404 |
| Kappa | PKLLIYDASNLASGVPSRFSGSGSGTEFTLTISDLECDDAAIY | |
| | YCQSVWYSSGAANLFGGGTEVVVK | |
| rab700_AP28_S3D4.7_ | AIVMTQTPSSKSVPVGDTVTISCQASESVYSDNRLAWFQQKPG | 405 |
| Kappa | QRPKLLIYYASTLASGVPSRFKGSGSGTQFTLTINDVVCDDAA | |
| | TYYCAGYKSDSPDGFAFGGGTEVVVK | |
| rab700_AP28_S35H11.8_ | DIVMTQAPASVEAAMGDTVTINCQASQNTGGWLAWYQQKAGQR | 406 |
| Kappa | PKLLIYEASKLASGVPSRFKGGGAGTEFTLHISDLECADAATY | |
| | YCQYTYGGSGGYGFGGGTEVVVK | |
| rab700_AP28_S40C11.1_ | AFELTQAPSSVEAAVGGTVTINCQASQNILYFLAWYQQKPGQR | 407 |
| Kappa | PKLLIYDASNLASGVPSRFSGSGSGTEFTLTISDLECADAAIY | |
| | YCQSVWYGSGAANLFGGGTEVVVT | |
| rab700_AP28_S9F8.3_ | DIVMTQTPASVEAAVGGTVTINCQASQNTGGWLAWYQQKPGQR | 408 |
| Kappa | PKLLIYEASKLASGVPSRFKGGGAGTEFTLNISDLECADAATY | |
| | YCQYTYGGSGGYGFGGGTEVVVK | |
| rab700_AP28_S39A3.1_ | DIVMTQTPASVEAAVGGTVTINCQASQNTGGWLAWYQQKPGQR | 409 |
| Kappa | PKLLIYEASKLASGVPSRFKGGGAGTEFTLNISDLECADAATY | |
| | YCQYTYGGSGGYGFGGGTEVVVK | |
| rab700_AP28_S3G1.5_ | DIVMTQTPASVEAAVGGTVTINCQASQNTGGWLAWYQQKPGQR | 410 |
| Kappa | PQLLIYEASKLASGVPSRFKGGGSGTDFTLTISDLECADAATY | |
| | YCQYTYGGSGGYGFGGGTEVVVK | |
| rab700_AP32_N8D7.1_ | AFELTQTPSSVEAAVGGTVSIKCQASQSIGAYFAWYQQKPGQP | 411 |
| Kappa | PKLLIHSASTLASGVSSRFKGSGSGTEFTLTISDLECADAATY | |
| | YCQSYYATSTNTFGGGTEVVVK | |
| rab700_AP32_700_ | AVVMTQTASPVSEPVGGTVTINCQASENIYSSLAWYQQKPGQP | 412 |
| S7F12.6_Kappa | PKLLIYDASDLASGVPSRFKGSGSGTDFTLTISGVQCDDAATY | |
| | YCQSYYGGTNVGYSFGGGTEVVVK | |

TABLE R3
HC and LC Designations for Rabbit Abs

| Rabbit antibodies | Rabbit Heavy Chains | Rabbit Light Chains |
|---|---|---|
| rab700_AP2729_1 | rab700_AP2729_1H2 | |
| rab700_AP2729_77 | rab700_AP2729_77H1 | rab700_AP2729_77H1_Kappa |
| rab700_AP2729_95 | rab700_AP2729_95H8 | rab700_AP2729_95H8_Kappa |
| rab700_AP2729_117 | rab700_AP2729_117H12 | rab700_AP2729_117H12_Kappa |
| rab700_AP28_30_3 | rab700_AP28_30_3H2 | rab700_AP28_30_3H2_Kappa |
| rab700_AP28_30_48 | rab700_AP28_30_48H1 | rab700_AP28_30_48H1_Kappa |
| rab700_AP28_30_79 | rab700_AP28_30_79H2 | rab700_AP28_30_79H2_Kappa |
| rab700_AP28_30_87 | rab700_AP28_30_87H2 | rab700_AP28_30_87H2_Kappa |
| rab700_AP28_30_90 | rab700_AP28_30_90H2 | rab700_AP28_30_90H2_Kappa |
| rab700_AP28_30_2 | rab700_AP28_30_2H1 | rab700_AP28_30_2H1_Kappa |
| rab700_AP28_30_3 | rab700_AP28_30_3H1 | rab700_AP28_30_3H1_Kappa |
| rab700_AP28_30_11 | rab700_AP28_30_11H2 | rab700_AP28_30_11H2_Kappa |
| rab700_AP28_30_12 | rab700_AP28_30_12H1 | rab700_AP28_30_12H1_Kappa |
| rab700_AP28_30_13 | rab700_AP28_30_13H2 | rab700_AP28_30_13H2_Kappa |
| rab700_AP28_30_17 | rab700_AP28_30_17H1 | rab700_AP28_30_17H1_Kappa |
| rab700_AP28_30_56 | rab700_AP28_30_56H1 | rab700_AP28_30_56H1_Kappa |
| rab700_AP28_30_120 | rab700_AP28_30_120H2 | rab700_AP28_30_120H2_Kappa |
| rab700_AP30_2A1 | rab700_AP30_2A1_H2 | rab700_AP30_2A1_H2_Kappa |
| rab700_AP30_2A12 | rab700_AP30_2A12H2 | rab700_AP30_2A12H2_Kappa |
| rab700_AP30_1F10 | rab700_AP30_1F10H2 | rab700_AP30_1F10H2_Kappa |
| rab700_AP30_1G9 | rab700_AP30_1G9H1 | rab700_AP30_1G9H1_Kappa |
| rab700_AP30_2H10 | rab700_AP30_2H10H1 | rab700_AP30_2H10H1_Kappa |
| rab700_AP30_4E10 | rab700_AP30_4E10H2 | rab700_AP30_4E10H2_Kappa |
| rab700_AP30_4G7 | rab700_AP30_4G7_H1 | rab700_AP30_4G7_H1_Kappa |
| rab700_AP30_5A12 | rab700_AP30_5A12H1 | rab700_AP30_5A12H1_Kappa |
| rab700_AP30_5C2 | rab700_AP30_5C2_H2 | rab700_AP30_5C2_H2_Kappa |
| rab700_AP3132_25 | rab700_AP3132_25H2 | rab700_AP3132_25H2_Kappa |
| rab700_AP3132_39 | rab700_AP3132_39H1 | rab700_AP3132_39H1_Kappa |
| rab700_AP3132_106 | rab700_AP3132_106H2 | rab700_AP3132_106H2_Kappa |
| rab700_AP3132_40 | rab700_AP3132_40H2 | rab700_AP3132_40H2_Kappa |
| rab700_AP3132_68 | rab700_AP3132_68H1 | rab700_AP3132_68H1_Kappa |
| rab700_AP3132_64 | rab700_AP3132_64H2 | rab700_AP3132_64H2_Kappa |
| rab700_AP3132_89 | rab700_AP3132_89H2 | rab700_AP3132_89H2_Kappa |
| rab700_AP3132_42 | rab700_AP3132_42H2 | rab700_AP3132_42H2_Kappa |
| rab700_AP3132_88 | rab700_AP3132_88H2 | rab700_AP3132_88H2_Kappa |
| rab700_AP3132_101 | rab700_AP3132_101H1 | rab700_AP3132_101H1_Kappa |
| rab700_AP3132_37 | rab700_AP3132_37H2 | rab700_AP3132_37H2_Kappa |
| rab700_AP3132_81 | rab700_AP3132_81H2 | rab700_AP3132_81H2_Kappa |
| rab700_AP3132_63 | rab700_AP3132_63H1 | rab700_AP3132_63H1_Kappa |
| rab700_AP3132_18 | rab700_AP3132_18H1 | rab700_AP3132_18H1_Kappa |
| rab700_AP3132_27 | rab700_AP3132_27H2 | rab700_AP3132_27H2_Kappa |
| rab700_AP3132_16 | rab700_AP3132_16H1 | rab700_AP3132_16H1_Kappa |
| rab700_AP3132_33 | rab700_AP3132_33H1 | rab700_AP3132_33H1_Kappa |
| rab700_AP3132_35 | rab700_AP3132_35H2 | rab700_AP3132_35H2_Kappa |
| rab700_AP3132_62 | rab700_AP3132_62H2 | rab700_AP3132_62H2_Kappa |
| rab700_AP3132_57 | rab700_AP3132_57H1 | rab700_AP3132_57H1_Kappa |
| rab700_AP3132_78 | rab700_AP3132_78H2 | rab700_AP3132_78H2_Kappa |
| rab700_AP3132_17 | rab700_AP3132_17H2 | rab700_AP3132_17H2_Kappa |
| rab700_AP3132_44 | rab700_AP3132_44H1 | rab700_AP3132_44H1_Kappa |
| rab700_AP3132_23 | rab700_AP3132_23H2 | rab700_AP3132_23H2_Kappa |
| rab700_AP3132_65 | rab700_AP3132_65H1 | rab700_AP3132_65H1_Kappa |
| rab700_AP3132_51 | rab700_AP3132_51H1 | rab700_AP3132_51H1_Kappa |
| rab700_AP3132_58 | rab700_AP3132_58H1 | rab700_AP3132_58H1_Kappa |
| rab700_AP3132_52 | rab700_AP3132_52H1 | rab700_AP3132_52H1_Kappa |
| rab700_AP3132_102 | rab700_AP3132_102H2 | rab700_AP3132_102H2_Kappa |
| rab700_AP3132_32 | rab700_AP3132_32H2 | rab700_AP3132_32H2_Kappa |
| rab700_AP3132_107 | rab700_AP3132_107H1 | rab700_AP3132_107H1_Kappa |
| rab700_AP3132_19 | rab700_AP3132_19H2 | rab700_AP3132_19H2_Kappa |
| rab700_AP3132_2 | rab700_AP3132_2H1 | rab700_AP3132_2H1_Kappa |
| rab700_AP3132_8 | rab700_AP3132_8H1 | rab700_AP3132_8H1_Kappa |
| rab700_AP3132_7 | rab700_AP3132_7H1 | rab700_AP3132_7H1_Kappa |
| rab700_AP3132_9 | rab700_AP3132_9H1 | rab700_AP3132_9H1_Kappa |
| rab700_AP3132_4 | rab700_AP3132_4H1 | rab700_AP3132_4H1_Kappa |
| rab700_AP3132_5 | rab700_AP3132_5H2 | rab700_AP3132_5H2_Kappa |
| rab700_AP030_2F9.6 | rab700_AP030_2F9.6H1 | rab700_AP030_2F9.6H1_Kappa |
| rab700_AP030_2F9.8 | rab700_AP030_2F9.8H1 | rab700_AP030_2F9.8H1_Kappa |
| rab700_AP030_50A8.8 | rab700_AP030_50A8.8H1 | rab700_AP030_50A8.8H1_Kappa |
| rab700_AP030_50A8.10 | rab700_AP030_50A8.10H1 | rab700_AP030_50A8.10H1_Kappa |
| rab700_AP030_63A5.4 | rab700_AP030_63A5.4H1 | rab700_AP030_63A5.4H1_Kappa |
| rab700_AP030_63A5.7 | rab700_AP030_63A5.7H1 | rab700_AP030_63A5.7H1_Kappa |
| rab700_AP28_N2A3.2 | rab700_AP28_N2A3.2H | rab700_AP28_N2A3.2_Kappa |
| rab700_AP28_S35B1.6 | rab700_AP28_S35B1.6H | rab700_AP28_S35B1.6_Kappa |
| rab700_AP28_S24F8.11 | rab700_AP28_S24F8.11H | rab700_AP28_S24F8.11_Kappa |
| rab700_AP28_S26G3.7 | rab700_AP28_S26G3.7H | rab700_AP28_S26G3.7_Kappa |
| rab700_AP28_S54E11.2 | rab700_AP28_S54E11.2H | rab700_AP28_S54E11.2_Kappa |
| rab700_AP28_S10C3.2 | rab700_AP28_S10C3.2H | rab700_AP28_S10C3.2_Kappa |
| rab700_AP28_S11B1.5 | rab700_AP28_S11B1.5H | rab700_AP28_S11B1.5_Kappa |
| rab700_AP28_S45A9.7 | rab700_AP28_S45A9.7H | rab700_AP28_S45A9.7_Kappa |
| rab700_AP28_S43F1.3 | rab700_AP28_S43F1.3H | rab700_AP28_S43F1.3_Kappa |
| rab700_AP28_S6G12.6 | rab700_AP28_S6G12.6H | rab700_AP28_S6G12.6_Kappa |
| rab700_AP28_S3D4.7 | rab700_AP28_S3D4.7H | rab700_AP28_S3D4.7_Kappa |
| rab700_AP28_S35H11.8 | rab700_AP28_S35H11.8H | rab700_AP28_S35H11.8_Kappa |
| rab700_AP28_S40C11.1 | rab700_AP28_S40C11.1H | rab700_AP28_S40C11.1_Kappa |
| rab700_AP28_S9F8.3 | rab700_AP28_S9F8.3H | rab700_AP28_S9F8.3_Kappa |
| rab700_AP28_S39A3.1 | rab700_AP28_S39A3.1H | rab700_AP28_S39A3.1_Kappa |
| rab700_AP28_S3G1.5 | rab700_AP28_S3G1.5H | rab700_AP28_S3G1.5_Kappa |
| rab700_AP32_N8D7.1 | rab700_AP32_N8D7.1H | rab700_AP32_N8D7.1_Kappa |
| rab700_AP32_S7F12.6 | rab700_AP32_S7F12.6H | rab700_AP32_700_S7F12.6_Kappa |

Thus, in certain embodiments, an antibody, or antigen-binding fragment thereof, binds to SIRPα and comprises a VH and a corresponding VL region selected from Table 112. In particular embodiments, the VH region comprises an amino acid sequence having at least 90%, 95%, 98%, 99%, or 100% identity to a sequence selected from SEQ ID NOs: 169, 171, 173, 175, 177, 179, 181, 183, 185, 187, 189, 191, 193, 195, 197, 199, 201, 203, 205, 207, 209, 211, 213, 215, 217, 219, 221, and 223. In some embodiments, the VL region comprises an amino acid sequence having at least 90%, 95%, 98%, 99%, or 100% identity to a sequence selected from SEQ ID NOs: 168, 170, 172, 174, 176, 178, 180, 182, 184 or 227, 186, 188, 190, 192, 194, 196, 198, 200, 202, 204, 206, 208, 210, 212, 214, 216, 218, 220, 222, and 224. In specific embodiments, an antibody, or antigen-binding fragment thereof, comprises:
the VH region set forth in SEQ ID NO: 169, and the VL region set forth in SEQ ID NO: 170;
the VH region set forth in SEQ ID NO: 171, and the VL region set forth in SEQ ID NO: 172;
the VH region set forth in SEQ ID NO: 173, and the VL region set forth in SEQ ID NO: 174;
the VH region set forth in SEQ ID NO: 175, and the VL region set forth in SEQ ID NO: 176;
the VH region set forth in SEQ ID NO: 177, and the VL region set forth in SEQ ID NO: 178;
the VH region set forth in SEQ ID NO: 179, and the VL region set forth in SEQ ID NO: 180;
the VH region set forth in SEQ ID NO: 181, and the VL region set forth in SEQ ID NO: 182;
the VH region set forth in SEQ ID NO: 183, and the VL region set forth in SEQ ID NO: 184 or 227;
the VH region set forth in SEQ ID NO: 185, and the VL region set forth in SEQ ID NO: 186;
the VH region set forth in SEQ ID NO: 187, and the VL region set forth in SEQ ID NO: 188;
the VH region set forth in SEQ ID NO: 189, and the VL region set forth in SEQ ID NO: 190;
the VH region set forth in SEQ ID NO: 191, and the VL region set forth in SEQ ID NO: 192;
the VH region set forth in SEQ ID NO: 193, and the VL region set forth in SEQ ID NO: 194;
the VH region set forth in SEQ ID NO: 195, and the VL region set forth in SEQ ID NO: 196;
the VH region set forth in SEQ ID NO: 197, and the VL region set forth in SEQ ID NO: 198;
the VH region set forth in SEQ ID NO: 199, and the VL region set forth in SEQ ID NO: 200;
the VH region set forth in SEQ ID NO: 201, and the VL region set forth in SEQ ID NO: 202;
the VH region set forth in SEQ ID NO: 203, and the VL region set forth in SEQ ID NO: 204;
the VH region set forth in SEQ ID NO: 205, and the VL region set forth in SEQ ID NO: 206;
the VH region set forth in SEQ ID NO: 207, and the VL region set forth in SEQ ID NO: 208;
the VH region set forth in SEQ ID NO: 209, and the VL region set forth in SEQ ID NO: 210;
the VH region set forth in SEQ ID NO: 211, and the VL region set forth in SEQ ID NO: 212;
the VH region set forth in SEQ ID NO: 213, and the VL region set forth in SEQ ID NO: 214;
the VH region set forth in SEQ ID NO: 215, and the VL region set forth in SEQ ID NO: 216;
the VH region set forth in SEQ ID NO: 217, and the VL region set forth in SEQ ID NO: 218;
the VH region set forth in SEQ ID NO: 219, and the VL region set forth in SEQ ID NO: 220;
the VH region set forth in SEQ ID NO: 221, and the VL region set forth in SEQ ID NO: 222; or

the VH region set forth in SEQ ID NO 223:, and the VL region set forth in SEQ ID NO: 224. In some embodiments, an antibody, or an antigen-binding fragment thereof, binds to SIRPα, and comprises a heavy chain variable (VH) region comprising VHCDR1, VHCDR2, and VHCDR3 regions selected from the underlined sequences in Table R1; and a corresponding (by clone name) light chain variable (VL) region comprising VLCDR1, VLCDR2, and VLCDR3 regions selected from underlined sequences in Table R2. In some embodiments, the VH region comprises an amino acid sequence selected from Table R1, and the VL region comprises a corresponding (by clone name) amino acid sequence selected from Table R2, as defined, for example, in Table R3.

In some embodiments, as noted above, an antibody, or an antigen-binding fragment thereof, binds to SIRPα, for example, soluble or cell-expressed SIRPα. In particular embodiments, the SIRPα is human SIRPα, or a domain or epitope thereof. Exemplary polypeptides, domains, and epitopes of human SIRPα are provided in Table S1 below.

TABLE S1
Exemplary SIRPα Sequences

| Domain and | | SEQ ID |
|---|---|---|
| Residues | Sequence | NO: |
| Extracellular | GVAGEEELQVIQPDKSVLVAAGETATLRCTATSLIPVGPIQWFRGAG | 228 |
| Domain | PGRELIYNQKEGHFPRVTTVSDLTKRNNMDFSIRIGNITPADAGTYY | |
| | CVKFRKGSPDDVEFKSGAGTELSVRAKPSAPVVSGPAARATPQHTVS | |
| | FTCESHGFSPRDITLKWFKNGNELSDFQTNVDPVGESVSYSIHSTAK | |
| | VVLTREDVHSQVICEVAHVTLQGDPLRGTANLSETIRVPPTLEVTQQ | |
| | PVRAENQVNVTCQVRKFYPQRLQLTWLENGNVSRTETASTVTENKDG | |
| | TYNWMSWLLVNVSAHRDDVKLTCQVEHDGQPAVSKSHDLKVSAHPKE | |
| | QGSNTAAENTGSNERNIY | |
| Ig-like V-type | EELQVIQPDKSVLVAAGETATLRCTATSLIPVGPIQWFRGAGPGREL | 229 |
| (32-137) | IYNQKEGHFPRVTTVSDLTKRNNMDFSIRIGNITPADAGTYYCVKFR | |
| | KGSPDDVEFKSG | |
| Ig-like C1-type | PSAPVVSGPAARATPQHTVSFTCESHGFSPRDITLKWFKNGNELSDF | 230 |
| 1 (148-247) | QTNVDPVGESVSYSIHSTAKVVLTREDVHSQVICEVAHVTLQGDPLR | |
| | GTANLS | |
| Ig-like C1-type | PTLEVTQQPVRAENQVNVTCQVRKFYPQRLQLTWLENGNVSRTETAS | 231 |
| 2 (254-348) | TVTENKDGTYNWMSWLLVNVSAHRDDVKLTCQVEHDGQPAVSKSHDL | |
| | K | |
| 14H2B Epitope | SDLTKRNNMDFS | 232 |
| 14H2B Epitope′ | KGSPDDVEFKSGAGT | 233 |
| 89H Epitope | HDLKVSAHPKEQGS | 234 |
| Epitope region | HFPRVTTVSDLTKRNNMDFSI | 235 |
| Epitope region | KGSPDDVEFKSGAGTELSVRA | 236 |
| Epitope region | DGQPAVSKSHDLKVSAHPKEQGSNTA | 237 |

In certain embodiments, an antibody, or an antigen-binding fragment thereof, specifically binds to a human SIRPα, for example, at least one human SIRPα polypeptide, domain, or epitope selected from Table S1. In some embodiments, an antibody, or an antigen-binding fragment thereof, binds to one or more SIRPα variants (for example, V1, V2, V3, V4, V5, V6, V7, V8, V9, V10 variants), including the V1, V2, and/or V8 SIRPα variants. In some embodiments, an antibody, or an antigen-binding fragment thereof, selectively binds to SIRPα with high affinity, and does not significantly bind to SIRPβ and/or SIRPγ. In some embodiments, an antibody, or an antigen-binding fragment thereof, binds to SIRPα and SIRPβ, and does not substantially bind to SIRPγ. In some embodiments, an antibody, or an antigen-binding fragment thereof, binds to SIRPα and SIRPγ, and does not substantially bind to SIRPβ. In some embodiments, an antibody, or an antigen-binding fragment thereof, binds to SIRPα, SIRPβ, and SIRPγ. In some embodiments, an antibody, or an antigen-binding fragment thereof, binds to SIRPα and SIRPβL. In certain embodiments, an antibody, or an antigen-binding fragment thereof, binds to SIRPα and does not substantially bind to SIRPβL. In some embodiments, an antibody, or an antigen-binding fragment thereof, binds to cynomolgus SIRPα, for example, it cross-reactively binds to a human SIRPα and cynomolgus SIRPα. In some embodiments, an antibody, or an antigen-binding fragment thereof, binds to myeloid cells, for example, monocytes, macrophages, dendritic cells, and/or neutrophils.

In certain embodiments, an antibody, or an antigen-binding fragment thereof, specifically binds to human SIRPα, for example, at least one human SIRPα peptide epitope selected from Table S1, for example, at least one, two, or three peptide epitopes selected from Table S1. In some embodiments, an antibody, or an antigen-binding fragment thereof, specifically binds to human SIRPα at a peptide epitope that comprises, consists, or consists essentially of one or more residues selected from S68, L70, T71, R73, S79, K100, S102, and T114, as defined by the human SIRPα sequence of SEQ ID NO: 228. In some embodiments, an antibody, or an antigen-binding fragment thereof, specifically binds to human SIRPα at a peptide epitope that comprises, consists, or consists essentially of one or more residues selected from SDLTKRNNMDFS (SEQ ID NO: 232) and KGSPDDVEFKSGAGT (SEQ ID NO: 233). In some embodiments, an antibody, or an antigen-binding fragment thereof, specifically binds to human SIRPβ and/or human SIRPβL at the corresponding, conserved epitope, for example, as defined by SDLTKRNNMDFS (SEQ ID NO: 232) and/or KGSPDDVEFKSGAGT (SEQ ID NO: 233). In some embodiments, an antibody, or an antigen-binding fragment thereof, specifically binds to human SIRPα at a peptide epitope that comprises, consists, or consists essentially of one or more residues selected from H319 and 5332, as defined by the human SIRPα sequence of SEQ ID NO: 228. In some embodiments, an antibody, or an antigen-binding fragment thereof, specifically binds to human SIRPα at a peptide epitope that comprises, consists, or consists essentially of HDLKVSAHPKEQGS (SEQ ID NO: 234). In some embodiments, an antibody, or an antigen-binding fragment thereof, specifically binds to human SIRPβ and/or human SIRPβL at the corresponding, conserved epitope, for example, as defined by HDLKVSAHPKEQGS (SEQ ID NO: 234).

In specific embodiments, an antibody, or antigen-binding fragment thereof, binds to a human SIRPα with a K_{D} of 0.4 nM or lower. In particular embodiments, an antibody, or antigen-binding fragment thereof, comprises an IgG1 Fc region and binds to SIRPα with a K_{D} of about 0.16 to about 2.5 nM. In specific embodiments, an antibody, or antigen-binding fragment thereof, comprises an IgG4 Fc region having an S228P substitution and binds SIRPα with a K_{D} of about 0.09 to about 1.66 nM, or about 0.088 nM, about 0.2643 nM, about 0.3778 nM, about 0.672 nM, about 0.6864 nM, or about 1.66 nM, for example, as measured by flow cytometric analysis of binding to cell surface SIRPα expressed on dendritic cells (see Example 1).

In some embodiments, an antibody, or antigen-binding fragment thereof, binds to SIRPα and is a SIRPα antagonist. For instance, in some embodiments, an antibody, or antigen-binding fragment thereof, inhibits or reduces SIRPα binding to CD47, and in some instances reduces SIRPα-CD47-mediated signaling activities, for example, with an IC₅₀ of 0.8 nM or higher. In some embodiments, an antibody, or antigen-binding fragment thereof, increases phagocytosis by innate immune cells such as, macrophages, dendritic cells, and/or neutrophils, for example, by inhibiting or reducing SIRPα-mediated inhibition of phagocytosis. In some embodiments, an antibody, or antigen-binding fragment thereof, increases antibody-dependent cell phagocytosis (ADCP). In some embodiments, an antibody, or antigen-binding fragment thereof, increases dendritic cell activation of cytotoxic T cells. Here, inhibition of SIRPα on dendritic cells induces spontaneous and enhanced production of IL12 and T cell costimulatory molecules (Liu et al., Oncoimmunology. 5 (9): e1183850, 2016), resulting in more potent cytotoxic T lymphocyte responses.

In some embodiments, an antibody, or antigen-binding fragment thereof, binds to SIRPα but does not significantly inhibit binding of SIRPα to its ligand CD47 and does not significantly inhibit SIRPα-CD47-mediated signaling.

In particular embodiments, as noted above, an antibody, or antigen-binding fragment thereof, binds to a SIRPα and does not significantly bind to SIRPγ. In some embodiments, an antibody, or antigen-binding fragment thereof, does not significantly inhibit or reduce SIRPγ binding to CD47, and does not significantly reduce SIRPγ-CD47-mediated signaling activities. In some embodiments, an antibody, or antigen-binding fragment thereof, does not significantly bind to primary T cells, and in some instances does not significantly inhibit T cell proliferation and/or trans-endothelial T cell migration.

In some embodiments, an antibody, or antigen-binding fragment thereof, binds to SIRPα and is a SIRPα agonist. In some embodiments, an antibody, or antigen-binding fragment thereof, binds to SIRPα and SIRPβ and is a SIRPα agonist. Certain anti-SIRPα agonist antibodies can be used, for example, to induce immune suppression, for instance, by reducing activation of and/or phagocytosis by innate immune cells such as macrophages, dendritic cells, and/or natural killer cells. Thus, in some embodiments, an antibody, or antigen-binding fragment thereof, inhibits innate immune cell-medicated phagocytosis, for example, macrophage-mediated phagocytosis.

Merely for illustrative purposes, the binding interactions between an antibody, or antigen-binding fragment thereof, and a SIRPα polypeptide can be detected and quantified using a variety of routine methods, including biacore assays (for example, with appropriately tagged soluble reagents, bound to a sensor chip), FACS analyses with cells expressing a SIRPα polypeptide on the cell surface (either native, or recombinant), immunoassays, fluorescence staining assays, ELISA assays, and microcalorimetry approaches such as ITC (Isothermal Titration calorimetry).

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. As used herein, the term encompasses not only intact polyclonal or monoclonal antibodies, but also fragments thereof (such as dAb, Fab, Fab′, F(ab′)2, Fv), single chain (scFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. “Diabodies”, multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “antigen-binding fragment” as used herein refers to a polypeptide fragment that contains at least one CDR of an immunoglobulin heavy and/or light chains that binds to the antigen of interest, in particular to SIRPα. In this regard, an antigen-binding fragment of the herein described antibodies may comprise 1, 2, 3, 4, 5, or all 6 CDRs of a VH and VL sequence set forth herein from antibodies that bind SIRPα.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an antibody is said to specifically bind an antigen when it preferentially recognizes its target antigen in a complex mixture of proteins and/or macromolecules. An antibody is said to specifically bind an antigen when the equilibrium dissociation constant is ≤10⁻⁷ or 10⁻⁸ M. In some embodiments, the equilibrium dissociation constant may be ≤10⁻⁹ M or ≤10⁻¹⁰ M.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures, regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments, etc., described herein under the definition of “antibody”.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFv antibodies are contemplated. For example, Kappa bodies (Ill et al., Prot. Eng. 10: 949-57 (1997); minibodies (Martin et al., EMBO J 13: 5305-9 (1994); diabodies (Holliger et al., PNAS 90: 6444-8 (1993); or Janusins (Traunecker et al., EMBO J 10: 3655-59 (1991) and Traunecker et al., Int. J. Cancer Suppl. 7: 51-52 (1992), may be prepared using standard molecular biology techniques following the teachings of the present application with regard to selecting antibodies having the desired specificity. Some embodiments include bispecific or chimeric antibodies. For example, a chimeric antibody may comprise CDRs and framework regions from different antibodies, while bispecific antibodies may be generated that bind specifically to SIRPα through one binding domain and to a second molecule through a second binding domain. These antibodies may be produced through recombinant molecular biological techniques or may be physically conjugated together.

A single chain Fv (scFv) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated, but chemically separated, light and heavy polypeptide chains from an antibody V region into an scFv molecule which will fold into a three dimensional structure significantly similar to the structure of an antigen-binding site. See, e.g., U.S. Pat. Nos. 5,091,513 and 5,132,405, to Huston et al.; and U.S. Pat. No. 4,946,778, to Ladner et al.

Certain embodiments include “probodies”, or antibodies where the binding site(s) are masked or otherwise inert until activated by proteolytic cleavage in target or disease tissue. Certain of these and related embodiments comprise one or more masking moieties that sterically hinder the antigen binding site(s) of the antibody, and which are fused to the antibody via one or more proteolytically-cleavable linkers (see, for example, Polu and Lowman, Expert Opin. Biol. Ther. 14:1049-1053, 2014).

In certain embodiments, a SIRPα binding antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific or multi-specific antibodies are to be used, these may be conventional bispecific antibodies, which can be manufactured in a variety of ways (Holliger, P. and Winter G. Current Opinion Biotechnol. 4, 446-449 (1993)), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., Protein Eng., 9, 616-621, 1996).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies described herein take the form of a Nanobody®. Nanobodies® are encoded by single genes and are efficiently produced in almost all prokaryotic and eukaryotic hosts e.g. E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254). The production process is scalable and multi-kilogram quantities of Nanobodies® have been produced. Nanobodies may be formulated as a ready-to-use solution having a long shelf life. The Nanoclone® method (see, e.g., WO 06/079372) is a proprietary method for generating Nanobodies against a desired target, based on automated high-throughput selection of B-cells.

In certain embodiments, the anti-SIRPα antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-SIRPα antibodies disclosed herein include the methods described in U.S. Pat. No. 7,462,697. Illustrative humanized antibodies according to certain embodiments comprise the humanized sequences provided in Table H1 and Table H2.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992)J Immunol 148:1149-1154. In some embodiments, humanized antibodies preserve all CDR sequences (for example, a humanized rabbit antibody which contains all six CDRs from the rabbit antibody). In some embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies are chimeric antibodies. In this regard, a chimeric antibody is comprised of an antigen-binding fragment of an anti-SIRPα antibody operably linked or otherwise fused to a heterologous Fc portion of a different antibody. In certain embodiments, the heterologous Fc domain is of human origin. In some embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In some embodiments, the heterologous Fc domain may be comprised of CH2 and CH3 domains from one or more of the different Ig classes. As noted above with regard to humanized antibodies, the anti-SIRPα antigen-binding fragment of a humanized antibody may comprise only one or more of the CDRs of the antibodies described herein (e.g., 1, 2, 3, 4, 5, or 6 CDRs of the antibodies described herein), or may comprise an entire variable domain (VL, VH or both).

In certain embodiments, a SIRPα-binding antibody comprises one or more of the CDRs of the antibodies described herein. In this regard, it has been shown in some cases that the transfer of only the VHCDR3 of an antibody can be performed while still retaining desired specific binding (Barbas et al., PNAS (1995) 92: 2529-2533). See also, McLane et al., PNAS (1995) 92:5214-5218, Barbas et al., J. Am. Chem. Soc. (1994) 116:2161-2162.

Marks et al. (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire may be combined with a CDR3 of a particular antibody. Using analogous techniques, the CDR3-derived sequences of the presently described antibodies may be shuffled with repertoires of VH or VL domains lacking a CDR3, and the shuffled complete VH or VL domains combined with a cognate VL or VH domain to provide an antibody or antigen-binding fragment thereof that binds SIRPα. The repertoire may then be displayed in a suitable host system such as the phage display system of WO 92/01047 so that suitable antibodies or antigen-binding fragments thereof may be selected. A repertoire may consist of at least from about 10⁴ individual members and upwards by several orders of magnitude, for example, to about from 10⁶ to 10⁸ or 10¹⁰ or more members. Analogous shuffling or combinatorial techniques are also disclosed by Stemmer (Nature, 1994, 370:389-391), who describes the technique in relation to a β-lactamase gene but observes that the approach may be used for the generation of antibodies.

A further alternative is to generate novel VH or VL regions carrying one or more CDR-derived sequences described herein using random mutagenesis of one or more selected VH and/or VL genes to generate mutations within the entire variable domain. Such a technique is described by Gram et al (1992, Proc. Natl. Acad. Sci., USA, 89:3576-3580), who used error-prone PCR. Another method which may be used is to direct mutagenesis to CDR regions of VH or VL genes. Such techniques are disclosed by Barbas et al., (1994, Proc. Natl. Acad. Sci., USA, 91:3809-3813) and Schier et al (1996, J. Mol. Biol. 263:551-567).

In certain embodiments, a specific VH and/or VL of the antibodies described herein may be used to screen a library of the complementary variable domain to identify antibodies with desirable properties, such as increased affinity for SIRPα. Such methods are described, for example, in Portolano et al., J. Immunol. (1993) 150:880-887; Clarkson et al., Nature (1991) 352:624-628.

Other methods may also be used to mix and match CDRs to identify antibodies having desired binding activity, such as binding to SIRPα. For example: Klimka et al., British Journal of Cancer (2000) 83: 252-260, describe a screening process using a mouse VL and a human VH library with CDR3 and FR4 retained from the mouse VH. After obtaining antibodies, the VH was screened against a human VL library to obtain antibodies that bound antigen. Beiboer et al., J. Mol. Biol. (2000) 296:833-849 describe a screening process using an entire mouse heavy chain and a human light chain library. After obtaining antibodies, one VL was combined with a human VH library with the CDR3 of the mouse retained. Antibodies capable of binding antigen were obtained. Rader et al., PNAS (1998) 95:8910-8915 describe a process similar to Beiboer et al above.

These just-described techniques are, in and of themselves, known as such in the art. The skilled person will, however, be able to use such techniques to obtain antibodies or antigen-binding fragments thereof according to several embodiments described herein, using routine methodology in the art.

Also disclosed herein is a method for obtaining an antibody antigen binding domain specific for a SIRPα antigen, the method comprising providing by way of addition, deletion, substitution or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations to identify a specific binding member or an antibody antigen binding domain specific for SIRPα and optionally with one or more desired properties. The VL domains may have an amino acid sequence which is significantly as set out herein. An analogous method may be employed in which one or more sequence variants of a VL domain disclosed herein are combined with one or more VH domains.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a SIRPα epitope is an antibody that binds one SIRPα epitope with greater affinity, avidity, more readily, and/or with greater duration than it binds to other SIRPα epitopes or non-SIRPα epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (K_{d}) of the interaction, wherein a smaller K_{d} represents a greater affinity Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

The term “immunologically active”, with reference to an epitope being or “remaining immunologically active”, refers to the ability of an anti-SIRPα antibody to bind to the epitope under different conditions, for example, after the epitope has been subjected to reducing and denaturing conditions.

An antibody or antigen-binding fragment thereof according to certain preferred embodiments of the present application may be one that competes for binding to SIRPα with any antibody described herein which both (i) specifically binds to the antigen and (ii) comprises a VH and/or VL domain disclosed herein, or comprises a VH CDR3 disclosed herein, or a variant of any of these. Competition between antibodies may be assayed easily in vitro, for example using ELISA and/or by tagging a specific reporter molecule to one antibody which can be detected in the presence of other untagged antibodies, to enable identification of specific antibodies which bind the same epitope or an overlapping epitope. Thus, there is provided herein a specific antibody or antigen-binding fragment thereof, comprising a human antibody antigen-binding site which competes with an antibody described herein that binds to SIRPα.

In this regard, as used herein, the terms “competes with”, “inhibits binding” and “blocks binding” (e.g., referring to inhibition/blocking of binding of a ligand (e.g., CD47) and/or counter-receptor to SIRPα, or referring to inhibition/blocking of binding of an anti-SIRPα antibody to CD47) are used interchangeably and encompass both partial and complete inhibition/blocking. The inhibition/blocking of a ligand and/or counter-receptor to SIRPα preferably reduces or alters the normal level or type of cell signaling that occurs when a ligand and/or counter-receptor binds to SIRPα without inhibition or blocking. Inhibition and blocking are also intended to include any measurable decrease in the binding of a ligand and/or counter-receptor to SIRPα when in contact with an anti-SIRPα antibody as disclosed herein as compared to the ligand not in contact with an anti-SIRPα antibody, e.g., the blocking of a ligand (e.g., CD47) and/or counter-receptor to SIRPα by at least about 10%, 20%, 30%, 40%, 50%, 60%, 65%, 70%, 75%, 80%, 85%, 90%, 91%, 92%, 93%, 94%, 95%, 96%, 97%, 98%, 99%, or 100%.

The constant regions of immunoglobulins show less sequence diversity than the variable regions, and are responsible for binding a number of natural proteins to elicit important biochemical events. In humans there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region.

The Fc region of an antibody interacts with a number of Fc receptors and ligands, imparting an array of important functional capabilities referred to as effector functions. In some embodiments, an anti-SIRPα antibody comprises an Fc region. In some embodiments, an anti-SIRPα antibody comprises a human IgG1 Fc region. In some embodiments, an anti-SIRPα antibody comprises a human IgG4 Fc region, for example, an IgG4 region having an S228P substitution.

For IgG the Fc region comprises Ig domains CH2 and CH3 and the N-terminal hinge leading into CH2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273.) (pdb accession codes 1IIS and 1IIX) (Radaev et al., 2001, J Biol Chem 276:16469-16477.)

The different IgG subclasses have different affinities for the FcγRs, with IgG1 and IgG3 typically binding significantly better to the receptors than IgG2 and IgG4 (Jefferis et al., 2002, Immunol Lett 82:57-65). All FcγRs bind the same region on IgG Fc, yet with different affinities: the high affinity binder FcγRI has a K_{d} for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical; however, FcγRIIIb does not have an intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Patients with the V158 allotype respond favorably to rituximab treatment; however, patients with the lower affinity F158 allotype respond poorly (Cartron et al., 2002, Blood 99:754-758). Approximately 10-20% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, that is, they have at least one allele of the F158 FcγRIIIa.

The Fc region is also involved in activation of the complement cascade. In the classical complement pathway, C1 binds with its C1q subunits to Fc fragments of IgG or IgM, which has formed a complex with antigen(s). In certain embodiments, modifications to the Fc region comprise modifications that alter (either enhance or decrease) the ability of a SIRPα-specific antibody as described herein to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Methods, 202:163 (1996)).

Certain embodiments provide anti-SIRPα antibodies having a modified Fc region with altered functional properties, such as reduced or enhanced CDC, ADCC, or ADCP activity, or enhanced binding affinity for a specific FcγR or increased serum half-life. Other modified Fc regions contemplated herein are described, for example, in issued U.S. Pat. Nos. 7,317,091; 7,657,380; 7,662,925; 6,538,124; 6,528,624; 7,297,775; 7,364,731; Published U.S. Applications US2009092599; US20080131435; US20080138344; and published International Applications WO2006/105338; WO2004/063351; WO2006/088494; WO2007/024249.

Thus, in certain embodiments, antibody variable domains with the desired binding specificities are fused to immunoglobulin constant domain sequences. In certain embodiments, the fusion is with an Ig heavy chain constant domain, comprising at least part of the hinge, C_{H}2, and C_{H}3 regions. In some instances, it is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The antibodies described herein (and antigen-binding fragments and variants thereof) may also be modified to include an epitope tag or label, e.g., for use in purification or diagnostic applications. There are many linking groups known in the art for making antibody conjugates, including, for example, those disclosed in U.S. Pat. No. 5,208,020 or EP Patent 0 425 235 B1, and Chari et al., Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

In some embodiments, a SIRPα-specific antibody as described herein may be conjugated or operably linked to another agent or therapeutic compound, referred to herein as a conjugate. The agent or therapeutic compound may be a polypeptide agent, a polynucleotide agent, cytotoxic agent, a chemotherapeutic agent, a cytokine, an anti-angiogenic agent, a tyrosine kinase inhibitor, a toxin, a radioisotope, or other therapeutically active agent. Chemotherapeutic agents, cytokines, anti-angiogenic agents, tyrosine kinase inhibitors, and other therapeutic agents have been described herein, and all of these aforementioned therapeutic agents may find use as antibody conjugates. Such conjugates can be used, for example, to target the agent or compound to a site of action, for example, a tumor or tumor microenvironment characterized by the expression of SIRPα.

In some embodiments, the antibody is conjugated or operably linked to a toxin, including but not limited to small molecule toxins, polypeptides, nucleic acids, and enzymatically active toxins of bacterial, fungal, plant or animal origin, including fragments and/or variants thereof. Small molecule toxins include but are not limited to saporin (Kuroda K, et al., The Prostate 70:1286-1294 (2010); Lip, W L. et al., 2007 Molecular Pharmaceutics 4:241-251; Quadros E V., et al., 2010 Mol Cancer Ther; 9(11); 3033-40; Polito L., et al. 2009 British Journal of Haematology, 147, 710-718), calicheamicin, maytansine (U.S. Pat. No. 5,208,020), trichothene, and CC1065. Toxins include but are not limited to RNase, gelonin, enediynes, ricin, abrin, diptheria toxin, cholera toxin, gelonin, Pseudomonas exotoxin (PE40), Shigella toxin, Clostridium perfringens toxin, and pokeweed antiviral protein.

In some embodiments, an antibody or antigen-binding fragment thereof is conjugated to one or more maytansinoid molecules. Maytansinoids are mitotic inhibitors that act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533 Immunoconjugates containing maytansinoids and their therapeutic use are disclosed, for example, in U.S. Pat. Nos. 5,208,020, 5,416,064 and European Patent EP 0 425 235 B1. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay.

Antibody-maytansinoid conjugates are prepared by chemically linking an antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and non-patent publications referred to hereinabove. Preferred maytansinoids are maytansinol and maytansinol analogues modified in the aromatic ring or at other positions of the maytansinol molecule, such as various maytansinol esters.

Another conjugate of interest comprises an antibody conjugated to one or more calicheamicin molecules. The calicheamicin family of antibiotics is capable of producing double-stranded DNA breaks at sub-picomolar concentrations. Structural analogues of calicheamicin that may also be used (Hinman et al., 1993, Cancer Research 53:3336-3342; Lode et al., 1998, Cancer Research 58:2925-2928) (U.S. Pat. Nos. 5,714,586; 5,712,374; 5,264,586; 5,773,001). Dolastatin 10 analogs such as auristatin E (AE) and monomethylauristatin E (MMAE) may find use as conjugates for the presently disclosed antibodies, or variants thereof (Doronina et al., 2003, Nat Biotechnol 21(7):778-84; Francisco et al., 2003 Blood 102(4):1458-65). Useful enzymatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, PCT WO 93/21232. Also included are embodiments in which a conjugate or fusion is formed between a SIRPα-specific antibody described herein and a compound with nucleolytic activity, for example a ribonuclease or DNA endonuclease such as a deoxyribonuclease (DNase).

In some embodiments, a herein-disclosed antibody may be conjugated or operably linked to a radioisotope to form a radioconjugate. A variety of radioactive isotopes are available for the production of radioconjugate antibodies. Examples include, but are not limited to ⁹⁰Y, ¹²³I, ¹²⁵I, ¹³¹I, ¹⁸⁶Re, ¹⁸⁸Re, ²¹¹At, and ²¹²Bi.

Antibodies described herein may in certain embodiments be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxel/paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. One exemplary cytotoxin is saporin (available from Advanced Targeting Systems, San Diego, CA). Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine).

Moreover, a SIRPα-specific antibody (including a functional fragment thereof as provided herein such as an antigen-binding fragment) may in certain embodiments be conjugated to therapeutic moieties such as a radioactive materials or macrocyclic chelators useful for conjugating radiometal ions. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50.

In some embodiments, an antibody may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In some embodiments, the antibody is conjugated or operably linked to an enzyme in order to employ Antibody Dependent Enzyme Mediated Prodrug Therapy (ADEPT). ADEPT may be used by conjugating or operably linking the antibody to a prodrug-activating enzyme that converts a prodrug (e.g. a peptidyl chemotherapeutic agent, see PCT WO 81/01145) to an active anti-cancer drug. See, for example, PCT WO 88/07378 and U.S. Pat. No. 4,975,278. The enzyme component of the immunoconjugate useful for ADEPT includes any enzyme capable of acting on a prodrug in such a way so as to convert it into its more active, cytotoxic form. Enzymes that are useful in the method of these and related embodiments include but are not limited to alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuramimidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, may be used to convert prodrugs into free active drugs (see, for example, Massey, 1987, Nature 328: 457-458). Antibody-abzyme conjugates can be prepared for delivery of the abzyme to a tumor cell population.

Immunoconjugates may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage. The linker may be a “cleavable linker” facilitating release of one or more cleavable components. For example, an acid-labile linker may be used (Cancer Research 52: 127-131 (1992); U.S. Pat. No. 5,208,020).

Other modifications of the antibodies (and polypeptides) are included. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

The desired functional properties of anti-SIRPα antibodies may be assessed using a variety of methods known to the skilled person affinity/binding assays (for example, surface plasmon resonance, competitive inhibition assays); cytotoxicity assays, cell viability assays, cell proliferation or differentiation assays, cancer cell and/or tumor growth inhibition using in vitro or in vivo models. Other assays may test the ability of antibodies described herein to block normal SIRPα-mediated responses. The antibodies described herein may also be tested for in vitro and in vivo efficacy. Such assays may be performed using well-established protocols known to the skilled person (see e. g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or commercially available kits.

Certain embodiments include an isolated nucleic acid encoding an antibody or antigen-binding fragment thereof as described herein, for instance, a nucleic acid which codes for a CDR or VH or VL domain as described herein. Nucleic acids include DNA and RNA. These and related embodiments may include polynucleotides encoding antibodies that bind SIRPα as described herein. The term “isolated polynucleotide” as used herein shall mean a polynucleotide of genomic, cDNA, or synthetic origin or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials. Polynucleotides may comprise a native sequence or may comprise a sequence that encodes a variant or derivative of such a sequence.

In some embodiments, polynucleotide variants may have substantial identity to a polynucleotide sequence encoding an anti-SIRPα antibody described herein. For example, a polynucleotide may be a polynucleotide comprising at least 70% sequence identity, preferably at least 75%, 80%, 85%, 90%, 95%, 96%, 97%, 98%, or 99% or higher, sequence identity compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of the antibody encoded by the variant polynucleotide is not significantly diminished relative to an antibody encoded by a polynucleotide sequence specifically set forth herein.

In certain embodiments, polynucleotide fragments may comprise or consist essentially of various lengths of contiguous stretches of sequence identical to or complementary to a sequence encoding an antibody as described herein. For example, polynucleotides are provided that comprise or consist essentially of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of a sequences the encodes an antibody, or antigen-binding fragment thereof, disclosed herein as well as all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding an antibody described herein or at both ends of a polynucleotide encoding an antibody described herein.

In some embodiments, polynucleotides are provided that are capable of hybridizing under moderate to high stringency conditions to a polynucleotide sequence encoding an antibody, or antigen-binding fragment thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in some embodiments, suitable highly stringent hybridization conditions include those described above, with the exception that the temperature of hybridization is increased, e.g., to 60° C.-65° C. or 65° C.-70° C.

In certain embodiments, the polynucleotides described above, e.g., polynucleotide variants, fragments and hybridizing sequences, encode antibodies that bind SIRPα, or antigen-binding fragments thereof. In other embodiments, such polynucleotides encode antibodies or antigen-binding fragments, or CDRs thereof, that bind to SIRPα at least about 50%, at least about 70%, and in certain embodiments, at least about 90% as well as an antibody sequence specifically set forth herein. In further embodiments, such polynucleotides encode antibodies or antigen-binding fragments, or CDRs thereof, that bind to SIRPα with greater affinity than the antibodies set forth herein, for example, that bind quantitatively at least about 105%, 106%, 107%, 108%, 109%, or 110% as well as an antibody sequence specifically set forth herein.

As described elsewhere herein, determination of the three-dimensional structures of representative polypeptides (e.g., variant SIRPα-specific antibodies as provided herein, for instance, an antibody protein having an antigen-binding fragment as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate polynucleotides encoding the amino acid sequence) within an antibody such that, for example, affinity is maintained or better affinity is achieved.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign™ program in the Lasergene® suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, C A (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encodes an antibody as described herein. Some of these polynucleotides bear minimal sequence identity to the nucleotide sequence of the native or original polynucleotide sequence that encode antibodies that bind to SIRPα. Nonetheless, polynucleotides that vary due to differences in codon usage are expressly included. In certain embodiments, sequences that have been codon-optimized for mammalian expression are specifically contemplated.

Therefore, in certain embodiments, a mutagenesis approach, such as site-specific mutagenesis, may be employed for the preparation of variants and/or derivatives of the antibodies described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode an antibody disclosed herein, or an antigen-binding fragment thereof, to alter one or more properties of the encoded polypeptide, such as the binding affinity of the antibody or the antigen-binding fragment thereof, or the function of a particular Fc region, or the affinity of the Fc region for a particular FcγR. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

In many embodiments, the nucleic acids encoding a subject monoclonal antibody are introduced directly into a host cell, and the cell incubated under conditions sufficient to induce expression of the encoded antibody. The antibodies described herein are prepared using standard techniques well known to those of skill in the art in combination with the polypeptide and nucleic acid sequences provided herein. The polypeptide sequences may be used to determine appropriate nucleic acid sequences encoding the particular antibody disclosed thereby. The nucleic acid sequence may be optimized to reflect particular codon “preferences” for various expression systems according to standard methods well known to those of skill in the art.

According to certain related embodiments there is provided a recombinant host cell which comprises one or more constructs as described herein; a nucleic acid encoding any antibody, CDR, VH or VL domain, or antigen-binding fragment thereof; and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

Antibodies or antigen-binding fragments thereof as provided herein, and encoding nucleic acid molecules and vectors, may be isolated and/or purified, e.g., from their natural environment, in significantly pure or homogeneous form, or, in the case of nucleic acid, free or significantly free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antigen-binding fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the herein described antibodies, and which further expresses or is capable of expressing a selected gene of interest, such as a gene encoding any herein described antibody. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In some embodiments, the nucleic acid is integrated into the genome (e.g., chromosome) of the host cell. Integration may be promoted by inclusion of sequences which promote recombination with the genome, in accordance-with standard techniques.

Certain embodiments include using a construct as described herein in an expression system in order to express a particular polypeptide such as a SIRPα-specific antibody as described herein. The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS IN MOLECULAR BIOLOGY, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The terms “polypeptide” “protein” and “peptide” and “glycoprotein” are used interchangeably and mean a polymer of amino acids not limited to any particular length. The term does not exclude modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the antibodies described herein that bind to SIRPα, and sequences that have deletions from, additions to, and/or substitutions of one or more amino acid of an anti-SIRPα antibody. Thus, a “polypeptide” or a “protein” can comprise one (termed “a monomer”) or a plurality (termed “a multimer”) of amino acid chains.

The term “isolated protein” referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is significantly free from proteins or polypeptides or other contaminants that are found in its natural environment that would interfere with its use (therapeutic, diagnostic, prophylactic, research or otherwise).

The term “polypeptide fragment” refers to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of an anti-SIRPα antibody, useful fragments include, but are not limited to: a CDR region, especially a CDR3 region of the heavy or light chain; a variable region of a heavy or light chain; a portion of an antibody chain or just its variable region including two CDRs; and the like.

Polypeptides may comprise a signal (or leader) sequence at the N-terminal end of the protein, which co-translationally or post-translationally directs transfer of the protein. Any polypeptide amino acid sequences provided herein that include a signal peptide are also contemplated for any use described herein without such a signal or leader peptide. As would be recognized by the skilled person, the signal peptide is usually cleaved during processing and is not included in the active antibody protein. The polypeptide may also be fused in-frame or conjugated to a linker or other sequence for ease of synthesis, purification or identification of the polypeptide (e.g., poly-His), or to enhance binding of the polypeptide to a solid support.

A peptide linker/spacer sequence may also be employed to separate multiple polypeptide components by a distance sufficient to ensure that each polypeptide folds into its secondary and/or tertiary structures, if desired. Such a peptide linker sequence can be incorporated into a fusion polypeptide using standard techniques well known in the art.

Certain peptide spacer sequences may be chosen, for example, based on: (1) their ability to adopt a flexible extended conformation; (2) their inability to adopt a secondary structure that could interact with functional epitopes on the first and second polypeptides; and/or (3) the lack of hydrophobic or charged residues that might react with the polypeptide functional epitopes.

In one illustrative embodiment, peptide spacer sequences contain, for example, Gly, Asn and Ser residues. Other near neutral amino acids, such as Thr and Ala, may also be included in the spacer sequence.

Other amino acid sequences which may be usefully employed as spacers include those disclosed in Maratea et al., Gene 40:39 46 (1985); Murphy et al., Proc. Natl. Acad. Sci. USA 83:8258 8262 (1986); U.S. Pat. Nos. 4,935,233 and 4,751,180.

Other illustrative spacers may include, for example, Glu-Gly-Lys-Ser-Ser-Gly-Ser-Gly-Ser-Glu-Ser-Lys-Val-Asp (SEQ ID NO: 413) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (SEQ ID NO: 414) (Bird et al., 1988, Science 242:423-426).

In some embodiments, spacer sequences are not required when the first and second polypeptides have non-essential N-terminal amino acid regions that can be used to separate the functional domains and prevent steric interference. Two coding sequences can be fused directly without any spacer or by using a flexible polylinker composed, for example, of the pentamer Gly-Gly-Gly-Gly-Ser (SEQ ID NO: 415) repeated 1 to 3 times. Such a spacer has been used in constructing single chain antibodies (scFv) by being inserted between VH and VL (Bird et al., 1988, Science 242:423-426; Huston et al., 1988, Proc. Natl. Acad. Sci. U.S.A. 85:5979-5883).

A peptide spacer, in certain embodiments, is designed to enable the correct interaction between two beta-sheets forming the variable region of the single chain antibody.

In certain illustrative embodiments, a peptide spacer is between 1 to 5 amino acids, between 5 to 10 amino acids, between 5 to 25 amino acids, between 5 to 50 amino acids, between 10 to 25 amino acids, between 10 to 50 amino acids, between 10 to 100 amino acids, or any intervening range of amino acids.

In other illustrative embodiments, a peptide spacer comprises about 1, 5, 10, 15, 20, 25, 30, 35, 40, 45, 50 or more amino acids in length.

Amino acid sequence modification(s) of the antibodies described herein are contemplated. For example, it may be desirable to improve the binding affinity and/or other biological properties of the antibody. For example, amino acid sequence variants of an antibody may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final antibody, provided that the final construct possesses the desired characteristics (e.g., high affinity binding to SIRPα). The amino acid changes also may alter post-translational processes of the antibody, such as changing the number or position of glycosylation sites. Any of the variations and modifications described above for polypeptides of the present disclosure may be included in antibodies of the present disclosure.

Also provided are variants of the antibodies disclosed herein. In certain embodiments, such variant antibodies or antigen-binding fragments, or CDRs thereof, bind to SIRPα at least about 50%, at least about 70%, and in certain embodiments, at least about 90% as well as an antibody sequence specifically set forth herein. In further embodiments, such variant antibodies or antigen-binding fragments, or CDRs thereof, bind to SIRPα with greater affinity than the antibodies set forth herein, for example, that bind quantitatively at least about 105%, 106%, 107%, 108%, 109%, or 110% as well as an antibody sequence specifically set forth herein.

Determination of the three-dimensional structures of representative polypeptides (e.g., variant SIRPα-specific antibodies as provided herein, for instance, an antibody protein having an antigen-binding fragment as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of SIRPα-specific antibodies antigen-binding domains thereof as provided herein, include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/. Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

In some embodiments, the anti-SIRPα antibodies and humanized versions thereof are derived from rabbit monoclonal antibodies, and in particular are generated using APXiMAB™ technology. These antibodies are advantageous as they require minimal sequence modifications, thereby facilitating retention of functional properties after humanization using mutational lineage guided (MLG) humanization technology (see e.g., U.S. Pat. No. 7,462,697). Thus, illustrative methods for making the anti-SIRPα antibodies of the present disclosure include the APXiMAB™ rabbit monoclonal antibody technology described, for example, in U.S. Pat. Nos. 5,675,063 and 7,429,487. In this regard, in certain embodiments, the anti-SIRPα antibodies of the disclosure are produced in rabbits. In particular embodiments, a rabbit-derived immortal B-lymphocyte capable of fusion with a rabbit splenocyte or peripheral B lymphocyte is used to produce a hybrid cell that produces an antibody. The immortal B-lymphocyte does not detectably express endogenous immunoglobulin heavy chain and may contain, in certain embodiments, an altered immunoglobulin heavy chain-encoding gene.

Compositions and Methods of Use

Certain embodiments include compositions comprising the SIRPα-specific antibodies, including antigen-binding fragments thereof, and administration of such composition in a variety of therapeutic settings, including the treatment of cancers and other diseases.

Administration of the SIRPα-specific antibodies described herein, in pure form or in an appropriate pharmaceutical composition, can be carried out via any of the accepted modes of administration of agents for serving similar utilities. The pharmaceutical compositions can be prepared by combining an antibody or antibody-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition. Administration may be achieved by a variety of different routes, including oral, parenteral, nasal, intravenous, intradermal, subcutaneous or topical. Preferred modes of administration depend upon the nature of the condition to be treated or prevented. An amount that, following administration, reduces, inhibits, prevents or delays the progression and/or metastasis of a cancer is considered effective.

In certain embodiments, the amount administered is sufficient to result in tumor regression, as indicated by a statistically significant decrease in the amount of viable tumor, for example, at least a 50% decrease in tumor mass, or by altered (e.g., decreased with statistical significance) scan dimensions.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The SIRPα-specific antibody-containing compositions may be administered alone or in combination with other known cancer treatments, such as radiation therapy, chemotherapy, transplantation, immunotherapy, hormone therapy, photodynamic therapy, etc. The compositions may also be administered in combination with antibiotics.

Typical routes of administering these and related pharmaceutical compositions thus include, without limitation, oral, topical, transdermal, inhalation, parenteral, sublingual, buccal, rectal, vaginal, intravitreal, and intranasal. The term parenteral as used herein includes subcutaneous injections, intravenous, intramuscular, intrasternal injection or infusion techniques. Pharmaceutical compositions according to certain embodiments are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described SIRPα-specific antibody in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an antibody described herein, for treatment of a disease or condition of interest in accordance with teachings herein.

A pharmaceutical composition may be in the form of a solid or liquid. In certain embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a SIRPα-specific antibody as herein disclosed such that a suitable dosage will be obtained. Typically, this amount is at least 0.01% of the antibody in the composition. When intended for oral administration, this amount may be varied to be between 0.1 and about 70% of the weight of the composition. Certain oral pharmaceutical compositions contain between about 4% and about 75% of the antibody. In certain embodiments, pharmaceutical compositions and preparations are prepared so that a parenteral dosage unit contains between 0.01 to 10% by weight of the antibody prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the antibody and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises a SIRPα-specific antibody as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the antibody composition so as to facilitate dissolution or homogeneous suspension of the antibody in the aqueous delivery system.

The compositions may be administered in a therapeutically effective amount, which will vary depending upon a variety of factors including the activity of the specific compound (e.g., SIRPα-specific antibody) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

Compositions comprising the SIRPα-specific antibodies may also be administered simultaneously with, prior to, or after administration of one or more other therapeutic agents. Such combination therapy may include administration of a single pharmaceutical dosage formulation which contains an antibody and one or more additional active agents, as well as administration of compositions comprising SIRPα-specific antibodies and each active agent in its own separate pharmaceutical dosage formulation. For example, an antibody as described herein and the other active agent can be administered to the patient together in a single oral dosage composition such as a tablet or capsule, or each agent administered in separate oral dosage formulations. Similarly, an antibody as described herein and the other active agent can be administered to the patient together in a single parenteral dosage composition such as in a saline solution or other physiologically acceptable solution, or each agent administered in separate parenteral dosage formulations. Where separate dosage formulations are used, the compositions comprising antibodies and one or more additional active agents can be administered at essentially the same time, i.e., concurrently, or at separately staggered times, i.e., sequentially and in any order; combination therapy is understood to include all these regimens.

Thus, in certain embodiments, also contemplated is the administration of anti-SIRPα antibody compositions in combination with one or more other therapeutic agents. Such therapeutic agents may be accepted in the art as a standard treatment for a particular disease state as described herein, such as rheumatoid arthritis, inflammation or cancer. Exemplary therapeutic agents contemplated include cytokines, growth factors, steroids, NSAIDs, DMARDs, anti-inflammatories, chemotherapeutics, radiotherapeutics, or other active and ancillary agents.

In certain embodiments, for treating cancer, the anti-SIRPα antibodies disclosed herein are administered in combination with one or more cancer immunotherapy agents. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), V-domain Ig suppressor of T cell activation (VISTA), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In certain embodiments, the agent is a VISTA antagonist or inhibitor. VISTA is approximately 50 kDa in size and belongs to the immunoglobulin superfamily (it has one IgV domain) and the B7 family. It is primarily expressed in white blood cells, and its transcription is partially controlled by p53. There is evidence that VISTA can act as both a ligand and a receptor on T cells to inhibit T cell effector function and maintain peripheral tolerance. VISTA is produced at high levels in tumor-infiltrating lymphocytes, such as myeloid-derived suppressor cells and regulatory T cells, and its blockade with an antibody results in delayed tumor growth in mouse models of melanoma and squamous cell carcinoma. Exemplary anti-VISTA antagonist antibodies include, for example, the antibodies described in WO 2018/237287, which is incorporated by reference in its entirety.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tcl function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun 33: 970-9, 2003; and Workman et al., Journal of Immun 172: 5450-5, 2004), and has been reported to play a role in Treg suppressive function (see, e.g., Huang et al., Immunity. 21: 503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derre et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include CD40, OX40, Glucocorticoid-Induced TNFR Family Related Gene (GITR), CD137 (4-1BB), CD27, CD28, CD226, and Herpes Virus Entry Mediator (HVEM).

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof. Specific examples of CD40 agonists include, but are not limited to, APX005 (see, e.g., US 2012/0301488) and APX005M (see, e.g., US 2014/0120103).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014; 3:e27255. doi:10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the anti-SIRPα antibodies disclosed herein are administered in combination with one or more bi-specific or multi-specific antibodies. For instance, certain bi-specific or multi-specific antibodies are able to (i) bind to and inhibit one or more inhibitory immune checkpoint molecules, and also (ii) bind to and agonize one or more stimulatory immune checkpoint molecules. In certain embodiments, a bi-specific or multi-specific antibody (i) binds to and inhibits one or more of PD-L1, PD-L2, PD-1, CTLA-4, IDO, TDO, TIM-3, LAG-3, BTLA, CD160, and/or TIGIT, and also (ii) binds to and agonizes one or more of CD40, OX40 Glucocorticoid-Induced TNFR Family Related Gene (GITR), CD137 (4-1BB), CD27, CD28, CD226, and/or Herpes Virus Entry Mediator (HVEM).

In some embodiments, the anti-SIRPα antibodies disclosed herein are administered in combination with one or more cancer vaccines. In certain embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, the anti-SIRPα antibodies disclosed herein are administered in combination with one or more oncolytic viruses. In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAdl, SEPREHVIR (HSV-1716), CGTG-102 (Ad5/3-D24-GMCSF), GL-ONC1, MV-NIS, and DNX-2401.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a T-cell based adoptive immunotherapy. In some embodiments, the cell-based immunotherapy comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015).

In some embodiments, the anti-SIRPα antibodies disclosed herein are used as part of adoptive immunotherapies, for example, autologous immunotherapies. Certain embodiments thus include methods of treating a cancer in a patient in need thereof, comprising:
(a) incubating ex vivo-derived immune cells with an anti-SIRPα antibody, or antigen-binding fragment thereof, described herein; and
(b) administering the autologous immune cells to the patient.

In some instances, the ex vivo-derived immune cells are autologous cells, which are obtained from the patient to be treated. In some embodiments, the autologous immune cells comprise lymphocytes, natural killer (NK) cells, macrophages, and/or dendritic cells (DCs). In some embodiments, the lymphocytes comprise T-cells, optionally cytotoxic T-lymphocytes (CTLs). See, for example, June, J Clin Invest. 117: 1466-1476, 2007; Rosenberg and Restifo, Science. 348:62-68, 2015; Cooley et al., Biol. of Blood and Marrow Transplant. 13:33-42, 2007; and Li and Sun, Chin J Cancer Res. 30:173-196, 2018, for descriptions of adoptive T-cell and NK cell immunotherapies. In some embodiments, the T-cells comprise comprise cancer antigen-specific T-cells, which are directed against at least one “cancer antigen”, as described herein. In certain embodiments, the anti-SIRPα antibody, or antigen-binding fragment thereof, enhances the efficacy of the adoptively transferred immune cells.

In certain embodiments, the anti-SIRPα antibodies disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the anti-SIRPα antibodies disclosed herein are administered in combination with an anti-tumor associated antigen (TAA) agent, including an anti-TAA antibody, or antigen-binding fragment thereof. Included are combination therapies with an epidermal growth factor receptor (EGFR) inhibitor, for example, an antibody, or antigen-binding fragment thereof, targeted against EGFR, such as cetuximab. In certain of these and related embodiments, the subject has metastatic colorectal cancer, metastatic non-small cell lung cancer, or head and neck cancer. Also included are combination therapies with an antibody, or antigen-binding fragment thereof, targeted against HER2/neu, such as trastuzumab. In certain embodiments, the subject has a HER2-positive or HER2-overexpressing cancer such as a breast cancer or stomach cancer. Some embodiments include combination therapies with an antibody, or antigen-binding fragment thereof, targeted against CD20, such as rituximab. In certain of these and related embodiments, the subject has an autoimmune disease or a cancer such as non-Hodgkin lymphoma, chronic lymphocytic leukemia, rheumatoid arthritis, granulomatosis with polyangiitis, idiopathic thrombocytopenic purpura, pemphigus vulgaris, myasthenia gravis, or an Epstein-Barr virus-positive mucocutaneous ulcer.

A variety of other therapeutic agents may be used in conjunction with the anti-SIRPα antibodies described herein. In some embodiments, the antibody is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the antibodies described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, the compositions comprising herein described SIRPα-specific antibodies are administered to an individual afflicted with a disease as described herein, including, but not limited to cancers. Cancers include, but are not limited to, lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g,. hepatocellular carcinoma), and skin (e.g., melanoma or malignant melanoma), among others. Thus, certain embodiments include methods for treating a patient having a cancer, comprising administering to the patient a composition described herein, thereby treating the cancer. In some embodiments, the cancer is associated with aberrant SIRPα and/or CD47 expression or signaling activity. In some embodiments, the cancer is associated with SIRPα-mediated and/or CD47-mediated immune suppression. In some embodiments, the immune suppression comprises inhibition of phagocytosis by innate immune cells, such as macrophages and/or dendritic cells. In certain embodiments, the antibody for use in treating cancer is a SIRPα antagonist, for example, a SIRPα-CD47 signaling antagonist.

In some embodiments, the methods and compositions described herein (for example, anti-SIRPα antibody, alone or in combination with at least one additional agent) increase an immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to a control or reference. In some instances, the immune response comprises macrophage or dendritic cell-mediated phagocytosis of cancer cells. In some embodiments, the immune response comprises antibody-dependent cell phagocytosis (ADCP) of cancer cells.

In some embodiments, the methods and compositions described herein (for example, anti-SIRPα antibody, alone or in combination with at least one additional agent) increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and therapeutic compositions described herein (for example, anti-SIRPα antibody, alone or in combination with at least one additional agent) increase median survival time of a subject by 1 year, 2 years, 3 years, or longer. In some embodiments, the methods and therapeutic compositions described herein (for example, anti-SIRPα antibody, alone or in combination with cancer immunotherapy agent) increase progression-free survival by 2 weeks, 3 weeks, 4 weeks, 5 weeks, 6 weeks, 7 weeks, 8 weeks, 9 weeks, 10 weeks or longer. In certain embodiments, the methods or therapeutic compositions described herein increase progression-free survival by 1 year, 2 years, 3 years, or longer.

In certain embodiments, the methods and compositions described herein (for example, anti-SIRPα antibody, alone or in combination with at least one additional agent) are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and compositions described herein (for example, anti-SIRPα antibody, alone or in combination with at least one additional agent) are sufficient to result in stable disease. In certain embodiments, the methods and compositions described herein (for example, anti-SIRPα antibody, alone or in combination with cancer immunotherapy agent) are sufficient to result in clinically relevant reduction in symptoms of a particular disease indication known to the skilled clinician.

For in vivo use for the treatment of human disease, the antibodies described herein are generally incorporated into a pharmaceutical composition prior to administration. A pharmaceutical composition comprises one or more of the antibodies described herein in combination with a physiologically acceptable carrier or excipient as described elsewhere herein. To prepare a pharmaceutical composition, an effective amount of one or more of the compounds is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

The compositions comprising SIRPα-specific antibodies may be prepared with carriers that protect the antibody against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

Provided herein are methods of treatment using the antibodies that bind SIRPα. In some embodiments, an antibody described herein is administered to a patient having a disease involving inappropriate expression of SIRPα and/or CD47, including diseases and disorders characterized by aberrant SIRPα and/or CD47 expression or activity, due for example to alterations (e.g., statistically significant increases or decreases) in the amount of a protein present, or the presence of a mutant protein, or both. An overabundance may be due to any cause, including but not limited to overexpression at the molecular level, prolonged or accumulated appearance at the site of action, or increased (e.g., in a statistically significant manner) activity of SIRPα and/or CD47 relative to that which is normally detectable. Such an overabundance of SIRPα and/or CD47 can be measured relative to normal expression, appearance, or activity of SIRPα and/or CD47 signaling events, and said measurement can in some instances play a role in the development and/or clinical testing of the antibodies described herein.

In particular, the present antibodies are useful for the treatment of a variety of cancers, including cancers associated with the expression or overexpression of SIRPα and/or CD47. For example, certain embodiments provide a method for the treatment of a cancer including, but not limited to, lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g., hepatocellular carcinoma), and skin (e.g., melanoma or malignant melanoma), among others, by administering to a cancer patient a therapeutically effective amount of a herein disclosed SIRPα-specific antibody. An amount that, following administration, inhibits, prevents, or delays the progression and/or metastasis of a cancer in a statistically significant manner (i.e., relative to an appropriate control as will be known to those skilled in the art) is considered effective.

Some embodiments provide a method for reducing or preventing metastasis of a cancer including, but not limited to, lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g., hepatocellular carcinoma), and skin (e.g., melanoma or malignant melanoma), among others, by administering to a cancer patient a therapeutically effective amount of a herein disclosed SIRPα-specific antibody (e.g., an amount that, following administration, inhibits, prevents or delays metastasis of a cancer in a statistically significant manner, i.e., relative to an appropriate control as will be known to those skilled in the art).

Some embodiments provide a method for preventing a cancer including, but not limited to, lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g., hepatocellular carcinoma), and skin (e.g., melanoma or malignant melanoma), among others, by administering to a cancer patient a therapeutically effective amount of a herein disclosed SIRPα-specific antibody.

Some embodiments provide a method for treating, inhibiting the progression of, ameliorating the symptoms of, or prevention of cancers such as lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g., hepatocellular carcinoma), and skin (e.g., melanoma or malignant melanoma), among others by administering to a patient afflicted by one or more of these diseases a therapeutically effective amount of a herein disclosed SIRPα-specific antibody.

Certain embodiments relate to the treatment of infections diseases. Thus, some embodiments include methods of treating, reducing the severity of, or preventing an infectious disease in a patient in need thereof, comprising administering to the patient the composition described herein, for example, wherein the antibody, or antigen-binding fragment thereof, is a SIRPα antagonist, thereby treating, reducing the severity of, or preventing the infectious disease. Infectious diseases include, but are not limited to, viral, bacterial, fungal optionally yeast, and protozoal infections.

Certain embodiments relate to the treatment of autoimmune or inflammatory diseases. Thus, some embodiments include methods of treating an autoimmune or inflammatory disease in a subject in need thereof, comprising administering to the patient a composition described herein, for example, wherein the antibody, or antigen-binding fragment thereof, is a SIRPα agonist, thereby treating the autoimmune or inflammatory disease. In some embodiments, the autoimmune or inflammatory disease is associated with aberrant macrophage activation and phagocytosis.

Some embodiments are provided to improve transplantation in a patient, for example, by reducing phagocytosis of transplanted cells. Some embodiments thus include methods of improving transplantation in a patient in need thereof, comprising administering to the patient a composition described herein in combination with transplanted cells, for instance, wherein the antibody, or antigen-binding fragment thereof, is a SIRPα agonist that reduces phagocytosis of the transplanted cells, thereby improving transplantation in the patient. In some embodiments, the transplanted cells comprises hematopoietic stem cells, progenitor stem cells, or a solid organ.

Hematopoietic stem cell transplantation (HCT) is the transplantation of multipotent hematopoietic stem cells, usually derived from bone marrow, peripheral blood, or umbilical cord blood. In certain embodiments, hematopoietic stem and progenitor cell transplantation are performed during the treatment of individuals following ablative radiation or chemotherapy. A need for transplantation may be caused by genetic or environmental conditions, e.g. chemotherapy, exposure to radiation, etc. The cells for transplantation may be mixtures of cells, for example, buffy coat lymphocytes from a donor, or may be partially or substantially pure. The cells may be autologous cells, particularly if removed prior to cytoreductive or other therapy, or allogeneic cells, and may be used for hematopoietic stem or progenitor cell isolation and subsequent transplantation.

The term “solid organ” transplantation refers to a procedure in which an organ from a donor (living or deceased) is in placed into the body of a recipient patient in the appropriate position and cardiovascular connections to be physiologically integrated into the recipient. Examples include transplantation of a kidney, pancreas (including pancreatic islet cells), heart, lungs, intestine, liver, and the like. The transplanted organ may be referenced as a “graft”, and the physiological integration of the organ may be referred to as engraftment.

Certain embodiments include administering the anti-SIRPα containing composition to the subject prior to administration of transplanted cells, concurrently with administration of transplanted cells, or shortly after administration of transplanted cells

In some embodiments, anti-SIRPα antibodies are used to determine the structure of bound antigen, e.g., conformational epitopes, which structure may then be used to develop compounds having or mimicking this structure, e.g., through chemical modeling and SAR methods.

Some embodiments relate, in part, to diagnostic applications for detecting the presence of cells or tissues expressing SIRPα. Thus, certain embodiments include methods of detecting SIRPα in a sample, such as detection of cells or tissues expressing SIRPα. Such methods can be applied in a variety of known detection formats, including, but not limited to immunohistochemistry (IHC), immunocytochemistry (ICC), in situ hybridization (ISH), whole-mount in situ hybridization (WISH), fluorescent DNA in situ hybridization (FISH), flow cytometry, enzyme immuno-assay (EIA), and enzyme linked immuno-assay (ELISA).

ISH is a type of hybridization that uses a labeled complementary DNA or RNA strand (i.e., primary binding agent) to localize a specific DNA or RNA sequence in a portion or section of a cell or tissue (in situ), or if the tissue is small enough, the entire tissue (whole mount ISH). One having ordinary skill in the art would appreciate that this is distinct from immunohistochemistry, which localizes proteins in tissue sections using an antibody as a primary binding agent. DNA ISH can be used on genomic DNA to determine the structure of chromosomes. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

In some embodiments, the antibodies described herein are conjugated to a detectable label that may be detected directly or indirectly. In this regard, an antibody “conjugate” refers to an anti-SIRPα antibody that is covalently linked to a detectable label. DNA probes, RNA probes, monoclonal antibodies, antigen-binding fragments thereof, and antibody derivatives thereof, such as a single-chain-variable-fragment antibody or an epitope tagged antibody, may all be covalently linked to a detectable label. In “direct detection”, only one detectable antibody is used, i.e., a primary detectable antibody. Thus, direct detection means that the antibody that is conjugated to a detectable label may be detected, per se, without the need for the addition of a second antibody (secondary antibody).

A “detectable label” is a molecule or material that can produce a detectable (such as visually, electronically or otherwise) signal that indicates the presence and/or concentration of the label in a sample. When conjugated to an antibody, the detectable label can be used to locate and/or quantify the target to which the specific antibody is directed. Thereby, the presence and/or concentration of the target in a sample can be detected by detecting the signal produced by the detectable label. A detectable label can be detected directly or indirectly, and several different detectable labels conjugated to different specific-antibodies can be used in combination to detect one or more targets.

Examples of detectable labels, which may be detected directly, include fluorescent dyes and radioactive substances and metal particles. In contrast, indirect detection requires the application of one or more additional antibodies, i.e., secondary antibodies, after application of the primary antibody. Thus, the detection is performed by the detection of the binding of the secondary antibody or binding agent to the primary detectable antibody. Examples of primary detectable binding agents or antibodies requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to antibodies include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), alpha-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to the antibodies described herein or to any other molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents or antibodies, etc. Moreover, the skilled artisan would appreciate that each additional binding agent or antibody used to characterize a biological marker of interest may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

Also provided are kits for detecting SIRPα or cells or tissues expressing SIRPα in a sample, wherein the kits contain at least one antibody, polypeptide, polynucleotide, vector or host cell as described herein. In certain embodiments, a kit may comprise buffers, enzymes, labels, substrates, beads or other surfaces to which the antibodies described herein are attached, and the like, and instructions for use.

### EXAMPLES

### Example 1

### Preparation and Testing of Antibodies

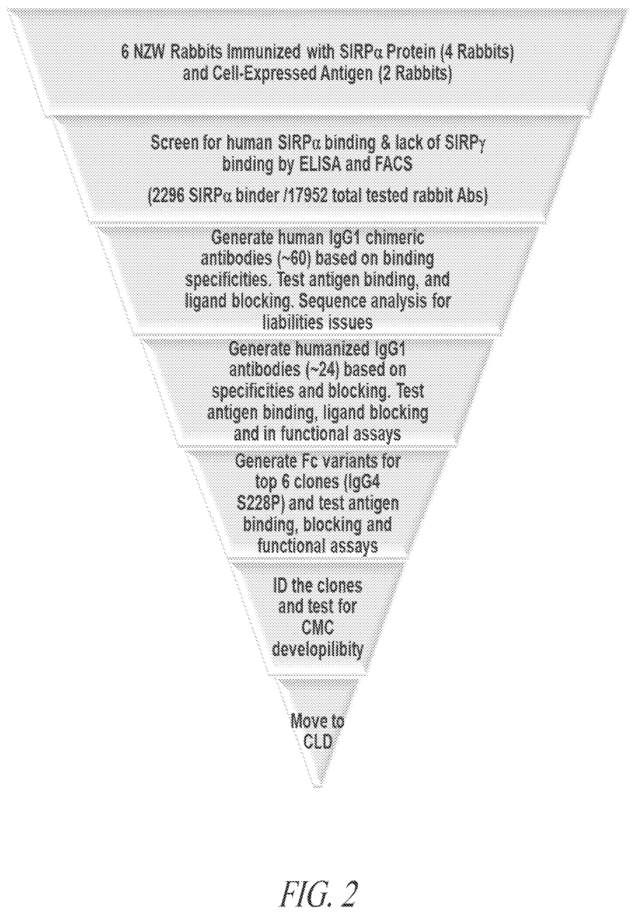

To prepare antibodies, a rabbit immunization and antibody screening strategy was performed, as illustrated in FIG. 2.

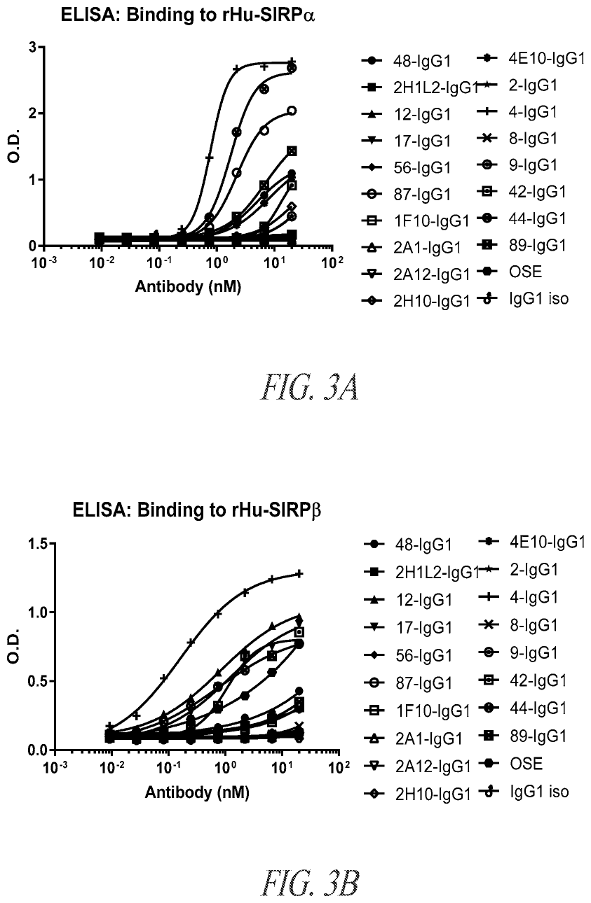

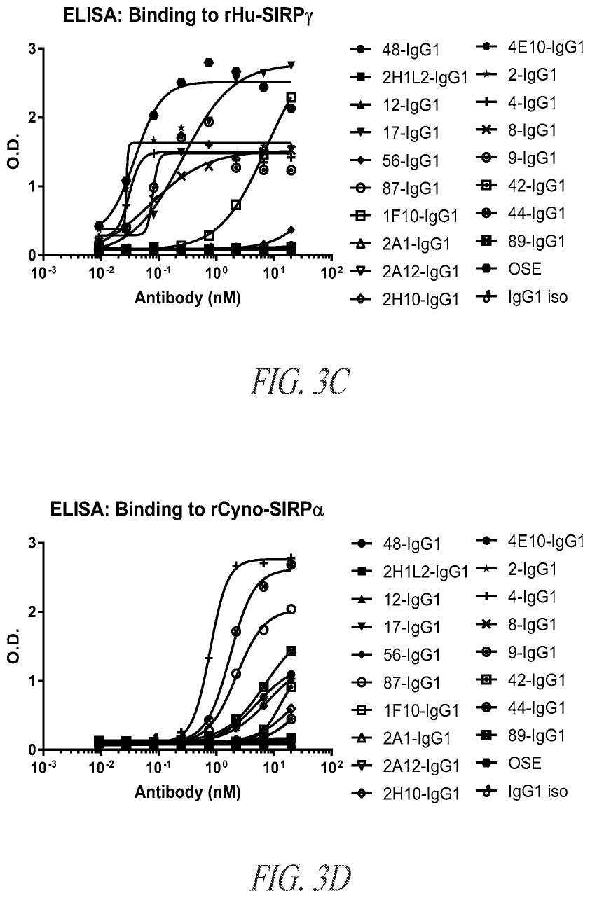

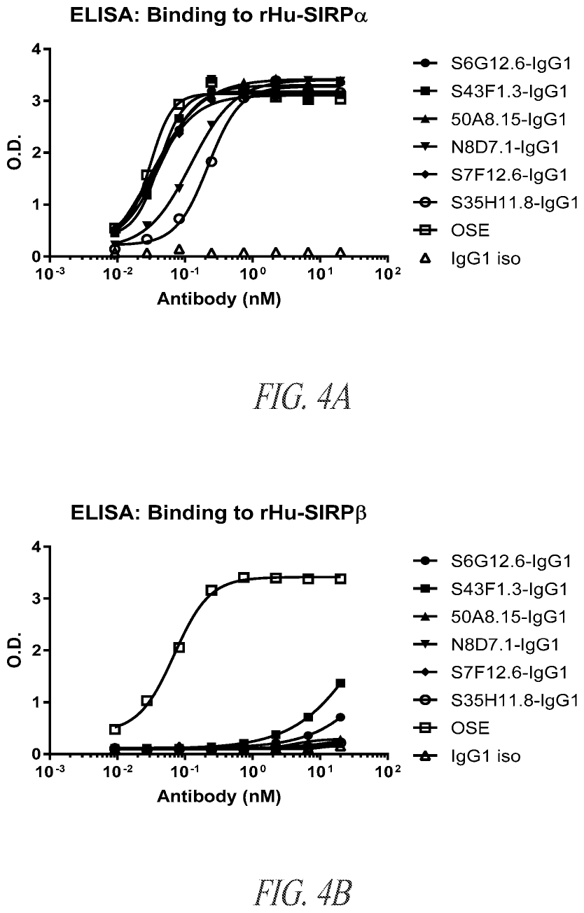

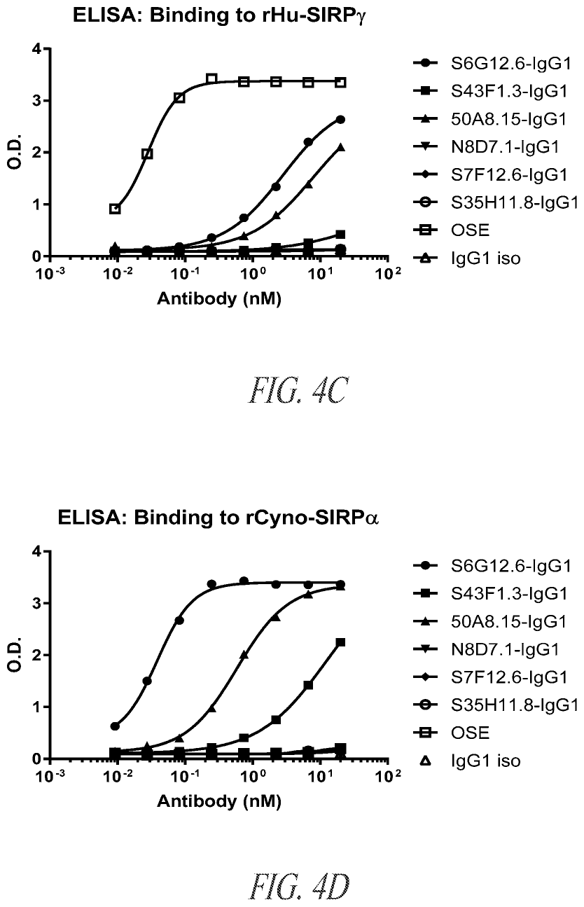

For initial binding analysis to recombinant human SIRPα, SIRPβ, and SIRPγ, and also recombinant cynomolgus SIRPα, humanized anti-SIRPα antibodies containing an IgG1 backbone were assessed for binding to the indicated SIRP family proteins immobilized on a plate. Briefly, recombinant SIRP family proteins were coated onto 96-well ELISA plates at 1 μg/mL by overnight incubation at 4° C. Excess unbound protein was washed off and the plates were blocked with 1% bovine serum albumin (BSA) for 1 hour at room temperature (RT). Serial dilutions of anti-SIRPα antibodies were prepared starting at 20 nM for an 8-point curve and then test samples were added and incubated for 1 hour at RT. Plates were washed and horse radish peroxidase (HRP) conjugated anti-human IgG secondary antibody added. Excess unbound secondary was washed and TMB substrate was added to the plates for approximately 10 minutes or until signal was visibly detectable. The reaction was stopped with acid and the plates were read immediately on the plate reader (Molecular Devices). The results are shown in FIGS. 3A-3D and FIGS. 4A-4D.

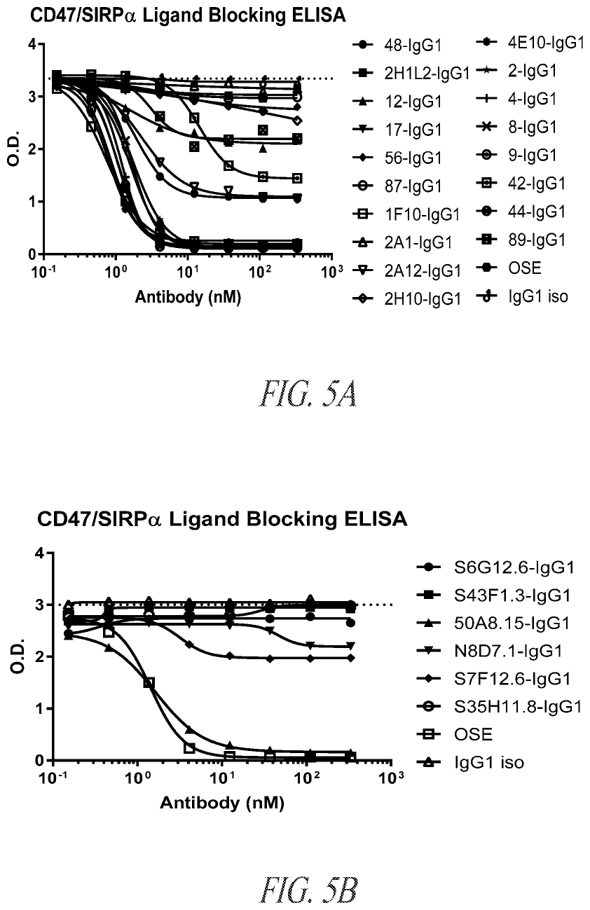

To assess ligand-blocking activity, humanized anti-SIRPα antibodies with an IgG1 backbone were assessed for blocking CD47 interaction with SIRPα immobilized on a plate. Briefly, recombinant human SIRPα-His was coated onto 96-well ELISA plates at 1 μg/mL by overnight incubation at 4° C. Excess unbound protein was washed off and the plates were blocked with 1% bovine serum albumin (BSA) for 1 hour at room temperature (RT). Serial dilutions of anti-SIRPα antibodies were prepared starting at 333.33 nM for an 8-point curve. These samples were added to the blocked plates and incubated for 1 hour at RT. Biotinylated CD47 (the ligand for SIRPα) was added to each well at 200 ng/mL and incubated at RT for 1 hour. The plates were washed and bound CD47 was detected using HRP-conjugated Streptavidin incubated for 1 hour at RT. Excess unbound secondary was washed and TMB substrate was added to the plates for approximately 15 minutes or until signal was visibly detectable. The reaction was stopped with acid and the absorbance (650 nm) measured immediately on a plate reader (Molecular Devices). The results are shown in FIGS. 5A-5B.

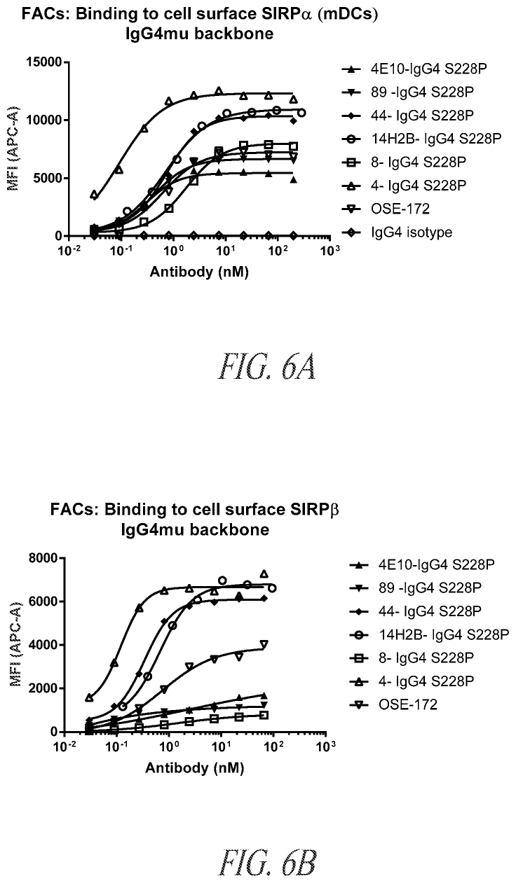

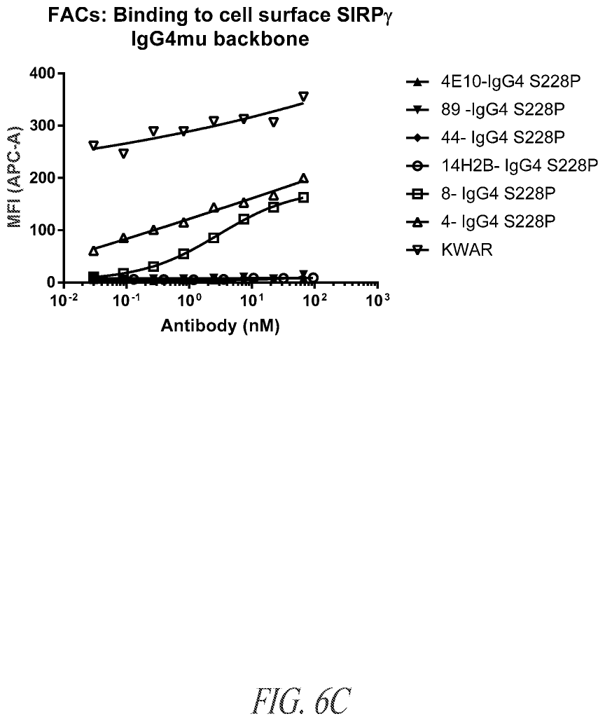

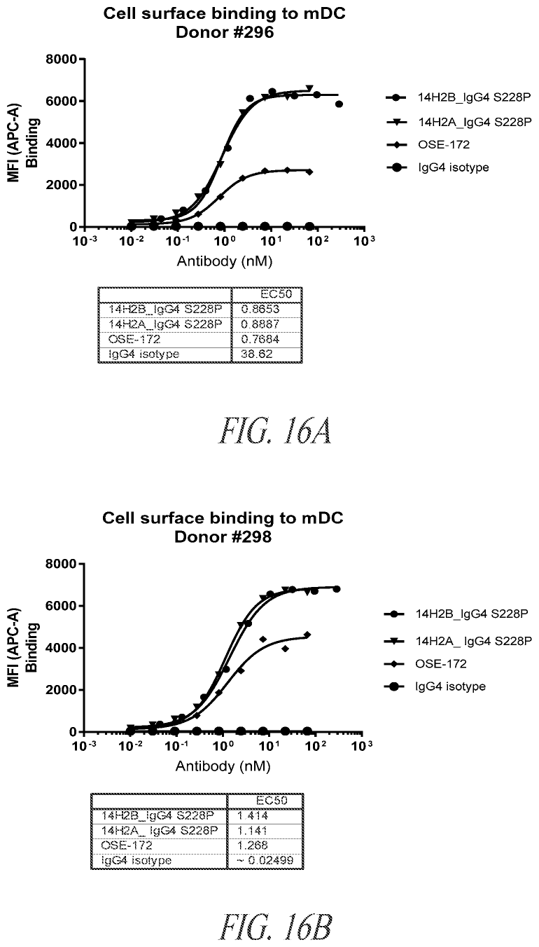

For cell-surface binding analysis, humanized anti-SIRPα antibodies containing an IgG4 S228P mutant backbone were assessed for binding to indicated native SIRP family proteins expressed on the cell surface. Assessment of the ability of anti-SIRPα antibodies to bind to cell surface expressed SIRPα was performed on human monocyte-derived dendritic cells (DCs), which express SIRPα but not SIRPβ or SIRPγ. Briefly, DCs were generated by culturing primary donor-derived CD14+ monocytes for 6-7 days in the presence of GM-CSF (100 ng/mL). DCs were plated in 96-well plates (— 100,000/well) and blocked with human TruStain FcX (Biolegend) in FACs buffer containing 5% donkey serum and 1% BSA. Serially diluted anti-SIRPα antibodies were added to the cells and incubated for 1 hour at 4° C. followed by three washes. Bound antibodies were detected using APC-conjugated donkey anti-human IgG F(ab). Samples were acquired on Cytoflex and analyzed using the FlowJo™ software. Similarly, the ability of anti-SIRPα antibodies to bind to cell surface SIRPβ was assessed using transfected HEK cells that overexpress SIRPβ but do not express SIRPα or SIRPγ. Assessment of anti-SIRPα antibodies' ability to bind to surface SIRPγ was performed on Jurkat cells that only express SIRPγ. The results are shown in FIGS. 6A-6C and FIGS. 16A-16B. Representative graphs from one experiment are shown. Each experiment was repeated at least twice. Table E1 provides the Kd values (nM) for the data in FIGS. 6A-6C.

TABLE E1

| Antibody | Kd (nM) |
|---|---|
| 4E10-IgG4 S228P | 0.2643 |
| 89H-IgG4 S228P | 0.3778 |
| 44H-IgG4 S228P | 0.672 |
| 14H2B-IgG4 S228P | 0.6864 |
| 8H- IgG4 S228P | 1.66 |
| 4H- IgG4 S228P | 0.08808 |
| OSE-172 | 0.5716 |
| IgG4 isotype | 0.002101 |

Table E2 provides the range of Kd values (nM) for mDC surface binding of exemplary anti-SIRPα antibodies. Representative data from 4 independent donors is shown.

TABLE E2

| Exp. # | 14H2B IgG4 S228P | 89H_b IgG4 S228P |
|---|---|---|
| 1 | 1.48 | 1.038 |
| 2 | 0.9528 | 0.9231 |
| 3 | 0.9901 | 0.8808 |
| 4 | 1.331 | 0.9077 |

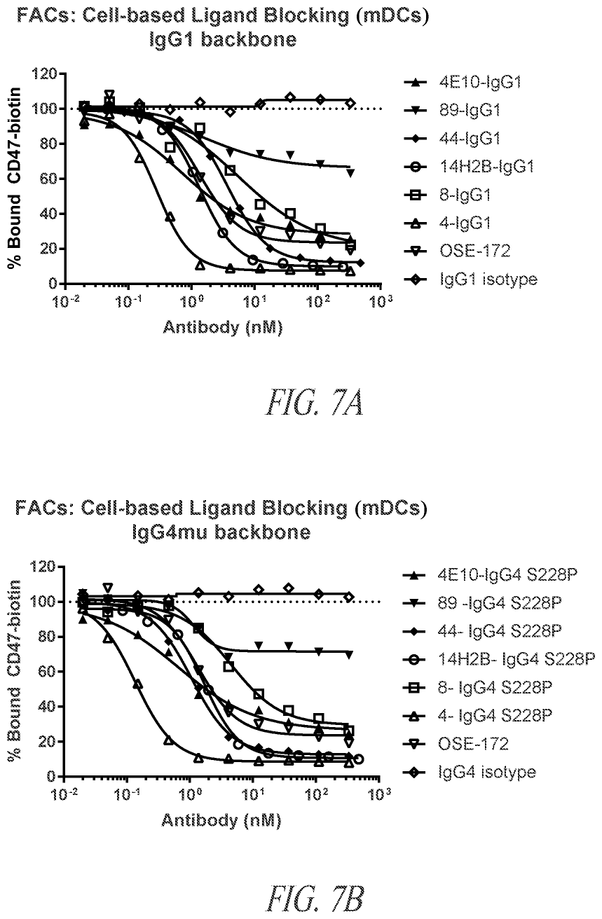

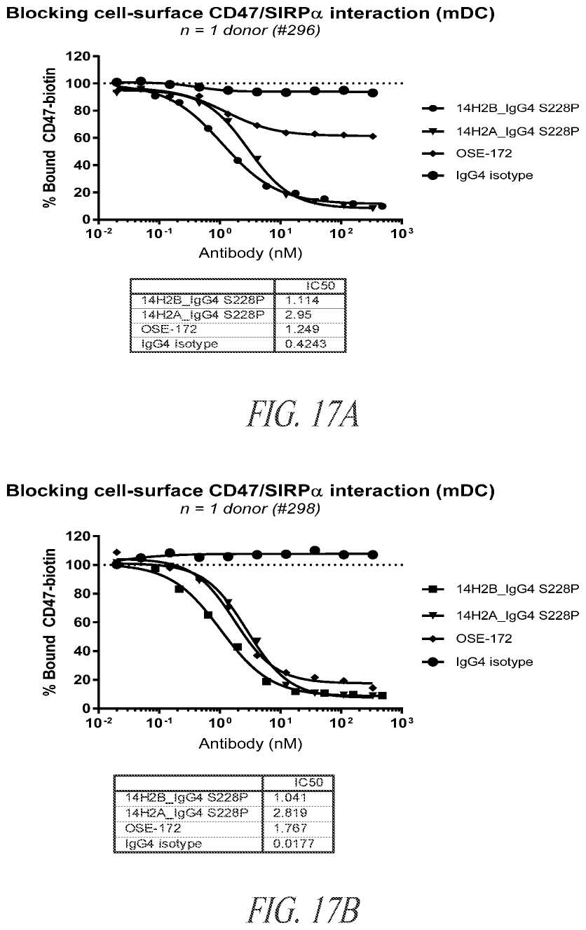

To assess ligand-blocking activity on the cell surface, DCs were generated by culturing primary donor-derived CD14+ monocytes for 6-7 days in the presence of GM-CSF (100 ng/mL). DCs were plated in 96-well plates (˜100,000/well) and blocked with human TruStain FcX™ (Biolegend) in FACs buffer containing 5% donkey serum and 1% BSA. Serially diluted anti-SIRPα antibodies were added to the cells and incubated for 1 hour at 4° C. Biotinylated CD47 (the ligand for SIRPα) was added to each well at 2 μg/mL and incubated for 1 hour at 4° C. The plates were washed and CD47 bound to cell surface was detected using PE-conjugated streptavidin (PE-SA) for 30 minutes at 4° C. Excess unbound PE-SA was removed by washing. Samples were acquired on Cytoflex and analyzed using the FlowJo software. The results are shown in FIGS. 7A-7B and FIGS. 17A-17B. Representative graphs from one experiment are shown. Each experiment was repeated at least twice. Table E3 provides the range of IC₅₀ values for mDC-based Cd47 blocking by anti-SIRPα antibodies. Representative data from 4 independent donors is shown.

TABLE E3

| Exp. # | 14H2B IgG4 S228P | 89H_b IgG4 S228P |
|---|---|---|
| 1 | 1.685 | 1.15 |
| 2 | 1.25 | 1.331 |
| 3 | 1.114 | 0.8495 |
| 4 | 1.041 | 0.7957 |

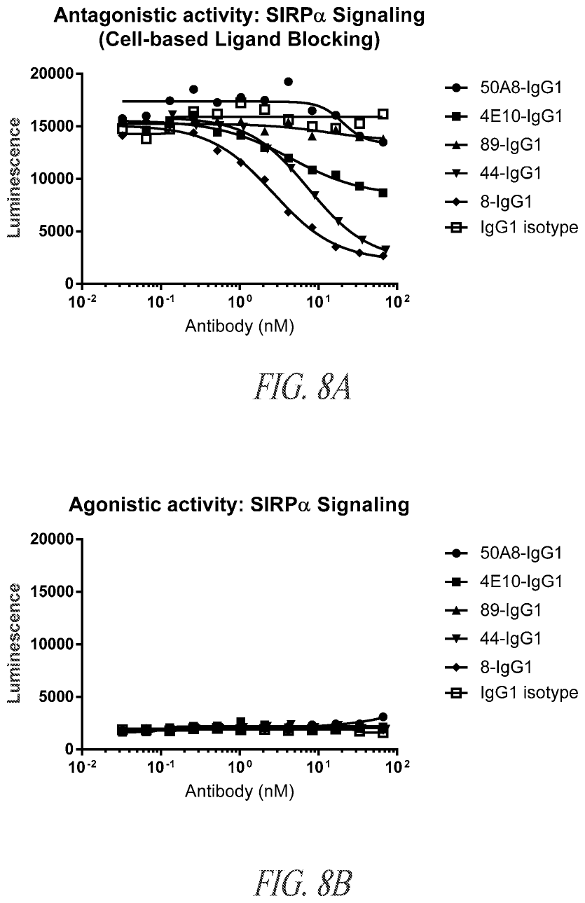

The antagonistic and agonistic activity of anti-SIRPα antibodies in the IgG1 backbone were assessed using the PathHunter™ Checkpoint Signaling assays from DiscoverX. The assays were performed as per manufacturer's instructions. To assess the antagonistic activity of the antibodies, the CD47 ligand-present cells were plated in a 96 well plate at 75,000 cells/well. Serial dilutions of anti-SIRPα antibodies were prepared and added to the cells. The SIRPα signaling cell line (in suspension) was added to the plate at 25,000 cells/well, and the plate was incubated overnight at 37° C. in a humidified incubator. The assay was concluded by adding the detection reagent and measuring chemiluminescence on a plate reader (Molecular Devices). The results of antagonistic activity testing are shown in FIG. 8A. To assess the agonistic activity of the antibodies the assay was performed in parallel as in A except no ligand presenting cells were added to the plate. The results of agonistic activity testing are shown in FIG. 8B.

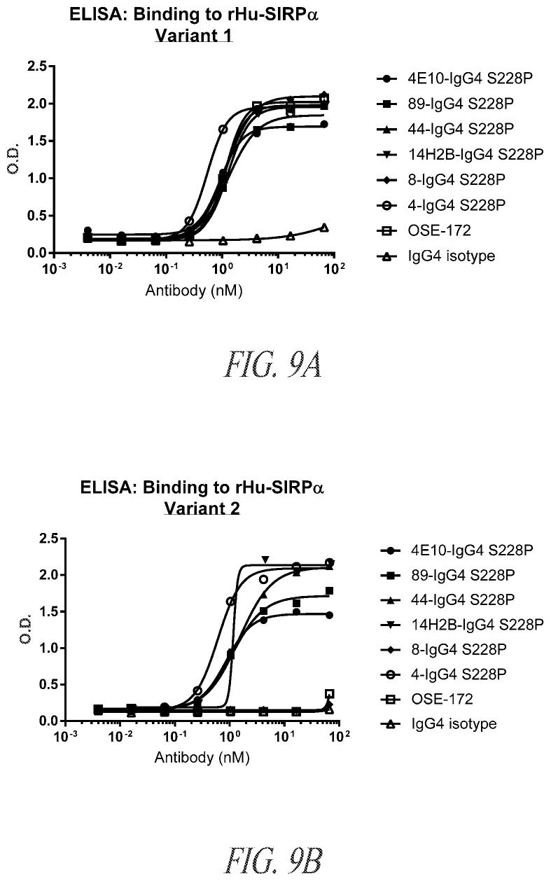

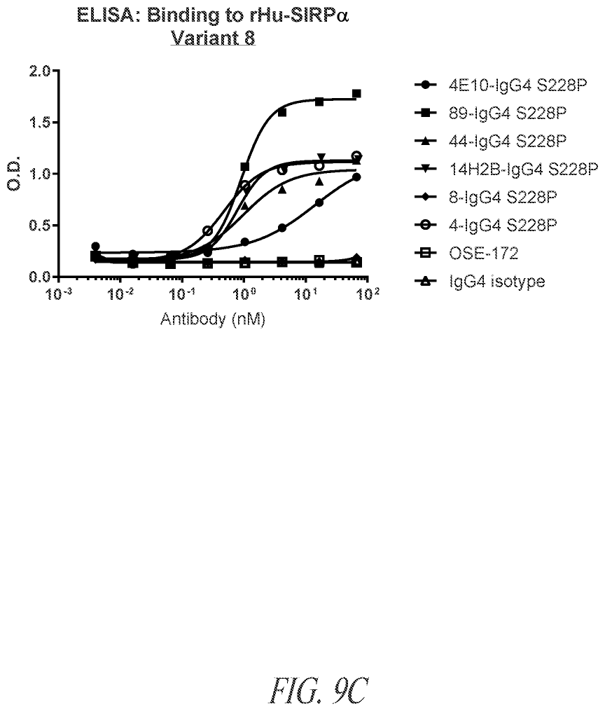

Humanized anti-SIRPα antibodies in the IgG4 S228P backbone were also assessed for binding to SIRPα variant proteins immobilized on a plate. Briefly, recombinant SIRPα variant proteins were coated onto 96-well ELISA plates at 1 μg/mL by overnight incubation at 4° C. Excess unbound protein was washed off and the plates were blocked with 1% bovine serum albumin (BSA) for 1 hour at room temperature (RT). Serial dilutions of anti-SIRPα antibodies were prepared and added to the blocked plates and incubated for 1 hour at RT. The plate was washed to remove excess unbound antibodies and bound antibodies were detected using horse radish peroxidase (HRP) conjugated anti-human IgG secondary antibody. Excess unbound secondary was washed and TMB substrate was added to the plates for approximately 10 minutes or until signal was visibly detectable. The reaction was stopped with acid and the absorbance (650 nm) measured immediately on a plate reader (Molecular Devices). The results are shown in FIGS. 9A-9C, and include binding to variant 1 (9A), variant 2 (9B), and variant 8 (9C).

Single agent phagocytosis by humanized anti-SIRPα antibodies was assessed in the IgG4 S228P backbone. Briefly, primary macrophages were generated by culturing primary donor-derived CD14+ monocytes for 6-8 days in the presence of M-CSF (100 ng/mL). Macrophages were stained with CFSE as per manufacturer's instructions, plated in V-bottom plates 96-well plates (50,000/well) and incubated with a serial dilution of test and comparison antibodies for 30 minutes on ice, including humanized anti-SIRPα antibodies, anti-CD47 antibody clone 5F9, OSE-172, and KWAR (see, for example, Liu et al., JCI Insight, 2020, 10.1172/jci.insight.134728; and U.S. Application No. 2019/0119396). The cells were transferred to ultra-low attachment U-bottom plates and 100,000 VTD-labeled cells (e.g., DLD-1 colorectal cancer cells, LS-174T colorectal cancer cells) were added per well. The cells were incubated for 2 hours at 37° C. in a humidified incubator, fixed and acquired on the Cytoflex. At least 25,000-35,000 events were recorded per well. The data was analyzed using the FlowJo™ software.

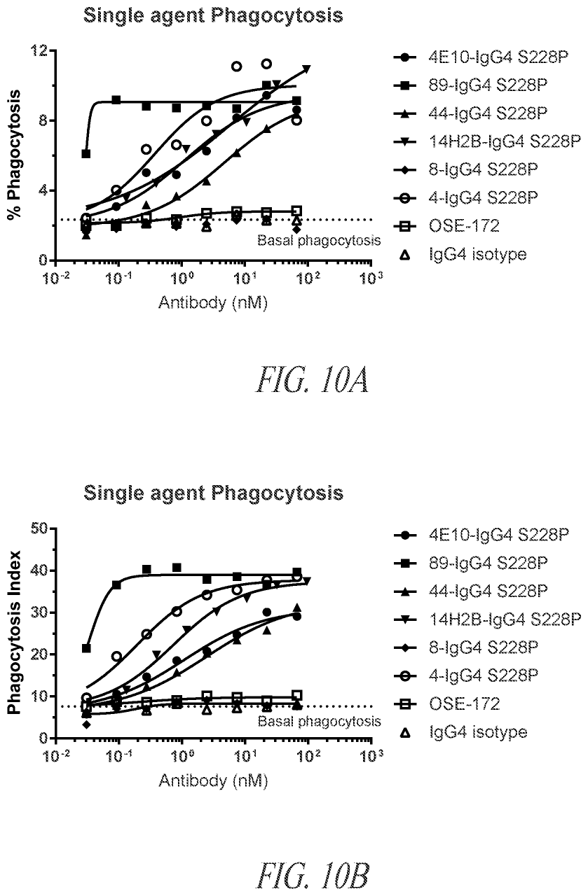

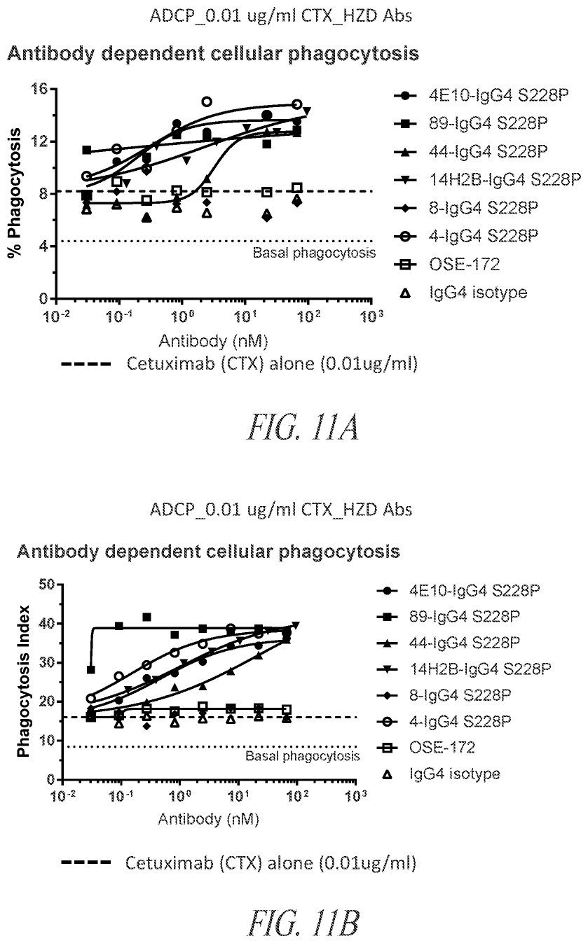

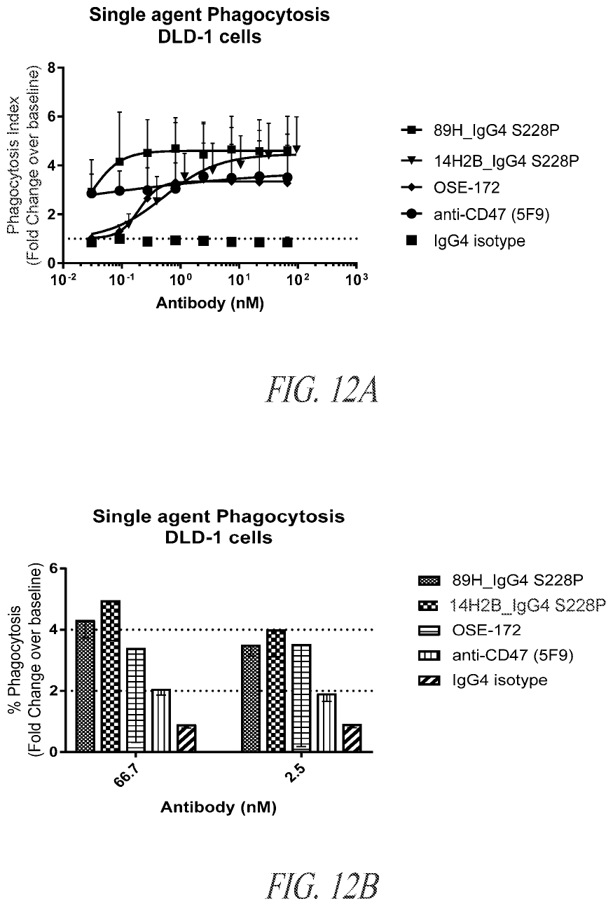

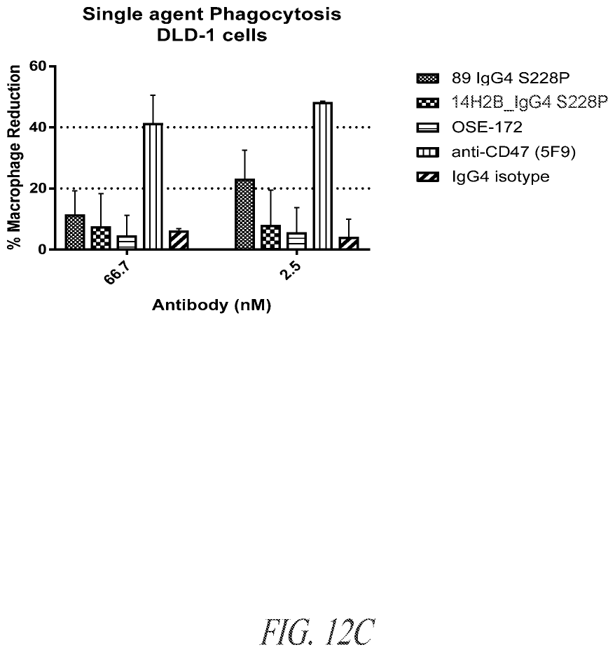

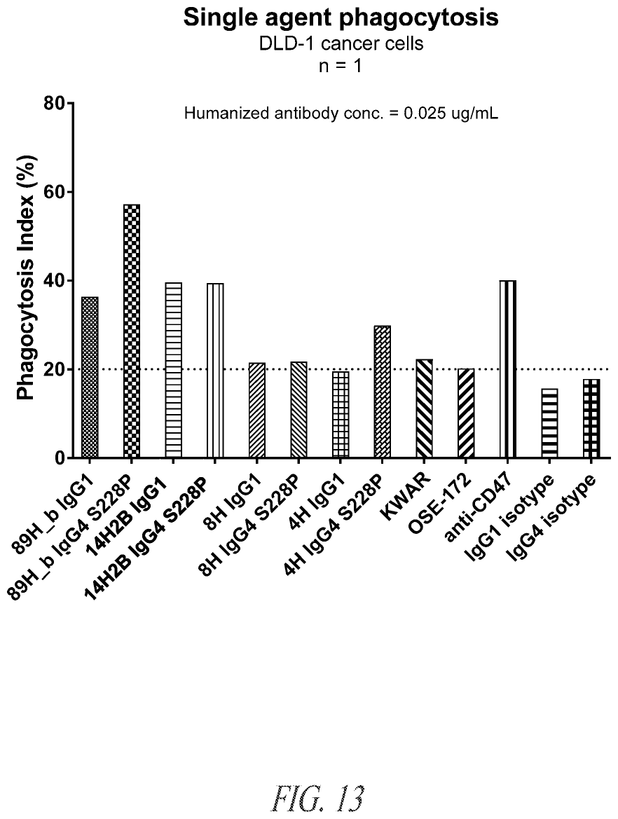

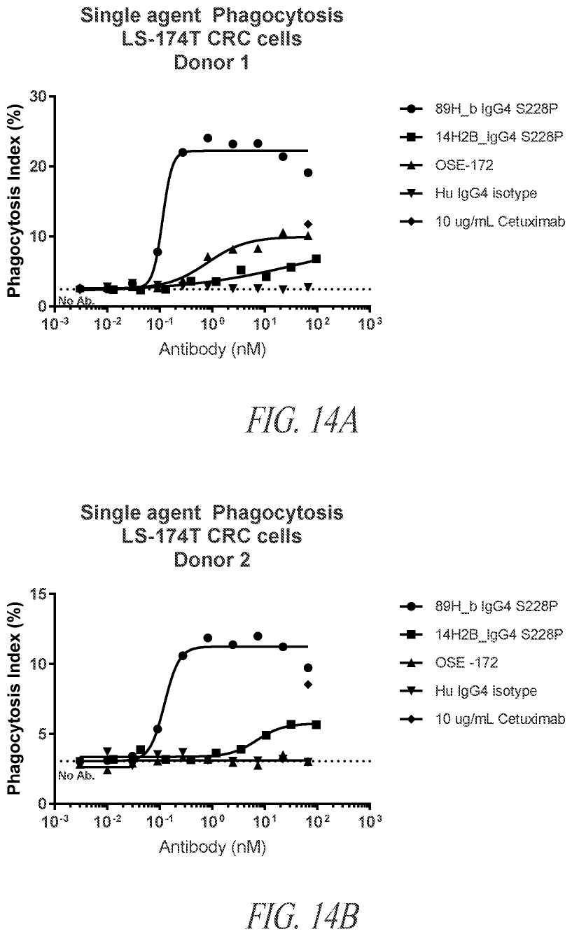

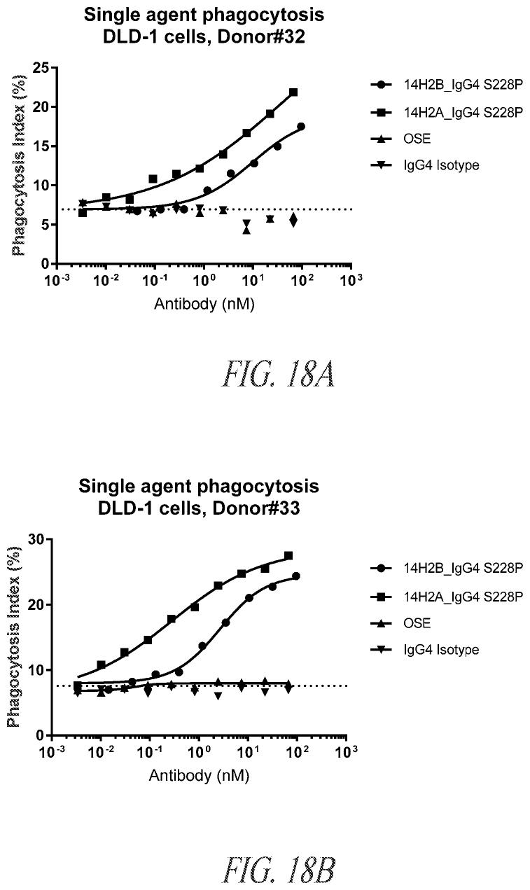

In FIG. 10A, percent (%) phagocytosis is reported as CFSE positive VTD positive cells (double positive cells) divided by total tumor cells (i.e., VTD positive+double positive cells) multiplied by one hundred. In FIG. 10B, the phagocytosis index is reported as CFSE positive VTD positive cells (double positive cells) divided by total macrophage cells (i.e., CFSE positive+double positive cells) multiplied by one hundred. Representative graphs from one experiment are shown. Each experiment was repeated using macrophages from 4 different donors. See also the results in FIGS. 12A-12C, which show comparisons to OSE-172 and anti-CD47 (5F9) antibodies in DLD-1 cells; FIG. 13, which shows comparisons in the IgG1 vs. IgG4 S228P backgrounds, and also to KWAR, OSE-172, and 5F9 antibodies; and FIGS. 14A-14B, which show comparisons to OSE-172 antibody in LS-174T colon cancer cells. FIGS. 18A-18B show a comparison of single agent phagocytosis by humanized anti-SIRPα antibodies 14H2A and 14H2B in the IgG4 S228P backbone in two donors.

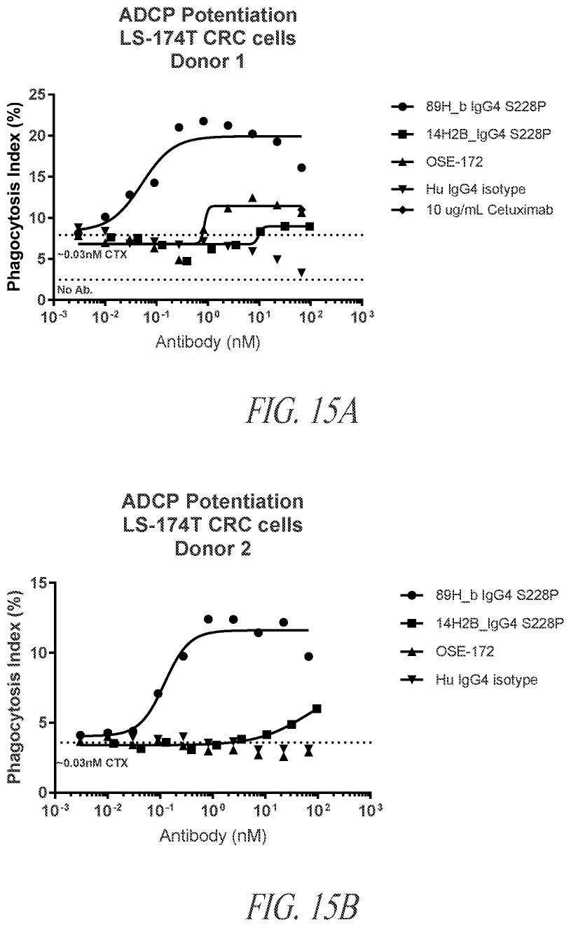

Antibody-dependent cellular phagocytosis (ADCP) by humanized anti-SIRPα antibodies was assessed in the IgG4 S228P backbone. Briefly, primary macrophages were generated by culturing primary donor-derived CD14+ monocytes for 6-8 days in the presence of M-CSF (100 ng/mL). Macrophages were stained with CFSE as per manufacturer's instructions, plated in V-bottom plates 96-well plates (˜50,000/well) and incubated with a serial dilution of test and comparison antibodies for 30 minutes on ice, including humanized anti-SIRPα antibodies and OSE-172. Simultaneously, DLD-1 colorectal cancer cells or LS-174T colorectal cancer cells were stained with VTD and incubated with 0.01 μg/mL cetuximab (anti-EGFR). The CFSE-labeled macrophages were transferred to ultra-low attachment U-bottom plates and 100,000 VTD-labeled cells (e.g., DLD-1 cells, LS-174T) pre-coated with cetuximab were added per well. The cells were incubated for 2 hours at 37° C. in a humidified incubator, fixed and acquired on the Cytoflex™ flow cytometer. At least 25,000-35,000 events were recorded per well. The data was analyzed using the FlowJo™ software. In FIG. 11A, percent (%) phagocytosis is reported as CFSE positive VTD positive cells (double positive cells) divided by total tumor cells (i.e. VTD positive+double positive cells) multiplied by hundred. In FIG. 11B, the phagocytosis index is reported as CFSE positive VTD positive cells (double positive cells) divided by total macrophage cells (i.e. CFSE positive+double positive cells) multiplied by hundred. Representative graphs from one experiment are shown. Each experiment was repeated using macrophages from 3 different donors. See also the results in FIGS. 15A-15B, which show ADCP potentiation by humanized anti-SIRPα antibodies in LS-174T colorectal cancer cells.

The effects of anti-SIRPα antibodies on phagocytosis were tested in combination with cetixumab, trastuzumab, and rituximab.

For combinations with cetuximab, human macrophages (SIRPα V1) were labeled with Cell Tracker Violet and plated in a low-attachment 96-well plate with A431 cells labeled with CFSE labeled in a 2:1 ratio. A431 cells were opsonized with Cetuximab at 1 μg/mL prior to plating. Anti-SIRPα antibodies were added at the increasing concentrations as indicated. Cultures were incubated at 37° C. for 2-3 hours. After incubation, cells were harvested, fixed and stained with viability dye and anti-EPCAM.

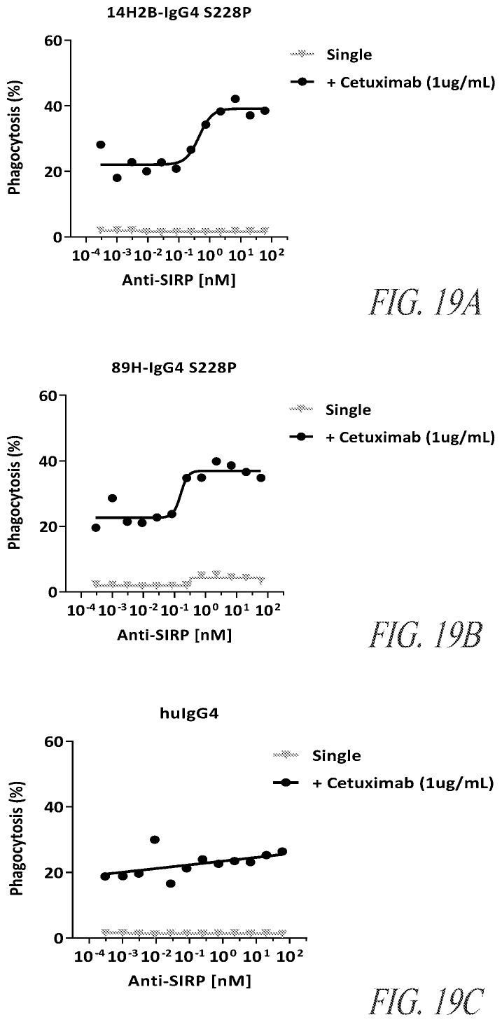

Phagocytosis was calculated as the percent of live/EPCAM-/Violet+/CFSE+ cells (phagocytosed tumor cells) out of total CFSE+ (tumor) population. FIGS. 19A-19C show that anti-SIRPα antibodies significantly enhanced the phagocytosis activity of cetuximab on A431 cells. Light colored lines indicate single agent titration of anti-SIRP antibodies and darker lines indicate combination with 1 μg/mL Cetuximab.

For combinations with trastuzumab, human macrophages (SIRPα V1) were labeled with Cell Tracker Violet and plated in a low-attachment 96-well plate with OE-19 cells labeled with CFSE labeled in a 2:1 ratio. OE-19 cells were opsonized with Trastuzumab at 1 μg/mL prior to plating. Anti-SIRPαantibodies were added at the increasing concentrations as indicated. Cultures were incubated at 37° C. for 2-3 hours. After incubation, cells were harvested, fixed and stained with viability dye and anti-EPCAM.

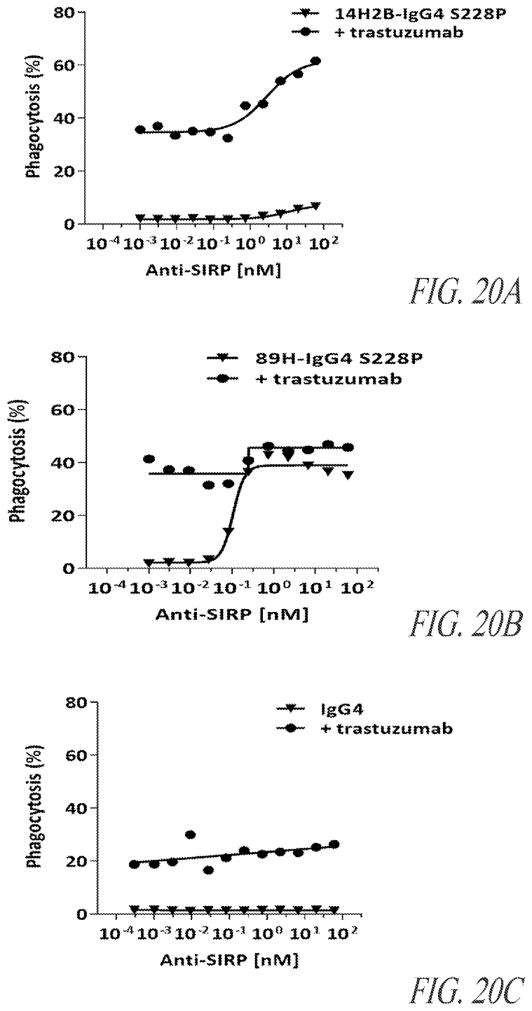

Phagocytosis was calculated as the percent of live/EPCAM-/Violet+/CFSE+ cells (phagocytosed tumor cells) out of total CFSE+(tumor) population. FIGS. 20A-20C show that anti-SIRPα antibodies significantly enhanced the phagocytosis activity of trastuzumab on OE-19 cells. Light colored lines indicate single agent titration of anti-SIRP antibodies and darker lines indicate combination with 1 μg/mL Trastuzumab.

For combinations with rituximab, human macrophages (SIRPα V2) were labeled with Cell Tracker Violet and plated in a low-attachment 96-well plate with Raji cells labeled with CFSE labeled in a 2:1 ratio. Raji cells were opsonized with Rituximab (Rit) at 6.6 nM prior to plating. Anti-SIRPα antibodies were added at 1 ug/mL. Cultures were incubated at 37° C. for 2-3 hours. After incubation, cells were harvested, fixed and stained with viability dye, anti-CD11b and anti-CD19.

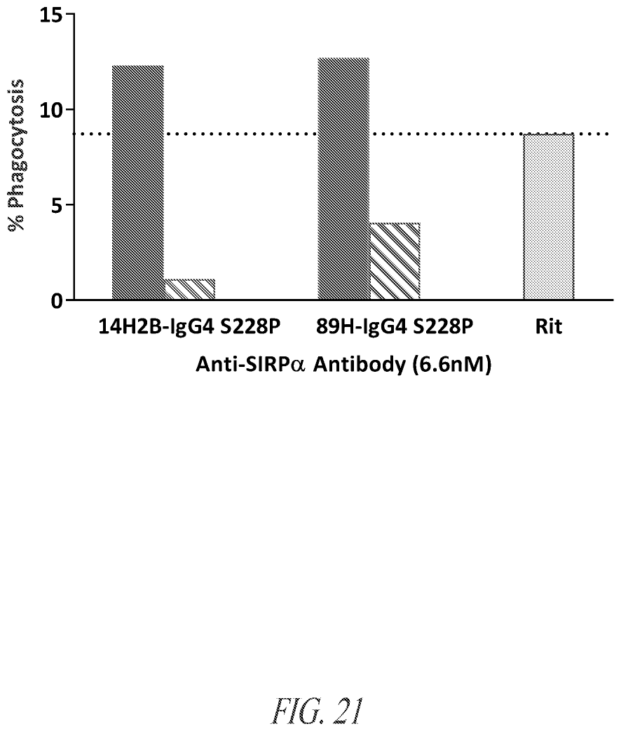

Percent phagocytosis was calculated as the percent of viable/CD19−/CD11b+/Cell Tracker+ cells (phagocytosing macrophages). FIG. 21 shows that anti-SIRPα antibodies significantly enhanced the phagocytosis activity of rituximab on Raji cells. Bars with slanted lines show single agent activity of anti-SIRPα antibodies. Rituximab only condition is shown light bar.

The binding of humanized anti-SIRPα antibodies to SIRP-βL was analyzed by ELISA. SIRP-βL is an isoform of SIRP-β (see Table E4).

TABLE E4
Exemplary SIRP-βL Sequence

| | | SEQ |
|---|---|---|
| | | ID |
| Identifier | Sequence | NO: |
| SIRPBL_ | EEELQVIQPDKSISVAAGESATLHCTVTSLIPVGPIQWFRGAGPGRELIYNQKEGH | 416 |
| Q5TFQ8 | FPRVTTVSDLTKRNNMDFSIRISNITPADAGTYYCVKFRKGSPDHVEFKSGAGTEL | |
| | SVRAKPSAPVVSGPAARATPQHTVSFTCESHGFSPRDITLKWFKNGNELSDFQTNV | |
| | DPAGDSVSYSIHSTAKVVLTREDVHSQVICEVAHVTLQGDPLRGTANLSETIRVPP | |
| | TLEVTQQPVRAENQVNVTCQVRKFYPQRLQLTWLENGNVSRTETASTLTENKDGTY | |
| | NWMSWLLVNVSAHRDDVKLTCQVEHDGQPAVSKSHDLKVSAHPKEQGSNTAPGPAL | |
| | ASAAPL | |

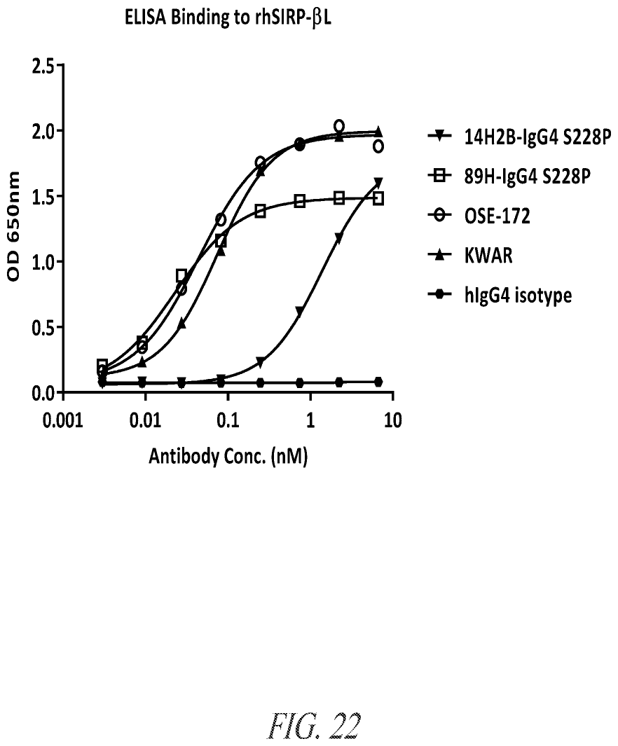

For binding to SIRP-βL, recombinant protein was coated onto 96-well ELISA plates at 1 μg/mL by overnight incubation at 4° C. Excess unbound protein was washed off and the plates were blocked with 1% bovine serum albumin (BSA) for 1 hour at room temperature (RT). Anti-SIRPα antibodies were added at the indicated concentrations and incubated for 1 hour at RT. Plates were washed and horse radish peroxidase (HRP) conjugated anti-human IgG secondary antibody added. Excess unbound secondary was washed and TMB substrate was added to the plates for approximately 10 minutes or until signal was visibly detectable. The reaction was stopped with acid and the plates were read immediately on the plate reader (Molecular Devices). The results are shown in FIG. 22.

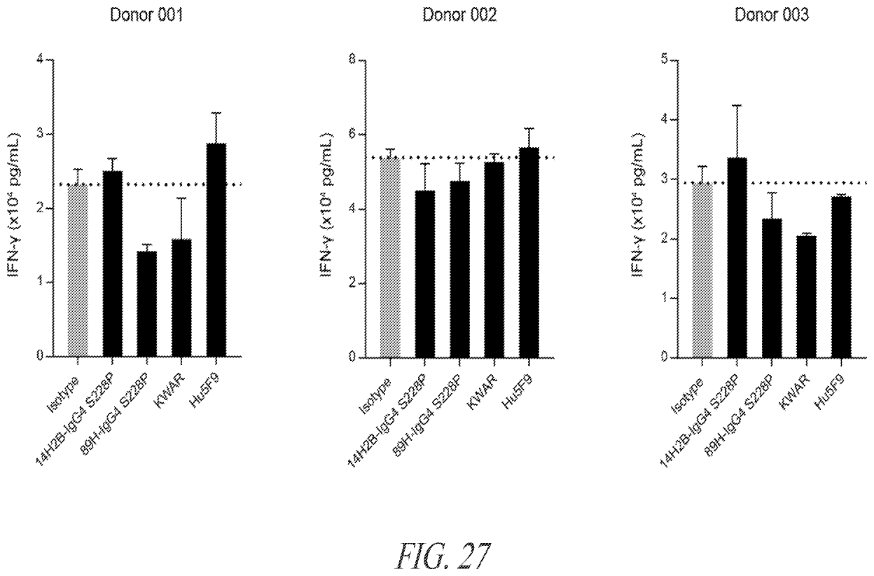

The SIRPγ-CD47 binding interaction is associated with normal T cell functions, such as migration, activation, and proliferation. To assess whether anti-SIRPα antibodies effect T cell activation, human PBMCs from three donors were stimulated with 1 ng/mL of staphylococcal enterotoxin B (SEB) in the presence of 6.6 nM of anti-SIRPα or anti-CD47 mAbs for 4 days. T cell activation was assessed by measuring IFN-γ from the harvested supernatant using an IFN-γ ELISA kit (R&D Systems). The results are shown in FIG. 27.

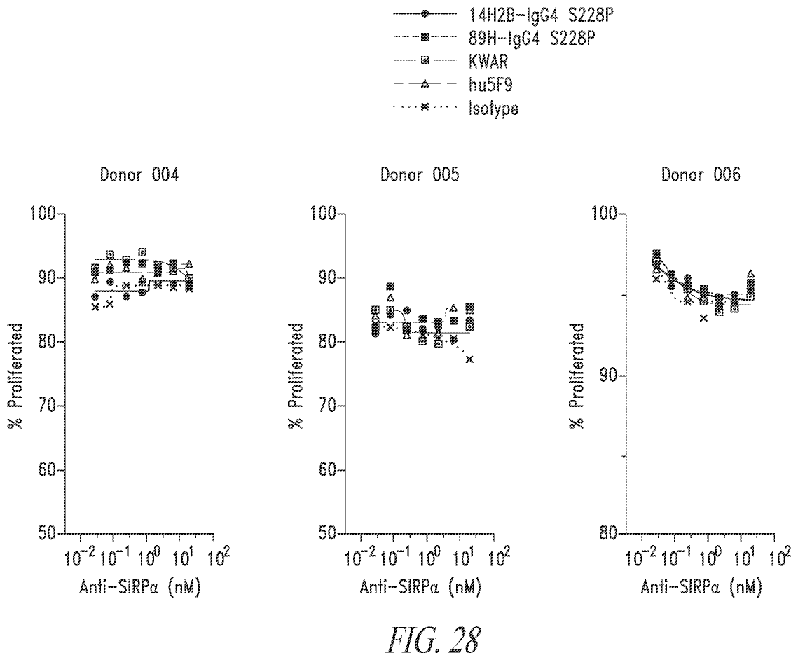

To determine the potential effects of anti-SIRPα antibodies on T cell proliferation, pan T cells were magnetically separated from PBMCs of three human donors (Miltenyi Biotech). Cells were subsequently stimulated with anti-CD3/anti-CD28 coated beads (Life Technologies) at a ratio of 2 cells per bead in the presence of titrated anti-SIRPα or anti-CD47 mAbs for 5 days. Proliferation was analyzed by flow cytometry using the presence of CellTrace Violet proliferation dye (Life Technologies) in daughter generations while dead cells were excluded using a fixable viability dye (Invitrogen) The results are shown in FIG. 28.

### Example 2

### Identification of Epitope Sites

Experiments were performed to determine the epitope of the SIRPα/14H2B and SIRPα/89H complexes with high resolution. The protein complexes were incubated with deuterated cross-linkers and subjected to multi-enzymatic cleavage. After enrichment of the cross-linked peptides, the samples were analyzed by high resolution mass spectrometry (nLC-LTQ-Orbitrap MS) and the data generated were analyzed using XQuest and Stavrox software.

Reduction Alkylation. 20 μL of the SIRPα-his/14H2B and SIRPα-his/89H mixtures prepared were mixed with 2 μL of DSS d0/d12 (2 mg/mL; DMF) before 180 minutes incubation time at room temperature. After incubation, reaction was stopped by adding 1 μL of Ammonium Bicarbonate (20 mM final concentration) before one hour incubation time at room temperature. Then, the solution was dried using a speedvac before H₂O 8M urea suspension (20 μL). After mixing, 2 μl of DTT (500 mM) were added to the solution. The mixture was then incubated one hour at 37° C. After incubation, 2 μl of iodoacetamide (1M) were added before one hour incubation time at room temperature, in a dark room. After incubation, 80 μl of the proteolytic buffer were added. The trypsin buffer contains 50 mM Ambic pH 8.5, 5% acetonitrile; the chymotrypsin buffer contains Tris HCl 100 mM, CaCl2 10 mM pH 7.8; the ASP-N buffer contains Phosphate buffer 50 mM pH 7.8; the elastase buffer contains Tris HCl 50 mM pH 8.0 and the thermolysin buffer contains Tris HCl 50 mM, CaCl2 0.5 mM pH 9.0.

Trypsin Proteolysis. 100 μl of the reduced/alkyled SIRPα-his/14H2B and SIRPα-his/89H mixtures were mixed with 0.7 μl of trypsin (Promega) with the ratio 1/100. The proteolytic mixtures were incubated overnight at 37° C.

Chymotrypsin Proteolysis. 100 μl of the reduced/alkyled SIRPα-his/14H2B and SIRPα-his/89H mixtures were mixed with 0.4 μl of chymotrypsin (Promega) with the ratio 1/200. The proteolytic mixtures were incubated overnight at 25° C.

ASP-N Proteolysis. 100 μl of the reduced/alkyled SIRPα-his/14H2B and SIRPα-his/89H mixtures were mixed with 0.4 μl of ASP-N (Promega) with the ratio 1/200. The proteolytic mixtures were incubated overnight at 37° C.

Elastase Proteolysis. 100 μl of the reduced/alkyled SIRPα-his/14H2B and SIRPα-his/89H mixtures were mixed with 0.7 μl of elastase (Promega) with the ratio 1/100. The proteolytic mixtures were incubated overnight at 37° C.

Thermolysin Proteolysis. 100 μl of the reduced/alkyled SIRPα-his/14H2B and SIRPα-his/89H mixtures were mixed with 1.4 μl of thermolysin (Promega) with a ratio 1/50. The proteolytic mixtures were incubated overnight at 70° C.

After digestion formic acid 1% final was added to the solution.

Data analysis. The cross-linked peptides were analyzed using Xquest version 2.0 and Stavrox 3.6. software.

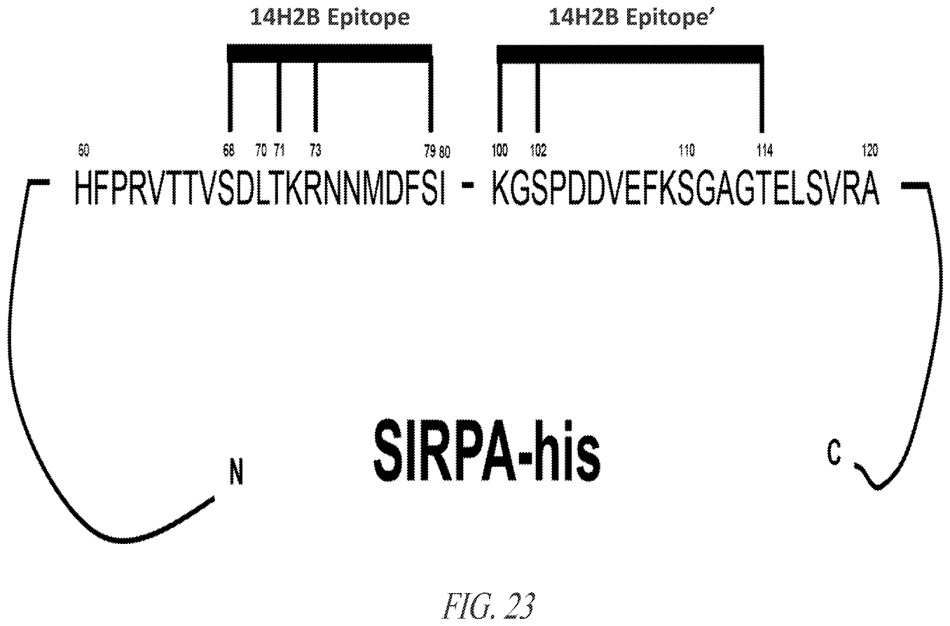

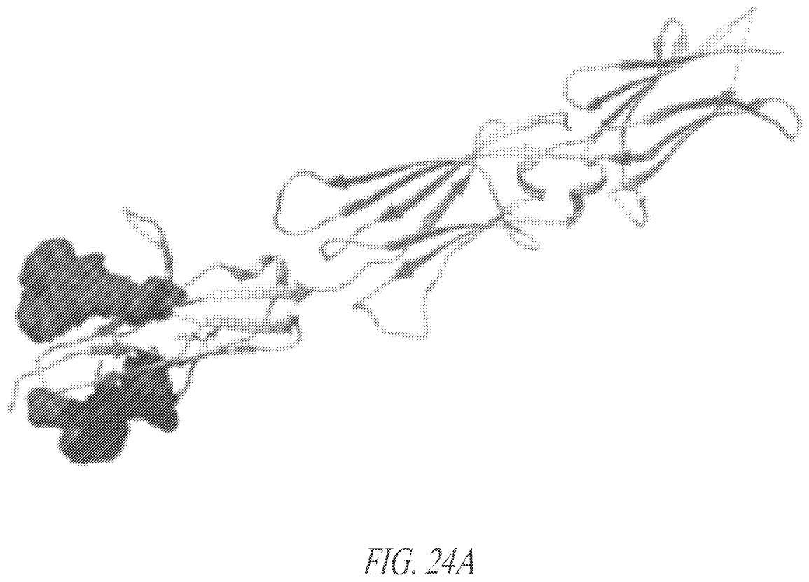

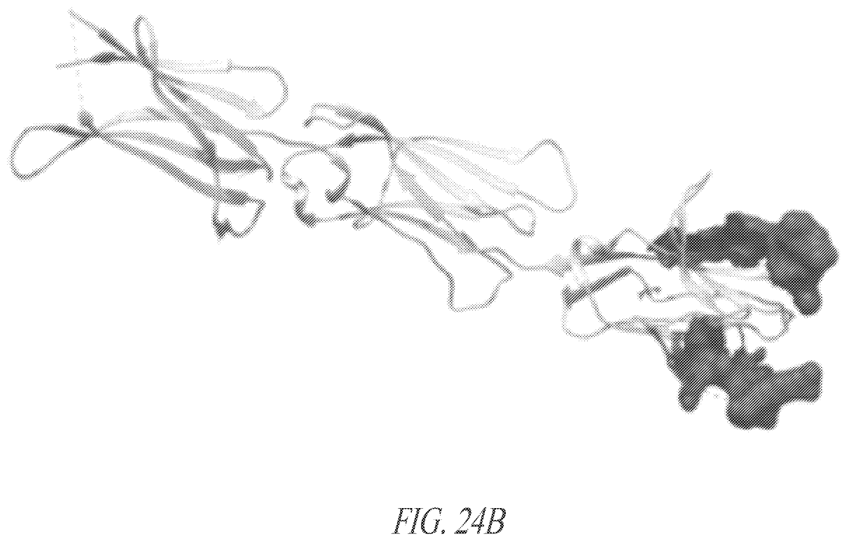

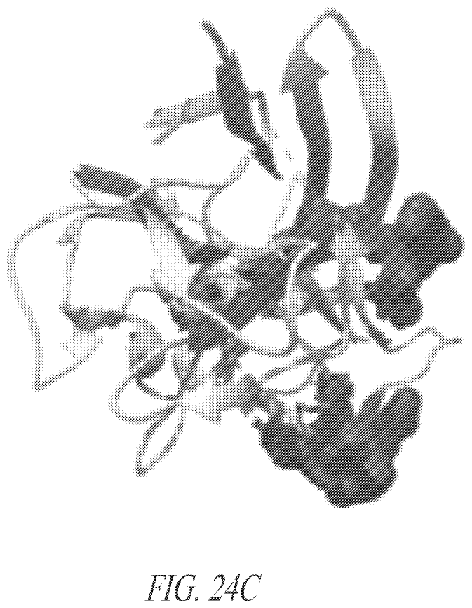

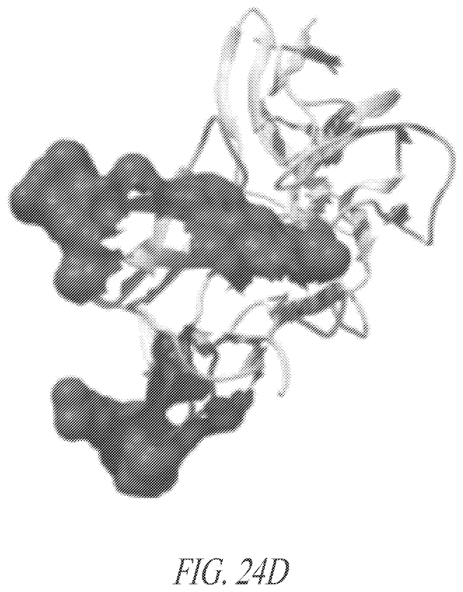

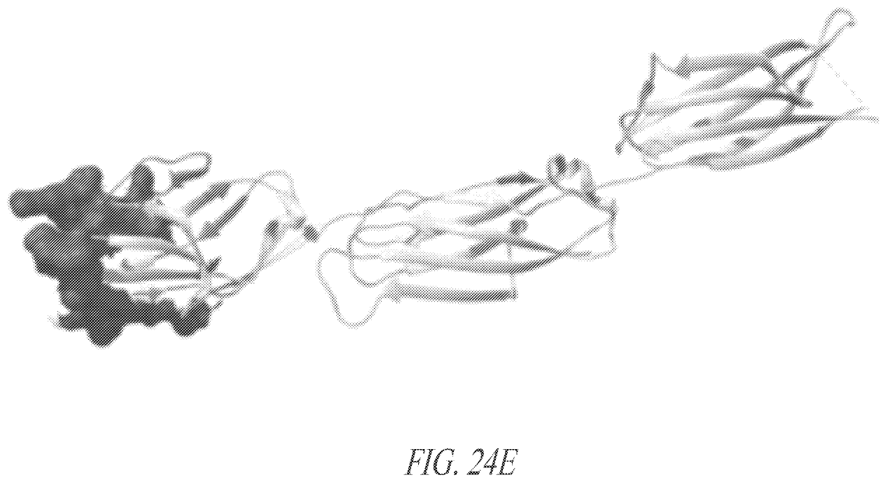

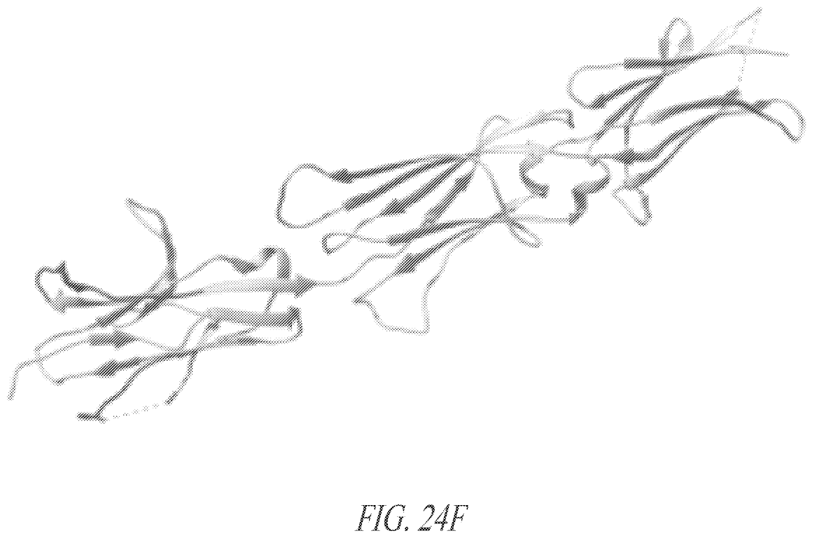

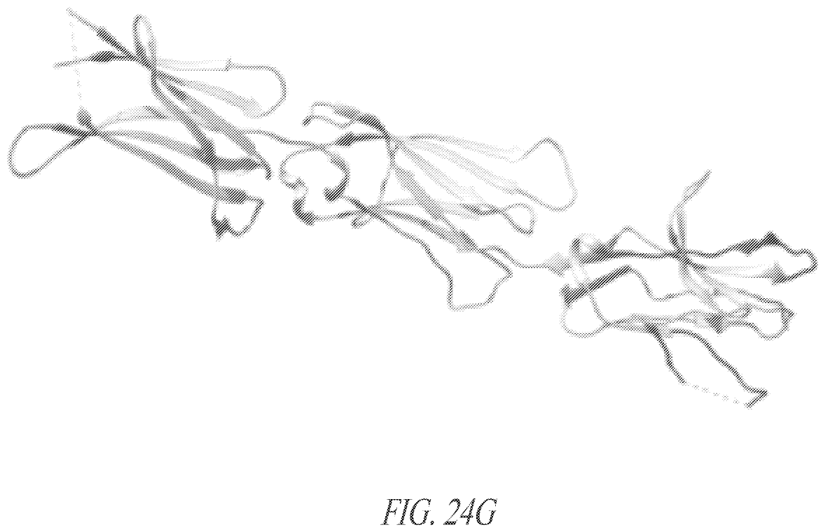

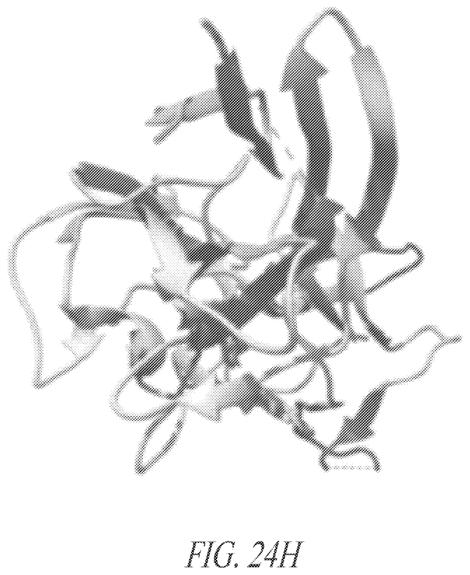

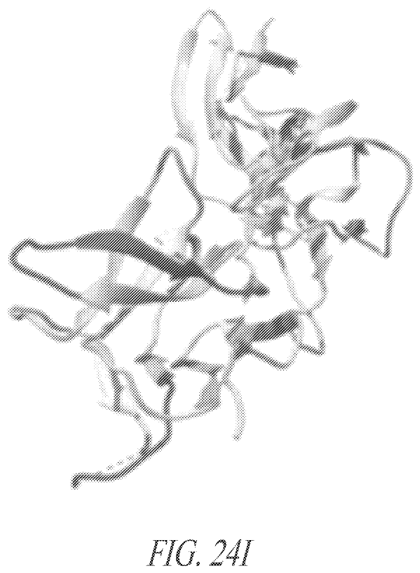

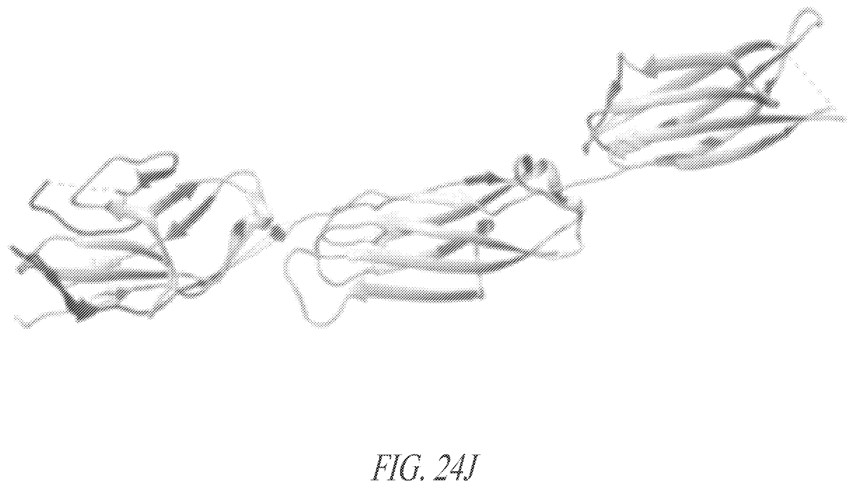

Results—Interaction between SIRPα-his/14H2B. After Trypsin, Chymotrypsin, ASP-N, Elastase, and Thermolysin proteolysis of the protein complex with deuterated d0d12, the nLC-orbitrap MS/MS analysis detected nine cross-linked peptides between SIRPα-His and the antibody 14H2B. Using chemical cross-linking, High-Mass MALDI mass spectrometry, and nLC-Orbitrap mass spectrometry the molecular interface between SIRPα-His and antibody 14H2B was characterized (see FIG. 23 and FIGS. 24A-24J), indicating that the interaction includes residues 68, 71, 73, 79, 79, 100, 102, 114 of human SIRPα (as defined by SEQ ID NO: 228).

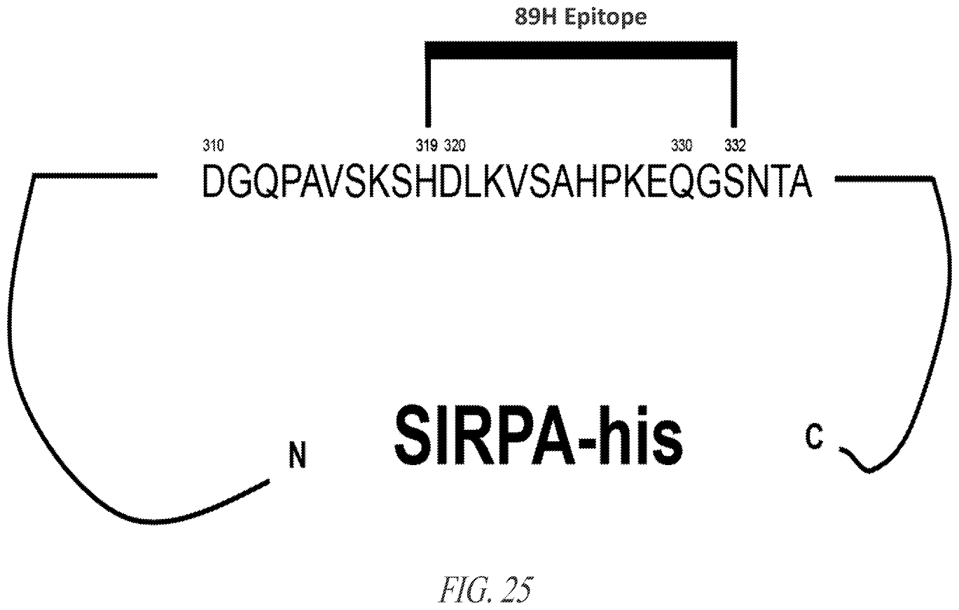

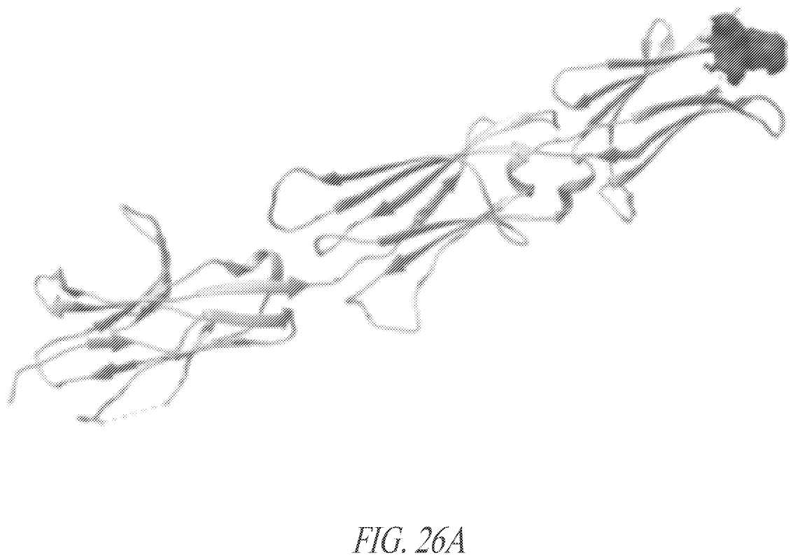

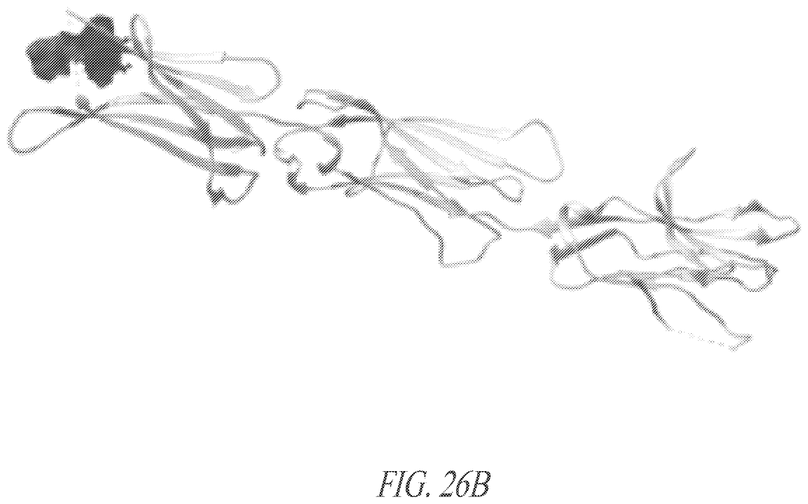

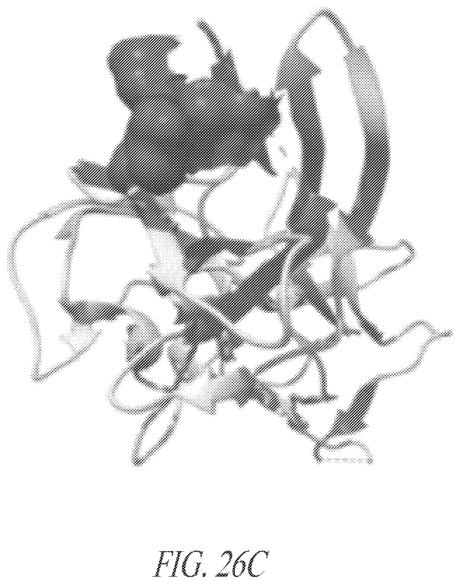

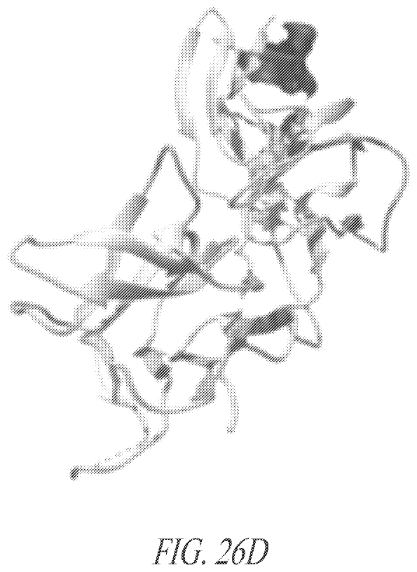

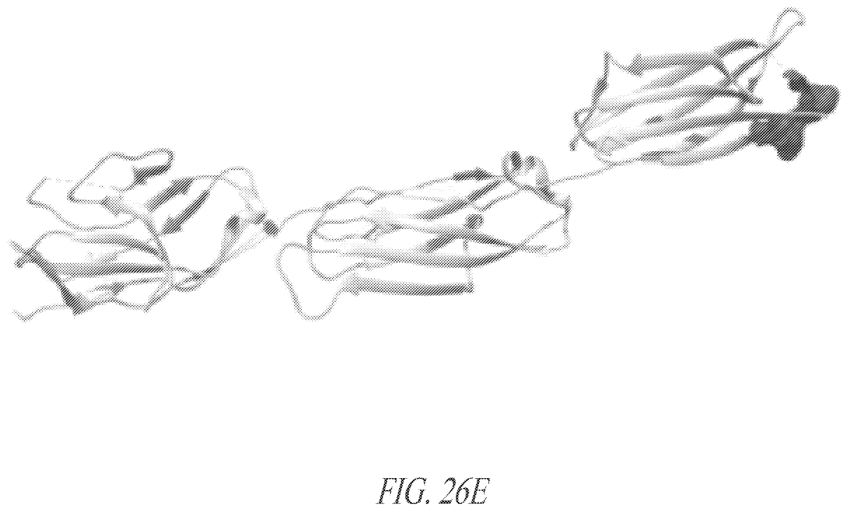

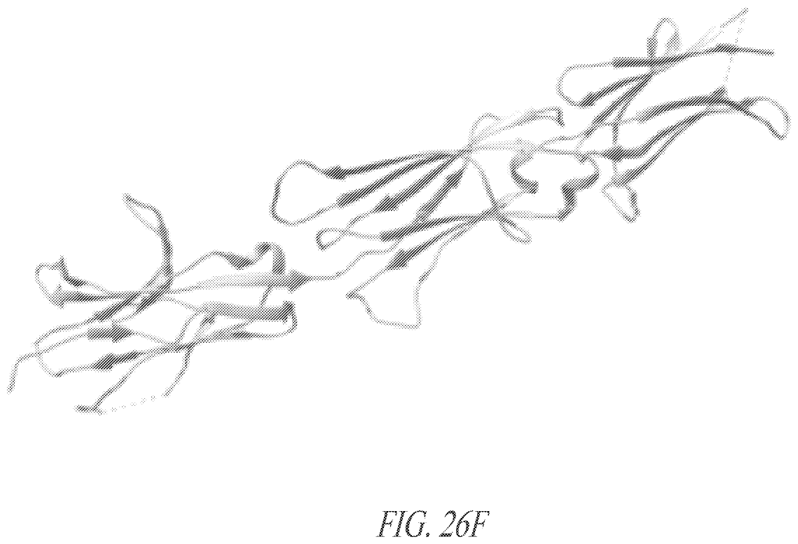

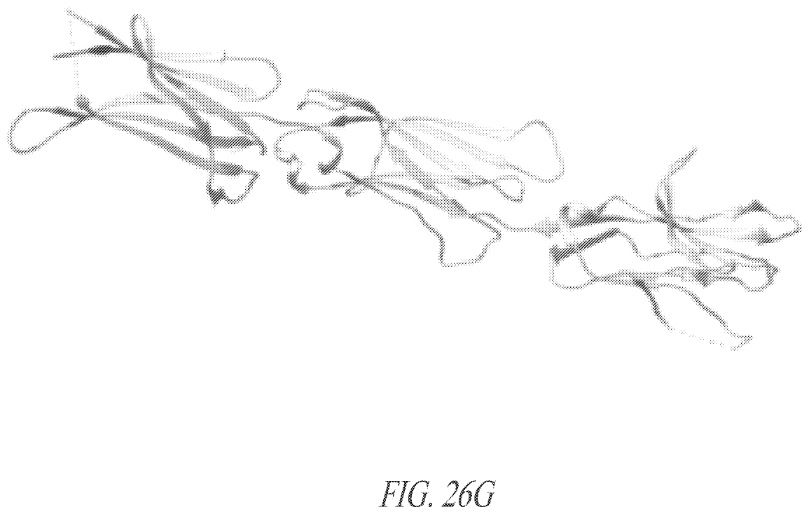

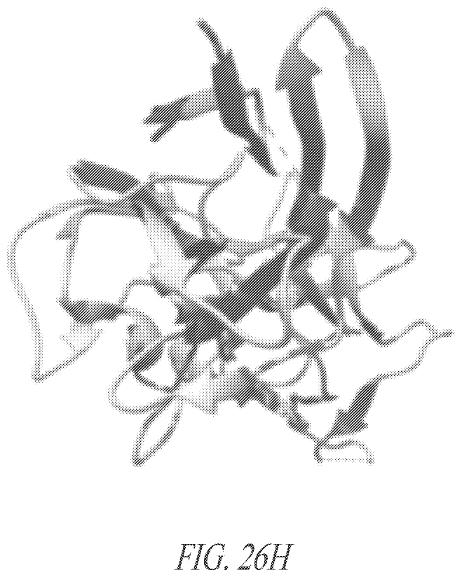

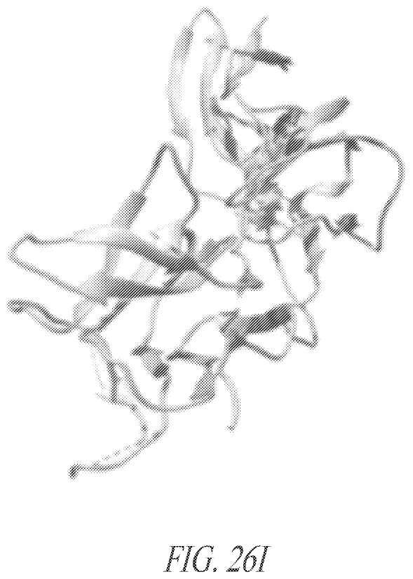

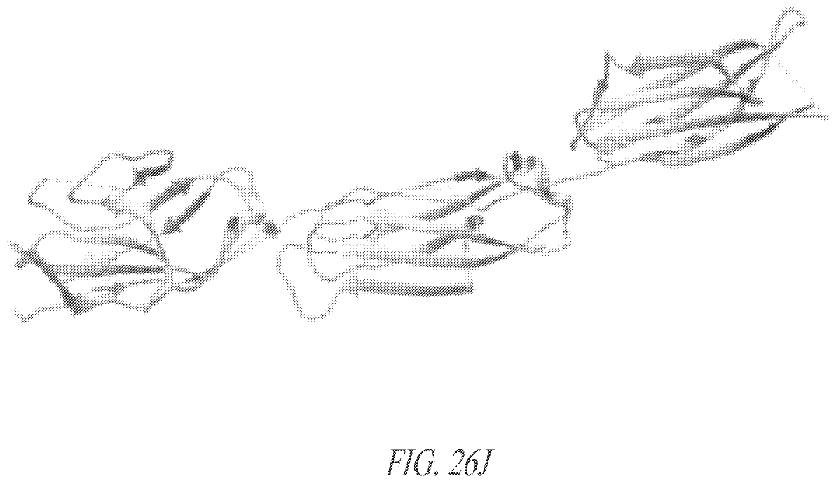

Results—Interaction between SIRPα-his/89H. After Trypsin, Chymotrypsin, ASP-N, Elastase, and Thermolysin proteolysis of the protein complex with deuterated d0d12, the nLC-orbitrap MS/MS analysis detected three crosslinked peptides between SIRPα-His and the antibody 89H. Using chemical cross-linking, High-Mass MALDI mass spectrometry, and nLC-Orbitrap mass spectrometry the molecular interface between SIRPα-His and antibody 89H was characterized (see FIG. 25 and FIGS. 26A-26J), indicating that the interaction includes residues 319 and 332 of human SIRPα (as defined by SEQ ID NO: 228).

Sequence analysis demonstrates that the epitope for antibody 14H2B is conserved in all major SIRP family members, including SIRPα V1, V2, and V8, SIRP-β1, SIRP-βL, and SIRP-γ; it also overlaps with the CD47 binding region. The epitope for antibody 89H is conserved in SIRPα V1, V2 and V8, and SIRP-βL, but is not conserved in SIRP-β1 and SIRP-γ; it also does not overlap with the CD47 binding region.

1. An isolated antibody, or an antigen-binding fragment thereof, that binds to signal regulatory protein α (SIRPα), comprising:
a heavy chain variable (VH) region comprising VHCDR1, VHCDR2, and VHCDR3 regions set forth respectively in SEQ ID NOs: 1-3; and a light chain variable (VL) region comprising VLCDR1, VLCDR2, and VLCDR3 regions set forth respectively in SEQ ID NOs: 4-6;
a VH region comprising VHCDR1, a VHCDR2, and VHCDR3 regions set forth respectively in SEQ ID NOs: 7-9; and a VL region comprising VLCDR1, VLCDR2, and VLCDR3 regions set forth respectively in SEQ ID NOs: 10-12;
a VH region comprising VHCDR1, VHCDR2, and VHCDR3 regions set forth respectively in SEQ ID NOs: 13-15; and a VL region comprising VLCDR1, VLCDR2, and VLCDR3 regions set forth respectively in SEQ ID NOs: 16-18;
a VH region comprising VHCDR1, VHCDR2, and VHCDR3 regions set forth respectively in SEQ ID NOs: 19-21; and a VL region comprising VLCDR1, VLCDR2, and VLCDR3 regions set forth respectively in SEQ ID NOs: 22-24;
a VH region comprising VHCDR1, VHCDR2, and VHCDR3 regions set forth respectively in SEQ ID NOs: 25-27; and a VL region comprising VLCDR1, VLCDR2, and VLCDR3 regions set forth respectively in SEQ ID NOs: 28-30;
a VH region comprising VHCDR1, VHCDR2, and VHCDR3 regions set forth respectively in SEQ ID NOs: 31-33; and a VL region comprising VLCDR1, VLCDR2, and VLCDR3 regions set forth respectively in SEQ ID NOs: 34-36;
a VH region comprising VHCDR1, VHCDR2, and VHCDR3 regions set forth respectively in SEQ ID NOs: 37-39; and a VL region comprising VLCDR1, VLCDR2, and VLCDR3 regions set forth respectively in SEQ ID NOs: 40-42;
a VH region comprising VHCDR1, VHCDR2, and VHCDR3 regions set forth respectively in SEQ ID NOs: 43-45; and a VL region comprising VLCDR1, VLCDR2, and VLCDR3 regions set forth respectively in SEQ ID NOs: 225 and 47-48, 226 and 47-48, or 46-48;
a VH region comprising VHCDR1, VHCDR2, and VHCDR3 regions set forth respectively in SEQ ID NOs: 49-51; and a VL region comprising VLCDR1, VLCDR2, and VLCDR3 regions set forth respectively in SEQ ID NOs: 52-54;
a VH region comprising VHCDR1, VHCDR2, and VHCDR3 regions set forth respectively in SEQ ID NOs: 55-57; and a VL region comprising VLCDR1, VLCDR2, and VLCDR3 regions set forth respectively in SEQ ID NOs: 58-60;
a VH region comprising VHCDR1, VHCDR2, and VHCDR3 regions set forth respectively in SEQ ID NOs: 61-63; and a VL region comprising VLCDR1, VLCDR2, and VLCDR3 regions set forth respectively in SEQ ID NOs: 64-66;
a VH region comprising VHCDR1, VHCDR2, and VHCDR3 regions set forth respectively in SEQ ID NOs: 67-69; and a VL region comprising VLCDR1, VLCDR2, and VLCDR3 regions set forth respectively in SEQ ID NOs: 70-72;
a VH region comprising VHCDR1, VHCDR2, and VHCDR3 regions set forth respectively in SEQ ID NOs: 73-75; and a VL region comprising VLCDR1, VLCDR2, and VLCDR3 regions set forth respectively in SEQ ID NOs: 76-78;
a VH region comprising VHCDR1, VHCDR2, and VHCDR3 regions set forth respectively in SEQ ID NOs: 79-81; and a VL region comprising VLCDR1, VLCDR2, and VLCDR3 regions set forth respectively in SEQ ID NOs: 82-84;
a VH region comprising VHCDR1, VHCDR2, and VHCDR3 regions set forth respectively in SEQ ID NOs: 85-87; and a VL region comprising VLCDR1, VLCDR2, and VLCDR3 regions set forth respectively in SEQ ID NOs: 88-90;
a VH region comprising VHCDR1, VHCDR2, and VHCDR3 regions set forth respectively in SEQ ID NOs: 91-93; and a VL region comprising VLCDR1, VLCDR2, and VLCDR3 regions set forth respectively in SEQ ID NOs: 94-96; or
a VH region comprising VHCDR1, VHCDR2, and VHCDR3 regions set forth respectively in SEQ ID NOs: 97-99; and a VL region comprising VLCDR1, VLCDR2, and VLCDR3 regions set forth respectively in SEQ ID NOs: 100-102;
a VH region comprising VHCDR1, VHCDR2, and VHCDR3 regions set forth respectively in SEQ ID NOs: 103-105; and a VL region comprising VLCDR1, VLCDR2, and VLCDR3 regions set forth respectively in SEQ ID NOs: 106-108;
a VH region comprising VHCDR1, VHCDR2, and VHCDR3 regions set forth respectively in SEQ ID NOs: 109-111; and a VL region comprising VLCDR1, VLCDR2, and VLCDR3 regions set forth respectively in SEQ ID NOs: 112-114;
a VH region comprising VHCDR1, VHCDR2, and VHCDR3 regions set forth respectively in SEQ ID NOs: 115-117; and a VL region comprising VLCDR1, VLCDR2, and VLCDR3 regions set forth respectively in SEQ ID NOs: 118-120;
a VH region comprising VHCDR1, VHCDR2, and VHCDR3 regions set forth respectively in SEQ ID NOs: 121-123; and a VL region comprising VLCDR1, VLCDR2, and VLCDR3 regions set forth respectively in SEQ ID NOs: 124-126;
a VH region comprising VHCDR1, VHCDR2, and VHCDR3 regions set forth respectively in SEQ ID NOs: 127-129; and a VL region comprising VLCDR1, VLCDR2, and VLCDR3 regions set forth respectively in SEQ ID NOs: 130-132;
a VH region comprising VHCDR1, VHCDR2, and VHCDR3 regions set forth respectively in SEQ ID NOs: 133-135; and a VL region comprising VLCDR1, VLCDR2, and VLCDR3 regions set forth respectively in SEQ ID NOs: 136-138;
a VH region comprising VHCDR1, VHCDR2, and VHCDR3 regions set forth respectively in SEQ ID NOs: 139-141; and a VL region comprising VLCDR1, VLCDR2, and VLCDR3 regions set forth respectively in SEQ ID NOs: 142-144;
a VH region comprising VHCDR1, VHCDR2, and VHCDR3 regions set forth respectively in SEQ ID NOs: 145-147; and a VL region comprising VLCDR1, VLCDR2, and VLCDR3 regions set forth respectively in SEQ ID NOs: 148-150;
a VH region comprising VHCDR1, VHCDR2, and VHCDR3 regions set forth respectively in SEQ ID NOs: 151-153; and a VL region comprising VLCDR1, VLCDR2, and VLCDR3 regions set forth respectively in SEQ ID NOs: 154-156;
a VH region comprising VHCDR1, VHCDR2, and VHCDR3 regions set forth respectively in SEQ ID NOs: 157-159; and a VL region comprising VLCDR1, VLCDR2, and VLCDR3 regions set forth respectively in SEQ ID NOs: 160-162;
a VH region comprising VHCDR1, VHCDR2, and VHCDR3 regions set forth respectively in SEQ ID NOs: 163-165; and a VL region comprising VLCDR1, VLCDR2, and VLCDR3 regions set forth respectively in SEQ ID NOs: 166-168, or
or a variant of said antibody, or an antigen-binding fragment thereof, comprising heavy and light chain variable regions identical to the heavy and light chain variable regions of (i) and (ii) except for up to 1, 2, 3, 4, 5, 6, 7, or 8 total amino acid substitutions across said CDR regions.

2. The isolated antibody, or antigen-binding fragment thereof, of claim 1, wherein the VH region comprises an amino acid sequence having at least 90%, 95%, 96%, 97%, 98%, 99%, or 100% identity to a sequence selected from SEQ ID NOs: 169, 171, 173, 175, 177, 179, 181, 183, 185, 187, 189, 191, 193, 195, 197, 199, 201, 203, 205, 207, 209, 211, 213, 215, 217, 219, 221, and 223.

3. The isolated antibody, or antigen-binding fragment thereof, of claim 1 or 2, wherein the VL region comprises an amino acid sequence having at least 90%, 95%, 96%, 97%, 98%, 99%, or 100% identity to a sequence selected from SEQ ID NOs: 168, 170, 172, 174, 176, 178, 180, 182, 184 or 227, 186, 188, 190, 192, 194, 196, 198, 200, 202, 204, 206, 208, 210, 212, 214, 216, 218, 220, 222, and 224.

4. The isolated antibody, or antigen-binding fragment thereof, of claim 2 or 3, comprising:
the VH region set forth in SEQ ID NO: 169, and the VL region set forth in SEQ ID NO: 170;
the VH region set forth in SEQ ID NO: 171, and the VL region set forth in SEQ ID NO: 172;
the VH region set forth in SEQ ID NO: 173, and the VL region set forth in SEQ ID NO: 174;
the VH region set forth in SEQ ID NO: 175, and the VL region set forth in SEQ ID NO: 176;
the VH region set forth in SEQ ID NO: 177, and the VL region set forth in SEQ ID NO: 178;
the VH region set forth in SEQ ID NO: 179, and the VL region set forth in SEQ ID NO: 180;
the VH region set forth in SEQ ID NO: 181, and the VL region set forth in SEQ ID NO: 182;
the VH region set forth in SEQ ID NO: 183, and the VL region set forth in SEQ ID NO: 184 or 227;
the VH region set forth in SEQ ID NO: 185, and the VL region set forth in SEQ ID NO: 186;
the VH region set forth in SEQ ID NO: 187, and the VL region set forth in SEQ ID NO: 188;
the VH region set forth in SEQ ID NO: 189, and the VL region set forth in SEQ ID NO: 190;
the VH region set forth in SEQ ID NO: 191, and the VL region set forth in SEQ ID NO: 192;
the VH region set forth in SEQ ID NO: 193, and the VL region set forth in SEQ ID NO: 194;
the VH region set forth in SEQ ID NO: 195, and the VL region set forth in SEQ ID NO: 196;
the VH region set forth in SEQ ID NO: 197, and the VL region set forth in SEQ ID NO: 198;
the VH region set forth in SEQ ID NO: 199, and the VL region set forth in SEQ ID NO: 200;
the VH region set forth in SEQ ID NO: 201, and the VL region set forth in SEQ ID NO: 202;
the VH region set forth in SEQ ID NO: 203, and the VL region set forth in SEQ ID NO: 204;
the VH region set forth in SEQ ID NO: 205, and the VL region set forth in SEQ ID NO: 206;
the VH region set forth in SEQ ID NO: 207, and the VL region set forth in SEQ ID NO: 208;
the VH region set forth in SEQ ID NO: 209, and the VL region set forth in SEQ ID NO: 210;
the VH region set forth in SEQ ID NO: 211, and the VL region set forth in SEQ ID NO: 212;
the VH region set forth in SEQ ID NO: 213, and the VL region set forth in SEQ ID NO: 214;
the VH region set forth in SEQ ID NO: 215, and the VL region set forth in SEQ ID NO: 216;
the VH region set forth in SEQ ID NO: 217, and the VL region set forth in SEQ ID NO: 218;
the VH region set forth in SEQ ID NO: 219, and the VL region set forth in SEQ ID NO: 220;
the VH region set forth in SEQ ID NO: 221, and the VL region set forth in SEQ ID NO: 222; or
the VH region set forth in SEQ ID NO 223:, and the VL region set forth in SEQ ID NO: 224.

5. An isolated antibody, or an antigen-binding fragment thereof, that binds to signal regulatory protein α (SIRPα), comprising a heavy chain variable (VH) region which comprises an amino acid sequence having at least 90%, 95%, 96%, 97%, 98%, 99%, or 100% identity to a sequence selected from SEQ ID NOs: 169, 171, 173, 175, 177, 179, 181, 183, 185, 187, 189, 191, 193, 195, 197, 199, 201, 203, 205, 207, 209, 211, 213, 215, 217, 219, 221, and 223, and, respectively, a light chain variable (VL) region which comprises an amino acid sequence having at least 90%, 95%, 96%, 97%, 98%, 99%, or 100% identity to a sequence selected from SEQ ID NO: 168, 170, 172, 174, 176, 178, 180, 182, 184 or 227, 186, 188, 190, 192, 194, 196, 198, 200, 202, 204, 206, 208, 210, 212, 214, 216, 218, 220, 222, and 224.

6. An isolated antibody, or an antigen-binding fragment thereof, that binds to signal regulatory protein α (SIRPα) at an epitope selected from:
(a) an epitope that comprises, consists, or consists essentially of one or more residues selected from S68, L70, T71, R73, S79, K100, S102, and T114, as defined by the human SIRPα sequence of SEQ ID NO: 228, optionally wherein the epitope comprises, consists, or consists essentially of one or more residues selected from SDLTKRNNMDFS (SEQ ID NO: 232) and KGSPDDVEFKSGAGT (SEQ ID NO: 233); and
(b) an epitope that comprises, consists, or consists essentially of one or more residues selected from H319 and 5332, as defined by the human SIRPα sequence of SEQ ID NO: 228, optionally wherein the epitope comprises, consists, or consists essentially of HDLKVSAHPKEQGS (SEQ ID NO: 234).

7. An isolated antibody, or an antigen-binding fragment thereof, that binds to signal regulatory protein β (SIRPβL) at an epitope selected from:
(a) an epitope that comprises, consists, or consists essentially of one or more residues selected from SDLTKRNNMDFS (SEQ ID NO: 232) and KGSPDDVEFKSGAGT (SEQ ID NO: 233); and
(b) an epitope that comprises, consists, or consists essentially of HDLKVSAHPKEQGS (SEQ ID NO: 234).

8. An isolated antibody, or an antigen-binding fragment thereof, that binds to signal regulatory protein βL (SIRPβL) at an epitope selected from:
(a) an epitope that comprises, consists, or consists essentially of one or more residues selected from SDLTKRNNMDFS (SEQ ID NO: 232) and KGSPDDVEFKSGAGT (SEQ ID NO: 233); and
(b) an epitope that comprises, consists, or consists essentially of HDLKVSAHPKEQGS (SEQ ID NO: 234).

9. The isolated antibody, or antigen-binding fragment thereof, of any one of claims 6-8, wherein
the antibody, or antigen-binding fragment thereof, of (a) comprises a heavy chain variable (VH) region comprising VHCDR1, VHCDR2, and VHCDR3 regions set forth respectively in SEQ ID NOs: 43, 44, and 45; and a light chain variable (VL) region comprising VLCDR1, VLCDR2, and VLCDR3 regions set forth respectively in SEQ ID NOs: 226, 47, and 48; or
the antibody, or antigen-binding fragment thereof, of (b) comprises VHCDR1, VHCDR2, and VHCDR3 regions set forth respectively in SEQ ID NOs: 49, 50, and 51; and VLCDR1, VLCDR2, and VLCDR3 regions set forth respectively in SEQ ID NOs: 52, 53, and 54,
including variants of said antibody, or antigen-binding fragment thereof, comprising up to 1, 2, 3, 4, 5, 6, 7, or 8 total amino acid substitutions across said CDR regions.

10. The isolated antibody, or antigen-binding fragment thereof, of any one of claims 6-9, wherein
the antibody, or antigen-binding fragment thereof, of (a) comprises a VH region having an amino acid sequence with at least 90%, 95%, 96%, 97%, 98%, 99%, or 100% identity to SEQ ID NO:
183, and/or a VL region having an amino acid sequence with at least 90%, 95%, 96%, 97%, 98%, 99%, or 100% identity to SEQ ID NO: 227; or
the antibody, or antigen-binding fragment thereof, of (b) comprises a VH region having an amino acid sequence with at least 90%, 95%, 96%, 97%, 98%, 99%, or 100% identity to SEQ ID NO: 185, and/or a VL region having an amino acid sequence with at least 90%, 95%, 96%, 97%, 98%, 99%, or 100% identity to SEQ ID NO: 186.

11. The isolated antibody, or antigen-binding fragment thereof, of any one of claims 6-10, wherein
the antibody, or antigen-binding fragment thereof, of (a) comprises the VH region set forth in SEQ ID NO: 183, and/or the VL region set forth in SEQ ID NO: 227; or
the antibody, or antigen-binding fragment thereof, of (b) comprises the VH region set forth in SEQ ID NO: 185, and/or the VL region set forth in SEQ ID NO: 186.

12. An isolated antibody, or an antigen-binding fragment thereof, that binds to signal regulatory protein α (SIRPα), comprising a heavy chain variable (VH) region comprising VHCDR1, VHCDR2, and VHCDR3 regions selected from the underlined sequences in Table R1; and, respectively, a light chain variable (VL) region comprising VLCDR1, VLCDR2, and VLCDR3 regions selected from underlined sequences in Table R2.

13. The isolated antibody, or an antigen-binding fragment thereof, of claim 12, comprising a VH region which comprises an amino acid sequence selected from Table R1, and, respectively, a VL region which comprises an amino acid sequence selected from Table R2, optionally as defined in Table R3.

14. The isolated antibody, or an antigen-binding fragment thereof, of any one of claims 1-13, which binds to human SIRPα, including soluble and cell-expressed human SIRPα, optionally the V1, V2, and/or V8 variants of human SIRPα.

15. The isolated antibody, or an antigen-binding fragment thereof, of claim 14, which binds to at least one human SIRPα polypeptide or domain or epitope selected from Table S1.

16. The isolated antibody of any one of claims 1-15, wherein the antibody is humanized.

17. The isolated antibody of any one of claims 1-16, wherein the antibody is selected from the group consisting of a whole antibody, a Fab or a Fab′ fragment, a F(ab′)2 fragment, a single chain antibody, a scFv, a univalent antibody lacking a hinge region, a minibody, and a probody.

18. The isolated antibody of any one of claims 1-17, comprising a human IgG constant domain, optionally wherein IgG constant domain comprises an IgG1 CH1 domain.

19. The isolated antibody of claim 18, wherein the IgG constant domain comprises an IgG1 Fc region or an IgG4 Fc, optionally a modified Fc region, optionally modified by one or more amino acid substitutions such as an S228P substitution.

20. The isolated antibody, or antigen-binding fragment thereof, of any one of claims 1-19, which binds to human SIRPα with a K_{D} of 0.4 nM or lower.

21. The isolated antibody, or antigen-binding fragment thereof, of claim 20, which binds to at least one human SIRPα polypeptide or domain or epitope from Table S1 with a K_{D} of 0.4 nM or lower.

22. The isolated antibody, or antigen-binding fragment thereof, of any one of claims 1-19, which comprises an IgG1 Fc region and binds to SIRPα, optionally at least one SIRPα polypeptide or domain or epitope from Table S1, with a K_{D} of about 0.16 to about 2.5 nM.

23. The isolated antibody, or antigen-binding fragment thereof, of any one of claims 1-19, which comprises an IgG4 Fc region having an S228P substitution and binds SIRPα, optionally at least one SIRPα polypeptide or domain or epitope from Table S1, with a K_{D} of about 0.09 to about 1.66 nM, or about 0.088 nM, about 0.2643 nM, about 0.3778 nM, about 0.672 nM, about 0.6864 nM, or about 1.66 nM, optionally as measured by flow cytometric analysis of binding to cell surface SIRPα expressed on dendritic cells.

24. The isolated antibody, or antigen-binding fragment thereof, of any one of claims 1-23, wherein the isolated antibody, or antigen-binding fragment thereof:
(a) selectively binds to SIRPα with high affinity, and does not significantly bind to SIRPβ and/or SIRPβ;
(b) binds to SIRPα variants including V1, V2, and/or V8 SIRPα variants;
(c) binds to SIRPα and SIRPβ, and does not substantially bind to SIRPβ;
(d) binds to SIRPα and SIRPγ, and does not substantially bind to SIRPβ;
(e) binds to SIRPα, SIRPβ, and SIRPβ;
(f) binds to SIRPα and SIRPβL;
(g) binds to SIRPα and does not substantially bind to SIRPβL;
(h) inhibits binding of SIRPα to its ligand CD47;
(i) inhibits SIRPα-CD47-mediated signaling;
induces and/or increases macrophage-mediated phagocytosis of cancer cells, optionally by reducing SIRPα-mediated inhibition of phagocytosis;
(k) increases antibody-dependent cell phagocytosis (ADCP);
(l) does not significantly inhibit binding of SIRPα to its ligand CD47 and does not significantly inhibit SIRPα-CD47-mediated signaling;
(m) inhibits macrophage-medicated phagocytosis;
(n) cross-reactively binds to human SIRPα and cynomolgus monkey SIRPα;
(o) binds to myeloid cells and does not significantly bind to primary T cells;
(p) increases dendritic cell activation of cytotoxic T cells; or
(q) a combination of any one or more of (a)-(o).

25. The isolated antibody, or antigen-binding fragment thereof, of any one of claims 1-24, which is a SIRPα antagonist.

26. The isolated antibody, or antigen-binding fragment thereof, of any one of claims 1-24, which is a SIRPα agonist.

27. The isolated antibody, or antigen-binding fragment thereof, of any one of claims 1-26, which is a bi-specific or multi-specific antibody.

28. An isolated polynucleotide encoding the isolated antibody, or antigen-binding fragment thereof, according to any one of claims 1-26, an expression vector comprising the isolated polynucleotide, or an isolated host cell comprising the vector.

29. A composition comprising a physiologically acceptable carrier and a therapeutically effective amount of the isolated antibody, or antigen-binding fragment thereof, according to any one of claims 1-26.

30. A method for treating, inhibiting the progression of, ameliorating the symptoms of, a cancer in a patient in need thereof, comprising administering to the patient the composition of claim 29, optionally wherein the antibody, or antigen-binding fragment thereof, is a SIRPα antagonist, thereby inhibiting the progression of, ameliorating the symptoms of, or treating the cancer.

31. The method of claim 30, wherein the cancer is associated with aberrant SIRPα and/or CD47 expression.

32. The method of claim 30 or 31, wherein the cancer is associated with SIRPα-mediated and/or CD47-mediated immune suppression.

33. The method of claim 32, wherein the immune suppression comprises inhibition of phagocytosis by innate immune cells, optionally macrophages and/or dendritic cells.

34. The method of any one of claims 30-33, wherein the composition increases an immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to a control or reference.

35. The method of claim 34, wherein the immune response comprises macrophage-mediated phagocytosis of cancer cells.

36. The method of claim 34, wherein the immune response comprises antibody-dependent cell phagocytosis (ADCP) of cancer cells.

37. The method of any one of claims 30-36, wherein the cancer is selected from one or more of lymphomas including non-Hodgkin's lymphomas, Hodgkin's lymphoma, and cutaneous T-cell lymphoma (e.g., Sézary disease), leukemias including chronic lymphocytic leukemias, acute myeloid leukemias, hairy cell leukemias, and acute lymphoblastic leukemias, multiple myeloma, and cancers or carcinomas of the pancreas, colon (e.g., colorectal cancer), gastric intestine, prostate, testis, bladder (e.g., urothelial cancer), kidney (e.g., renal cell carcinoma), ovary, cervix, breast (e.g., breast carcinoma), lung, brain (e.g., glioma), nasopharynx, head and neck, liver (e.g., hepatocellular carcinoma), and skin (e.g., melanoma or malignant melanoma).

38. A method of treating, reducing the severity of, or preventing an infectious disease in a patient in need thereof, comprising administering to the patient the composition of claim 29, optionally wherein the antibody, or antigen-binding fragment thereof, is a SIRPα antagonist, thereby treating, reducing the severity of, or preventing the infectious disease.

39. The method of claim 38, wherein the infectious disease is selected from viral, bacterial, fungal optionally yeast, and protozoal infections.

40. A method of treating an autoimmune or inflammatory disease in a subject in need thereof, comprising administering to the patient a composition of claim 29, optionally wherein the antibody, or antigen-binding fragment thereof, is a SIRPα agonist, thereby treating the autoimmune or inflammatory disease.

41. The method of claim 40, wherein the autoimmune or inflammatory disease is associated with aberrant macrophage activation and/or phagocytosis.

42. A method of improving transplantation in a patient in need thereof, comprising administering to the patient a composition of claim 27 in combination with transplanted cells, optionally wherein the antibody, or antigen-binding fragment thereof, is a SIRPα agonist that reduces phagocytosis of the transplanted cells, thereby improving transplantation in the patient.

43. The method of claim 42, wherein the transplanted cells comprises hematopoietic stem cells, progenitor stem cells, or a solid organ.

44. The method of claim 42 or 43, comprising administering the composition to the subject prior to administration of transplanted cells, concurrently with administration of transplanted cells, or shortly after administration of transplanted cells.


SEQUENCE LISTING (416 sequences)

SEQ ID NO: 1 - protein, 6 aa, Artificial Sequence
  Made in Lab - Heavy CDR1
GSDYIC

SEQ ID NO: 2 - protein, 17 aa, Artificial Sequence
  Made in Lab - Heavy CDR2
CINVGIDNTYYASWATG

SEQ ID NO: 3 - protein, 14 aa, Artificial Sequence
  Made in Lab - Heavy CDR3
GYSASAWGLDYFNL

SEQ ID NO: 4 - protein, 11 aa, Artificial Sequence
  Made in Lab - Light CDR1
QASESIGNYLA

SEQ ID NO: 5 - protein, 7 aa, Artificial Sequence
  Made in Lab - Light CDR2
GASTLES

SEQ ID NO: 6 - protein, 13 aa, Artificial Sequence
  Made in Lab - Light CDR3
QCTYGTTSISTYT

SEQ ID NO: 7 - protein, 6 aa, Artificial Sequence
  Made in Lab - Heavy CDR1
SYYFMC

SEQ ID NO: 8 - protein, 18 aa, Artificial Sequence
  Made in Lab - Heavy CDR2
CIDAGYSGGSHYASWAKG

SEQ ID NO: 9 - protein, 11 aa, Artificial Sequence
  Made in Lab - Heavy CDR3
AYEYGYGGYDL

SEQ ID NO: 10 - protein, 11 aa, Artificial Sequence
  Made in Lab - Light CDR1
QASQSIDSNLA

SEQ ID NO: 11 - protein, 7 aa, Artificial Sequence
  Made in Lab - Light CDR2
RASGLES

SEQ ID NO: 12 - protein, 15 aa, Artificial Sequence
  Made in Lab - Light CDR3
QCTYGSNDSGNYANA

SEQ ID NO: 13 - protein, 6 aa, Artificial Sequence
  Made in Lab - Heavy CDR1
SSDYMC

SEQ ID NO: 14 - protein, 17 aa, Artificial Sequence
  Made in Lab - Heavy CDR2
CICTGHGDTAYATWAKG

SEQ ID NO: 15 - protein, 15 aa, Artificial Sequence
  Made in Lab - Heavy CDR3
GWTSSADYYIYYLNL

SEQ ID NO: 16 - protein, 11 aa, Artificial Sequence
  Made in Lab - Light CDR1
QASESIYNYLS

SEQ ID NO: 17 - protein, 7 aa, Artificial Sequence
  Made in Lab - Light CDR2
GASNLES

SEQ ID NO: 18 - protein, 12 aa, Artificial Sequence
  Made in Lab - Light CDR3
QQGYSGNNIDNI

SEQ ID NO: 19 - protein, 6 aa, Artificial Sequence
  Made in Lab - Heavy CDR1
SSDYMC

SEQ ID NO: 20 - protein, 17 aa, Artificial Sequence
  Made in Lab - Heavy CDR2
CINSGSDTTSYATWAKG

SEQ ID NO: 21 - protein, 19 aa, Artificial Sequence
  Made in Lab - Heavy CDR3
GYGSAGANGYVIGMKYLNL

SEQ ID NO: 22 - protein, 11 aa, Artificial Sequence
  Made in Lab - Light CDR1
QASQSIGSNLA

SEQ ID NO: 23 - protein, 7 aa, Artificial Sequence
  Made in Lab - Light CDR2
TASTLES

SEQ ID NO: 24 - protein, 12 aa, Artificial Sequence
  Made in Lab - Light CDR3
QCTYGSSSSGYP

SEQ ID NO: 25 - protein, 6 aa, Artificial Sequence
  Made in Lab - Heavy CDR1
GSEYMS

SEQ ID NO: 26 - protein, 18 aa, Artificial Sequence
  Made in Lab - Heavy CDR2
CIYFGISGSTYYANWAKG

SEQ ID NO: 27 - protein, 13 aa, Artificial Sequence
  Made in Lab - Heavy CDR3
GYGDKYNLYYFNL

SEQ ID NO: 28 - protein, 11 aa, Artificial Sequence
  Made in Lab - Light CDR1
QASEDIESYLA

SEQ ID NO: 29 - protein, 7 aa, Artificial Sequence
  Made in Lab - Light CDR2
GASNLES

SEQ ID NO: 30 - protein, 15 aa, Artificial Sequence
  Made in Lab - Light CDR3
QCTYGSSTTSNYGDA

SEQ ID NO: 31 - protein, 5 aa, Artificial Sequence
  Made in Lab - Heavy CDR1
SYAMS

SEQ ID NO: 32 - protein, 17 aa, Artificial Sequence
  Made in Lab - Heavy CDR2
IISASGANTWYATWAKG

SEQ ID NO: 33 - protein, 11 aa, Artificial Sequence
  Made in Lab - Heavy CDR3
ISSGGWDYFNI

SEQ ID NO: 34 - protein, 11 aa, Artificial Sequence
  Made in Lab - Light CDR1
QASQSISSYLS

SEQ ID NO: 35 - protein, 7 aa, Artificial Sequence
  Made in Lab - Light CDR2
YSSTLAS

SEQ ID NO: 36 - protein, 15 aa, Artificial Sequence
  Made in Lab - Light CDR3
QGYYYDRDSSSYGWA

SEQ ID NO: 37 - protein, 5 aa, Artificial Sequence
  Made in Lab - Heavy CDR1
SYSMI

SEQ ID NO: 38 - protein, 16 aa, Artificial Sequence
  Made in Lab - Heavy CDR2
IIYTGSSASYASWAKG

SEQ ID NO: 39 - protein, 9 aa, Artificial Sequence
  Made in Lab - Heavy CDR3
ENSHDTFDP

SEQ ID NO: 40 - protein, 12 aa, Artificial Sequence
  Made in Lab - Light CDR1
QSSQSVYTNYLS

SEQ ID NO: 41 - protein, 7 aa, Artificial Sequence
  Made in Lab - Light CDR2
AASTLAS

SEQ ID NO: 42 - protein, 12 aa, Artificial Sequence
  Made in Lab - Light CDR3
LGSYVSSGWYYA

SEQ ID NO: 43 - protein, 5 aa, Artificial Sequence
  Made in Lab - Heavy CDR1
SYSMI

SEQ ID NO: 44 - protein, 16 aa, Artificial Sequence
  Made in Lab - Heavy CDR2
IIYTGGSTDYASWAKG

SEQ ID NO: 45 - protein, 9 aa, Artificial Sequence
  Made in Lab - Heavy CDR3
ENSHDTFDP

SEQ ID NO: 46 - protein, 12 aa, Artificial Sequence
  Made in Lab - Light CDR1
QSSQSVYTNYLS

SEQ ID NO: 47 - protein, 7 aa, Artificial Sequence
  Made in Lab - Light CDR2
AASTLAS

SEQ ID NO: 48 - protein, 12 aa, Artificial Sequence
  Made in Lab - Light CDR3
LGSYVSSGWYYA

SEQ ID NO: 49 - protein, 5 aa, Artificial Sequence
  Made in Lab - Heavy CDR1
TYAMI

SEQ ID NO: 50 - protein, 16 aa, Artificial Sequence
  Made in Lab - Heavy CDR2
IISPSGTTYYATWAKG

SEQ ID NO: 51 - protein, 11 aa, Artificial Sequence
  Made in Lab - Heavy CDR3
AGSGGWDYFNI

SEQ ID NO: 52 - protein, 11 aa, Artificial Sequence
  Made in Lab - Light CDR1
AGSGGWDYFNI

SEQ ID NO: 53 - protein, 7 aa, Artificial Sequence
  Made in Lab - Light CDR2
YASNLAS

SEQ ID NO: 54 - protein, 12 aa, Artificial Sequence
  Made in Lab - Light CDR3
QSYYYSNNYNWA

SEQ ID NO: 55 - protein, 5 aa, Artificial Sequence
  Made in Lab - Heavy CDR1
SYWMC

SEQ ID NO: 56 - protein, 17 aa, Artificial Sequence
  Made in Lab - Heavy CDR2
CIYDGDGNTWYAYWVNG

SEQ ID NO: 57 - protein, 14 aa, Artificial Sequence
  Made in Lab - Heavy CDR3
GGAGYLGYGYPFNL

SEQ ID NO: 58 - protein, 11 aa, Artificial Sequence
  Made in Lab - Light CDR1
QASQSITSYLS

SEQ ID NO: 59 - protein, 7 aa, Artificial Sequence
  Made in Lab - Light CDR2
RASTLAS

SEQ ID NO: 60 - protein, 15 aa, Artificial Sequence
  Made in Lab - Light CDR3
QSYYFTSSNIYSYNA

SEQ ID NO: 61 - protein, 6 aa, Artificial Sequence
  Made in Lab - Heavy CDR1
SYYYMC

SEQ ID NO: 62 - protein, 18 aa, Artificial Sequence
  Made in Lab - Heavy CDR2
CIYAGDTGTTYYASWAKG

SEQ ID NO: 63 - protein, 18 aa, Artificial Sequence
  Made in Lab - Heavy CDR3
GGYSYGYGGATYPTYFDL

SEQ ID NO: 64 - protein, 11 aa, Artificial Sequence
  Made in Lab - Light CDR1
QASQSSSSYLA

SEQ ID NO: 65 - protein, 7 aa, Artificial Sequence
  Made in Lab - Light CDR2
RASTLAS

SEQ ID NO: 66 - protein, 13 aa, Artificial Sequence
  Made in Lab - Light CDR3
QSYYYSSSTSYDT

SEQ ID NO: 67 - protein, 5 aa, Artificial Sequence
  Made in Lab - Heavy CDR1
SYAMS

SEQ ID NO: 68 - protein, 16 aa, Artificial Sequence
  Made in Lab - Heavy CDR2
IIISSANTYYASWAKG

SEQ ID NO: 69 - protein, 14 aa, Artificial Sequence
  Made in Lab - Heavy CDR3
GLAGFGYGGGAFKD

SEQ ID NO: 70 - protein, 12 aa, Artificial Sequence
  Made in Lab - Light CDR1
QASQSISADYLA

SEQ ID NO: 71 - protein, 7 aa, Artificial Sequence
  Made in Lab - Light CDR2
KASTLAS

SEQ ID NO: 72 - protein, 12 aa, Artificial Sequence
  Made in Lab - Light CDR3
QYTGYDSVYIGA

SEQ ID NO: 73 - protein, 5 aa, Artificial Sequence
  Made in Lab - Heavy CDR1
TYTMG

SEQ ID NO: 74 - protein, 16 aa, Artificial Sequence
  Made in Lab - Heavy CDR2
IIYSGGNSYYASWAKG

SEQ ID NO: 75 - protein, 11 aa, Artificial Sequence
  Made in Lab - Heavy CDR3
SSYDGYEYFNI

SEQ ID NO: 76 - protein, 11 aa, Artificial Sequence
  Made in Lab - Light CDR1
QASQSISTYLA

SEQ ID NO: 77 - protein, 7 aa, Artificial Sequence
  Made in Lab - Light CDR2
AASTLAS

SEQ ID NO: 78 - protein, 12 aa, Artificial Sequence
  Made in Lab - Light CDR3
QQGYATTYVDNV

SEQ ID NO: 79 - protein, 6 aa, Artificial Sequence
  Made in Lab - Heavy CDR1
SSYWGF

SEQ ID NO: 80 - protein, 18 aa, Artificial Sequence
  Made in Lab - Heavy CDR2
SINGGTSGSTYYASWAKG

SEQ ID NO: 81 - protein, 11 aa, Artificial Sequence
  Made in Lab - Heavy CDR3
DAGSSGYFFNL

SEQ ID NO: 82 - protein, 11 aa, Artificial Sequence
  Made in Lab - Light CDR1
QASQSIGSNLA

SEQ ID NO: 83 - protein, 7 aa, Artificial Sequence
  Made in Lab - Light CDR2
RASSLAS

SEQ ID NO: 84 - protein, 13 aa, Artificial Sequence
  Made in Lab - Light CDR3
QNNYGITDNFGAA

SEQ ID NO: 85 - protein, 6 aa, Artificial Sequence
  Made in Lab - Heavy CDR1
NMDTMC

SEQ ID NO: 86 - protein, 17 aa, Artificial Sequence
  Made in Lab - Heavy CDR2
CIYIGDGVTFYASWVNG

SEQ ID NO: 87 - protein, 12 aa, Artificial Sequence
  Made in Lab - Heavy CDR3
DAGDGGWYSFGL

SEQ ID NO: 88 - protein, 11 aa, Artificial Sequence
  Made in Lab - Light CDR1
QASQSISGWLS

SEQ ID NO: 89 - protein, 7 aa, Artificial Sequence
  Made in Lab - Light CDR2
SASTLAS

SEQ ID NO: 90 - protein, 14 aa, Artificial Sequence
  Made in Lab - Light CDR3
QKNYGSSRGSSTNP

SEQ ID NO: 91 - protein, 5 aa, Artificial Sequence
  Made in Lab - Heavy CDR1
TYTIS

SEQ ID NO: 92 - protein, 16 aa, Artificial Sequence
  Made in Lab - Heavy CDR2
IIYTSGTTSYASWVKG

SEQ ID NO: 93 - protein, 12 aa, Artificial Sequence
  Made in Lab - Heavy CDR3
DRSDGWYGTFNP

SEQ ID NO: 94 - protein, 11 aa, Artificial Sequence
  Made in Lab - Light CDR1
QSSDNIGTYLA

SEQ ID NO: 95 - protein, 7 aa, Artificial Sequence
  Made in Lab - Light CDR2
QASTLAS

SEQ ID NO: 96 - protein, 12 aa, Artificial Sequence
  Made in Lab - Light CDR3
QQGYAVGHVDNT

SEQ ID NO: 97 - protein, 5 aa, Artificial Sequence
  Made in Lab - Heavy CDR1
SYAMG

SEQ ID NO: 98 - protein, 16 aa, Artificial Sequence
  Made in Lab - Heavy CDR2
IISPGGITYYATWAKG

SEQ ID NO: 99 - protein, 7 aa, Artificial Sequence
  Made in Lab - Heavy CDR3
GWDYFNI

SEQ ID NO: 100 - protein, 11 aa, Artificial Sequence
  Made in Lab - Light CDR1
QASQSISSYLA

SEQ ID NO: 101 - protein, 7 aa, Artificial Sequence
  Made in Lab - Light CDR2
DASKLAS

SEQ ID NO: 102 - protein, 13 aa, Artificial Sequence
  Made in Lab - Light CDR3
QSAYYGSSSTVNN

SEQ ID NO: 103 - protein, 5 aa, Artificial Sequence
  Made in Lab - Heavy CDR1
NYYMC

SEQ ID NO: 104 - protein, 18 aa, Artificial Sequence
  Made in Lab - Heavy CDR2
CIYSGSGGSTYHASWARG

SEQ ID NO: 105 - protein, 16 aa, Artificial Sequence
  Made in Lab - Heavy CDR3
AYGVSVSGAYGHYFNL

SEQ ID NO: 106 - protein, 11 aa, Artificial Sequence
  Made in Lab - Light CDR1
QASQSISTYLA

SEQ ID NO: 107 - protein, 7 aa, Artificial Sequence
  Made in Lab - Light CDR2
GASNLES

SEQ ID NO: 108 - protein, 13 aa, Artificial Sequence
  Made in Lab - Light CDR3
QSAYYGSSSTVNT

SEQ ID NO: 109 - protein, 5 aa, Artificial Sequence
  Made in Lab - Heavy CDR1
NYEMI

SEQ ID NO: 110 - protein, 16 aa, Artificial Sequence
  Made in Lab - Heavy CDR2
IIYVSRSTHYASWAKG

SEQ ID NO: 111 - protein, 9 aa, Artificial Sequence
  Made in Lab - Heavy CDR3
YLASGGFNI

SEQ ID NO: 112 - protein, 11 aa, Artificial Sequence
  Made in Lab - Light CDR1
QASHNIYSNLA

SEQ ID NO: 113 - protein, 7 aa, Artificial Sequence
  Made in Lab - Light CDR2
DASKLAS

SEQ ID NO: 114 - protein, 9 aa, Artificial Sequence
  Made in Lab - Light CDR3
QSYYGNSYP

SEQ ID NO: 115 - protein, 5 aa, Artificial Sequence
  Made in Lab - Heavy CDR1
SYGVS

SEQ ID NO: 116 - protein, 17 aa, Artificial Sequence
  Made in Lab - Heavy CDR2
YIHPDAGRIDYVNWVNG

SEQ ID NO: 117 - protein, 14 aa, Artificial Sequence
  Made in Lab - Heavy CDR3
GNYDDYGDYFYFDL

SEQ ID NO: 118 - protein, 11 aa, Artificial Sequence
  Made in Lab - Light CDR1
QASDFIYGNLA

SEQ ID NO: 119 - protein, 7 aa, Artificial Sequence
  Made in Lab - Light CDR2
DASDLAS

SEQ ID NO: 120 - protein, 14 aa, Artificial Sequence
  Made in Lab - Light CDR3
QSAYYSTSADMRNA

SEQ ID NO: 121 - protein, 5 aa, Artificial Sequence
  Made in Lab - Heavy CDR1
SYAMG

SEQ ID NO: 122 - protein, 16 aa, Artificial Sequence
  Made in Lab - Heavy CDR2
IIYIGDSAAYANWAKG

SEQ ID NO: 123 - protein, 11 aa, Artificial Sequence
  Made in Lab - Heavy CDR3
SSYDGYEYFNI

SEQ ID NO: 124 - protein, 11 aa, Artificial Sequence
  Made in Lab - Light CDR1
QASQSISSYLA

SEQ ID NO: 125 - protein, 7 aa, Artificial Sequence
  Made in Lab - Light CDR2
AASTLAS

SEQ ID NO: 126 - protein, 12 aa, Artificial Sequence
  Made in Lab - Light CDR3
QQGYATTYVDNP

SEQ ID NO: 127 - protein, 6 aa, Artificial Sequence
  Made in Lab - Heavy CDR1
RTYYLC

SEQ ID NO: 128 - protein, 18 aa, Artificial Sequence
  Made in Lab - Heavy CDR2
CIYVSDNGKTYYASWARG

SEQ ID NO: 129 - protein, 11 aa, Artificial Sequence
  Made in Lab - Heavy CDR3
DYSDDSVFLGL

SEQ ID NO: 130 - protein, 11 aa, Artificial Sequence
  Made in Lab - Light CDR1
QASQNILYFLA

SEQ ID NO: 131 - protein, 7 aa, Artificial Sequence
  Made in Lab - Light CDR2
DASNLAS

SEQ ID NO: 132 - protein, 12 aa, Artificial Sequence
  Made in Lab - Light CDR3
QSVWYSSGAANL

SEQ ID NO: 133 - protein, 6 aa, Artificial Sequence
  Made in Lab - Heavy CDR1
SGYWIC

SEQ ID NO: 134 - protein, 17 aa, Artificial Sequence
  Made in Lab - Heavy CDR2
CISIHTGKAHYASRAKG

SEQ ID NO: 135 - protein, 10 aa, Artificial Sequence
  Made in Lab - Heavy CDR3
QGSDYADYNL

SEQ ID NO: 136 - protein, 12 aa, Artificial Sequence
  Made in Lab - Light CDR1
QSSQSLSNAHVA

SEQ ID NO: 137 - protein, 7 aa, Artificial Sequence
  Made in Lab - Light CDR2
FASRLAS

SEQ ID NO: 138 - protein, 13 aa, Artificial Sequence
  Made in Lab - Light CDR3
QGEFDCSHGDCDA

SEQ ID NO: 139 - protein, 6 aa, Artificial Sequence
  Made in Lab - Heavy CDR1
SSYWIC

SEQ ID NO: 140 - protein, 18 aa, Artificial Sequence
  Made in Lab - Heavy CDR2
CISASSIPTTYYASWAKG

SEQ ID NO: 141 - protein, 10 aa, Artificial Sequence
  Made in Lab - Heavy CDR3
YHTTNWAVDF

SEQ ID NO: 142 - protein, 12 aa, Artificial Sequence
  Made in Lab - Light CDR1
QSSPSVASNYLS

SEQ ID NO: 143 - protein, 7 aa, Artificial Sequence
  Made in Lab - Light CDR2
AASTLAS

SEQ ID NO: 144 - protein, 12 aa, Artificial Sequence
  Made in Lab - Light CDR3
AGGYTGNIDTFV

SEQ ID NO: 145 - protein, 5 aa, Artificial Sequence
  Made in Lab - Heavy CDR1
SYVMN

SEQ ID NO: 146 - protein, 16 aa, Artificial Sequence
  Made in Lab - Heavy CDR2
IIYTGGSASYANWAKG

SEQ ID NO: 147 - protein, 11 aa, Artificial Sequence
  Made in Lab - Heavy CDR3
SPYDGTYYMDI

SEQ ID NO: 148 - protein, 11 aa, Artificial Sequence
  Made in Lab - Light CDR1
QASQSIGAYFA

SEQ ID NO: 149 - protein, 7 aa, Artificial Sequence
  Made in Lab - Light CDR2
SASTLAS

SEQ ID NO: 150 - protein, 10 aa, Artificial Sequence
  Made in Lab - Light CDR3
QSYYATSTNT

SEQ ID NO: 151 - protein, 5 aa, Artificial Sequence
  Made in Lab - Heavy CDR1
NYDVT

SEQ ID NO: 152 - protein, 16 aa, Artificial Sequence
  Made in Lab - Heavy CDR2
VITDDGHTYYAGWVNG

SEQ ID NO: 153 - protein, 7 aa, Artificial Sequence
  Made in Lab - Heavy CDR3
DWRYFHI

SEQ ID NO: 154 - protein, 11 aa, Artificial Sequence
  Made in Lab - Light CDR1
QASENIYSSLA

SEQ ID NO: 155 - protein, 7 aa, Artificial Sequence
  Made in Lab - Light CDR2
DASDLAS

SEQ ID NO: 156 - protein, 12 aa, Artificial Sequence
  Made in Lab - Light CDR3
QSYYGGTNVGYS

SEQ ID NO: 157 - protein, 6 aa, Artificial Sequence
  Made in Lab - Heavy CDR1
RGYYIC

SEQ ID NO: 158 - protein, 17 aa, Artificial Sequence
  Made in Lab - Heavy CDR2
CIGVSSENVYYASWAKG

SEQ ID NO: 159 - protein, 8 aa, Artificial Sequence
  Made in Lab - Heavy CDR3
GGSPGYSL

SEQ ID NO: 160 - protein, 11 aa, Artificial Sequence
  Made in Lab - Light CDR1
QASQNTGGWLA

SEQ ID NO: 161 - protein, 7 aa, Artificial Sequence
  Made in Lab - Light CDR2
EASKLAS

SEQ ID NO: 162 - protein, 11 aa, Artificial Sequence
  Made in Lab - Light CDR3
QYTYGGSGGYG

SEQ ID NO: 163 - protein, 6 aa, Artificial Sequence
  Made in Lab - Heavy CDR1
NSYWMR

SEQ ID NO: 164 - protein, 17 aa, Artificial Sequence
  Made in Lab - Heavy CDR2
YINTGSGGTYYASWAKG

SEQ ID NO: 165 - protein, 4 aa, Artificial Sequence
  Made in Lab - Heavy CDR3
NGNL

SEQ ID NO: 166 - protein, 13 aa, Artificial Sequence
  Made in Lab - Light CDR1
QSSQSVYDNNALA

SEQ ID NO: 167 - protein, 7 aa, Artificial Sequence
  Made in Lab - Light CDR2
TASTLAS

SEQ ID NO: 168 - protein, 10 aa, Artificial Sequence
  Made in Lab - Light CDR3
AGDFSGYIYD

SEQ ID NO: 169 - protein, 124 aa, Artificial Sequence
  Made in Lab - humanized heavy chain sequence
EVQLVESGGGLVQPGGSLRLSCAASGFSFSGSDYICWVRQAPGKGLEWVACINVGIDNTYYASWATGRFTISRDNSKNTLYLQMNSLRAEDTAVYYCARGYSASAWGLDYFNLWGQGTLVTVSS

SEQ ID NO: 170 - protein, 111 aa, Artificial Sequence
  Made in Lab - humanized light chain sequence
DIQMTQSPSSLSASVGDRVTITCQASESIGNYLAWYQQKPGKAPKLLIYGASTLESGVPSRFSGSGSGTDFTLTISSLQPEDFATYYCQCTYGTTSISTYTFGGGTKVEIK

SEQ ID NO: 171 - protein, 122 aa, Artificial Sequence
  Made in Lab - humanized heavy chain sequence
EVQLVESGGGLVQPGGSLRLSCAASGFSFSSYYFMCWVRQAPGKGLEWVACIDAGYSGGSHYASWAKGRFTISRDNSKNTLYLQMNSLRAEDTAVYYCARAYEYGYGGYDLWGQGTLVTVSS

SEQ ID NO: 172 - protein, 113 aa, Artificial Sequence
  Made in Lab - humanized light chain sequence
DIQMTQSPSSLSASVGDRVTITCQASQSIDSNLAWYQQKPGKAPKLLIYRASGLESGVPSRFSGSGSGTDFTLTISSLQPEDFATYYCQCTYGSNDSGNYANAFGGGTKVEIK

SEQ ID NO: 173 - protein, 125 aa, Artificial Sequence
  Made in Lab - humanized heavy chain sequence
EVQLVESGGGLVQPGGSLRLSCAASGFSFSSSDYMCWVRQAPGKGLEWVACICTGHGDTAYATWAKGRFTISRDNSKNTLYLQMNSLRAEDTAVYYCARGWTSSADYYIYYLNLWGQGTLVTVSS

SEQ ID NO: 174 - protein, 110 aa, Artificial Sequence
  Made in Lab - humanized light chain sequence
AIQMTQSPSSLSASVGDRVTITCQASESIYNYLSWYQQKPGKAPKLLIYGASNLESGVPSRFSGSGSGTDFTLTISSLQPEDFATYYCQQGYSGNNIDNIFGGGTKVEIK

SEQ ID NO: 175 - protein, 129 aa, Artificial Sequence
  Made in Lab - humanized heavy chain sequence
EVQLVESGGGLVQPGGSLRLSCAASGFSFSSSDYMCWVRQAPGKGLEWVGCINSGSDTTSYATWAKGRFTISRDNAKNTLYLQMNSLRAEDTAVYYCARGYGSAGANGYVIGMKYLNLWGQGTLVTVSS

SEQ ID NO: 176 - protein, 110 aa, Artificial Sequence
  Made in Lab - humanized light chain sequence
DIQMTQSPSSLSASVGDRVTITCQASQSIGSNLAWYQQKPGKAPKLLIYTASTLESGVPSRFSGSGSGTDFTLTISSLQPEDFATYYCQCTYGSSSSGYPFGGGTKVEIK

SEQ ID NO: 177 - protein, 124 aa, Artificial Sequence
  Made in Lab - humanized heavy chain sequence
EVQLVESGGGLVQPGGSLRLSCAASGFSFSGSEYMSWVRQAPGKGLEWVGCIYFGISGSTYYANWAKGRFTISRDNSKNTLYLQMNSLRAEDTAVYYCARGYGDKYNLYYFNLWGQGTLVTVSS

SEQ ID NO: 178 - protein, 113 aa, Artificial Sequence
  Made in Lab - humanized light chain sequence
DIQMTQSPSSLSASVGDRVTITCQASEDIESYLAWYQQKPGKAPKLLIYGASNLESGVPSRFSGSGSGTDFTLTISSLQPEDFATYYCQCTYGSSTTSNYGDAFGGGTKVEIK

SEQ ID NO: 179 - protein, 120 aa, Artificial Sequence
  Made in Lab - humanized heavy chain sequence
EVQLVESGGGLVQPGGSLRLSCAASGVDLSSYAMSWVRQAPGKGLEWVGIISASGANTWYATWAKGRFTISRDNSKNTLYLQMNSLRAEDTAVYYCAGISSGGWDYFNIWGQGTLVTVSS

SEQ ID NO: 180 - protein, 113 aa, Artificial Sequence
  Made in Lab - humanized light chain sequence
DIQMTQSPSSLSASVGDRVTITCQASQSISSYLSWYQQKPGKAPKLLIYYSSTLASGVPSRFSGSGSGTDFTLTISSLQPEDFATYYCQGYYYDRDSSSYGWAFGGGTKVEIK

SEQ ID NO: 181 - protein, 117 aa, Artificial Sequence
  Made in Lab - humanized heavy chain sequence
EVQLVESGGGLVQPGGSLRLSCAASGFSLSSYSMIWVRQAPGKGLEWVGIIYTGSSASYASWAKGRFTISRDNSKNTLYLQMNSLRAEDTAVYYCARENSHDTFDPWGQGTLVTVSS

SEQ ID NO: 182 - protein, 111 aa, Artificial Sequence
  Made in Lab - humanized light chain sequence
DIQMTQSPSSLSASVGDRVTITCQSSQSVYTNYLSWYQQKPGKAPKLLIYAASTLASGVPSRFSGSGSGTDFTLTISSLQPEDFATYYCLGSYVSSGWYYAFGGGTKVEIK

SEQ ID NO: 183 - protein, 117 aa, Artificial Sequence
  Made in Lab - humanized heavy chain sequence
EVQLVESGGGLVQPGGSLRLSCAASGIDLSSYSMIWVRQAPGKGLEWIGIIYTGGSTDYASWAKGRFTISRDNSKNTLYLQMNSLRAEDTAVYYCARENSHDTFDPWGQGTLVTVSS

SEQ ID NO: 184 - protein, 111 aa, Artificial Sequence; identical to SEQ ID NO: 182
  Made in Lab - humanized light chain sequence

SEQ ID NO: 185 - protein, 119 aa, Artificial Sequence
  Made in Lab - humanized heavy chain sequence
EVQLVESGGGLVQPGGSLRLSCAASGFSLSTYAMIWVRQAPGKGLEWVGIISPSGTTYYATWAKGRFTISRDNSKNTLYLQMNSLRAEDTAVYYCARAGSGGWDYFNIWGQGTLVTVSS

SEQ ID NO: 186 - protein, 110 aa, Artificial Sequence
  Made in Lab - humanized light chain sequence
DIQMTQSPSSLSASVGDRVTITCQASQSISSYLSWYQQKPGKAPKLLIYYASNLASGVPSRFSGSGSGTDFTLTISSLQPEDFATYYCQSYYYSNNYNWAFGGGTKVEIK

SEQ ID NO: 187 - protein, 123 aa, Artificial Sequence
  Made in Lab - humanized heavy chain sequence
QVQLVESGGGLVQPGGSLRLSCSASGFTLSSYWMCWVRQAPGKGLELIACIYDGDGNTWYAYWVNGRFTISRDNSKNTLYLQMNSLRAEDTAVYYCARGGAGYLGYGYPFNLWGQGTLVTVSS

SEQ ID NO: 188 - protein, 113 aa, Artificial Sequence
  Made in Lab - humanized light chain sequence
DIQMTQSPSSLSASVGDRVTITCQASQSITSYLSWYQQKPGKAPKLLIYRASTLASGVPSRFSGSGSGTDFTLTISSLQPEDFATYYCQSYYFTSSNIYSYNAFGGGTKVEIK

SEQ ID NO: 189 - protein, 129 aa, Artificial Sequence
  Made in Lab - humanized heavy chain sequence
EVQLVESGGGLVQPGGSLRLSCAASGFDLSSYYYMCWVRQAPGKGLGLIACIYAGDTGTTYYASWAKGRFTISRDNSKNTLYLQMNSLRAEDTAVYYCARGGYSYGYGGATYPTYFDLWGQGTLVTVSS

SEQ ID NO: 190 - protein, 111 aa, Artificial Sequence
  Made in Lab - humanized light chain sequence
DIQMTQSPSTLSASVGDRVTITCQASQSSSSYLAWYQQKPGKAPKLLIYRASTLASGVPSRFSGSGSGTEFTLTISSLQPDDFATYYCQSYYYSSSTSYDTFGGGTKVEIK

SEQ ID NO: 191 - protein, 122 aa, Artificial Sequence
  Made in Lab - humanized heavy chain sequence
EVQLVESGGGLVQPGGSLRLSCAASGIDLSSYAMSWVRQAPGKGLEWVGIIISSANTYYASWAKGRFTISRDNSKNTLYLQMNSLRAEDTAVYYCARGLAGFGYGGGAFKDWGQGTLVTVSS

SEQ ID NO: 192 - protein, 111 aa, Artificial Sequence
  Made in Lab - humanized light chain sequence
DIQMTQSPSSVSASVGDRVTITCQASQSISADYLAWYQQKPGKAPKLLIYKASTLASGVPSRFSGSGSGTDFTLTISSLQPEDFATYYCQYTGYDSVYIGAFGGGTKVEIK

SEQ ID NO: 193 - protein, 119 aa, Artificial Sequence
  Made in Lab - humanized heavy chain sequence
EVQLVESGGGLVQPGGSLRLSCAASGFSLSTYTMGWVRQAPGKGLEWVGIIYSGGNSYYASWAKGRFTISRDNSKNTLYLQMNSLRAEDTAVYYCARSSYDGYEYFNIWGQGTLVTVSS

SEQ ID NO: 194 - protein, 110 aa, Artificial Sequence
  Made in Lab - humanized light chain sequence
DIQMTQSPSSLSASVGDRVTITCQASQSISTYLAWYQQKPGKAPKQLIYAASTLASGVPSRFSGSGSGTDFTLTISSLQPEDFATYYCQQGYATTYVDNVFGGGTKVEIK

SEQ ID NO: 195 - protein, 122 aa, Artificial Sequence
  Made in Lab - humanized heavy chain sequence
EVQLVESGGGLVQPGGSLRLSCAASGFSFSSSYWGFWVRQAPGKGLEWVASINGGTSGSTYYASWAKGRFTISRDNSKNTLYLQMNSLRAEDTAVYYCARDAGSSGYFFNLWGQGTLVTVSS

SEQ ID NO: 196 - protein, 111 aa, Artificial Sequence
  Made in Lab - humanized light chain sequence
DIQMTQSPSSVSASVGDRVTITCQASQSIGSNLAWYQQKPGKAPKLLIYRASSLASGVPSRFSGSGSGTDFTLTISSLQPEDFATYYCQNNYGITDNFGAAFGGGTKVEIK

SEQ ID NO: 197 - protein, 122 aa, Artificial Sequence
  Made in Lab - humanized heavy chain sequence
EVQLVESGGGLVQPGGSLRLSCAASGFIFSNMDTMCWVRQAPGKGLEWVGCIYIGDGVTFYASWVNGRFTISRDNSKNTLYLQMNSLRAEDTAVYYCARDAGDGGWYSFGLWGQGTLVTVSS

SEQ ID NO: 198 - protein, 112 aa, Artificial Sequence
  Made in Lab - humanized light chain sequence
DIQMTQSPSTLSASVGDRVTITCQASQSISGWLSWYQQKPGKAPKRLIYSASTLASGVPSRFSGSGSGTEFTLTISSLQPDDFATYYCQKNYGSSRGSSTNPFGGGTKVEIK

SEQ ID NO: 199 - protein, 120 aa, Artificial Sequence
  Made in Lab - humanized heavy chain sequence
EVQLVESGGGLVQPGGSLRLSCAASGIDLSTYTISWVRQAPGKGLEWVGIIYTSGTTSYASWVKGRFTISRDNSKNTLYLQMNSLRAEDTAVYYCARDRSDGWYGTFNPWGPGTLVTVSS

SEQ ID NO: 200 - protein, 110 aa, Artificial Sequence
  Made in Lab - humanized light chain sequence
DIQMTQSPSSLSASVGDRVTITCQSSDNIGTYLAWYQQKPGKAPKLLIYQASTLASGVPSRFSGSGSGTDFTLTISSLQPEDFATYYCQQGYAVGHVDNTFGGGTKVEIK

SEQ ID NO: 201 - protein, 115 aa, Artificial Sequence
  Made in Lab - humanized heavy chain sequence
EVQLVESGGGLVQPGGSLRLSCAASGIDLSSYAMGWVRQAPGKGLEYVGIISPGGITYYATWAKGRFTISRDNSKNTLYLQMNSLRAEDTAVYYCARGWDYFNIWGQGTLVTVSS

SEQ ID NO: 202 - protein, 111 aa, Artificial Sequence
  Made in Lab - humanized light chain sequence
AIQLTQSPSSLSASVGDRVTITCQASQSISSYLAWYQQKPGKAPKLLIYDASKLASGVPSRFSGSGSGTDFTLTISSLQPEDFATYYCQSAYYGSSSTVNNFGGGTKVEIK

SEQ ID NO: 203 - protein, 126 aa, Artificial Sequence
  Made in Lab - humanized heavy chain sequence
EVQLVESGGGLVQPGGSLRLSCAASGSDISNYYMCWVRQAPGKGLEWVACIYSGSGGSTYHASWARGRFTISRDNSKNTLYLQMNSLRAEDTAVYYCARAYGVSVSGAYGHYFNLWGQGTLVTVSS

SEQ ID NO: 204 - protein, 111 aa, Artificial Sequence
  Made in Lab - humanized light chain sequence
AIQLTQSPSSLSASVGDRVTITCQASQSISTYLAWYQQKPGKAPKLLIYGASNLESGVPSRFSGSGSGTDFTLTISSLQPEDFATYYCQSAYYGSSSTVNTFGGGTKVEIK

SEQ ID NO: 205 - protein, 117 aa, Artificial Sequence
  Made in Lab - humanized heavy chain sequence
EVQLVESGGGLVQPGGSLRLSCAASGFSLSNYEMIWVRQAPGKGLEWVGIIYVSRSTHYASWAKGRFTISRDNSKNTLYLQMNSLRAEDTAVYYCGRYLASGGFNIWGQGTLVTVSS

SEQ ID NO: 206 - protein, 107 aa, Artificial Sequence
  Made in Lab - humanized light chain sequence
DIQMTQSPSTLSASVGDRVTITCQASHNIYSNLAWYQQKPGKAPKLLIYDASKLASGVPSRFSGSGSGTEFTLTISSLQPDDFATYYCQSYYGNSYPFGGGTKVEIK

SEQ ID NO: 207 - protein, 123 aa, Artificial Sequence
  Made in Lab - humanized heavy chain sequence
EVQLVESGGGLVQPGGSLRLSCAASGIDFSSYGVSWVRQAPGKGLEWVAYIHPDAGRIDYVNWVNGRFTISRDNAKNSLYLQMNSLRAEDTAVYYCTIGNYDDYGDYFYFDLWGQGTLVTVSS

SEQ ID NO: 208 - protein, 112 aa, Artificial Sequence
  Made in Lab - humanized light chain sequence
DIQMTQSPSTLSASVGDRVTITCQASDFIYGNLAWYQQKPGKAPKLLIYDASDLASGVPSRFSGSGSGTEFTLTISSLQPDDFATYYCQSAYYSTSADMRNAFGGGTKVEIK

SEQ ID NO: 209 - protein, 119 aa, Artificial Sequence
  Made in Lab - humanized heavy chain sequence
EVQLVESGGGLVQPGGSLRLSCAASGFSLSSYAMGWVRQAPGKGLEWVGIIYIGDSAAYANWAKGRFTISRDNSKNTLYLQMNSLRAEDTAVYYCARSSYDGYEYFNIWGQGTLVTVSS

SEQ ID NO: 210 - protein, 110 aa, Artificial Sequence
  Made in Lab - humanized light chain sequence
DIQMTQSPSSVSASVGDRVTITCQASQSISSYLAWYQQKPGKAPKQLIYAASTLASGVPSRFSGSGSGTDFTLTISSLQPEDFATYYCQQGYATTYVDNPFGGGTKVEIK

SEQ ID NO: 211 - protein, 122 aa, Artificial Sequence
  Made in Lab - humanized heavy chain sequence
EVQLVESGGGLVQPGGSLRLSCTASGFSVSRTYYLCWVRQAPGKGLEWIACIYVSDNGKTYYASWARGRFTISRDNSKNTLYLQMNSLRAEDTAVYFCARDYSDDSVFLGLWGPGTLVTVSS

SEQ ID NO: 212 - protein, 110 aa, Artificial Sequence
  Made in Lab - humanized light chain sequence
DIQMTQSPSSLSASVGDRVTINCQASQNILYFLAWYQQKPGQRPKLLIYDASNLASGVPSRFSGSGSGTDFTLTISSLQPEDVATYYCQSVWYSSGAANLFGGGTKVEIK

SEQ ID NO: 213 - protein, 120 aa, Artificial Sequence
  Made in Lab - humanized heavy chain sequence
EVQLVESGGGLVQPGGSLRLSCTASGFSFSSGYWICWVRQAPGKGLEWIGCISIHTGKAHYASRAKGRFTISRDNSKNTLYLQMNSLRAEDTAVYFCARQGSDYADYNLWGPGTLVTVSS

SEQ ID NO: 214 - protein, 112 aa, Artificial Sequence
  Made in Lab - humanized light chain sequence
AIQLTQSPSSLSASVGDRVTINCQSSQSLSNAHVAWYQQKPGKPPKLLIYFASRLASGVPSRFSGSGSGTDFTLTISSLQPEDFATYYCQGEFDCSHGDCDAFGGGTKVEIK

SEQ ID NO: 215 - protein, 121 aa, Artificial Sequence
  Made in Lab - humanized heavy chain sequence
EVQLVESGGGLVQPGGSLRLSCAASGLDFSSSYWICWVRQAPGKGLEWIACISASSIPTTYYASWAKGRFTISRDNSKNTLYLQMNSLRAEDTAVYYCWRYHTTNWAVDFWGPGTLVTVSS

SEQ ID NO: 216 - protein, 111 aa, Artificial Sequence
  Made in Lab - humanized light chain sequence
DIQMTQSPSSLSASVGDRVTITCQSSPSVASNYLSWYQQKPGKAPKLLIYAASTLASGVPSRFSGSGSGTDFTLTISSLQPEDFATYYCAGGYTGNIDTFVFGGGTKVEIK

SEQ ID NO: 217 - protein, 119 aa, Artificial Sequence
  Made in Lab - humanized heavy chain sequence
EVQLVESGGGLVQPGGSLRLSCTASGIDLSSYVMNWVRQAPGKGLEYIGIIYTGGSASYANWAKGRFTISRDNSKNTLYLQMNSLRAEDTAVYFCARSPYDGTYYMDIWGPGTLVTVSS

SEQ ID NO: 218 - protein, 108 aa, Artificial Sequence
  Made in Lab - humanized light chain sequence
DIQMTQSPSSLSASVGDRVTIKCQASQSIGAYFAWYQQKPGQPPKLLIHSASTLASGVPSRFSGSGSGTDFTLTISSLQPEDVATYYCQSYYATSTNTFGGGTKVEIK

SEQ ID NO: 219 - protein, 115 aa, Artificial Sequence
  Made in Lab - humanized heavy chain sequence
EVQLVESGGGLVQPGGSLRLSCTASGFSLNNYDVTWVRQAPGKGLEYIGVITDDGHTYYAGWVNGRFTISRDNSKNTLYLQMNSLRAEDTATYFCARDWRYFHIWGPGTLVTVSS

SEQ ID NO: 220 - protein, 110 aa, Artificial Sequence
  Made in Lab - humanized light chain sequence
DIQMTQSPSSLSASVGDRVTINCQASENIYSSLAWYQQKPGQPPKLLIYDASDLASGVPSRFSGSGSGTDFTLTISSLQPEDVATYYCQSYYGGTNVGYSFGGGTKVEIK

SEQ ID NO: 221 - protein, 118 aa, Artificial Sequence
  Made in Lab - humanized heavy chain sequence
EVQLLESGGGLVQPGGSLRLSCTASGFSFSRGYYICWVRQAPGKGLEWIACIGVSSENVYYASWAKGRFTISRDNSKNTLYLQMNSLRAEDTAVYYCARGGSPGYSLWGPGTLVTVSS

SEQ ID NO: 222 - protein, 109 aa, Artificial Sequence
  Made in Lab - humanized light chain sequence
DIQMTQSPSSLSASVGDRVTINCQASQNTGGWLAWYQQKPGQRPKLLIYEASKLASGVPSRFSGSGSGTDFTLTISSLQPEDVATYYCQYTYGGSGGYGFGGGTKVEIK

SEQ ID NO: 223 - protein, 114 aa, Artificial Sequence
  Made in Lab - humanized heavy chain sequence
EVQLVESGGGLVQPGGSLRLSCTASGFTLSNSYWMRWVRQAPGKGLEWIGYINTGSGGTYYASWAKGRFTISRDNSKNTLYLQMNSLRAEDTAVYFCARNGNLWGPGTLVTVSS

SEQ ID NO: 224 - protein, 110 aa, Artificial Sequence
  Made in Lab - humanized light chain sequence
AIQLTQSPSSLSASVGDRVTITCQSSQSVYDNNALAWYQQKPGKAPKLLIHTASTLASGVPSRFSGSGSGTDFTLTISSLQPEDFATYYCAGDFSGYIYDYGGGTKVEIK

SEQ ID NO: 225 - protein, 12 aa, Artificial Sequence
  Made in Lab - Light CDR1
  MOD_RES (11)..(11): Xaa is any amino acid
QSSQSVYTNYXS

SEQ ID NO: 226 - protein, 12 aa, Artificial Sequence
  Made in Lab - Light CDR1
QSSQSVYTNYQS

SEQ ID NO: 227 - protein, 111 aa, Artificial Sequence
  Made in Lab - humanized light chain sequence
DIQMTQSPSSLSASVGDRVTITCQSSQSVYTNYQSWYQQKPGKAPKLLIYAASTLASGVPSRFSGSGSGTDFTLTISSLQPEDFATYYCLGSYVSSGWYYAFGGGTKVEIK

SEQ ID NO: 228 - protein, 347 aa, Homo sapiens
GVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 229 - protein, 106 aa, Homo sapiens
EELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSG

SEQ ID NO: 230 - protein, 100 aa, Homo sapiens
PSAPVVSGPAARATPQHTVSFTCESHGFSPRDITLKWFKNGNELSDFQTNVDPVGESVSYSIHSTAKVVLTREDVHSQVICEVAHVTLQGDPLRGTANLS

SEQ ID NO: 231 - protein, 95 aa, Homo sapiens
PTLEVTQQPVRAENQVNVTCQVRKFYPQRLQLTWLENGNVSRTETASTVTENKDGTYNWMSWLLVNVSAHRDDVKLTCQVEHDGQPAVSKSHDLK

SEQ ID NO: 232 - protein, 12 aa, Homo sapiens
SDLTKRNNMDFS

SEQ ID NO: 233 - protein, 15 aa, Homo sapiens
KGSPDDVEFKSGAGT

SEQ ID NO: 234 - protein, 14 aa, Homo sapiens
HDLKVSAHPKEQGS

SEQ ID NO: 235 - protein, 21 aa, Homo sapiens
HFPRVTTVSDLTKRNNMDFSI

SEQ ID NO: 236 - protein, 21 aa, Homo sapiens
KGSPDDVEFKSGAGTELSVRA

SEQ ID NO: 237 - protein, 26 aa, Homo sapiens
DGQPAVSKSHDLKVSAHPKEQGSNTA

SEQ ID NO: 238 - protein, 119 aa, Oryctolagus cuniculus
QEQLEESGGDLVKPEGSLTLTCTASGFSFSSGYWICWVRQAPGKGLEWIGCISIANGKAYYASRAKGRFTISKTSSTTVTLQMTSLTGADTATYFCARQGSDYADYNLWGPGTLVTVSS

SEQ ID NO: 239 - protein, 116 aa, Oryctolagus cuniculus
QSLEESGGRLVTPGTPLTLTCTVSGIDLSSYTMGWVRQAPGKGLEYIGIIYTGGSTYHTTWAKGRFTISKTSTAVDLKITSPTTEDTATYFCARSSYDGYEYFNIWGPGTLVTVSS

SEQ ID NO: 240 - protein, 124 aa, Oryctolagus cuniculus
QSLEESGGDLVKPGASLTLTCTASGSDISNYYMCWVRQAPGKGLEWIACIYAGSGGSTYYASWARGRFTISKTSSTTVTLQMTSLTAADTATYFCARAYGVSVSGAYGHYFNLWGPGTLVTVSS

SEQ ID NO: 241 - protein, 116 aa, Oryctolagus cuniculus
QSLEESGGRLVTPGTPLTLTCTVSGFSLSTYAMGWVRQAPGKGLEYIGFIYTGDSTYYPSWAKGRFTISKTSTTVDLKITSPTTEDTATYFCARSSYSGYEYFNIWGPGTLVTVSS

SEQ ID NO: 242 - protein, 121 aa, Oryctolagus cuniculus
QERVEESGGDLVQPEGSLTLTCTASGFSFSSSYWGFWVRQAPGKGLEWIACINGGTSGSTYYASWAKGRFTISKTSSTTVTLQMTSLTAADTATYFCARDAGSSGYFFNLWGPGTLVTVSS

SEQ ID NO: 243 - protein, 120 aa, Oryctolagus cuniculus
QSLEESGGDLVKPGASLTLTCTASGFIFSNMDTMCWVRQAPRKGLEWIGCIYIGDGVTFYASWVNGRFTISKTSSTTVTLQMTSLTDADTATYFCARDAGDGGWYSFGLWGPGTLVTVSS

SEQ ID NO: 244 - protein, 117 aa, Oryctolagus cuniculus
QSVEESGGRLVTPGGSLTLTCTVSGIDLNTYTISWVRQAPGKGLEWIGIIYTSGSTSYASWVNGRFTISKTSTTVDLKMTSLTAADTATYFCARDRSNGWYGIFNPWGPGTLVTVSS

SEQ ID NO: 245 - protein, 117 aa, Oryctolagus cuniculus
QSVEESGGRLVTPGTPLTLTCTVSGIDLSTYTISWVRQAPGKGLEWIGIIYTSGTTSYASWVKGRFTISKTSTTVDLKMTSLTAADTATYFCARDRSDGWYGTFNPWGPGTLVTVSS

SEQ ID NO: 246 - protein, 117 aa, Oryctolagus cuniculus; identical to SEQ ID NO: 245

SEQ ID NO: 247 - protein, 123 aa, Oryctolagus cuniculus
QQQLEESGGGLVKPGGTLTLTCKASEFTLSSYWMCWVRQAPGKGLELIACIYDGDGNTWYAYWVNGRFTISRSASLNTVTLQMTSLTAADTATYFCARGGAGYLGYGYPFNLWGPGTLVTVSS

SEQ ID NO: 248 - protein, 124 aa, Oryctolagus cuniculus
QSLEESGGDLVKPGASLTLTCTVSGFSFSANIYMCWVRQAPGKGLEWVACIYAGSSGSTYYASWAKGRFTISKSSSTTVTLQMTSLTAADTATYFCARGWTGIYGDSPYYFNLWGPGTLVTVSS

SEQ ID NO: 249 - protein, 122 aa, Oryctolagus cuniculus
QSLEESGGDLVKPGASLTLTCTASGFSFSLYYYMCWVRQAPGKGLELIACIYTNSDSAYYASWAKGRFTISKTSSTTVTLQMASLTAADTATYFCARDSYDDDGYFLIFSLWGPGTLVTVSS

SEQ ID NO: 250 - protein, 127 aa, Oryctolagus cuniculus
QSLEESGGDLVKPGASLTLTCKASGFDLSSYYYMCWVRQAPGKGLGLIACIYAGDTGTTYYASWAKGRFTISKTSSTTVTLQMTSLTAADTATYFCARGGYSYGYGGATYPTYFDLWGPGTLVTVSS

SEQ ID NO: 251 - protein, 119 aa, Oryctolagus cuniculus
QSLEESGGDLVKPGASLTLTCTASGFSFSSSYWICWVRQAPGKGLELIACIKGGSSGSTYHASWAKGRFTISKTSSTTVTLQMTSLTAADTATYFCARANDSTTYFNLWGPGTLVTVSS

SEQ ID NO: 252 - protein, 119 aa, Oryctolagus cuniculus
QSVEESGGRLVTPGTPLTLTCTVSGIDLSSYAMSWVRQAPGKGLEWIGIIISSANTYYASWAKGRFTISKTSTTVSLQMNSPTTEDTATYFCARGLAGFGYGGGAFKDWGPGTLVTVSS

SEQ ID NO: 253 - protein, 116 aa, Oryctolagus cuniculus
QSLEESGGRLVTPGTPLTLTCTVSGFSLSTYTMGWVRQAPGKGLEYIGIIYSGGNSYYASWAKGRFTISKTSTTVDLKITSPTTEDTATYFCARSSYDGYEYFNIWGPGTLVTVSS

SEQ ID NO: 254 - protein, 123 aa, Oryctolagus cuniculus
QEQLVESGGGLVQPEGSLTLTCTVSGFSFSLYYYMCWVRQAPGKGLEWIGCIYTNNGVTWYASWAKGRFTISKTSSTTVTLQLNSLTAADTATYFCARDNYDDSGYYFLFNLWGPGTLVTVSS

SEQ ID NO: 255 - protein, 112 aa, Oryctolagus cuniculus
QSLEESGGRLVTPGTPLTLTCTVSGIDLSSYAMGWVRQAPGKGLEYIGIISPGGITYYATWAKGRFTISKTSTTVDLRVTDLQPSDTATYFCARGWDYFNIWGPGTLVTVSS

SEQ ID NO: 256 - protein, 124 aa, Oryctolagus cuniculus
QSLEESGGDLVKPGASLTLTCTASRSDISNYYMCWVRQAPGKGLEWIACIYSGSGGSTYHASWARGRFTISNASSTTVTLEMTSLTAADTATYFCARAYGVSVSGAYGHYFNLWGPGTLVTVSS

SEQ ID NO: 257 - protein, 123 aa, Oryctolagus cuniculus
QEQLVESGGGLVTLGGSLKLSCKASGIDFSSYGVSWVRQAPGKGLEWIAYIHPDAGRIDYVNWVNGRFTISLDNAQNTVFLQMTSLTAADTATYFCTIGNYDDYGDYFYFDLWGPGTLVTVSS

SEQ ID NO: 258 - protein, 125 aa, Oryctolagus cuniculus
QSLEESGGDLVKPGASLTLTCKASGFSFSGSDYLCWVRQAPGKGLEWIACTTIGYNGGTYYASWVNGRFTIAITSTVDLKMSTLTAADTATYFCARGFADYNNYGDGGINYFNLWGPGTLVTVSS

SEQ ID NO: 259 - protein, 116 aa, Oryctolagus cuniculus
QSLEESGGRLVTPGTPLTLTCTVSGFSLSSYAMGWVRQAPGKGLEYIGIIYIGDSAAYANWAKGRFTISKTSTTVDLEITSPTTEDTATYFCARSSYDGYEYFNIWGPGTLVTVSS

SEQ ID NO: 260 - protein, 114 aa, Oryctolagus cuniculus
QSVEESGGRLVTPGTPLTLTCTASGFSLSNYEMIWVRQAPGKGLEWIGIIYVSRSTHYASWAKGRITISKTSTTVDLKITSPTTEDTATYFCGRYLASGGFNIWGPGTLVTVSS

SEQ ID NO: 261 - protein, 116 aa, Oryctolagus cuniculus
QSVEESGGRLVTPGTPLTLTCTASGFSLSSYAMSWVRQAPGKGLEWIGTISPGGVIYYASWAKGRFTISKTSTTVDLKMTSPTTEDTATYFCARGYGSSSGAYNIWGPGTLVTVSS

SEQ ID NO: 262 - protein, 120 aa, Oryctolagus cuniculus
ESLEESGGDLVKPGASLTLTCTASGFSFSINYYVCWVRQAPGKGLEWIACIYAGSGDHTYYATWVNGRFTISKTSSTTVTLQMTSLTAADTATYFCARDLTYDTGTFTFWGPGTLVTVSS

SEQ ID NO: 263 - protein, 117 aa, Oryctolagus cuniculus
QSVEESGGGLVTPGGTLTLTCTVSGFSLSTYAMGWVRQAPGKGLEWIGIIDSSGRTYYASWAKGRFTISKTSSTTVDLKIASPTTEDTATYFCGRYNGDNGGYFNIWGPGTLVTVSS

SEQ ID NO: 264 - protein, 116 aa, Oryctolagus cuniculus
QSVEESGGRLVTPGTPLTLTCTVSGFSISSYAMIWVRQAPGKGLEWIGVISGLARTYYASWAKGRFTISKTSTTVDLEITSPTTEDTATYFCARGGSGGWDYFNIWGPGTLVTVSS

SEQ ID NO: 265 - protein, 116 aa, Oryctolagus cuniculus
QSVEESGGRLVTPGTPLTLTCTVSGFSLSSYAMIWVRQAPGEGLEWIGTISANSGSTWYASWAKGRFTISKTSTTVDLKITSPTTEDTATYLCARAGTGWDYFNIWGPGTLVTVSS

SEQ ID NO: 266 - protein, 117 aa, Oryctolagus cuniculus
QSVEESGGRLVTPGTPLTLTCTVSGFSLSNYAMSWVRQAPGKGLEYIGIIVSASGSTYYASWAKGRFTISKTSTTVDLKITSPTTEDTATYFCARADAGYSEYFNIWGPGTLVTVSS

SEQ ID NO: 267 - protein, 117 aa, Oryctolagus cuniculus
QSVEESGGRLVTPGTPLTLTCTVSGFSLSNYAMSWVRQAPGKGLEYIGIIVSASGSTYYASWAKGRFTISKTSTAVDLKITSPTTEDTATYFCARADAGYSEYFNIWGPGTLVTVSS

SEQ ID NO: 268 - protein, 117 aa, Oryctolagus cuniculus; identical to SEQ ID NO: 266

SEQ ID NO: 269 - protein, 116 aa, Oryctolagus cuniculus
QSVEESGGRLVTPGTPLTLTCTVSGFSLSNYAMSWVRQAPGKGLEYIGIITASGGTYYASWAKGRFTISKTSTTVDLKITSPTTEDTATYFCARADGAYSEYFNIWGPGTLVTVSS

SEQ ID NO: 270 - protein, 116 aa, Oryctolagus cuniculus
QSVEESGGRLVTPGTPLTLTCTASGFSLSTYAMIWVRQAPGKGLEYIGIISPSGTTYYATWAKGRFTISKSSTTVGLKITSPTTEDTATYFCARAGSGGWDYFNIWGPGTLVTVSS

SEQ ID NO: 271 - protein, 117 aa, Oryctolagus cuniculus
QSVEESGGRLVKPDETLTLTCTVSGVDLSSYAMSWVRQAPGKGLEWIGIISASGANTWYATWAKGRFTLSKTSTTMDLKITSPTTEDTATYFCAGISSGGWDYFNIWGPGTLVTVSS

SEQ ID NO: 272 - protein, 117 aa, Oryctolagus cuniculus
QSVEESGGRLVTPGTPLTLTCTVSGVDLSIYAMSWVRQAPGKGLEWIGIISASGANTWYASWAKGRFTISKASTTMDLKITSPTTEDTATYFCAGISSGGWDYFNIWGPGTLVTVSS

SEQ ID NO: 273 - protein, 116 aa, Oryctolagus cuniculus
QSVEESGGRLVMPGTPLTVTCTVSGFSLSSYAMIWVRQAPGKGLEWIGIISSGGNTWYASWVKGRFSVSKTSTTVDLKITSPTSGDTATYFCARASDIGVDVYNIWGPGTLVTVSS

SEQ ID NO: 274 - protein, 116 aa, Oryctolagus cuniculus
QSVEESGGRLVTPGTPLTLTCTVSGFSISSYAMIWVRQAPGEGLEYIGFISAGGTTTYASWAKGRFTISKTSTTVDLKITSPTTEDTATYFCARALSVGIDAFDPWGPGTLVTVSS

SEQ ID NO: 275 - protein, 116 aa, Oryctolagus cuniculus
QSVEESGGRLVTPGTPLTLTCTVSGFSISSYAMIWVRQAPGEGLEYIGFISAGGTTSYASWAKGRFTISKTSTTVDLKITSPTTEDTATYFCARALSVGIDAFDPWGPGTLVTVSS

SEQ ID NO: 276 - protein, 116 aa, Oryctolagus cuniculus
QSVEESGGRLVTPGTPLTLTCTASGFSLSNYAMIWVRQAPGQGLEWIGMISSGGNTYYASWAKGRFTISKTSTTVDLKMTSLTTEDTATYFCAKAFNLGIDAFDPWGPGTLVTVSS

SEQ ID NO: 277 - protein, 120 aa, Oryctolagus cuniculus
QSVEESGGRLVTPGTPLTLTCTVSGFSLSSYHINWVRQAPGKGLEWIGFIYGSGSVGYASWAKGRFTISEASTTVDLKITSPTTEDTATYFCAGGYGGQSGSGYDRLDLWGQGTLVTVSS

SEQ ID NO: 278 - protein, 120 aa, Oryctolagus cuniculus
QSVEESGGRLVTPGTPLTLTCTVSGFSLSSYHINWVRQAPGKGLEWIGFIYGSGSIGYANWAKGRFTISETSTTVDLKITSPTTEDTATYFCAGGYGGQSGSGYDRLDLWGQGTLVTVSS

SEQ ID NO: 279 - protein, 121 aa, Oryctolagus cuniculus
QSVEESGGRLVTPGTPLTLTCTASGFSLSNYHIEWVRQAPGKGLEWIGFIYGSGSVGYANWAKGRFTISKTSSTTVDLKMTSLTTEDTATYFCAGGYGGQSGSGYDRLDLWGQGTLVTVSS

SEQ ID NO: 280 - protein, 117 aa, Oryctolagus cuniculus
QSVEESGGRLVTPGTPLTLTYTASGFSLTSHAMTWVRQAPGKGLVWIGIIYGGGSTAYASWATGRFTISRTSTTVDLRITSPTTEDTATYFCARLFDSGWGDRLDLWGQGTLVTVSS

SEQ ID NO: 281 - protein, 117 aa, Oryctolagus cuniculus
QSVEESGGRLVTPGTPLTLTCTASGFSLTSHAMTWVRQAPGKGLEWIGIIYGGGSTAYASWAKGRFTISRTSTTVDLGITSPTTEDTATYFCARLFDSGWGDRLDLWGQGTLVTVSS

SEQ ID NO: 282 - protein, 117 aa, Oryctolagus cuniculus
QSVEESGGRLVTPGTPLTLTCTASGFSLSSHAMTWVRQAPGKGLEWIGLIYGGGSTAYARWAKGRFTISKTSTTVDLRITSPTTEDTATYFCARLFDSGWGDRLDLWGQGTLVTVSS

SEQ ID NO: 283 - protein, 116 aa, Oryctolagus cuniculus
QSVEESGGRLVTPGTPLTLTCTASGFSLSSYGMAWVRQAPGKGLEYIGIIYIGGSTAYASWAKGRFTISKTSTTVDLKITSPTTEDTATYFCARYADNSYDYFNIWGPGTLVTVSS

SEQ ID NO: 284 - protein, 116 aa, Oryctolagus cuniculus
QSVEESGGRLVTPGTPLTLTCTASGFSLTSYGMSWVRQAPGKGLEYIGIIYIGGSASYASWAKGRFTISKTSTTVDLKITSPTTEDTATYFCARYADNSYDYFNIWGPGTLVTVSS

SEQ ID NO: 285 - protein, 116 aa, Oryctolagus cuniculus
QSLEESGGRLVTPGTPLTLTCTISGFSLSNYGMSWVRQAPGKGLEYIGIIYIGGSTWYASWAKGRFTISKSSTTVDLKITSPTTEDTATYFCARYGDNSYDHFNIWGPGTLVTVSS

SEQ ID NO: 286 - protein, 114 aa, Oryctolagus cuniculus
QSVEESGGRLVTPGTPLTLTCTVSGFSLSSYSMIWVRQAPGKGLEWIGIIYTGSSASYASWAKGRFTISKTSTTVDLKITSPTTEDTATYFCARENSHDTFDPWGPGTLVTVSS

SEQ ID NO: 287 - protein, 116 aa, Oryctolagus cuniculus
QSVEESGGRLVTPGTPLTLTCTVSGIDLSSYAMGWVRQAPGKGLEYIGIIYTGGSASYASWVKGRFTISKTSTTVDLQMTSLPTEDTATYFCARFSSDGYSYFNIWGPGTLVTVSS

SEQ ID NO: 288 - protein, 116 aa, Oryctolagus cuniculus
QSVEESGGRLVTPGTPLTLTCTVSGIDLSSYAMGWVRQAPGKGLEYIGIIYTGGSASYASWVKGRFTISKTSTTVDLKMTSLPTEDTATYFCARFSSDGYSYFNIWGPGTLVTVSS

SEQ ID NO: 289 - protein, 116 aa, Oryctolagus cuniculus
QSLEESGGRLVTPGTPLTLTCTVSGIDLSSYAMGWVRQAPGKGLEYIGIIYTGGSASYASWVKGRFTISKTSTTVDLKMTSLPTEDTATYFCARFSSDGYSYFNIWGPGTLVTVSS

SEQ ID NO: 290 - protein, 116 aa, Oryctolagus cuniculus
QSLEESGGRLVTPGTPLTLTCTVSGIDLSSYAMGWVRQAPGKGLEYIGIIYTGGSASYGSWVKGRFTISKTSTTVDLKMTSLPTEDTATYFCARFSSDGYSYFNIWGPGTLVTVSS

SEQ ID NO: 291 - protein, 116 aa, Oryctolagus cuniculus
QSLEESGGRLVTPGTPLTLTCTVSGIDLSSYAMGWVRQAPGKGLEYIGIIYTGGSASYASWVKGRFTISKTSTTVDLKMTSLPTEDTATYFCAGFSSDGYSYFNIWGPGTLVTVSS

SEQ ID NO: 292 - protein, 116 aa, Oryctolagus cuniculus
QSVEESGGRLVTPGTPLTLTCTVSGIDLSSYAMGWVRQAPGKGLEYIGIIYTGGSSSYASWVKGRFTISKTSTTVDLKMTSLPTEDTATYFCARFSSDGYSYFNIWGPGTLVTVSS

SEQ ID NO: 293 - protein, 116 aa, Oryctolagus cuniculus
QSVEESGGRLVTPGTPLTLTCTVSGIDLSSYAMGWVCQAPGKGLEYIGIIYTGGSASYASWVKGRFTISKTSTTVDLKMTSLPTEDTATYFCARFSSEGYSYFNIWGPGTLVTVSS

SEQ ID NO: 294 - protein, 116 aa, Oryctolagus cuniculus
QSVEESGGRLVTPGTPLTLTCTVSGFSLSSYAMGWVRQAPGKGLEWIGIMYAGGSTYYASWAKGRFTISKTSTTLDLKVNSLTTEDTATYFCARHISSGWDYFNIWGPGTLVTVSS

SEQ ID NO: 295 - protein, 116 aa, Oryctolagus cuniculus
RSVEESGGRLVTPDETLTITCTVSGIDLHSNSMTWVRQAPGKGLEWVGIIYVNDNTDYASWAKGRFTISKTSTTVDLKMTSLTTADTATYFCVRDLYPSTDYYNIWGPGTLVTVSS

SEQ ID NO: 296 - protein, 122 aa, Oryctolagus cuniculus
QSLEESGGDLVQPGASLTLTCTASGFSFSGSDYICWVRQAPGSGLEWIACINVGIDNTYYASWATGRFPISRTSSTTVTLQMTSLTAADTATYFCARGYSASAWGLDYFNLWGPGTLVTVSS

SEQ ID NO: 297 - protein, 127 aa, Oryctolagus cuniculus
QSLEESGGDLVKPGASLTLTCTASGFSFSSSDYMCWVRQAPGKGLEWIGCINSGSDTTSYATWAKGRFTISRTSSFTVTLQMTSLTAADTATYFCARGYGSAGANGYVIGMKYLNLWGPGTLVTVSS

SEQ ID NO: 298 - protein, 123 aa, Oryctolagus cuniculus
QSVEESGGDLVKPEGSLTLTCTASGFSFSSSDYMCWVRQAPGKGLEWIACICTGHGDTAYATWAKGRFTISRTSSTTVTLQMTSLTAADTATYFCARGWTSSADYYIYYLNLWGPGTLVTVSS

SEQ ID NO: 299 - protein, 122 aa, Oryctolagus cuniculus
QSLEESGGGLVQPEGSLTLTCKVSGFSFSGSEYMSWVRQAPGKGLEWIGCIYFGISGSTYYANWAKGRFTISKTSSTTVTLQMTSLTAADMATYFCARGYGDKYNLYYFNLWGPGTLVTVSS

SEQ ID NO: 300 - protein, 121 aa, Oryctolagus cuniculus
QEQLVESGGGLVQPEGSLTLTCTASGFSFSSYYFMCWVRQAPGKGLEWIACIDAGYSGGSHYASWAKGRLIISKTSSTTVTLQMTGLTAADTATYFCARAYEYGYGGYDLWGPGTLVTVSS

SEQ ID NO: 301 - protein, 118 aa, Oryctolagus cuniculus
EVQLVESGGGLVQPGGSLRLSCAASGFSFSGSYYMCWVRQAPGKGLEWIVCIYIGSGRTWYASWAKGRFTISRDNAKNSLYLQMNSLRDEDTAVYFCARGGWVNWGLWGPGTLVTVSS

SEQ ID NO: 302 - protein, 115 aa, Oryctolagus cuniculus
QSLEESGGRLVTPGTFLTLTCTASGFTISNKHMSWVRQAPGKGLEWIGIIDDGGKTWYANWATGRFTISKTSPTTVALMIISPTTEDTATYFCARGGGNDGFDPWGPGTLVTVSS

SEQ ID NO: 303 - protein, 115 aa, Oryctolagus cuniculus; identical to SEQ ID NO: 302

SEQ ID NO: 304 - protein, 120 aa, Oryctolagus cuniculus
QEQLEESGGDLVKPEGSLTLTCKASGLDFSSSYWICWVRQAPGKGLEWIACISASSIPTTYYASWAKGRFTISRTSSTTVTLQMTSLTAADTATYFCWRYHTTNWAVDFWGPGTLVTVSS

SEQ ID NO: 305 - protein, 120 aa, Oryctolagus cuniculus; identical to SEQ ID NO: 304

SEQ ID NO: 306 - protein, 116 aa, Oryctolagus cuniculus
QSVEESGGRLVTPGTPLTLTCTVSGFSLSSYAMGWVRQAPGKGLEWIGTITTGGGTYYATWAKGQFTISKTSTTVYLKMTSPTTEDTATYFCGRRYRDYSDAFDIWGPGTLVTVSS

SEQ ID NO: 307 - protein, 116 aa, Oryctolagus cuniculus; identical to SEQ ID NO: 306

SEQ ID NO: 308 - protein, 117 aa, Oryctolagus cuniculus
QEQLEESGGGLVYPADSLTLTCKADGFSFSRGYYICWVRQAPGKGLEWIACIGVSSENIYYPSWAKGRFTISKTSSTTVTLRMASLTAADTATYFCARGGSPGYSLWGPGTLVTVSS

SEQ ID NO: 309 - protein, 120 aa, Oryctolagus cuniculus
QSLEESGGDLVKPEGSLTLTCTASGFSISSTYYICWVRQAPGRGLEWIACIYVANNGKTYYASWARGRFTISKTSSTTVTLQMTSLTAADTATYFCARDYSDDSTYLGLWGPGTLVTVSS

SEQ ID NO: 310 - protein, 112 aa, Oryctolagus cuniculus
QSLEESGGDLVKPGASLTLTCTVSGFTLTNSYWMRWVRQAPGKGLEWIGYMHTGSGGTYYASWAKGRFTISRTSSTTVTLQMTGLTAADTATYFCARNGSLWGPGTLVTVSS

SEQ ID NO: 311 - protein, 112 aa, Oryctolagus cuniculus
QSLEESGGDLVKPGASLTLTCTASGFTLSNSYWMRWVRQAPGKGLEWIGYINTGSGGTYYASWAKGRFTISKTSSTTVTLQMTGLTAADTATYFCARNGNLWGPGTLVTVSS

SEQ ID NO: 312 - protein, 117 aa, Oryctolagus cuniculus
QEQLEESGGGLVHPADSLTLTCTASGFSFSRGYYICWVRQAPGKGLEWIACIGVSSENIYYASWAKGRFTISKTSSTTVTLKTARLTAADTATYFCARGGSPGYSLWGPGTLVTVYS

SEQ ID NO: 313 - protein, 117 aa, Oryctolagus cuniculus
QEQLEESGGGLVYPADSLTLTCKASGFSFSRGYYICWVRQAPGKGLEWIACIGVSSENIYYPSWAKGRFTISKTSSTTVTLRMASLTAADTATYFCARGGSPGYSLWGPGTLVTVSS

SEQ ID NO: 314 - protein, 112 aa, Oryctolagus cuniculus; identical to SEQ ID NO: 311

SEQ ID NO: 315 - protein, 117 aa, Oryctolagus cuniculus
QEQLEESGGGLVYPADSLTLTCKASGFSFSRGYYICWVRQAPGKGLEWIACIGVSSENIYYPSWAKGRFTISKTSSTTVTLRMARLTAADTATYFCARGGSPGYSLWGPGTLVTVSS

SEQ ID NO: 316 - protein, 119 aa, Oryctolagus cuniculus
QGQLVESGGGLVQPEGSLTLTCTASGFSFSSGYWICWVRQAPGKGLEWIGCISIHTGKAHYASRAKGRFTISKTSSTTVTLQMTSLTGADTANYFCARQGSDYADYNLWGPGTLVTVSS

SEQ ID NO: 317 - protein, 120 aa, Oryctolagus cuniculus
QSLEESGGDLVKPEGSLTLTCTASGFSVSRTYYLCWVRQAPGRGLEWIACIYVSDNGKTYYASWARGRFTISKTSSTTVTLQMTGLTAADTATYFCARDYSDDSVFLGLWGPGTLVTVSS

SEQ ID NO: 318 - protein, 119 aa, Oryctolagus cuniculus
QSLEESGGGLVQPEGSLTLTCKASGLSFSSMYYMCWVRQAPGKGLEWIGCSYIGSDGSAYYASWAKGRFSISRTSSTTVTLQMTSLTAADTATYFCARGNSAGVCVDLWGPGTLVTVSS

SEQ ID NO: 319 - protein, 117 aa, Oryctolagus cuniculus
QEQLEESGGGLVQPAGSLTLTCTASGFSFSRGYYICWVRQAPGKGLEWIACIGVSSENVYYASWAKGRFTISKTSSTTVTLQMARLTAADTATYFCARGGSPGYSLWGPGTLVTVSS

SEQ ID NO: 320 - protein, 120 aa, Oryctolagus cuniculus
QSLEESGGDLVKPEGSLTLTCKASGFDLSRTYYLCWVRQAPGRGLEWVACIYVSDVGKTYYASWARGRFTISKTSSTTVSLQMTGLTAADTATYFCARDYSDDSVFLGLWGPGTLVTVSS

SEQ ID NO: 321 - protein, 117 aa, Oryctolagus cuniculus; identical to SEQ ID NO: 313

SEQ ID NO: 322 - protein, 117 aa, Oryctolagus cuniculus
QEQLEESGGGLVHPADSLTLTCTASGFSFSRGYYICWVRQAPGKGLEWIACIGVSSENIYYASWAKGRFTISKTSSTTVTLKMASLTAADTATYFCARGGSPGYSLWGPGTLVTVSS

SEQ ID NO: 323 - protein, 117 aa, Oryctolagus cuniculus
QEQLEESGGGLVYPAGSLTLTCKASGFSFSRGYYICWVRQAPGKGLEWIACIGVSSENIYYPSWAKGRFTISKTSSTTVTLRMASLTAADTATYFCARGGSPGYSLWGPGTLVTVSS

SEQ ID NO: 324 - protein, 116 aa, Oryctolagus cuniculus
QSVEESGGRLVTPGTPLTLTCTVSGIDLSSYVMNWVRQAPGKGLEYIGIIYTGGSASYANWAKGRFTISKTSTTVDLTITSPTTADTATYFCARSPYDGTYYMDIWGPGTLVTVSS

SEQ ID NO: 325 - protein, 112 aa, Oryctolagus cuniculus
QSLEESGGRLVTPGTPLTLTCTVSGFSLNNYDVTWVRQTPGKGLEYIGVITDDGHTYYAGWVNGRFTISKTSTTVDLKITSPTSEDTATYFCARDWRYFHIWGPGTLVTVSS

SEQ ID NO: 326 - protein, 110 aa, Oryctolagus cuniculus
QIVMTQTPASVSEPVGGTVTIKCQASQSISYYLAWYQQKPGQPPKLLIYDASKLASGVPSRFSGSGSGTEFTLTISDLECADAATYYCQQGYGTTNVENPFGGGTEVVVK

SEQ ID NO: 327 - protein, 111 aa, Oryctolagus cuniculus
DVVMTQTPASVSEPVGGTVTIKCQASQSINGWLSWYQQRPGQRPKLLIYGASYLASGVPSRFTGSGSGTEYTLTISDLECDDAATYYCQSAYYGSSSTVNTFGGGTEVVVK

SEQ ID NO: 328 - protein, 110 aa, Oryctolagus cuniculus
QIVMTQTPASVSEPVGGTVTIKCQASQSISSYLAWYQQKPGQPPNQLIYAASTLASGVPSRFKGSGSGTQFTLTISDLECADAATYYCQQGYGTTNVDNPFGGGTEVVVK

SEQ ID NO: 329 - protein, 112 aa, Oryctolagus cuniculus
ADIVMTQTPSSVSAAVGGTVTIKCQASQSIGSNLAWYQQKPGQRPKLLIYRASSLASGVPSRFKGSGSGTEYTLTISDLECDDAATYYCQNNYGITDNFGAAFGGGTEVVVK

SEQ ID NO: 330 - protein, 113 aa, Oryctolagus cuniculus
ADIVMTQTPASVEAAVGGTVTIKCQASQSISGWLSWYQQKPGQPPKRLIYSASTLASGVPSRFKGSGSGTEYTLTISDLECADAATYYCQKNYGSSRGSSTNPFGGGTEVVVK

SEQ ID NO: 331 - protein, 110 aa, Oryctolagus cuniculus
ALVMTQTPASVSAALGDTVTIKCQASQSISTYLSWYQQKPGQPPKLLIYQASTVASGVPSRFSSSGSGTEFTLTISGVECADAATYYCQQGYAVGHVDNTFGGGTEVVVK

SEQ ID NO: 332 - protein, 110 aa, Oryctolagus cuniculus
ALVMTQTPASVEVAVGDTVTINCQSSDNIGTYLAWYQQKPGQPPKLLIYQASTLASGVPSRFSGSGSGTEFTLTISGVECGDAATYYCQQGYAVGHVDNTFGGGTEVVVK

SEQ ID NO: 333 - protein, 110 aa, Oryctolagus cuniculus; identical to SEQ ID NO: 332

SEQ ID NO: 334 - protein, 114 aa, Oryctolagus cuniculus
ADIVMTQTPASVEAAVGGTVTIKCQASQSITSYLSWYQQKPGQPPKLLIYRASTLASGVSSRFKGSGSGTEFTLTISDLECADAATYACQSYYFTSSNIYSYNAFGGGTEVVVK

SEQ ID NO: 335 - protein, 111 aa, Oryctolagus cuniculus
DAVMTQTPASVEAAVGGTVTIKCQASQSISSYLAWYQQKPGQPPKLLIYRASTLESGVPSRFKGSGSGTEFALTISDLECADAATYYCQCTYGTTAGSSFTFGGGTEVVVK

SEQ ID NO: 336 - protein, 109 aa, Oryctolagus cuniculus
QVLTQTPSSVSAAVGGTVTINCQASEDIERYLAWYQQKPGQRPKLLIYRASTLASGVSSRFKGSGSGTQFTLTISDLECDDAATYYCQQSYSISNLDNGFGGGTEVVVK

SEQ ID NO: 337 - protein, 112 aa, Oryctolagus cuniculus
VDIVMTQTPASVEAAVGGTVTIKCQASQSSSSYLAWYQQKPGQPPKLLIYRASTLASGVPSRFKGSGSGTQFTLTISDLECADAATYYCQSYYYSSSTSYDTFGGGTEVVVK

SEQ ID NO: 338 - protein, 111 aa, Oryctolagus cuniculus
DVVMTQTPASVSAAVGGTVTIKCQASQSMSSSYLAWYQQKPGQPPKLLIYKASTLTSGVSSRFKGGGSGTEFTLTISDLECADAATYYCQGYVAVSGSSYPFGGGTEVVVK

SEQ ID NO: 339 - protein, 111 aa, Oryctolagus cuniculus
DVVMTQTPASVSEPVGGAVTIKCQASQSISADYLAWYQQKPGQPPNLLIYKASTLASGVSSRFKGSGSGTEFTLTISDLECADAATYFCQYTGYDSVYIGAFGGGTEVVVK

SEQ ID NO: 340 - protein, 110 aa, Oryctolagus cuniculus
QIVMTQTPASVSEPVGGTVTIKCQASQSISTYLAWYQQKPGQPPKQLIYAASTLASGVPSRFKGSGSGTEFTLTISDLECADAATYYCQQGYATTYVDNVFGGGTEVVVK

SEQ ID NO: 341 - protein, 108 aa, Oryctolagus cuniculus
AFELTQTPSSVSEPVGGTVTIKCQASENIYSSLAWYQQKPGQPPKLLIYQASTLASGVSSRFRGSGSGTQFTLTISDLECADAATYYCQNYYSTDSGAFGGGTEVVVK

SEQ ID NO: 342 - protein, 111 aa, Oryctolagus cuniculus
AFELTQTPSSVEAAVGGTVTINCQASQSISSYLAWYQQKPGQPPKLLIYDASKLASGVPSRFSGSGSGTEYTLTISDLECDDAATYYCQSAYYGSSSTVNNFGGGTEVVVK

SEQ ID NO: 343 - protein, 111 aa, Oryctolagus cuniculus
AFELTQTPASVEAAVGGTVTIKCQASQSISTYLAWYQQKPGQPPKLLIYGASNLESGVPSRFTGSGSGTEFTLTISDLECDDAATYYCQSAYYGSSSTVNTFGGGTEVVVK

SEQ ID NO: 344 - protein, 111 aa, Oryctolagus cuniculus
IEMTQTPFSVSAALGGTVTINCQASDFIYGNLAWYQQKPGQPPKLLIYDASDLASGVPSRFKGSGSGTEYTLTISGVQCADAATYYCQSAYYSTSADMRNAFGGGTEVVVK

SEQ ID NO: 345 - protein, 111 aa, Oryctolagus cuniculus
ADVVMTQTPASVEAAVGGTVTINCQASQNIRSYLVWYQQKPGQRPELLIYRASNLESGVPSRFSGSGSGTEFTLTISDLECADAATYYCQNYYNIANYGNAFGGGTEVVVK

SEQ ID NO: 346 - protein, 110 aa, Oryctolagus cuniculus
QIVMTQTPASVSDPVGGTVTIKCQASQSISSYLAWYQQKPGQPPKQLIYAASTLASGVPSRFKGSGSGTEFTLTISDLECADAATYYCQQGYATTYVDNPFGGGTEVVVK

SEQ ID NO: 347 - protein, 107 aa, Oryctolagus cuniculus
DSVMTQTLASVSEPVGGTVTIKCQASHNIYSNLAWYQQRPGQRPKLLIYDASKLASGVPSRFKGSGSGTQFTLTISDLECDDAATYYCQSYYGNSYPFGGGTEVVVK

SEQ ID NO: 348 - protein, 110 aa, Oryctolagus cuniculus
DVVMTQTPASVSEPVGGTVTIKCQASQNIGSELAWYQQKPGQPPKLLIYVASYLASGVSSRFKGSGSGTDFTLTISDLECADAATYYCQCTYYGGIPIGTFGGGTEVVVK

SEQ ID NO: 349 - protein, 110 aa, Oryctolagus cuniculus
ADVVMTQTPASVSDPVGGTVTIKCQASQNIGSNLAWYQQKPGQRPKLLIYDASKLASGVPSRFSGSGYGTRFTLTISDLECADAATYYCQCTYYNGIDYVFGGGTEVVVK

SEQ ID NO: 350 - protein, 111 aa, Oryctolagus cuniculus
ADVVMTQTPASVSEPVGGTVTIKCQASQSISSYLSWYQQKPGQPPKLLMYKASTLASGVSSRFKGSESGTEFTLTISDLECADAATYYCQNYYIIGIDGGAFGGGTEVVVK

SEQ ID NO: 351 - protein, 114 aa, Oryctolagus cuniculus
ADIVMTQTPASVEAAVGGTVTIKCQASQSIGSYLNWYQQKPGQPPKLLIYYTSNLASGVSSRFSGSGSGTQFTLTISDLECADAATYYCQSYYYSSTNSSYSWPFGGGTEVVVK

SEQ ID NO: 352 - protein, 113 aa, Oryctolagus cuniculus
ADIVMTQTPASVEAAVGGTVTIKCQASQTISSFLAWYQQKPGQPPKLLIYYASTLASGVPSRFKGSRSETQFTLTISDLECADAATYYCQSYYDSSSYNYAWAFGGGTEVVVK

SEQ ID NO: 353 - protein, 110 aa, Oryctolagus cuniculus
AIKMTQTPSSVSAAVGGTVTINCRASEDIESYLAWYQQKPGQRPKLQIYAASTLASGVPSRFRGSGSGTEYTLTISGVQCDDAATYYCQSAYYSSSADNAFGGGTEVVVK

SEQ ID NO: 354 - protein, 110 aa, Oryctolagus cuniculus
AIKMTQTPSSVSAAVGGTVTINCRASEDIESYLAWYQQKPGQRPKLLIYAASTLASGVPSRFKGSGSGTEYTLTISGVQCDDAATYYCQSAYYSSSADNAFGGGTEVVVK

SEQ ID NO: 355 - protein, 109 aa, Oryctolagus cuniculus
IKMTQTPSSVSAAVGGTVTINCQASEDIESYLAWYQQKPGQRPKLQIYAASTLASGVPSRFRGSGSGTEYTLTISGVQCDDAATYYCQSAYYSSSADNAFGGGTEVVVK

SEQ ID NO: 356 - protein, 109 aa, Oryctolagus cuniculus
IKMTQTPSSVSAAVGGTVTINCRASEDIESYLAWYQQKPGQRPKLLIYSASTLASGVPSRFKGSGSGTEYTLTISGVQCDDAATYYCQSAYYSSSADNAFGGGTEVVVK

SEQ ID NO: 357 - protein, 111 aa, Oryctolagus cuniculus
ADIVMTQTPASVSEPVGGTVTIKCQASQSISSYLSWYQQKSGQPPKLLIYYASNLASGVPSRFSGSGSGTEFTLTISGVQCDDAATYYCQSYYYSNNYNWAFGGGTEVVVK

SEQ ID NO: 358 - protein, 114 aa, Oryctolagus cuniculus
ADIVMTQTPASVSEPVGGTVTIKCQASQSISSYLSWYQQKPGQRPKLLIYYSSTLASGVSSRFSGSGSGTEFTLTISDLECDDAATYYCQGYYYDRDSSSYGWAFGGGTEVVVK

SEQ ID NO: 359 - protein, 114 aa, Oryctolagus cuniculus
ADIVMTQTPASVEAAVGGTVTIKCQASQSISSYLSWYQQKPGQRPKLLIYYSSTLASGVSSRFSGSGSGTEFTLTISDLECDDAATYYCQGYYYNRDSSSYGWAFGGGTEVVVK

SEQ ID NO: 360 - protein, 111 aa, Oryctolagus cuniculus
ADIVVTQTPASVEAAVGGTVTIKCQASQSISSYLSWYQQKPGQPPKVLIYYASTLASGVPSWFKGSGSGTEFTLTISDLECADAATYYCQSYDWDSSPTYTFGGGTEVVVK

SEQ ID NO: 361 - protein, 111 aa, Oryctolagus cuniculus
ADIVMTQTPASVEAAVGGTVTMKCQASQSISNYCSWYQQKPGQPPKLLIYYASTLASGVPSRFKGSGSGTQFTLTISDLECDDAATYYCQSYDWNSSPTYAFGGGTEVVVK

SEQ ID NO: 362 - protein, 111 aa, Oryctolagus cuniculus
ADIVMTQTPASVEAAVGGTVTMKCQASQSISNYCSWYQQKPGQPPKLLIYYASTLASGVPSRFKGSGSGTQFTLTISDLECADAATYYCQSYDWNSSPTYAFGGGTEVVVK

SEQ ID NO: 363 - protein, 111 aa, Oryctolagus cuniculus
ADIVMTQTPASVEAAVGGTVTIKCQASQTISSYLSWYQQKPGQPPKLLIYYASTLASGVPSRFSGSGSGTQFTLTISDLECADAATYYCQSYDWNSSPSYVFGGGTEVVVK

SEQ ID NO: 364 - protein, 110 aa, Oryctolagus cuniculus
AYDMTQTPASVSAAVGGTVTIKCQASQSIDSWLSWYQQKPGQPPKLLIYRASTLASGVPSRFKGSGSGTEYSLTISGVECADAATYYCQQGYMTNNVDNAFGGGTEVVVK

SEQ ID NO: 365 - protein, 110 aa, Oryctolagus cuniculus
AYDMTQTPASVSAAVGGTVTIKCQASQSIDSWLSWYQQKPGQPPKLLIYKASTLASGVPSRFKGSGSGTEYSLTISGVECAGAATYYCQQGYMTNNVDNAFGGGTEVVVK

SEQ ID NO: 366 - protein, 110 aa, Oryctolagus cuniculus
AYDMTQTPASVSEPVGGTVTINCQASESISSYLSWYQQKSGQPPKLLIYRASDLASGVPSRFKGSGSGTEFTLTISDLECADAATYYCQQGYNTNLVDNAFGGGTEVVVK

SEQ ID NO: 367 - protein, 110 aa, Oryctolagus cuniculus
QIVMTQTPASVSAAVGGTVTIKCQASQSIDSWLAWYQQKPGQHPRLLIYRASTLASGVSSRFKGSGSGTEFTLTISDLECADAATYYCQQGYAISYVHNVFGGGTEVVVK

SEQ ID NO: 368 - protein, 110 aa, Oryctolagus cuniculus
QIVMTQTPASVSAAVGGTVTIKCQARQSIDSWLAWYQQKPGQHPRLLIYRASTLASGVSSRFKGSGSGTEFTLTISDLECADAATYYCQQGYGISYVHNVFGGGTEVVVK

SEQ ID NO: 369 - protein, 110 aa, Oryctolagus cuniculus
QIVMTQTPASVSAAVGGTVTIKCQASQSIGSWLAWYQQKPGQHPKLLIYRASTLASGVSSRFKGSGSGTQFTLTISGVECADAATYYCQQGYAISYVDNVFGGGTEVVVK

SEQ ID NO: 370 - protein, 110 aa, Oryctolagus cuniculus
AIDMTQTPASVEAAVGGTVTVKCQASQSISTYLNWYQQKPGQPPKLLIYRASTLASGVSSRFKGSGSGTQFTLTISGVECADAATYYCQQGYSNTNLDNSFGGGTEVVVK

SEQ ID NO: 371 - protein, 110 aa, Oryctolagus cuniculus
AIDMTQTPASVEAAVGGTVTVKCQASQSISSYLNWYQQKPGQPPKLLIYRASTLASGVSSRFKGSGSGTQFTLTISGVECADAATYYCQQGYSNTNLYNSFGGGTEVVVK

SEQ ID NO: 372 - protein, 110 aa, Oryctolagus cuniculus
AIDMTQTPASVEAAVGGTVTISCQASQSISSYLNWYQQKPGQPPKLLIYRASTLASGVSSRFKGSGSGTQFTLTISGVECADAATYYCQQGYSNTNVDNTFGGGTEVVVK

SEQ ID NO: 373 - protein, 112 aa, Oryctolagus cuniculus
ADVVMTQTPSSVSAAVGGTVTINCQSSQSVYTNYLSWYQQKPGQPPKLLIYAASTLASGVPSRFKGSGSGTQFTLTISGVQCDDAAAYYCLGSYVSSGWYYAFGGGTEVVVK

SEQ ID NO: 374 - protein, 110 aa, Oryctolagus cuniculus
ALVMTQTPASVEAAVGGTVTINCQASQNIGSWLAWYQQKPGQPPKLLIYDASKLASGVPSRFKGSGSGTEFTLTISGVQCDDAATYYCQQGYATSYIDNAFGGGTEVVVK

SEQ ID NO: 375 - protein, 110 aa, Oryctolagus cuniculus
ALVMTQTPASVEAAVGGTVTINCQASQNIGSWLAWYQQKPGQPPKLLIYQASKLSSGVPSRFSGSGSGTEFTLTISGVQCDDAATYYCQQGYASSYIDNAFGGGTEVVVK

SEQ ID NO: 376 - protein, 110 aa, Oryctolagus cuniculus
ALVMTQTPASVEAAVGGTVTINCQASQNIGSWLAWYQQKPGQPPKLLIYQASKLSSGVPSRFSGSGSGTEFTLTISGVQCDDAATYYCQQGYATSYIDNAFGGGTEVVVK

SEQ ID NO: 377 - protein, 110 aa, Oryctolagus cuniculus; identical to SEQ ID NO: 376

SEQ ID NO: 378 - protein, 110 aa, Oryctolagus cuniculus; identical to SEQ ID NO: 376

SEQ ID NO: 379 - protein, 110 aa, Oryctolagus cuniculus; identical to SEQ ID NO: 376

SEQ ID NO: 380 - protein, 110 aa, Oryctolagus cuniculus
ALVMTQTPASVEAAVGGTVTINCQASQNIGSWLAWYQQKPGQPPKLLIYQASKLSSGVPSRFSGSGSGTEFTLTISGVQCDDAATYYCQQGYATSYIDNAFGGGNEVVVK

SEQ ID NO: 381 - protein, 110 aa, Oryctolagus cuniculus
QIVMTQTPASVSAAVGGTVTINCQASQSINSWLSWYQQKPGQRPKLLIYSASTLASGVSSRFRGSGSGTEFTLTISDLECDDAATYYCQQCYGVSFVDNTFGGGTEVVVK

SEQ ID NO: 382 - protein, 110 aa, Oryctolagus cuniculus
ALVMTQTPSPVSAAVGGTVTISCQASQSISNWLAWYQQKAGQRPKLLIYYTSNLASGVPSRFSGSGSGTQFTLTISDVQCADAATYNCAGYKSYSNDDNGFGGGTEVVVK

SEQ ID NO: 383 - protein, 111 aa, Oryctolagus cuniculus
DVVMTQTPASVEATVGGTVTIKCQASESIGNYLAWYQQKPGQPPKLLIYGASTLESGVPSRFSGSGSGTEFTLTISDLECADAATYYCQCTYGTTSISTYTFGGGTEVVVK

SEQ ID NO: 384 - protein, 110 aa, Oryctolagus cuniculus
DVVMTQTPASVEAAVGGTVTINCQASQSIGSNLAWYEQKPGQPPKLLIYTASTLESGVPSRFKGSGSGTEFTLTISDLECADAATYYCQCTYGSSSSGYPFGGGTEVVVK

SEQ ID NO: 385 - protein, 110 aa, Oryctolagus cuniculus
AYDMTQTPSSVSEPVGGTVTINCQASESIYNYLSWYQQKPGQPPKLLIYGASNLESGVPSRFKGSGSGTEYTLTISDLECADAATYFCQQGYSGNNIDNIFGGGTEVVVK

SEQ ID NO: 386 - protein, 113 aa, Oryctolagus cuniculus
DVVMTQTPASVEAAVGGTVTIKCQASEDIESYLAWYQQKPGQRPKLLIYGASNLESGVSSRFKGSGSGTEYTLTISDLECADAATYYCQCTYGSSTTSNYGDAFGGGTEVVVK

SEQ ID NO: 387 - protein, 114 aa, Oryctolagus cuniculus
ADIVMTQTPASVEAAVGGTVTIKCQASQSIDSNLAWYQQKPGQPPKLLIYRASGLESGVPSRFRGSGSGTEFTLTISDLECADAATYYCQCTYGSNDSGNYANAFGGGTEVVVK

SEQ ID NO: 388 - protein, 109 aa, Oryctolagus cuniculus
AFELTQTPSSVEAAVGGTVTIKCQASQSISNYLAWYQQKPGQPPKLLIYGASTLASGVSSRFKGSGSGTEFTLTISDLECADAATYACQTTYANTIGWAFGGGTEVVVK

SEQ ID NO: 389 - protein, 112 aa, Oryctolagus cuniculus
ADIVMTQTPGSVEAAVGGTVAIKCQASQYINGYLSWYQQKPGQPPKLLIYRASTLASGVSSRFKGSGSGTEYTLTISDLECADAATFYCQSYYFNGGTLINPFGGGTEVVVK

SEQ ID NO: 390 - protein, 112 aa, Oryctolagus cuniculus; identical to SEQ ID NO: 389

SEQ ID NO: 391 - protein, 111 aa, Oryctolagus cuniculus
AAVLTQIPSSVSAGVGGTVTISCQSSPSVASNYLSWYQQKPGQRPKLLIYAASTLASGVPSRFKGSGSGTQFTLTISDVQCADAATYYCAGGYTGNIDTFVFGGGTEVVVK

SEQ ID NO: 392 - protein, 111 aa, Oryctolagus cuniculus
AAVLTQTPSSVSAGVGGTVTISCQSSPSVASNYLSWYQQKPGQRPKLLIYAASTLASGVPSRFKGSGSGTQFTLTISDVQCADAATYYCAGGYTGNIDTFVFGGGTEVVVK

SEQ ID NO: 393 - protein, 112 aa, Oryctolagus cuniculus
DVVMTQTPASVEAAVGGTVTIKCQASQSINSWFSWYQQKPGQRPKLLIYAAANLASGVPSRFKGSRSGTEYTLTISDLECGDAATYYCQCNAGSDIDSNGDAFGGGTEVVVK

SEQ ID NO: 394 - protein, 112 aa, Oryctolagus cuniculus; identical to SEQ ID NO: 393

SEQ ID NO: 395 - protein, 109 aa, Oryctolagus cuniculus
DIVMTQTPASVEAAVGGTVTINCQASQNTGGWLAWYQQRPGQRPKLLIYEASKLASGVPSRFKGGGAGTEFTLNISDLECADAATYYCQYTYGGSGGYGFGGGTEVVVK

SEQ ID NO: 396 - protein, 110 aa, Oryctolagus cuniculus
AFELTQAPSSVEAAVGGTVTINCQASQSILYFLAWYQQKPGQRPKLLIYDASDLASGVPSRFSGSGSGTEFTLTISDLECDDAAIYYCQSVWYSSGAANIFGGGTEVVVK

SEQ ID NO: 397 - protein, 110 aa, Oryctolagus cuniculus
AQVLTQTSSPVSAAVGGTVTINCKTSQSVYDNNALAWYQQKPGQPPKLLIHTASTLASGVPSRFRGSGSGTQFTLTISDLECDDAATYYCGGDFNGYIYDYGGGTEVVVK

SEQ ID NO: 398 - protein, 110 aa, Oryctolagus cuniculus
AQVLTQTASPVSAAVGGTITIKCQSSQSVYDNNALAWYQQKPGQPPKLLIHTASTLASGVPSRFKGSGSGTEFTLTISDLECDDAATYYCAGDFSGYIYDYGGGTEVVVK

SEQ ID NO: 399 - protein, 109 aa, Oryctolagus cuniculus
DIVMTQTPASVEAAVGGTVTINCQASQNTGGWLAWYQKKPGQRPKLLIYEASKLTSGVPSRFKGGGSGTEFTLNISDLECADAAAYYCQYTYGGSGGYGFGGGTEVVVK

SEQ ID NO: 400 - protein, 109 aa, Oryctolagus cuniculus
DIVMTQTPASVEAAVGGTVTINCQASQNTGGWLAWYQQKPGQRPKLLIYEASKLASGVPSRFKGGGAGTEFTLNISDLECADAATYYCQYTYGGSGGYGFGGGTEVVVK

SEQ ID NO: 401 - protein, 110 aa, Oryctolagus cuniculus; identical to SEQ ID NO: 398

SEQ ID NO: 402 - protein, 109 aa, Oryctolagus cuniculus
DIVMTQTPASVEAAVGGTVTINCQASQNTGGWLAWYQQKPGQPPKLLIYEASKLASGVPSRFKGSGAGTEFTLNISDLECADAAAYYCQYTYGGSGGYGFGGGTEVVVK

SEQ ID NO: 403 - protein, 112 aa, Oryctolagus cuniculus
AQVLTQTASPVSAAVGGTVTINCQSSQSLSNAHVAWYQQKPGQPPKLLIYFASRLASGVSSRFTGSGSGTQFTLTISDLECDDAATYYCQGEFDCSHGDCDAFGGGTEVVVR

SEQ ID NO: 404 - protein, 110 aa, Oryctolagus cuniculus
AFELTQAPSSVEAAVGGTVTINCQASQNILYFLAWYQQKPGQRPKLLIYDASNLASGVPSRFSGSGSGTEFTLTISDLECDDAAIYYCQSVWYSSGAANLFGGGTEVVVK

SEQ ID NO: 405 - protein, 112 aa, Oryctolagus cuniculus
AIVMTQTPSSKSVPVGDTVTISCQASESVYSDNRLAWFQQKPGQRPKLLIYYASTLASGVPSRFKGSGSGTQFTLTINDVVCDDAATYYCAGYKSDSPDGFAFGGGTEVVVK

SEQ ID NO: 406 - protein, 109 aa, Oryctolagus cuniculus
DIVMTQAPASVEAAMGDTVTINCQASQNTGGWLAWYQQKAGQRPKLLIYEASKLASGVPSRFKGGGAGTEFTLHISDLECADAATYYCQYTYGGSGGYGFGGGTEVVVK

SEQ ID NO: 407 - protein, 110 aa, Oryctolagus cuniculus
AFELTQAPSSVEAAVGGTVTINCQASQNILYFLAWYQQKPGQRPKLLIYDASNLASGVPSRFSGSGSGTEFTLTISDLECADAAIYYCQSVWYGSGAANLFGGGTEVVVT

SEQ ID NO: 408 - protein, 109 aa, Oryctolagus cuniculus; identical to SEQ ID NO: 400

SEQ ID NO: 409 - protein, 109 aa, Oryctolagus cuniculus; identical to SEQ ID NO: 400

SEQ ID NO: 410 - protein, 109 aa, Oryctolagus cuniculus
DIVMTQTPASVEAAVGGTVTINCQASQNTGGWLAWYQQKPGQRPQLLIYEASKLASGVPSRFKGGGSGTDFTLTISDLECADAATYYCQYTYGGSGGYGFGGGTEVVVK

SEQ ID NO: 411 - protein, 108 aa, Oryctolagus cuniculus
AFELTQTPSSVEAAVGGTVSIKCQASQSIGAYFAWYQQKPGQPPKLLIHSASTLASGVSSRFKGSGSGTEFTLTISDLECADAATYYCQSYYATSTNTFGGGTEVVVK

SEQ ID NO: 412 - protein, 110 aa, Oryctolagus cuniculus
AVVMTQTASPVSEPVGGTVTINCQASENIYSSLAWYQQKPGQPPKLLIYDASDLASGVPSRFKGSGSGTDFTLTISGVQCDDAATYYCQSYYGGTNVGYSFGGGTEVVVK

SEQ ID NO: 413 - protein, 14 aa, Artificial Sequence
  Spacer sequence
EGKSSGSGSESKVD

SEQ ID NO: 414 - protein, 18 aa, Artificial Sequence
  Spacer sequence
KESGSVSSEQLAQFRSLD

SEQ ID NO: 415 - protein, 5 aa, Artificial Sequence
  Spacer sequence
GGGGS

SEQ ID NO: 416 - protein, 342 aa, Homo sapiens
EEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDHVEFKSGAGTELSVRAKPSAPVVSGPAARATPQHTVSFTCESHGFSPRDITLKWFKNGNELSDFQTNVDPAGDSVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAPGPALASAAPL